(19) United States
(12) Patent Application Publication
(10) Pub. No.: US 2025/0268906 A1
(43) Pub. Date: Aug. 28, 2025

(54) MOLECULAR GLUE COMPOUND BASED ON CEREBLON PROTEIN DESIGN AND USE THEREOF

(71) Applicant: GLUETACS THERAPEUTICS (SHANGHAI) CO., LTD., Shanghai (CN)

(72) Inventors: Xiaobao YANG, Shanghai (CN); Renhong SUN, Shanghai (CN)

(73) Assignee: GLUETACS THERAPEUTICS (SHANGHAI) CO., LTD., Shanghai (CN)

(21) Appl. No.: 19/072,374

(22) Filed: Mar. 6, 2025

Related U.S. Application Data
(63) Continuation of application No. PCT/CN2023/117399, filed on Sep. 7, 2023.

(30) Foreign Application Priority Data
Sep. 8, 2022 (CN) 202211094076.6

(51) Int. Cl. A61K 31/5377; A61K 31/4439; A61K 31/444; A61P 17/06; A61P 19/02; A61P 35/00; A61P 37/02; C07D 211/88; C07D 265/30; C07D 277/28

(52) U.S. Cl. CPC A61K 31/5377; A61K 31/4439; A61K 31/444; A61P 17/06; A61P 19/02; A61P 35/00; A61P 37/02; C07D 211/88; C07D 265/30; C07D 277/28

(57) ABSTRACT

The present disclosure relates to a compound of Formula (I) or a salt, enantiomer, diastereomer, isotopically enriched analogue, solvate, prodrug or polymorph thereof, and the use thereof. Further provided in the present disclosure are a pharmaceutical composition comprising, as an active ingredient, the compound of Formula (I) or a salt, enantiomer, diastereomer, isotopically enriched analogue, solvate, prodrug or polymorph thereof, and the use thereof. A series of compounds designed and synthesized in the present disclosure can effectively prevent and/or treat diseases or disorders associated with cereblon protein.

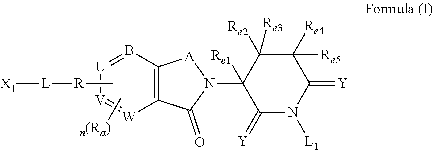

### TECHNICAL FIELD

The present disclosure generally relates to a compound of Formula (I) or salts, enantiomers, diastereoisomer, isotopically enriched analogs, solvates, prodrugs, or polymorphs thereof, and their use in the prevention and/or treatment of diseases or disorders associated with cereblon protein.

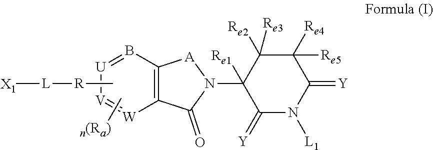

### BACKGROUND

Although Thalidomide, lenalidomide and other phthalimide immunomodulatory drugs (IMiDs) have shown significant therapeutic efficacy in the treatment of multiple myeloma and autoimmune diseases, it was not until 2010 that the E3 ubiquitin ligase cereblon (CRBN) was identified as a direct target of IMiDs. Subsequent studies have revealed that these drugs function as molecular glues, recruiting substrate proteins (such as transcription factors IKZF1/3) to CRBN, thereby facilitating protein-protein interactions between CRBN and the substrate proteins and ultimately leading to the ubiquitination of the substrate proteins. The polyubiquitinated substrate proteins are then recognized and degraded by the proteasome, resulting in pharmacological effects including antitumor and immunomodulatory activities. Molecular glue degraders directly target and degrade the target protein, offering potential advantages such as targeting “undruggable” proteins. Moreover, molecular glue degraders typically have low molecular weight and good drug-like properties, making them highly attractive for drug development. Based on the CRBN E3 ubiquitin ligase and molecular glue degradation mechanism, a series of compounds have been developed, including pomalidomide, which has been launched, and CC-122, CC-220, CC-90009, CC-99282, and CC-92480 from Bristol-Myers Squibb (BMS), DKY709 from Novartis and CFT7455 from C4 Therapeutics, which are undergoing clinical trials. The degradation substrates of these molecular glues have expanded from the initially discovered transcription factors IKZF1/3 to include casein kinase 1α (CK1α), zinc finger protein 91 (ZFP91) and translation factor GSPT1. Currently, the structural novelty of discovered molecular glue candidates remains limited, making the design of diverse scaffolds and development of novel molecular glues a key research focus in this field.

Therefore, there is an urgent need for a series of novel CRBN E3 ubiquitin ligase ligands that can serve as effective molecular glue degraders for the treatment and/or prevention of diseases or disorders mediated by or associated with degraded proteins.

### SUMMARY OF INVENTION

In view of the above, the objectives of the present disclosure are to provide novel molecular glue protein degraders, their applications and usage methods.

To achieve the above objectives and other related goals, in one aspect, the present disclosure provides a compound of Formula (I):

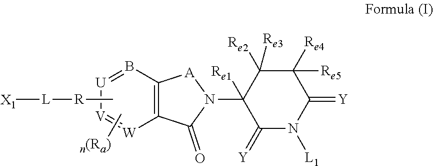

or salts, enantiomers, diastereoisomer, isotopically enriched analogs, solvates, prodrugs, or polymorphs thereof, wherein
A represents CH₂ or C(O);
B, U, V, and W are the same or different and each independently represent CH or N, wherein B, U, V, and W are not N at the same time;
Y represents O or S;
L₁ represents hydrogen or C₁₋₃ alkyl;
Rₑ₁, Rₑ₂, Rₑ₃, Rₑ₄, and Rₑ₅ are the same or different and each independently represent hydrogen or halogen;
(Rₐ)ₙ denotes that the ring containing B, U, V, and W is substituted with n Rₐ, where Rₐ groups are the same or different and each independently represent deuterium, halogen, hydroxy, mercapto, nitro, amino, cyano, optionally deuterated C₁₋₆ alkyl, optionally deuterated C₁₋₆ alkoxy, halogenated C₁₋₆alkyl, optionally substituted C₃₋₆ cycloalkyl, C₂₋₆ alkenyl, or C₂₋₆ alkynyl, and n represents an integer of 1, 2, or 3; and
R represents SH, and L and X₁ are absent; or
R represents S, L represents an optionally substituted linear or branched alkyl, and X₁ is absent; or
R represents S(O) or S(O)₂, L represents an optionally substituted linear or branched alkyl or amino, and X₁ is absent; or
R represents S, S(O), S(O)₂, O, NH or CH₂,
L represents optionally substituted linear or branched alkylene, wherein one or more (e.g., 1-15, 1-10, 1-8, 1-7, 1-6, 1-5, 1-4, 1-3, 1-2 or 1) pairs of adjacent carbon atoms in the backbone carbon chain of the linear or branched alkylene is optionally interrupted by one or more (e.g., 1-30, 1-20, 1-15, 1-10, 1-8, 1-7, 1-6, 1-5, 1-4, 1-3, 1-2 or 1) groups selected from the group consisting of: R_{b}, R_{c}, and any combination thereof, wherein R_{b} groups are each selected from the group consisting of O, C(O), OC(O), S, S(O), S(O)₂, S(O)₂N(R₁), N(R₁)S(O)₂, C(O)N(R₁), N(R₁)C(O), N(R₁), and N(R₁)C(O)N(R₁), where R₁ groups each independently represent H or C₁₋₃ alkyl, and when two or more groups R_{b} are inserted into the backbone carbon chain of the linear or branched alkylene group, the two or more groups R_{b} are not directly connected to each other; and wherein Rₑ groups are each selected from the group consisting of optionally substituted cycloalkylene, optionally substituted arylene, optionally substituted heterocyclylene, optionally substituted heteroarylene, and any combination thereof; and
X₁ represents NR₂R₃, C(O)NR₄R₅, NHC(O)R₆, NHC(O)NR₇R₈, OR₉, SR₁₀, quaternary ammonium salt group, linear or branched alkyl optionally substituted with one or more (e.g., 1-30, 1-20, 1-15, 1-10, 1-8, 1-7, 1-6, 1-5, 1-4, 1-3, 1-2 or 1) fluorine, optionally substituted cycloalkyl, optionally substituted aryl, optionally substituted heterocyclyl or optionally substituted heteroaryl, where R₂, R₃, R₄, R₅, R₇, R₈, R₉, and R₁₀ each independently represent H, optionally substituted linear or branched alkyl, optionally substituted cycloalkyl, optionally substituted aryl, optionally substituted heterocyclyl or optionally substituted heteroaryl, wherein R₂ and R₃ are not H at the same time, and wherein R₆ represents optionally substituted linear or branched alkyl, optionally substituted cycloalkyl, optionally substituted aryl, optionally substituted heteroaryl or optionally substituted heterocyclyl, or wherein R₄ and R₅ together with the nitrogen atom to which they are attached form an optionally substituted 5- to 8-membered heterocyclyl; or
X₁ represents a group of Form a (G₁)

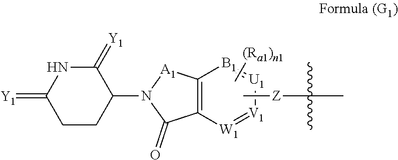

wherein A₁ represents CH₂ or C(O);
B₁, U₁, V₁, and W₁ are the same or different and each independently represent CH or N, wherein B1, U₁, V₁, and W₁ are not N at the same time;
Y₁ represents O or S; and
Z represents S, S(O), S(O)₂, O, NH or CH₂; and
(Rₐ₁)ₙ₁ denotes that the ring containing B₁, U₁, V₁, and W₁ is substituted with n1 Rₐ₁, where Rₐ₁ groups are the same or different and each independently represent deuterium, halogen, hydroxy, mercapto, nitro, amino, cyano, optionally deuterated C₁₋₆ alkyl, optionally deuterated C₁₋₆ alkoxy, halogenated C₁₋₆ alkyl, optionally substituted C₃₋₆ cycloalkyl, C₂₋₆ alkenyl, or C₂₋₆ alkynyl, and n represents an integer of 0, 1, 2, or 3.

In another aspect, the present disclosure provides a pharmaceutical composition comprising the compound of Formula (I) or pharmaceutically acceptable salts, enantiomers, diastereoisomers, isotopically enriched analogs, solvates, prodrugs, or polymorphs thereof, and at least one pharmaceutically acceptable carrier.

In a further aspect, the present disclosure further provides a medicine kit or reagent kit comprising: the compound of Formula (I) or a pharmaceutically acceptable salt, enantiomers, diastereoisomers, isotopically enriched analogs, solvates, prodrugs, or polymorphs thereof, or the pharmaceutical composition of the present disclosure.

In a further aspect, the present disclosure provides the compound of Formula (I) or pharmaceutically acceptable salts, enantiomers, diastereoisomers, isotopically enriched analogs, solvates, prodrugs, or polymorphs thereof, for use as a medicament.

In a further aspect, the present disclosure provides the compound of Formula (I) or pharmaceutically acceptable salts, enantiomers, diastereoisomers, isotopically enriched analogs, solvates, prodrugs, or polymorphs thereof, or the pharmaceutical composition of the present disclosure for use in the prevention and/or treatment of diseases or disorders associated with cereblon protein.

In a further aspect, the present disclosure provides use of the compound of Formula (I) or pharmaceutically acceptable salts, enantiomers, diastereoisomers, isotopically enriched analogs, solvates, prodrugs, or polymorphs thereof, or the pharmaceutical composition of the present disclosure for the manufacture of a medicament for the prevention and/or treatment of diseases or disorders associated with cereblon protein.

In a further aspect, the present disclosure provides a method for treating or preventing diseases or disorders associated with cereblon protein in a subject, comprising administering to the subject a therapeutically effective amount of the compound of Formula (I) or pharmaceutically acceptable salts, enantiomers, diastereoisomers, isotopically enriched analogs, solvates, prodrugs, or polymorphs thereof, or the pharmaceutical composition.

### DESCRIPTION OF DRAWINGS

FIG. 1 illustrates the degradation of substrate proteins, including IKZF1/3 proteins, by the compounds of the present invention in Human peripheral blood mononuclear cells (hPBMC).

### DETAILED DESCRIPTION OF THE INVENTION

The following detailed description is provided as exemplary specific embodiments to assist those skilled in the art in understanding and practicing the present disclosure. It should be appreciated, however, that such description is not intended to limit the scope of the present disclosure, and that various modifications and changes may be made to the specific embodiments described in the present disclosure without departing from the spirit and scope of the present disclosure. Such changes and modifications are to be understood as being included within the scope of the present invention as defined by the appended claims.

### I. Compounds

The present disclosure provides the compound of Formula (I).

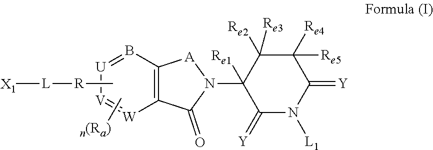

or salts, enantiomers, diastereoisomer, isotopically enriched analogs, solvates, prodrugs, or polymorphs thereof, wherein
A represents CH₂ or C(O);
B, U, V, and W are the same or different and each independently represent CH or N, wherein B, U, V, and W are not N at the same time;
Y represents O or S;
L₁ represents hydrogen or C₁₋₃ alkyl;
Rₑ₁, Rₑ₂, Rₑ₃, Rₑ₄, and Rₑ₅ are the same or different and each independently represent hydrogen or halogen (e.g., fluorine, chlorine, bromine, or iodine);
(Rₐ)ₙ denotes that the ring containing B, U, V, and W is substituted with n Rₐ, where Rₐ groups are the same or different and each independently represent deuterium, halogen, hydroxy, mercapto, nitro, amino, cyano, optionally deuterated C₁₋₆ alkyl, optionally deuterated C₁₋₆ alkoxy, halogenated C₁₋₆ alkyl, optionally substituted C₃₋₆ cycloalkyl, C₂₋₆ alkenyl, or C₂₋₆ alkynyl, and n represents an integer of 1, 2, or 3; and
R represents SH, and L and X₁ are absent; or
R represents S, L represents an optionally substituted linear or branched alkyl, and X₁ is absent; or
R represents S(O) or S(O)₂, L represents an optionally substituted linear or branched alkyl or amino, and X₁ is absent; or
R represents S, S(O), S(O)₂, O, NH or CH₂,
L represents an optionally substituted linear or branched alkylene, wherein one or more (e.g., 1-15, 1-10, 1-8, 1-7, 1-6, 1-5, 1-4, 1-3, 1-2 or 1) pairs of adjacent carbon atoms in the backbone carbon chain of the linear or branched alkylene is optionally interrupted by one or more (e.g., 1-30, 1-20, 1-15, 1-10, 1-8, 1-7, 1-6, 1-5, 1-4, 1-3, 1-2 or 1) groups selected from the group consisting of: R_{b}, R_{c}, and any combination thereof, wherein R_{b} groups are each selected from the group consisting of O, C(O), OC(O), S, S(O), S(O)₂, S(O)₂N(R₁), N(R₁)S(O)₂, C(O)N(R₁), N(R₁)C(O), N(R₁), and N(R₁)C(O)N(R₁), where R₁ groups each independently represent H or C₁₋₃ alkyl, and when two or more groups R_{b} are inserted into the backbone carbon chain of the linear or branched alkylene group, the two or more groups R_{b} are not directly connected to each other; and wherein R_{c} groups are each selected from the group consisting of optionally substituted cycloalkylene, optionally substituted arylene, optionally substituted heterocyclylene, optionally substituted heteroarylene, and any combination thereof; and
X₁ represents NR₂R₃, C(O)NR₄R₅, NHC(O)R₆, NHC(O)NR₇R₈, OR₉, SR₁₀, quaternary ammonium salt group, linear or branched alkyl optionally substituted with one or more (e.g., 1-30, 1-20, 1-15, 1-10, 1-8, 1-7, 1-6, 1-5, 1-4, 1-3, 1-2 or 1) fluorine atoms, optionally substituted cycloalkyl, optionally substituted aryl, optionally substituted heterocyclyl or optionally substituted heteroaryl, where R₂, R₃, R₄, R₅, R₇, R₈, R₉, and R₁₀ each independently represent H, optionally substituted linear or branched alkyl, optionally substituted cycloalkyl, optionally substituted aryl, optionally substituted heterocyclyl or optionally substituted heteroaryl, wherein R₂ and R₃ are not H at the same time, and wherein R₆ represents optionally substituted linear or branched alkyl, optionally substituted cycloalkyl, optionally substituted aryl, optionally substituted heteroaryl or optionally substituted heterocyclyl, or wherein R₄ and R₅ together with the nitrogen atom to which they are attached form optionally substituted 5- to 8-membered heterocyclyl; or
X₁ represents a group of Formula (G₁):

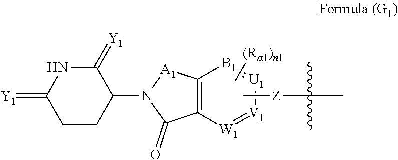

wherein A₁ represents CH₂ or C(O);
B₁, U₁, V₁, and W₁ are the same or different and each independently represent CH or N, wherein B₁, U₁, V₁, and W₁ are not N at the same time;
Y₁ represents O or S; and
Z represents S, S(O), S(O)₂, O, NH or CH₂; and
(Rₐ₁)ₙ₁ denotes that the ring containing B₁, U₁, V₁, and W₁ is substituted with n1 Rₐ₁, where Rₐ₁ groups are the same or different and each independently represent deuterium, halogen, hydroxy, mercapto, nitro, amino, cyano, optionally deuterated C₁₋₆ alkyl, optionally deuterated C₁₋₆ alkoxy, halogenated C₁₋₆ alkyl, optionally substituted C₃₋₆ cycloalkyl, C₂₋₆ alkenyl, or C₂₋₆ alkynyl, and n represents an integer of 0, 1, 2, or 3.

The compounds of Formula (I), or salts (including pharmaceutically acceptable salts), stereoisomers (including enantiomers), solvates, isotopically enriched analogs, prodrugs, or polymorphs thereof of the present disclosure, exhibit strong binding affinity to CRBN. The compounds of Formula (I), or salts (including pharmaceutically acceptable salts), stereoisomers (including enantiomers), solvates, isotopically enriched analogs, prodrugs, or polymorphs thereof of the present disclosure are also capable of degrading substrate proteins (e.g., IKZF1/2/3/4 proteins, WEE1 protein, CK1α protein, GSPT1 protein, etc.). The compounds of Formula (I), or salts (including pharmaceutically acceptable salts), stereoisomers (including enantiomers), solvates, isotopically enriched analogs, prodrugs, or polymorphs thereof of the present disclosure can effectively prevent and/or treat cereblon protein-associated diseases or disorders, including tumors, autoimmune diseases, and inflammatory diseases, or exhibit excellent pharmacokinetic properties, and can be used as therapeutic agents for cancer patients or patients with autoimmune diseases.

The present disclosure provides the compound of Formula (I-1):

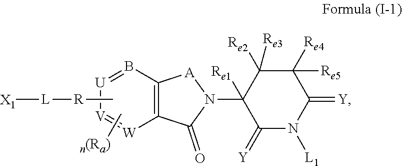

or salts, enantiomers, diastereoisomer, isotopically enriched analogs, solvates, prodrugs, or polymorphs thereof, wherein A, B, U, V, W, Y, L₁, Rₑ₁, Rₑ₂, Rₑ₃, Rₑ₄, Rₑ₅, and (Rₐ)ₙ are as defined in the compounds of Formula (I) above and its various sub-embodiments described herein; and R represents SH, and L and X₁ are absent.

The present disclosure provides the compound of Formula (I-2):

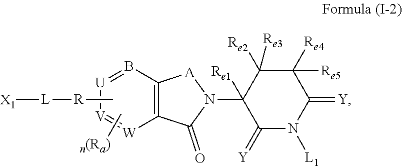

or salts, enantiomers, diastereoisomer, isotopically enriched analogs, solvates, prodrugs, or polymorphs thereof, wherein A, B, U, V, W, Y, L₁, Rₑ, and (Rₐ)ₙ are as defined in the compounds of Formula (I) above and its various sub-embodiments described herein; and R represents S, L represents an optionally substituted linear or branched alkyl, and X₁ is absent.

The present disclosure provides the compound of Formula (I-3):

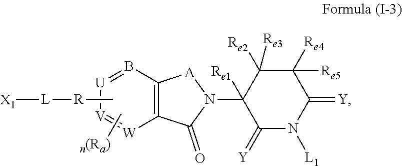

or salts, enantiomers, diastereoisomer, isotopically enriched analogs, solvates, prodrugs, or polymorphs thereof, wherein A, B, U, V, W, Y, L₁, Rₑ, and (Rₐ)ₙ are as defined in the compounds of Formula (I) above and its various sub-embodiments described herein; and R represents S(O) or S(O)₂, L represents an optionally substituted linear or branched alkyl or amino, and X₁ is absent.

The present disclosure provides the compound of Formula (I-4):

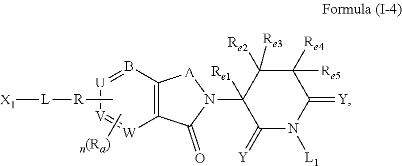

or salts, enantiomers, diastereoisomer, isotopically enriched analogs, solvates, prodrugs, or polymorphs thereof, wherein
A, B, U, V, W, Y, L₁, Rₑ, and (Rₐ)ₙ are as defined in the compounds of Formula (I) above and its various sub-embodiments described herein; and
R represents S, S(O), S(O)₂, O, NH or CH₂,
L represents optionally substituted linear or branched alkylene, wherein one or more (e.g., 1-15, 1-10, 1-8, 1-7, 1-6, 1-5, 1-4, 1-3, 1-2 or 1) pairs of adjacent carbon atoms in the backbone carbon chain of the linear or branched alkylene is optionally interrupted by one or more (e.g., 1-30, 1-20, 1-15, 1-10, 1-8, 1-7, 1-6, 1-5, 1-4, 1-3, 1-2 or 1) groups selected from the group consisting of: R_{b}, R_{c}, and any combination thereof, wherein R_{b} groups are each selected from the group consisting of O, C(O), OC(O), S, S(O), S(O)₂, S(O)₂N(R₁), N(R₁)S(O)₂, C(O)N(R₁), N(R₁)C(O), N(R₁), and N(R₁)C(O)N(R₁), where R₁ groups each independently represent H or C₁₋₃ alkyl, and when two or more groups R_{b} are inserted into the backbone carbon chain of the linear or branched alkylene group, the two or more groups R_{b} are not directly connected to each other; and wherein R_{c} groups are each selected from the group consisting of optionally substituted cycloalkylene, optionally substituted arylene, optionally substituted heterocyclylene, optionally substituted heteroarylene, and any combination thereof; and
X₁ represents NR₂R₃, C(O)NR₄R₅, NHC(O)R₆, NHC(O)NR₇R₈, OR₉, SR₁₀, quaternary ammonium salt group, linear or branched alkyl optionally substituted with one or more (e.g., 1-30, 1-20, 1-15, 1-10, 1-8, 1-7, 1-6, 1-5, 1-4, 1-3, 1-2 or 1) fluorine atoms, optionally substituted cycloalkyl, optionally substituted aryl, optionally substituted heterocyclyl or optionally substituted heteroaryl, where R₂, R₃, R₄, R₅, R₇, R₈, R₉, and R₁₀ each independently represent H, optionally substituted linear or branched alkyl, optionally substituted cycloalkyl, optionally substituted aryl, optionally substituted heterocyclyl or optionally substituted heteroaryl, wherein R₂ and R₃ are not H at the same time, and wherein R₆ represents optionally substituted linear or branched alkyl, optionally substituted cycloalkyl, optionally substituted aryl, optionally substituted heteroaryl or optionally substituted heterocyclyl, or wherein R₄ and R₅ together with the nitrogen atom to which they are attached form optionally substituted 5- to 8-membered heterocyclyl; or
X₁ represents a group of Formula (G₁):

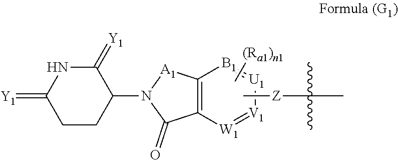

wherein A₁ represents CH₂ or C(O);
B₁, U₁, V₁, and W₁ are the same or different and each independently represent CH or N, wherein B₁, U₁, V₁, and W₁ are not N at the same time;
Y₁ represents O or S; and
Z represents S, S(O), S(O)₂, O, NH or CH₂; and
(Rₐ₁)ₙ₁ denotes that the ring containing B₁, U₁, V₁, and W₁ is substituted with n1 Rₐ₁, where Rₐ₁ groups are the same or different and each independently represent deuterium, halogen, hydroxy, mercapto, nitro, amino, cyano, optionally deuterated C₁₋₆ alkyl, optionally deuterated C₁₋₆ alkoxy, halogenated C₁₋₆alkyl, optionally substituted C₃₋₆ cycloalkyl, C₂₋₆ alkenyl, or C₂₋₆ alkynyl, and n represents an integer of 0, 1, 2, or 3.

In some embodiments of the compounds of Formula (I), Formula (I-1), Formula (I-2), Formula (I-3), and Formula (I-4) of the present disclosure, B, U, V, and W are identical, each being CH.

In some embodiments of the compounds of Formula (I), Formula (I-1), Formula (I-2), Formula (I-3), and Formula (I-4) of the present disclosure, one of B, U, V, and W is N, and the remaining are CH.

In some embodiments of the compounds of Formula (I), Formula (I-1), Formula (I-2), Formula (I-3), and Formula (I-4) of the present disclosure, two of B, U, V, and W are N, and the remaining are CH.

In some embodiments of the compounds of Formula (I), Formula (I-1), Formula (I-2), Formula (I-3), and Formula (I-4) of the present disclosure, three of B, U, V, and W are N, and the remaining is CH.

In some embodiments of the compounds of Formula (I), Formula (I-1), Formula (I-2), Formula (I-3), and Formula (I-4) of the present disclosure, Y represents O.

In some embodiments of the compounds of Formula (I), Formula (I-1), Formula (I-2), Formula (I-3), and Formula (I-4) of the present disclosure, Y represents S.

In some embodiments of the compounds of Formula (I), Formula (I-1), Formula (I-2), Formula (I-3), and Formula (I-4) of the present disclosure, A represents C(O).

In some embodiments of the compounds of Formula (I), Formula (I-1), Formula (I-2), Formula (I-3), and Formula (I-4) of the present disclosure, A represents CH₂.

In some embodiments of the compounds of Formula (I), Formula (I-1), Formula (I-2), Formula (I-3), and Formula (I-4) of the present disclosure, L₁ represents hydrogen.

In some embodiments of the compounds of Formula (I), Formula (I-1), Formula (I-2), Formula (I-3), and Formula (I-4) of the present disclosure, L₁ represents C₁₋₃ alkyl (e.g., methyl).

In some embodiments of the compounds of Formula (I), Formula (I-1), Formula (I-2), Formula (I-3), and Formula (I-4) of the present disclosure, one of Rₑ₁, Rₑ₂, Rₑ₃, Rₑ₄, and Rₑ represents halogen (e.g., fluorine), and the remaining represent hydrogen.

In some embodiments of the compounds of Formula (I), Formula (I-1), Formula (I-2), Formula (I-3), and Formula (I-4) of the present disclosure, two of Rₑ₁, Rₑ₂, Rₑ₃, Rₑ₄, and Rₑ₅ represent halogen (e.g., fluorine), and the remaining represent hydrogen.

In some embodiments of the compounds of Formula (I), Formula (I-1), Formula (I-2), Formula (I-3), and Formula (I-4) of the present disclosure, three of Rₑ₁, Rₑ₂, Rₑ₃, Rₑ₄, and Rₑ₅ represent halogen (e.g., fluorine), and the remaining represent hydrogen.

In some embodiments of the compounds of Formula (I), Formula (I-1), Formula (I-2), Formula (I-3), and Formula (I-4) of the present disclosure, (Rₐ)ₙ denotes that the ring containing B, U, V, and W is substituted with n Rₐ, where Rₐ groups are the same or different and each independently represent deuterium, halogen, hydroxy, mercapto, nitro, amino, cyano, optionally deuterated C₁₋₆ alkyl, optionally deuterated C₁₋₆ alkoxy, halogenated C₁₋₆ alkyl, optionally substituted C₃₋₆ cycloalkyl, C₂₋₆ alkenyl, or C₂₋₆ alkynyl, and n represents an integer of 1, 2, or 3.

In some embodiments of the compounds of Formula (I) and Formula (I-2) of the present disclosure, R in Formula (I) represents S, L represents an optionally substituted linear or branched alkyl, and X₁ is absent. In a sub-embodiment, R represents S, L represents an optionally substituted linear or branched C₁₋₄₀ alkyl (especially C₁₋₃₀ alkyl), and X₁ is absent. In a sub-embodiment, R represents S, L represents the following optionally substituted groups: methyl, ethyl, n-propyl, isopropyl, n-butyl, isobutyl, sec-butyl, tert-butyl, pentyl, isopentyl, neopentyl, tert-amyl, hexyl, heptyl, octyl, nonyl, decyl, undecyl, dodecyl, tridecyl, tetradecyl, or pentadecyl, and X₁ is absent. In a sub-embodiment, L represents an optionally substituted linear or branched alkyl, which is optionally substituted by one or more (e.g., 1-30, 1-20, 1-15, 1-10, 1-8, 1-7, 1-6, 1-5, 1-4, 1-3, 1-2 or 1) substituents selected from the group consisting of halogen, cyano, hydroxy, amino, mercapto, or any combination thereof.

In some embodiments of the compounds of Formula (I) and Formula (I-3) of the present disclosure, R in Formula (I) represents S(O) or S(O)₂, L represents an optionally substituted linear or branched alkyl or amino, and X₁ is absent. In a sub-embodiment, R represents S(O) or S(O)₂, L represents an optionally substituted linear or branched C₁₋₄₀ alkyl (especially C₁₋₃₀ alkyl) or amino, and X₁ is absent. In a sub-embodiment, R represents S(O) or S(O)₂, L represents the following optionally substituted groups: methyl, ethyl, n-propyl, isopropyl, n-butyl, isobutyl, sec-butyl, tert-butyl, pentyl, isopentyl, neopentyl, tert-amyl, hexyl, heptyl, octyl, nonyl, decyl, undecyl, dodecyl, tridecyl, tetradecyl, pentadecyl, or amino, and X₁ is absent. In a sub-embodiment, L represents an optionally substituted linear or branched alkyl, which is optionally substituted by one or more (e.g., 1-30, 1-20, 1-15, 1-10, 1-8, 1-7, 1-6, 1-5, 1-4, 1-3, 1-2 or 1) substituents selected from the group consisting of halogen, cyano, hydroxy, amino, mercapto, or any combination thereof.

In some embodiments of the compounds of Formula (I) and Formula (I-1) of the present disclosure, R represents SH, and L and X₁ are absent.

In some embodiments of the compounds of Formula (I) and Formula (I-4) of the present disclosure,
R represents S, S(O), S(O)₂, O, NH, or CH₂;
L represents optionally substituted linear or branched C₁₋₄₀ alkylene, wherein one or more (e.g., 1-15, 1-10, 1-8, 1-7, 1-6, 1-5, 1-4, 1-3, 1-2 or 1) pairs of adjacent carbon atoms in the backbone carbon chain of the linear or branched C₁₋₄₀ alkylene is optionally interrupted by one or more (e.g., 1-30, 1-20, 1-15, 1-10, 1-8, 1-7, 1-6, 1-5, 1-4, 1-3, 1-2 or 1) groups selected from the group consisting of: R_{b}, R_{c}, and any combination thereof, wherein R_{b} groups are each selected from the group consisting of O, C(O), OC(O), S, S(O), S(O)₂, S(O)₂N(R₁), N(R₁)S(O)₂, C(O)N(R₁), N(R₁)C(O), N(R₁), and N(R₁)C(O)N(R₁), where R₁ groups each independently represent H or C₁₋₃alkyl, and when two or more groups R_{b} are inserted into the backbone carbon chain of the linear or branched C₁₋₄₀ alkylene group, the two or more groups R_{b} are not directly connected to each other; and wherein R_{c} groups are each selected from the group consisting of optionally substituted cycloalkylene, optionally substituted arylene, optionally substituted heterocyclylene, optionally substituted heteroarylene, and any combination thereof; and
X₁ represents NR₂R₃, C(O)NR₄R₅, NHC(O)R₆, NHC(O)NR₇R₈, OR₉, SR₁₀, quaternary ammonium salt group, linear or branched C₁₋₁₀ alkyl optionally substituted with one or more (e.g., 1-30, 1-20, 1-15, 1-10, 1-8, 1-7, 1-6, 1-5, 1-4, 1-3, 1-2 or 1) fluorine atoms, optionally substituted cycloalkyl, optionally substituted aryl, optionally substituted heterocyclyl or optionally substituted heteroaryl, where R₂, R₃, R₄, R₅, R₇, R₈, R₉, and R₁₀ each independently represent H, optionally substituted linear or branched C₁₋₁₀ alkyl, optionally substituted cycloalkyl, optionally substituted aryl, optionally substituted heterocyclyl, or optionally substituted heteroaryl, wherein R₂ and R₃ are not H at the same time, and wherein R₆ represents optionally substituted linear or branched C₁₋₈ alkyl, optionally substituted cycloalkyl, optionally substituted aryl, optionally substituted heteroaryl, or optionally substituted heterocyclyl, or wherein R₄ and R₅ together with the nitrogen atom to which they are attached form optionally substituted 5- to 8-membered heterocyclyl; or
X₁ represents a group of Formula (G₁):

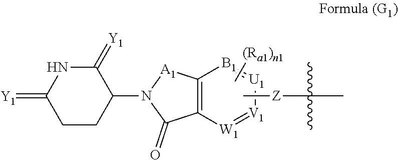

wherein A₁ represents CH₂ or C(O);
B₁, U₁, V₁, and W₁ are the same or different and each independently represent CH or N, wherein
B₁, U₁, V₁, and W₁ are not N at the same time;
Y₁ represents O or S; and
Z represents S, S(O) or S(O)₂; and
(Rₐ₁)ₙ₁ denotes that the ring containing B₁, U₁, V₁, and W₁ is substituted with n1 Rₐ₁, where Rₐ₁ groups are the same or different and each independently represent deuterium, halogen, hydroxy, mercapto, nitro, amino, cyano, optionally deuterated C₁₋₆ alkyl, optionally deuterated C₁₋₆ alkoxy, halogenated C₁₋₆ alkyl, optionally substituted C₃₋₆ cycloalkyl, C₂₋₆ alkenyl, or C₂₋₆ alkynyl, and n represents an integer of 0, 1, 2, or 3.

In some embodiments of the compounds of Formula (I) and Formula (I-4) of the present disclosure,
R represents S, S(O), S(O)₂, O, NH, or CH₂;
L represents linear or branched C₁₋₄₀ alkylene optionally substituted with one or more (e.g., 1-30, 1-20, 1-15, 1-10, 1-8, 1-7, 1-6, 1-5, 1-4, 1-3, 1-2 or 1) substituents selected from the group consisting of halogen, cyano, hydroxy, amino, mercapto, C₁₋₃ alkyl, C₁₋₃ alkoxy, halogenated C₁₋₃alkyl (e.g., trifluoromethyl), C₅₋₁₀ aryl, C₅₋₁₀ heteroaryl, C₃₋₁₂ cycloalkyl, 3- to 12-membered heterocyclyl, or any combination thereof, wherein one or more (e.g., 1-30, 1-20, 1-15, 1-10, 1-8, 1-7, 1-6, 1-5, 1-4, 1-3, 1-2 or 1) pairs of adjacent carbon atoms in the backbone carbon chain of the linear or branched C₁₋₄₀ alkylene is optionally interrupted by one or more (e.g., 1-30, 1-20, 1-15, 1-10, 1-8, 1-7, 1-6, 1-5, 1-4, 1-3, 1-2 or 1) groups selected from the group consisting of: R_{b}, R_{c}, and any combination thereof, wherein R_{b} groups are each selected from the group consisting of O, C(O), OC(O), S, S(O), S(O)₂, S(O)₂N(R₁), N(R₁)S(O)₂, C(O)N(R₁), N(R₁)C(O), N(R₁) and N(R₁)C(O)N(R₁), where R₁ groups each independently represent H or C₁₋₃ alkyl, and when two or more groups R_{b} are inserted into the backbone carbon chain of the linear or branched alkylene group, the two or more groups R_{b} are not directly connected to each other; and wherein R_{c} groups are each selected from the group consisting of cycloalkylene (e.g., C₃₋₂₀ cycloalkylene), arylene (e.g., C₅₋₂₀ arylene), heterocyclylene (e.g., 4- to 20-membered heterocyclylene), heteroarylene (e.g., 5- to 20-membered heteroarylene), and any combination thereof, wherein the cycloalkylene and heterocyclylene are each independently optionally substituted with one or more (e.g., 1-30, 1-20, 1-15, 1-10, 1-8, 1-7, 1-6, 1-5, 1-4, 1-3, 1-2 or 1) substituents selected from the group consisting of halogen, cyano, hydroxy, amino, mercapto, oxo, C₁₋₃ alkoxy, halogenated C₁₋₃ alkyl, methanesulfonyloxy, trifluoromethanesulfonyloxy, p-toluenesulfonyloxy, optionally deuterated C₁₋₆ alkyl, optionally deuterated C₃₋₆ cycloalkyl, or any combination thereof; and the arylene and heteroarylene are each independently optionally substituted with one or more (e.g., 1-30, 1-20, 1-15, 1-10, 1-8, 1-7, 1-6, 1-5, 1-4, 1-3, 1-2 or 1) substituents selected from the group consisting of halogen, cyano, hydroxy, amino, mercapto, C₁₋₃ alkoxy, halogenated C₁₋₃alkyl, methanesulfonyloxy, trifluoromethanesulfonyloxy, p-toluenesulfonyloxy, optionally deuterated C₁₋₆ alkyl, optionally deuterated C₃₋₆ cycloalkyl, or any combination thereof; and
X₁ represents:
   NR₂R₃, C(O)NR₄R₅, NHC(O)R₆, NHC(O)NR₇R₈, OR₉, SR₁₀, or quaternary ammonium salt group, where R₂, R₃, R₄, R₅, R₇, R₈, R₉, and R₁₀ each independently represent H, optionally substituted linear or branched C₁₋₁₀ alkyl, optionally substituted cycloalkyl, optionally substituted aryl, optionally substituted heterocyclyl, or optionally substituted heteroaryl, wherein R₂ and R₃ are not H at the same time, and wherein R₆ represents optionally substituted linear or branched C₁₋₆ alkyl, optionally substituted cycloalkyl, optionally substituted aryl, optionally substituted heteroaryl, or optionally substituted heterocyclyl, or wherein R₄ and R₅ together with the nitrogen atom to which they are attached form optionally substituted 5- to 8-membered heterocyclyl; or
   linear or branched C₁₋₁₀ alkyl, optionally substituted with one or more (e.g., 1-30, 1-20, 1-15, 1-10, 1-8, 1-7, 1-6, 1-5, 1-4, 1-3, 1-2 or 1) fluorine; or
   C₃-C₁₂ cycloalkyl, optionally substituted with one or more (e.g., 1-30, 1-20, 1-15, 1-10, 1-8, 1-7, 1-6, 1-5, 1-4, 1-3, 1-2 or 1) substituents selected from the group consisting of: halogen, cyano, amino, hydroxy, mercapto, oxo, C₁₋₃ alkoxy, halogenated C₁₋₃ alkoxy, halogenated C₁₋₃alkyl, C₁₋₆ alkylsulfonyl, bis(C₁₋₆ alkyl)phosphono, methanesulfonyloxy, trifluoromethanesulfonyloxy, p-toluenesulfonyloxy, optionally substituted 5- or 6-membered heterocyclyl, optionally substituted C₅-C₂₀ aryl, optionally substituted 5- or 6-membered heteroaryl, C₁₋₆alkyl, C₁₋₃ alkoxycarbonyl, halogenated C₂₋₄ alkenyl, or any combination thereof, wherein the 5- or 6-membered heterocyclyl is optionally substituted with one or more (e.g., 1-30, 1-20, 1-15, 1-10, 1-8, 1-7, 1-6, 1-5, 1-4, 1-3, 1-2 or 1) substituents selected from the group consisting of: halogen, cyano, amino, hydroxy, mercapto, oxo, C₁₋₆ alkyl, halogenated C₁₋₃ alkyl, or any combination thereof; and the C₅-C₂₀ aryl is optionally substituted with one or more (e.g., 1-30, 1-20, 1-15, 1-10, 1-8, 1-7, 1-6, 1-5, 1-4, 1-3, 1-2, or 1) substituents selected from the group consisting of: halogen, cyano, amino, hydroxy, mercapto, C₁₋₆ alkyl, halogenated C₁₋₃ alkyl, or any combination thereof; and the 5- or 6-membered heteroaryl is optionally substituted with one or more (e.g., 1-30, 1-20, 1-15, 1-10, 1-8, 1-7, 1-6, 1-5, 1-4, 1-3, 1-2, or 1) substituents selected from the group consisting of halogen, cyano, amino, hydroxy, mercapto, oxo, C₁₋₆ alkyl, halogenated C₁₋₃ alkyl, or any combination thereof; or
   C₅-C₁₄ aryl, optionally substituted with one or more (e.g., 1-30, 1-20, 1-15, 1-10, 1-8, 1-7, 1-6, 1-5, 1-4, 1-3, 1-2, or 1) substituents selected from the group consisting of halogen, cyano, amino, hydroxy, mercapto, C₁₋₃ alkoxy, halogenated C₁₋₃ alkoxy, halogenated C₁₋₃alkyl, C₁₋₆ alkylsulfonyl, bis(C₁₋₆ alkyl)phosphono, methanesulfonyloxy, trifluoromethanesulfonyloxy, p-toluenesulfonyloxy, optionally substituted 5- or 6-membered heterocyclyl, optionally substituted C₅-C₂₀ aryl, optionally substituted 5- or 6-membered heteroaryl, C₁₋₆ alkyl, C₁₋₃ alkoxycarbonyl, halogenated C₂₋₄ alkenyl, or any combination thereof, wherein the 5- or 6-membered heterocyclyl is optionally substituted with one or more (e.g., 1-30, 1-20, 1-15, 1-10, 1-8, 1-7, 1-6, 1-5, 1-4, 1-3, 1-2, or 1) substituents selected from the group consisting of halogen, cyano, amino, hydroxy, mercapto, oxo, C₁₋₆ alkyl, halogenated C₁₋₃ alkyl, or any combination thereof; and the C₅-C₂₀ aryl is optionally substituted with one or more (e.g., 1-30, 1-20, 1-15, 1-10, 1-8, 1-7, 1-6, 1-5, 1-4, 1-3, 1-2, or 1) substituents selected from the group consisting of halogen, cyano, amino, hydroxy, mercapto, C₁₋₆alkyl, halogenated C₁₋₃alkyl, or any combination thereof; and the 5- or 6-membered heteroaryl is optionally substituted with one or more (e.g., 1-30, 1-20, 1-15, 1-10, 1-8, 1-7, 1-6, 1-5, 1-4, 1-3, 1-2, or 1) substituents selected from the group consisting of halogen, cyano, amino, hydroxy, mercapto, oxo, C₁₋₆alkyl, halogenated C₁₋₃ alkyl, or any combination thereof; or
   3- to 12-membered heterocyclyl, optionally substituted with one or more (e.g., 1-30, 1-20, 1-15, 1-10, 1-8, 1-7, 1-6, 1-5, 1-4, 1-3, 1-2, or 1) substituents selected from the group consisting of halogen, cyano, amino, hydroxy, mercapto, oxo, C₁₋₃ alkoxy, halogenated C₁₋₃ alkoxy, halogenated C₁₋₃ alkyl, C₁₋₆ alkylsulfonyl, bis(C₁₋₆ alkyl)phosphono, methanesulfonyloxy, trifluoromethanesulfonyloxy, p-toluenesulfonyloxy, optionally substituted 5- or 6-membered heterocyclyl, optionally substituted C₅-C₂₀ aryl, optionally substituted 5- or 6-membered heteroaryl, C₁₋₆ alkyl, C₁₋₃ alkoxycarbonyl, halogenated C₂₋₄ alkenyl, or any combination thereof, wherein the 5- or 6-membered heterocyclyl is optionally substituted with one or more (e.g., 1-30, 1-20, 1-15, 1-10, 1-8, 1-7, 1-6, 1-5, 1-4, 1-3, 1-2, or 1) substituents selected from the group consisting of halogen, cyano, amino, hydroxy, mercapto, oxo, C₁₋₆ alkyl, halogenated C₁₋₃ alkyl, or any combination thereof; and the C₅-C₂₀ aryl is optionally substituted with one or more (e.g., 1-30, 1-20, 1-15, 1-10, 1-8, 1-7, 1-6, 1-5, 1-4, 1-3, 1-2, or 1) substituents selected from the group consisting of halogen, cyano, amino, hydroxy, mercapto, C₁₋₆ alkyl, halogenated C₁₋₃ alkyl, or any combination thereof; and the 5- or 6-membered heteroaryl is optionally substituted with one or more (e.g., 1-30, 1-20, 1-15, 1-10, 1-8, 1-7, 1-6, 1-5, 1-4, 1-3, 1-2, or 1) substituents selected from the group consisting of halogen, cyano, amino, hydroxy, mercapto, oxo, C₁₋₆alkyl, halogenated C₁₋₃alkyl, or any combination thereof, or
   5- to 10-membered heteroaryl, optionally substituted with one or more (e.g., 1-30, 1-20, 1-15, 1-10, 1-8, 1-7, 1-6, 1-5, 1-4, 1-3, 1-2, or 1) substituents selected from the group consisting of halogen, cyano, amino, hydroxy, mercapto, C₁₋₃ alkoxy, halogenated C₁₋₃ alkoxy, halogenated C₁₋₃alkyl, bis(C₁₋₆ alkyl)phosphono, C₁₋₆ alkylsulfonyl, methanesulfonyloxy, trifluoromethanesulfonyloxy, p-toluenesulfonyloxy, C₁₋₆ alkyl, optionally substituted 5- or 6-membered heterocyclyl, optionally substituted C₅-C₂₀ aryl, optionally substituted 5- or 6-membered heteroaryl, C₁₋₆ alkyl, C₁₋₃ alkoxycarbonyl, halogenated C₂₋₄ alkenyl, or any combination thereof, wherein the 5- or 6-membered heterocyclyl is optionally substituted with one or more (e.g., 1-30, 1-20, 1-15, 1-10, 1-8, 1-7, 1-6, 1-5, 1-4, 1-3, 1-2, or 1) substituents selected from the group consisting of C₁₋₆ alkyl, cyano, halogenated C₁₋₃alkyl, amino, hydroxy, mercapto, halogen, or any combination thereof; and the C₅-C₂₀ aryl is optionally substituted with one or more (e.g., 1-30, 1-20, 1-15, 1-10, 1-8, 1-7, 1-6, 1-5, 1-4, 1-3, 1-2, or 1) substituents selected from the group consisting of C₁₋₆ alkyl, cyano, halogenated C₁₋₃ alkyl, amino, hydroxy, mercapto, halogen, or any combination thereof; and the 5- or 6-membered heteroaryl is optionally substituted with one or more (e.g., 1-30, 1-20, 1-15, 1-10, 1-8, 1-7, 1-6, 1-5, 1-4, 1-3, 1-2, or 1) substituents selected from the group consisting of C₁₋₆ alkyl, halogenated C₁₋₃alkyl, amino, hydroxy, mercapto, halogen, or any combination thereof; or
X₁ represents a group of Formula (G₁):

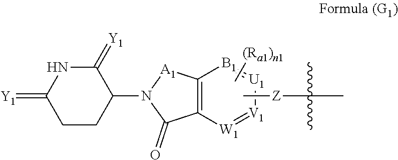

wherein A₁ represents CH₂ or C(O);
B₁, U₁, V₁, and W₁ are the same or different and each independently represent CH or N, wherein B₁, U₁, V₁, and W₁ are not N at the same time;
Y₁ represents O or S; and
Z represents S, S(O), or S(O)₂; and
(Rₐ₁)ₙ₁ denotes that the ring containing B₁, U₁, V₁, and W₁ is substituted with n1 Rₐ₁, where Rₐ₁ groups are the same or different and each independently represent deuterium, halogen, hydroxy, mercapto, nitro, amino, cyano, optionally deuterated C₁₋₆ alkyl, optionally deuterated C₁₋₆ alkoxy, halogenated C₁₋₆ alkyl, optionally substituted C₃₋₆ cycloalkyl, C₂₋₆ alkenyl, or C₂₋₆ alkynyl, and n represents an integer of 0, 1, 2, or 3, wherein the C₃₋₆ cycloalkyl is optionally substituted with a substituent selected from the group consisting of: C₁₋₆ alkyl, halogenated C₁₋₃ alkyl, amino, hydroxy, halogen, or any combination thereof.

In some embodiments of the compounds of Formula (I) and Formula (I-4) of the present disclosure, L represents the structure of the following formula:
linear or branched C₁₋₄₀ alkylene;
*—(C(Rₐ₂)(Rₐ₃))ₙ₂—(R_{b}—(C(Rₐ₄)(Rₐ₅))ₙ₃)ₘ₁;
*—(C(Rₐ₂)(Rₐ₃))ₙ₂—(R_{b}—(C(Rₐ₄)(Rₐ₅))ₙ₃)ₘ₁—(R_{b}—(C(Rₐ₆)(Rₐ₇))ₙ₄)ₘ₂—;
*—(C(R₂)(Rₐ₃))ₙ₂—(R_{b}—(C(Rₐ₄)(Rₐ₅))ₙ₃)ₘ₁—(R_{b}—(C(Rₐ)(Rₐ))ₙ₄)ₘ₂—(R_{b}—(C(Rₐ₈)(Rₐ₉))ₙ₅)ₘ₃—;
*—(C(Rₐ₂)(Rₐ₃))ₙ₂—(R_{c}—(C(Rₐ₄)(Rₐ₅))ₙ₃)ₘ₁—;
*—(C(Rₐ₂)(Rₐ₃))ₙ₂—(R_{c}—(C(Rₐ₄)(Rₐ₅))ₙ₃)ₘ₁—(R_{c}—(C(Rₐ₆)(Rₐ₇))ₙ₄)ₘ₂—;
*—(C(Rₐ₂)(Rₐ₃))ₙ₂—(R_{c}—(C(Rₐ₄)(Rₐ₅))ₙ₃)ₘ₁—(R_{c}—(C(Rₐ₆)(Rₐ₇))ₙ₄)ₘ₂—(R_{c}—(C(Rₐ₈)(Rₐ₉))ₙ₅)ₘ₃—;
*—(C(Rₐ₂)(Rₐ₃))ₙ₂—(R_{b}—R_{c}—(C(Rₐ₄)(Rₐ₅))ₙ₃)ₘ₁—;
*—(C(Rₐ₂)(Rₐ₃))ₙ₂—(R_{b}—(C(Rₐ₄)(Rₐ₅))ₙ₃)ₘ₁—(R_{c}—(C(Rₐ₆)(Rₐ₇))ₙ₄)ₘ₂—;
*—(C(Rₐ₂)(Rₐ₃))ₙ₂—(R_{c}—R_{b}—(C(Rₐ₄)(Rₐ₅))ₙ₃)ₘ₁—; or
*—(C(Rₐ₂)(Rₐ₃))ₙ₂—(R_{c}—(C(Rₐ₄)(Rₐ₅))ₙ₃)ₘ₁—(R_{b}—(C(Rₐ₆)(Rₐ₇))ₙ₄)ₘ₂—;
wherein * indicates the point of attachment to R;
a hydrogen of one or more CH₂ groups (e.g., 1-30, 1-20, 1-15, 1-10, 1-8, 1-7, 1-6, 1-5, 1-4, 1-3, 1-2, or 1 CH₂) of the linear or branched C₁₋₄₀ alkylene is optionally replaced with a substituent selected from the group consisting of: halogen, cyano, C₁₋₃ alkyl, C₁₋₃ alkoxy, trifluoromethyl, C₅₋₁₀ aryl, C₅₋₁₀ heteroaryl, C₃₋₁₂ cycloalkyl, 3- to 12-membered heterocyclyl, or any combination thereof;
R_{b} groups are each independently selected from the group consisting of O, C(O), OC(O), S, S(O), S(O)₂, S(O)₂N(R₁), N(R₁)S(O)₂, C(O)N(R₁), N(R₁)C(O), N(R₁) and N(R₁)C(O)N(R₁), wherein R₁ groups each independently represent H or C₁₋₃ alkyl;
R_{c} groups are each independently selected from the group consisting of optionally substituted cycloalkylene (e.g., C₃₋₂₀ cycloalkylene), optionally substituted arylene (e.g., C₅₋₂₀ arylene), optionally substituted heterocyclylene (e.g., 4- to 20-membered heterocyclylene), optionally substituted heteroarylene (e.g., 5- to 20-membered heteroarylene), and any combination thereof, wherein the cycloalkylene and heterocyclylene are each independently optionally substituted with a substituent selected from the group consisting of halogen, cyano, hydroxy, amino, mercapto, oxo, C₁₋₃ alkoxy, halogenated C₁₋₃ alkyl, methanesulfonyloxy, trifluoromethanesulfonyloxy, p-toluenesulfonyloxy, optionally deuterated C₁₋₆ alkyl, optionally deuterated C₃₋₆ cycloalkyl, or any combination thereof; and the arylene and heteroarylene are each independently optionally substituted with a substituent selected from the group consisting of halogen, cyano, hydroxy, amino, mercapto, C₁₋₃ alkoxy, halogenated C₁-3 alkyl, methanesulfonyloxy, trifluoromethanesulfonyloxy, p-toluenesulfonyloxy, optionally deuterated C₁₋₆ alkyl, optionally deuterated C₃₋₆ cycloalkyl, or any combination thereof;
Rₐ₂, Rₐ₃, Rₐ₄, Rₐ₅, Rₐ₆, Rₐ₇, Rₐ₈, and Rₐ₉ each independently represent H, halogen, cyano, hydroxy, amino, mercapto, C₁₋₃ alkyl, C₁₋₃ alkoxy, trifluoromethyl, C₅₋₁₀ aryl, C₅₋₁₀ heteroaryl, C₃₋₁₂ cycloalkyl, or 3- to 12-membered heterocyclyl; and
n2, n3, n4, n5, m1, m2, and m3 each independently represent an integer of 1, 2, 3, 4, 5, 6, 7, 8, 9, 10, 11, 12, 13, 14, or 15.

In some embodiments of the compounds of Formula (I) and Formula (I-4) of the present disclosure, L represents the structure of the following formula:
*—(C(Rₐ₂)(Rₐ₃))ₙ₂—(C₅₋₂₀ arylene-(C(Rₐ₄)(Rₐ₅))ₙ₃)ₘ₁—;
*—(C(Rₐ₂)(Rₐ₃))ₙ₂—(C₅₋₂₀ arylene-(C(Rₐ₄)(Rₐ₅))ₙ₃)ₘ₁—(C₅₋₂₀ arylene-(C(Rₐ₆)(Rₐ₇))ₙ₄)ₘ₂—;
*—(C(Rₐ₂)(Rₐ₃))ₙ₂—(C₅₋₂₀ arylene-(C(Rₐ₄)(Rₐ₅))ₙ₃)ₘ₁—(C₅₋₂₀ arylene-(C(Rₐ₆)(Rₐ₇))ₙ₄)ₘ₂—(C₅₋₂₀ arylene-(C(Rₐ₈)(Rₐ₉))ₙ₅)ₘ₃—;
*—(C(Rₐ₂)(Rₐ₃))ₙ₂-(5- to 20-membered heteroarylene-(C(Rₐ₄)(Rₐ₅))ₙ₃)ₘ₁—;
*—(C(Rₐ₂)(Rₐ₃))ₙ₂-(5- to 20-membered heteroarylene-(C(Rₐ₄)(Rₐ₅))ₙ₃)ₘ₁-(5- to 20-membered heteroarylene-(C(Rₐ₆)(Rₐ₇))ₙ₄)ₘ₂—;
*—(C(Rₐ₂)(Rₐ₃))ₙ₂-(5- to 20-membered heteroarylene-(C(Rₐ₄)(Rₐ₅))ₙ₃)ₘ₁-(5- to 20-membered heteroarylene-(C(Rₐ₆)(Rₐ₇))ₙ₄)ₘ₂-(5- to 20-membered heteroarylene-(C(Rₐ₈)(Rₐ₉))ₙ₅)ₘ₃—;
*—(C(Rₐ₂)(Rₐ₃))ₙ₂-(4- to 20-membered heterocyclylene-(C(Rₐ₄)(Rₐ₅))ₙ₃)ₘ₁—;
*—(C(Rₐ₂)(Rₐ₃))ₙ₂-(4- to 20-membered heterocyclylene-(C(Rₐ₄)(Rₐ₅))ₙ₃)ₘl-(4- to 20-membered heterocyclylene-(C(Rₐ₆)(Rₐ₇))ₙ₄)ₘ₂—;
*—(C(Rₐ₂)(Rₐ₃))ₙ₂—(O—(C(Rₐ₄)(Rₐ₅))ₙ₃)ₘ₁—;
*—(C(Rₐ₂)(Rₐ₃))ₙ₂—(O—(C(Rₐ₄)(Rₐ₅))ₙ₃)ₘ₁—(O—(C(Rₐ₆)(Rₐ₇))ₙ₄)ₘ₂—;
*—(C(Rₐ₂)(Rₐ₃))ₙ₂—(O—(C(Rₐ₄)(Rₐ₅))ₙ₃)ₘ₁—(O—(C(Rₐ₆)(Rₐ₇))ₙ₄)ₘ₂—(O—(C(Rₐ₈)(Rₐ₉))ₙ₅)ₘ₃—;
*—(C(Rₐ₂)(Rₐ₃))ₙ₂—(S—(C(Rₐ₄)(Rₐ₅))ₙ₃)ₘ₁—;
*—(C(Rₐ₂)(Rₐ₃))ₙ₂—(S—(C(Rₐ₄)(Rₐ₅))ₙ₃)ₘ₁—(S—(C(Rₐ₆)(Rₐ₇))ₙ₄)ₘ₂—;
*—(C(Rₐ₂)(Rₐ₃))ₙ₂—(S—(C(Rₐ₄)(Rₐ₅))ₙ₃)ₘ₁—(S—(C(Rₐ₆)(Rₐ₇))ₙ₄)ₘ₂—(S—(C(Rₐ₈)(Rₐ₉))ₙ₅)ₘ₃—;
*—(C(Rₐ₂)(Rₐ₃))ₙ₂—(S(O)—(C(Rₐ₄)(Rₐ₅))ₙ₃)ₘ₁—;
*—(C(Rₐ₂)(Rₐ₃))ₙ₂—(S(O)₂—(C(Rₐ₄)(Rₐ₅))ₙ₃)ₘ₁—;
*—(C(Rₐ₂)(Rₐ₃))ₙ₂—(S—(C(Rₐ₄)(Rₐ₅))ₙ₃)ₘ₁—;
*—(C(Rₐ₂)(Rₐ₃))ₙ₂—(S(O)₂N(R₁)—(C(Rₐ₄)(Rₐ₅))ₙ₃)ₘ₁—;
*—(C(Rₐ₂)(Rₐ₃))ₙ₂—(N(R₁)S(O)₂—(C(Rₐ₄)(Rₐ₅))ₙ₃)ₘ₁—;
*—(C(Rₐ₂)(Rₐ₃))ₙ₂—(C(O)N(R₁)—(C(Rₐ₄)(Rₐ₅))ₙ₃)ₘ₁—;
*—(C(Rₐ₂)(Rₐ₃))ₙ₂—(N(R₁)C(O)—(C(Rₐ₄)(Rₐ₅))ₙ₃)ₘ₁—;
*—(C(Rₐ₂)(Rₐ₃))ₙ₂—(N(R₁)—(C(Rₐ₄)(Rₐ₅))ₙ₃)ₘ₁—;
*—(C(Rₐ₂)(Rₐ₃))ₙ₂—(N(R₁)C(O)N(R₁)—(C(Rₐ₄)(Rₐ₅))ₙ₃)ₘ₁—;
wherein * indicates the point of attachment to R;
R₁ groups each independently represent H or C₁₋₃alkyl;
Rₐ₂, Rₐ₃, Rₐ₄, Rₐ₅, Rₐ₆, Rₐ₇, Rₐ₈, and Rₐ₉ each independently represent H, halogen, cyano, hydroxy, amino, mercapto, C₁₋₃ alkyl, C₁₋₃ alkoxy, trifluoromethyl, C₅₋₁₀aryl, C₅₋₁₀ heteroaryl, C₃₋₁₂ cycloalkyl or 3- to 12-membered heterocyclyl;
n2, n3, n4, n5, m1, m2, and m3 each independently represent an integer of 1, 2, 3, 4, 5, 6, 7, 8, 9, 10, 11, 12, 13, 14, or 15; and
the 4- to 20-membered heterocyclylene is optionally substituted with a substituent selected from the group consisting of halogen, cyano, hydroxy, amino, mercapto, oxo, C₁₋₃ alkoxy, halogenated C₁₋₃ alkyl, methanesulfonyloxy, trifluoromethanesulfonyloxy, p-toluenesulfonyloxy, optionally deuterated C₁₋₆ alkyl, optionally deuterated C₃₋₆ cycloalkyl, or any combination thereof; and the C₅₋₂₀ arylene and the 5- to 20-membered heteroarylene are each independently optionally substituted with a substituent selected from the group consisting of halogen, cyano, hydroxy, amino, mercapto, C₁₋₃ alkoxy, halogenated C₁₋₃ alkyl, methanesulfonyloxy, trifluoromethanesulfonyloxy, p-toluenesulfonyloxy, optionally deuterated C₁₋₆ alkyl, optionally deuterated C₃₋₆ cycloalkyl, or any combination thereof.

In some embodiments of the compounds of Formula (I) and Formula (I-4) of the present disclosure, L represents the structure of the following formula:
linear or branched C₁-C₄₀ alkylene, *—(CH₂)ₙ₂—(O—(CH₂)ₙ₃)ₘ₁—, *—(CH₂)ₙ₂—(O—(CH₂)ₙ₃)ₘ₁—(O—(CH₂)ₙ₄)ₘ₂—, *—(CH₂)ₙ₂—(O—(CH₂)ₙ₃)ₘ₁—(O—(CH₂)ₙ₄)ₘ₂—(O—(CH₂)ₙ₅)ₘ₃—, *—(CH₂)ₙ₂—S—(CH₂)ₙ₃—, *—(CH₂)ₙ₂—S(O)—(CH₂)ₙ₃—, *—(CH₂)ₙ₂—S(O)₂—(CH₂)ₙ₃—, *—(CH₂)ₙ₂—N(R₁)S(O)₂—(CH₂)ₙ₃—, *—(CH₂)ₙ₂—S(O)₂N(R₁)—(CH₂)ₙ₃—, *—(CH₂)ₙ₂—N(R₁)—(CH₂)ₙ₃—, *—(CH₂)ₙ₂—N(R₁)C(O)—(CH₂)ₙ₃—, *—(CH₂)ₙ₂—C(O)N(R₁)—(CH₂)ₙ₃—, *—(CH₂)ₙ₂—N(R₁)C(O)N(R₁)—(CH₂)ₙ₃—, *—(CH₂)ₙ₂—(N(R₁)C(O)—(CH₂)ₙ₃)ₘ₁—, *—(CH₂)ₙ₂-piperazinylene-(CH₂)ₙ₃—, *—(CH₂)ₙ₂-phenylene-(CH₂)ₙ₃—, *—(CH₂)ₙ₂-phenylene-(CH₂)ₙ₃—N(R₁)—(CH₂)ₙ₄—, *—(CH₂)ₙ₂-furanylene-(CH₂)ₙ₃—, *—(CH₂)ₙ₂-furanylene-(CH₂)ₙ₃—N(R₁)—(CH₂)ₙ₄—, *—(CH₂)ₙ₂-thiazolylene-(CH₂)ₙ₃—, *—(CH₂)ₙ₂-thiazolylene-C(O)N(R₁)—(CH₂)ₙ₃—, or *—(CH₂)ₙ₂-thiazolylene-(CH₂)ₙ₃—N(R₁)—(CH₂)ₙ₄—;
wherein a hydrogen of one or more CH₂ groups (e.g., 1-30, 1-20, 1-15, 1-10, 1-8, 1-7, 1-6, 1-5, 1-4, 1-3, 1-2, or 1 CH₂) of the linear or branched C₁₋₄₀ alkylene is optionally replaced with a substituent selected from the group consisting of: halogen, cyano, C₁₋₃ alkyl, C₁₋₃ alkoxy, trifluoromethyl, C₅₋₁₀aryl, 5- to 10-membered heteroaryl, C₃₋₁₂ cycloalkyl, 3- to 12-membered heterocyclyl, or any combination thereof;
* indicates the point of attachment to R;
R₁ groups each independently represent H or C₁₋₃ alkyl;
n2, n3, n4, n5, m1, m2, and m3 each independently represent an integer of 1, 2, 3, 4, 5, 6, 7, 8, 9, 10, 11, 12, 13, 14, or 15;
the piperazinylene is optionally substituted with a substituent selected from the group consisting of halogen, cyano, hydroxy, amino, mercapto, oxo, C₁₋₃ alkoxy, halogenated C₁₋₃ alkyl, methanesulfonyloxy, trifluoromethanesulfonyloxy, p-toluenesulfonyloxy, optionally deuterated C₁₋₆ alkyl, optionally deuterated C₃₋₆ cycloalkyl, or any combination thereof; and the phenylene, the furanylene, and the thiazolylene are each independently optionally substituted with a substituent selected from the group consisting of halogen, cyano, hydroxy, amino, mercapto, C₁₋₃ alkoxy, halogenated C₁₋₃ alkyl, methanesulfonyloxy, trifluoromethanesulfonyloxy, p-toluenesulfonyloxy, optionally deuterated C₁₋₆ alkyl, optionally deuterated C₃₋₆ cycloalkyl, or any combination thereof.

In some embodiments of the compounds of Formula (I) and Formula (I-4) of the present disclosure, L represents the following groups:
—CH₂—; —(CH₂)₂—; —(CH₂)₃—; —(CH₂)₄—; —(CH₂)₅—; —(CH₂)₆—; —(CH₂)₇—; —(CH₂)₈—; —(CH₂)₉—; —(CH₂)₁₀—; —(CH₂)₁₁—; —(CH₂)₁₂—; —(CH₂)₁₃—; —(CH₂)₁₄—; —(CH₂)₁₅—; —(CH₂)₁₆—; —(CH₂)₁₇—; —(CH₂)₁₈—; —(CH₂)₁₉—; or —(CH₂)₂₀—;
*—CH₂CH₂O(CH₂)₂—; *—(CH₂CH₂O)₂—(CH₂)₂—; *—(CH₂CH₂O)₃—(CH₂)₂—; *—(CH₂CH₂O)₄—(CH₂)₂—; *—(CH₂CH₂O)—(CH₂)₂—; *—(CH₂CH₂O)—(CH₂)₂—; *—(CH₂CH₂O)₇—(CH₂)₂—; *—(CH₂CH₂O)₈—(CH₂)₂—; *—(CH₂CH₂O)₉(CH₂)₂—; *—(CH₂CH₂O)₁₀(CH₂)₂—; *—CH₂CH₂OCH₂—; *—(CH₂CH₂O)₂—CH₂—; *—(CH₂CH₂O)₃—CH₂—; *—(CH₂CH₂O)₄—CH₂—; *—(CH₂CH₂O)₅—CH₂—; *—(CH₂CH₂O)₆—CH₂—; *—(CH₂CH₂O)₇—CH₂—; *—(CH₂CH₂O)₈—CH₂—; *—(CH₂CH₂O)₉—CH₂—; *—(CH₂CH₂O)₁₀—CH₂—; *—CH₂CH₂O(CH₂)₃—; *—(CH₂CH₂O)₂—(CH₂)₃—; *—(CH₂CH₂O)₃—(CH₂)₃—; *—(CH₂CH₂O)₄—(CH₂)₃—; *—(CH₂CH₂O)₅—(CH₂)₃—; *—(CH₂CH₂O)₆—(CH₂)₃—; *—(CH₂CH₂O)₇—(CH₂)₃—; *—(CH₂CH₂O)₈—(CH₂)₃—; *—(CH₂CH₂O)₉(CH₂)₃—; *—(CH₂CH₂O)₁₀(CH₂)₃—; *—CH₂CH₂OCH₂CH₂CH₂OCH₂—; *—CH₂CH₂OCH₂CH₂CH₂O—(CH₂)₂—; *—CH₂CH₂OCH₂CH₂CH₂O—(CH₂)₃—; *—(CH₂CH₂O)₂(CH₂CH₂CH₂O)(CH₂)₃—; *—(CH₂CH₂O)₂(CH₂CH₂CH₂O)₂(CH₂)₃—; *—(CH₂)₁O(CH₂)₁—; *—(CH₂)₁O(CH₂)₂—; *—(CH₂)₂O(CH₂)₂—; *—(CH₂)₂O(CH₂)₁—; *—(CH₂)₂O(CH₂)₃—; *—(CH₂)₂O(CH₂)₄—; *—(CH₂)₂O(CH₂)₅—; *—(CH₂)₂O(CH₂)₆—; *—(CH₂)₃O(CH₂)₁—; *—(CH₂)₃O(CH₂)₂—; *—(CH₂)₃O(CH₂)₃—; *—(CH₂)₄O(CH₂)₁—; *—(CH₂)₄O(CH₂)₂—; *—(CH₂)₄O(CH₂)₃—; *—(CH₂)₅O(CH₂)₁—; *—(CH₂)₅O(CH₂)₂—; *—(CH₂)₅O(CH₂)₃—; *—(CH₂)₅O(CH₂)₄—; or *—(CH₂)₅O(CH₂)₅—;
*—(CH₂)₁—NH—(CH₂)₁—; *—(CH₂)₂—NH—(CH₂)₁—; *—(CH₂)₂—NH—(CH₂)₂—; *—(CH₂)₂—NH—(CH₂)₃—; *—(CH₂)₂—NH—(CH₂)₄—; *—(CH₂)₂—NH—(CH₂)₅—; *—(CH₂)₂—NH—(CH₂)₆—; *—(CH₂)₂—NH—(CH₂)₇—; *—(CH₂)₂—NH—(CH₂)₈—; *—(CH₂)₂—NH—(CH₂)₉—; *—(CH₂)₂—NH—(CH₂)₁₀—; *—(CH₂)₂—NH—(CH₂)₁₁—; *—(CH₂)₂—NH—(CH₂)₁₂—; *—(CH₂)₃—NH—(CH₂)₁—; *—(CH₂)₃—NH—(CH₂)₂—; *—(CH₂)₃—NH—(CH₂)₃—; *—(CH₂)₄—NH—(CH₂)₁—; *—(CH₂)₄—NH—(CH₂)₂—; *—(CH₂)₅—NH—(CH₂)₃—; *—(CH₂)₅—NH—(CH₂)₁—; *—(CH₂)₅—NH—(CH₂)₂—; *—(CH₂)₅—NH—(CH₂)₂—; *—(CH₂)₅—NH—(CH₂)₃—; *—(CH₂)—NH—(CH₂)₄—; *—(CH₂)₅—NH—(CH₂)₅—; *—(CH₂)₁—N(CH₃)—(CH₂)₅—; *—(CH₂)₂—N(CH₃)—(CH₂)₁—; *—(CH₂)₃—N(CH₃)—(CH₂)₁—; *—(CH₂)₄—N(CH₃)—(CH₂)₁—; *—(CH₂)₅—N(CH₃)—(CH₂)₁—; *—(CH₂)₆—N(CH₃)—(CH₂)₁—; *—(CH₂)₂—N(CH₃)—(CH₂)₂—; *—(CH₂)₂—N(CH₃)—(CH₂)₃—; *—(CH₂)₂—N(CH₃)—(CH₂)₄—; *—(CH₂)₂—N(CH₃)—(CH₂)₅—; *—(CH₂)₂—N(CH₃)—(CH₂)₆—; *—(CH₂)₂—N(CH₃)—(CH₂)₇—; *—(CH₂)₂—N(CH₃)—(CH₂)₈—; *—(CH₂)₂—N(CH₃)—(CH₂)₉—; *—(CH₂)₂—N(CH₃)—(CH₂)₁₀—; *—(CH₂)₂—N(CH₃)—(CH₂)₁₁—; *—(CH₂)₂—N(CH₃)—(CH₂)₁₂—; *—(CH₂)₂—NHC(O)—CH₂—; *—(CH₂)₂—NHC(O)—(CH₂)₂—; *—(CH₂)₂—NHC(O)—(CH₂)₃—; *—(CH₂)₂—NHC(O)—(CH₂)₄—; *—(CH₂)₂—NHC(O)—(CH₂)₅—; *—(CH₂)₂—NHC(O)—(CH₂)₆—; *—(CH₂)₂—NHC(O)—(CH₂)₇—; *—(CH₂)₂—NHC(O)—(CH₂)₈—; *—(CH₂)₂—NHC(O)—(CH₂)₉—; *—(CH₂)₂—NHC(O)—(CH₂)₁₀—; *—(CH₂)₂—NHC(O)—(CH₂)₁₁—; *—(CH₂)₂—NHC(O)—(CH₂)₁₂—; *—(CH₂)₂—NHC(O)—(CH₂)₁₃—; *—(CH₂)₂—NHC(O)—(CH₂)₁₄—; *—(CH₂)₂—NHC(O)—(CH₂)₁₅—; *—(CH₂)₃—NHC(O)—CH₂—; *—(CH₂)₃—NHC(O)—(CH₂)₂—; *—(CH₂)₃—NHC(O)—(CH₂)₃—; *—(CH₂)₃—NHC(O)—(CH₂)₄—; *—(CH₂)₃—NHC(O)—(CH₂)₅—; *—(CH₂)₃—NHC(O)—(CH₂)₆—; *—(CH₂)₃—NHC(O)—(CH₂)₇—; *—(CH₂)₃—NHC(O)—(CH₂)₈—; *—(CH₂)₃—NHC(O)—(CH₂)₉—; *—(CH₂)₃—NHC(O)—(CH₂)₁₀—; *—(CH₂)₃—NHC(O)—(CH₂)₁—; *—(CH₂)₃—NHC(O)—(CH₂)₁₂—; *—(CH₂)₃—NHC(O)—(CH₂)₁₃—; *—(CH₂)₃—NHC(O)—(CH₂)₁₄—; *—(CH₂)₃—NHC(O)—(CH₂)₁₅—; *—(CH₂)₄NHC(O)(CH₂)₁—; *—(CH₂)₄NHC(O)(CH₂)₂—; *—(CH₂)₄NHC(O)(CH₂)₃—; *—(CH₂)₄NHC(O)(CH₂)₄—; *—(CH₂)₄NHC(O)(CH₂)₅—; *—(CH₂)₄NHC(O)(CH₂)₆—; *—(CH₂)₄NHC(O)(CH₂)₇—; *—(CH₂)₄NHC(O)(CH₂)₈—; *—(CH₂)₄NHC(O)(CH₂)₉—; *—(CH₂)₄NHC(O)(CH₂)₁₀—; *—(CH₂)₈NHC(O)(CH₂)₁—; *—(CH₂)₈NHC(O)(CH₂)₂—; *—(CH₂)₂—N(CH₃)C(O)—CH₂—; *—(CH₂)₂—N(CH₃)C(O)—(CH₂)₂—; *—(CH₂)₂—N(CH₃)C(O)—(CH₂)₃—; *—(CH₂)₂—N(CH₃)C(O)—(CH₂)₄—; *—(CH₂)₂—N(CH₃)C(O)—(CH₂)₅—; *—(CH₂)₂—N(CH₃)C(O)—(CH₂)₆—; *—(CH₂)₂—N(CH₃)C(O)—(CH₂)₇—; *—(CH₂)₂—N(CH₃)C(O)—(CH₂)₈—; *—(CH₂)₂—N(CH₃)C(O)—(CH₂)₉—; *—(CH₂)₂—N(CH₃)C(O)—(CH₂)₁₀—; *—(CH₂)₂—N(CH₃)C(O)—(CH₂)₁₁—; *—(CH₂)₂—N(CH₃)C(O)—(CH₂)₁₂—; *—(CH₂)₂—N(CH₃)C(O)—(CH₂)₁₃—; *—(CH₂)₂—N(CH₃)C(O)—(CH₂)₁₄—; *—(CH₂)₂—N(CH₃)C(O)—(CH₂)₁₅—; *—(CH₂)₂—C(O)NH—CH₂—; *—(CH₂)₂—C(O)NH—(CH₂)₂—; *—(CH₂)₂—C(O)NH—(CH₂)₃—; *—(CH₂)₂—C(O)NH—(CH₂)₄—; *—(CH₂)₂—C(O)NH—(CH₂)₅—; *—(CH₂)₂—C(O)NH—(CH₂)₆—; *—(CH₂)₂—C(O)NH—(CH₂)₇—; *—(CH₂)₂—C(O)NH—(CH₂)₈—; *—(CH₂)₂—C(O)NH—(CH₂)₉—; *—(CH₂)₂—C(O)NH—(CH₂)₁₀—; *—(CH₂)₂—C(O)NH—(CH₂)₁—; *—(CH₂)₂—C(O)NH—(CH₂)₁₂—; *—(CH₂)₂—C(O)NH—(CH₂)₁₃—; *—(CH₂)₂—C(O)NH—(CH₂)₁₄—; *—(CH₂)₂—C(O)NH—(CH₂)₁₅—; *—(CH₂)₃—C(O)NH—CH₂—; *—(CH₂)₃—C(O)NH—(CH₂)₂—; *—(CH₂)₃—C(O)NH—(CH₂)₃—; *—(CH₂)₃—C(O)NH—(CH₂)₄—; *—(CH₂)₃—C(O)NH—(CH₂)₅—; *—(CH₂)₃—C(O)NH—(CH₂)₆—; *—(CH₂)₃—C(O)NH—(CH₂)₇—; *—(CH₂)₃—C(O)NH—(CH₂)₈—; *—(CH₂)₃—C(O)NH—(CH₂)₉—; *—(CH₂)₃—C(O)NH—(CH₂)₁₀—; *—(CH₂)₃—C(O)NH—(CH₂)₁₁—; *—(CH₂)₃—C(O)NH—(CH₂)₁₂—; *—(CH₂)₃—C(O)NH—(CH₂)₁₃—; *—(CH₂)₃—C(O)NH—(CH₂)₁₄—; *—(CH₂)₃—C(O)NH—(CH₂)₁₅—; *—(CH₂)₄C(O)NH(CH₂)₁—; *—(CH₂)₄C(O)NH(CH₂)₂—; *—(CH₂)₄C(O)NH(CH₂)₃—; *—(CH₂)₄C(O)NH(CH₂)₄—; *—(CH₂)₄C(O)NH(CH₂)₅—; *—(CH₂)₄C(O)NH(CH₂)₆—; *—(CH₂)₄C(O)NH(CH₂)₇—; *—(CH₂)₄C(O)NH(CH₂)₈—; *—(CH₂)₄C(O)NH(CH₂)₉—; *—(CH₂)₄C(O)NH(CH₂)₁₀—; *—(CH₂)₂—C(O)N(CH₃)—CH₂—; *—(CH₂)₂—C(O)N(CH₃)—(CH₂)₂—; *—(CH₂)₂—C(O)N(CH₃)—(CH₂)₃—; *—(CH₂)₂—C(O)N(CH₃)—(CH₂)₄—; *—(CH₂)₂—C(O)N(CH₃)—(CH₂)₅—; *—(CH₂)₂—C(O)N(CH₃)—(CH₂)₆—; *—(CH₂)₂—C(O)N(CH₃)—(CH₂)₇—; *—(CH₂)₂—C(O)N(CH₃)—(CH₂)₈—; *—(CH₂)₂—C(O)N(CH₃)—(CH₂)₉—; *—(CH₂)₂—C(O)N(CH₃)—(CH₂)₁₀—; *—(CH₂)₂—C(O)N(CH₃)—(CH₂)₁₃—; *—(CH₂)₂—C(O)N(CH₃)—(CH₂)₁₂—; *—(CH₂)₂—C(O)N(CH₃)—(CH₂)₁₃—; *—(CH₂)₂—C(O)N(CH₃)—(CH₂)₁₄—; *—(CH₂)₂—C(O)N(CH₃)—(CH₂)₁₅—; *—(CH₂)₂—NHC(O)NH—(CH₂)₄—; *—(CH₂)₄—NHC(O)NH—(CH₂)₂—; *—CH₂—NHC(O)NH—(CH₂)₂—; *—(CH₂)₂—NHC(O)NH—CH₂—; *—(CH₂)₂—NHC(O)NH—(CH₂)₂—; *—(CH₂)₂—NHC(O)NH—(CH₂)₃—; *—(CH₂)₃—NHC(O)NH—(CH₂)₂—; *—CH₂-piperazinylene-CH₂—; *—(CH₂)₂-piperazinylene-(CH₂)₂—; *—(CH₂)₂-piperazinylene-(CH₂)₃—; *—(CH₂)₂-piperazinylene-(CH₂)₄—; *—(CH₂)₂-piperazinylene-(CH₂)₅—; *—(CH₂)₃-piperazinylene-CH₂—; *—(CH₂)₃-piperazinylene-(CH₂)₂—; *—(CH₂)₃-piperazinylene-(CH₂)₃—; *—(CH₂)₄-piperazinylene-CH₂—; *—(CH₂)₄-piperazinylene-(CH₂)₂—; *—(CH₂)₄-piperazinylene-(CH₂)₃—; *—(CH₂)₈-piperazinylene-CH₂—; *—(CH₂)₈-piperazinylene-(CH₂)₂—; *—(CH₂)₈-piperazinylene-(CH₂)₃—; *—(CH₂)₈-piperazinylene-(CH₂)₄—; *—(CH₂)₈-piperazinylene-(CH₂)₅—; *—(CH₂)₈-piperazinylene-(CH₂)₆—; *—(CH₂)₈-piperazinylene-(CH₂)₇—; *—(CH₂)₈-piperazinylene-(CH₂)₈—; *—CH₂-piperazinylene-(CH₂)₈—; *—(CH₂)₂-piperazinylene-(CH₂)₈—; *—(CH₂)₃-piperazinylene-(CH₂)₈—; *—(CH₂)₄-piperazinylene-(CH₂)₈—; *—(CH₂)₅-piperazinylene-(CH₂)₈—; *—(CH₂)₆-piperazinylene-(CH₂)₈—; *—(CH₂)₇-piperazinylene-(CH₂)₈—; *—CH₂-phenylene-CH₂—; *—(CH₂)₂-phenylene-(CH₂)₂—; *—(CH₂)₂-phenylene-(CH₂)₃—; *—(CH₂)₂-phenylene-(CH₂)₄—; *—(CH₂)₂-phenylene-(CH₂)₅—; *—(CH₂)₃-phenylene-CH₂—; *—(CH₂)₃-phenylene-(CH₂)₂—; *—(CH₂)₃-phenylene-(CH₂)₃—; *—(CH₂)₄-phenylene-CH₂—; *—(CH₂)₄-phenylene-(CH₂)₂—; *—(CH₂)₄-phenylene-(CH₂)₃—; *—(CH₂)₅-phenylene-(CH₂)₃—; *—(CH₂)₆-phenylene-(CH₂)₃—; *—(CH₂)₇-phenylene-(CH₂)₃—; *—(CH₂)₈-phenylene-CH₂—; *—(CH₂)₈-phenylene-(CH₂)₂—; *—(CH₂)₈-phenylene-(CH₂)₃—; *—(CH₂)₈-phenylene-(CH₂)₄—; *—(CH₂)₈-phenylene-(CH₂)₅—; *—(CH₂)₈-phenylene-(CH₂)₆—; *—(CH₂)₈-phenylene-(CH₂)₇—; *—(CH₂)₈-phenylene-(CH₂)₈—; *—CH₂-phenylene-(CH₂)₈—; *—(CH₂)₂-phenylene-(CH₂)₈—; *—(CH₂)₃-phenylene-(CH₂)₈—; *—(CH₂)₄-phenylene-(CH₂)₈—; *—(CH₂)₅-phenylene-(CH₂)₈—; *—(CH₂)₆-phenylene-(CH₂)₈—; *—(CH₂)₇-phenylene-(CH₂)₈—; *—(CH₂)₁S(CH₂)₁—; *—(CH₂)₂S(CH₂)₂—; *—(CH₂)₂S(CH₂)₁—; *—(CH₂)₁S(CH₂)₂—; *—(CH₂)₁S(CH₂)₃—; *—(CH₂)₁S(CH₂)₄—; *—(CH₂)₂S(CH₂)₃—; *—(CH₂)₂S(CH₂)₄—; *—(CH₂)₂S(CH₂)₅—; *—(CH₂)₃S(CH₂)₁—; *—(CH₂)₃S(CH₂)₂—; *—(CH₂)₃S(CH₂)₃—; *—(CH₂)₄S(CH₂)₁—; *—(CH₂)₄S(CH₂)₂—; *—(CH₂)₄S(CH₂)₃—; *—(CH₂)₅S(CH₂)₁—; *—(CH₂)₅S(CH₂)₂—; *—(CH₂)₅S(CH₂)₃—; *—(CH₂)₆S(CH₂)—; *(CH₂)₂—; *—(CH₂)S(CH₂)₃—; *—(CH₂)₇S(CH₂)₁—; *—(CH₂)₇S(CH₂)₂—; *—(CH₂)₇S(CH₂)₃—; *—(CH₂)₈S(CH₂)₁—; *—(CH₂)₈S(CH₂)₂—; *—(CH₂)₈S(CH₂)₃—; *—(CH₂)₉S(CH₂)₁—; *—(CH₂)₉S(CH₂)₂—; *—(CH₂)₉S(CH₂)₃—; *—(CH₂)₁S(O) (CH₂)₁—; *—(CH₂)₂S(O)(CH₂)₂—; *—(CH₂)₂S(O)(CH₂)₁—; *—(CH₂)₁S(O)(CH₂)₂—; *—(CH₂)₁S(O)(CH₂)₃—; *—(CH₂)₁S(O)(CH₂)₄—; *—(CH₂)₂S(O)(CH₂)₃—; *—(CH₂)₂S(O)(CH₂)₄—; *—(CH₂)₂S(O)(CH₂)₅—; *—(CH₂)₃S(O)(CH₂)₁—; *—(CH₂)₃S(O)(CH₂)₂—; *—(CH)₃S(O)(CH)₃—; *—(CH₂)₄S(O)(CH)₁—; *—(CH)₄S(O)(CH)₂—; *—(CH₂)₄S(O)(CH₂)₃—; *—(CH₂)₅S(O)(CH₂)₁—; *—(CH₂)₅S(O)(CH₂)₂; *—(CH₂)₅S(O)(CH₂)₃; *—(CH₂)₆S(O)(CH₂)₁—; *—(CH₂)₆S(O)(CH₂)₂; *—(CH₂)₆S(O)(CH₂)₃; *—(CH₂)₇S(O)(CH₂)₁—; *—(CH₂)₇S(O)(CH₂)₂—; *—(CH₂)₇S(O)(CH₂)₃—; *—(CH₂)₈S(O)(CH₂)₁—; *—(CH₂)₈S(O)(CH₂)₂—; *—(CH₂)₈S(O)(CH₂)₃—; *—(CH₂)₉S(O)(CH₂)₁—; *—(CH₂)₉S(O)(CH₂)₂—; *—(CH₂)₉S(O)(CH₂)₃—; *—(CH₂)₁S(O)₂(CH₂)₁—; *—(CH₂)₂S(O)₂(CH₂)₂—; *—(CH₂)₂S(O)₂(CH₂)₁—; *—(CH₂)₁S(O)₂(CH₂)₂—; *—(CH₂)₁S(O)₂(CH₂)₃—; *—(CH₂)₁S(O)₂(CH₂)₄—; *—(CH₂)₂S(O)₂(CH₂)₃—; *—(CH₂)₂S(O)₂(CH₂)₄—; *—(CH₂)₂S(O)₂(CH₂)₅—; *—(CH₂)₃S(O)₂(CH₂)₁—; *—(CH₂)₃S(O)₂(CH₂)₂—; *—(CH₂)₃S(O)₂(CH₂)₃—; *—(CH₂)₄S(O)₂(CH₂)₁—; *—(CH₂)₄S(O)₂(CH₂)₂—; *—(CH₂)₄S(O)₂(CH₂)₃—; *—(CH₂)₅S(O)₂(CH₂)₁—; *—(CH₂)₅S(O)₂(CH₂)₂—; *—(CH₂)₅S(O)(CH₂)₃—; *—(CH₂)₆S(O)₂(CH₂)₁—; *—(CH₂)₆S(O)₂(CH₂)₂—; *—(CH₂)₆S(O)₂(CH₂)₃—; *—(CH₂)₇S(O)₂(CH₂)₁—; *—(CH₂)₇S(O)₂(CH₂)₂—; *—(CH₂)₇S(O)₂(CH₂)₃—; *—(CH₂)₈S(O)₂(CH₂)₁—; *—(CH₂)₈S(O)₂(CH₂)₂—; *—(CH₂)₈S(O)₂(CH₂)₃—; *—(CH₂)₉S(O)₂(CH₂)₁—; *—(CH₂)₉S(O)₂(CH₂)₂—; or*—(CH₂)₉S(O)₂(CH₂)₃—;
*—(CH₂)₆—NH—(CH₂)₁—; *—(CH₂)₆—NH—(CH₂)₃—; *—(CH₂)₆—NH—(CH₂)₄—; *—(CH₂)₆—NH—(CH₂)₅—; *—(CH₂)₇—NH—(CH₂)₁—; *—(CH₂)₇—NH—(CH₂)₂—; *—(CH₂)₇—NH—(CH₂)₃—; *—(CH₂)₇—NH—(CH₂)₄—; *—(CH₂)₆—NH—CH(CH₃)—; *—(CH₂)₅—NH—CH(CH₃)—; *—(CH₂)₄—NH—CH(CH₃)—; *—(CH₂)₃—NH—CH(CH₃)—; *—(CH₂)₂—NH—CH(CH₃)—; *—(CH₂)₇—NH—CH(CH₃)—; *—(CH₂)₈—NH—CH(CH₃)—; *—(CH₂)₇—NH—CH(CF₃)—; *—(CH₂)₆—NH—CH(CF₃)—; *—(CH₂)₅—NH—CH(CF₃)—; *—(CH₂)₄—NH—CH(CF₃)—; *—(CH₂)₃—NH—CH(CF₃)—; *—(CH₂)₂—NH—CH(CF₃)—; *—(CH₂)₈—NH—CH(CF₃)—; *—CH₂—C(O)NH—(CH₂)₄—; *—CH₂—C(O)NH—(CH₂)₂—; *—CH₂—C(O)NH—(CH₂)₃—; *—CH₂—C(O)NH—(CH₂)₅—; *—(CH₂)₂-phenylene-(CH₂)₁—; *—(CH₂)₁-phenylene-(CH₂)₂—; *—(CH₂)₁-phenylene-(CH₂)₃—; *—(CH₂)₃-phenylene-(CH₂)₁—; *—(CH₂)₄-phenylene-(CH₂)₁—; *—(CH₂)₁-phenylene-(CH₂)₄—; *—(CH₂)₂-phenylene-(CH₂)₂—; *—CH₂-phenylene-CH₂—NH—CH(CH₃)—; *—CH₂-phenylene-(CH₂)₂—NH—CH(CH₃)—; *—CH₂-phenylene-CH₂—NH—CH₂—; *—CH₂-phenylene-(CH₂)₂—NH—CH₂—; *—(CH₂)₁—C(O)NH—(CH₂)₄—; *—(CH₂)₁-furanylene-(CH₂)₁—; *—(CH₂)₁-furanylene-(CH₂)₂—; *—(CH₂)₁-furanylene-(CH₂)₃—; *—(CH₂)₂-furanylene-(CH₂)₁—; *—(CH₂)₂-furanylene-(CH₂)₂—; *—(CH₂)₃-furanylene-(CH₂)₁—; *—(CH₂)₃-furanylene-(CH₂)₂—; *—(CH₂)₁-thiazolylene-(CH₂)₁—; *—(CH₂)₁-thiazolylene-(CH₂)₂—; *—(CH₂)₁-thiazolylene-(CH₂)₃—; *—(CH₂)₂-thiazolylene-(CH₂)₁—; *—(CH₂)₂-thiazolylene-(CH₂)₂—; *—(CH₂)₃-thiazolylene-(CH₂)₁—; *—(CH₂)₃-thiazolylene-(CH₂)₂—; or *—CH₂-thiazolylene-CH₂—NH—CH₂—;
wherein a hydrogen of one or more CH₂ (e.g., 1-30, 1-20, 1-15, 1-10, 1-8, 1-7, 1-6, 1-5, 1-4, 1-3, 1-2, or 1 CH₂) of the groups is optionally replaced with a substituent selected from the group consisting of: halogen, cyano, hydroxy, amino, mercapto, C₁₋₃alkyl, C₁₋₃ alkoxy, trifluoromethyl, C₅₋₁₀ aryl, 5- to 10-membered heteroaryl, C₃₋₁₂cycloalkyl, 3- to 12-membered heterocyclyl, or any combination thereof;
the piperazinylene is optionally substituted with a substituent selected from the group consisting of halogen, cyano, hydroxy, amino, mercapto, oxo, C₁₋₃ alkoxy, halogenated C₁₋₃alkyl, methanesulfonyloxy, trifluoromethanesulfonyloxy, p-toluenesulfonyloxy, optionally deuterated C₁₋₆ alkyl, optionally deuterated C₃₋₆ cycloalkyl, or any combination thereof,
the phenylene, the furanylene, and the thiazolylene are each independently optionally substituted with a substituent selected from the group consisting of halogen, cyano, hydroxy, amino, mercapto, C₁₋₃ alkoxy, halogenated C₁₋₃alkyl, methanesulfonyloxy, trifluoromethanesulfonyloxy, p-toluenesulfonyloxy, optionally deuterated C₁₋₆ alkyl, optionally deuterated C₃₋₆ cycloalkyl, or any combination thereof; and
* indicates the point of attachment to R.

In some embodiments of the compounds of Formula (I) and Formula (I-4) of the present disclosure, L represents the following optionally substituted groups:

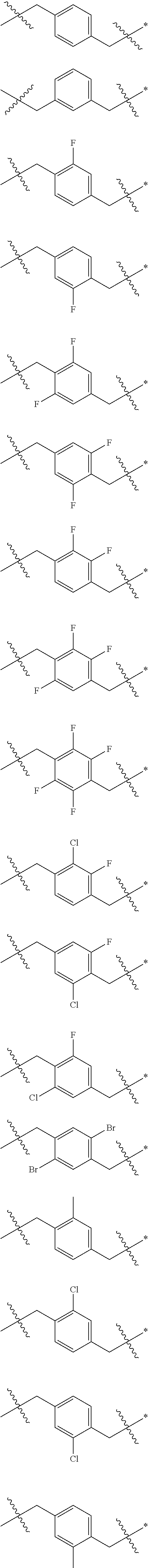

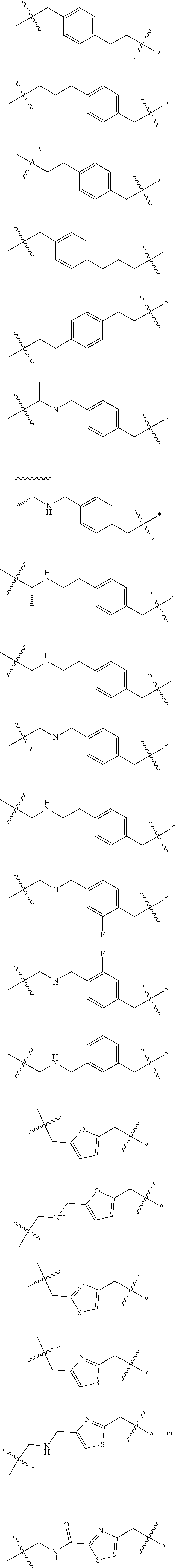

wherein * indicates the point of attachment to R.

In some embodiments of the compounds of Formula (I) and Formula (I-4) of the present disclosure, X₁ represents linear or branched C₁₋₁₀ alkyl (e.g., C₁₋₁₀ alkyl, C₁₋₆ alkyl, C₁₋₃ alkyl, fluorinated C₁₋₁₀ alkyl, fluorinated C₁₋₆ alkyl, or fluorinated C₁₋₃ alkyl, such as F₃C—, FCH₂—, F₂CH—, CF₃CF₂—, CF₃CHF—, CHF₂CF₂—, CHF₂CHF—, or CF₃CH₂—) optionally substituted with one or more (e.g., 1-15, 1-10, 1-9, 1-8, 1-7, 1-6, 1-5, 1-4, 1-3, or 1-2, or 1) fluorine.

In embodiments of the present disclosure, the number of substituents is not limited in principle, or is automatically limited by the size of the building units.

In some embodiments of the compounds of Formula (I) and Formula (I-4) of the present disclosure, X₁ represents C₃-C₁₂ cycloalkyl, which is optionally substituted with one or more (e.g., 1-10, 1-8, 1-7, 1-6, 1-5, 1-4, 1-3, 1-2, or 1) substituents selected from the group consisting of halogen, cyano, amino, hydroxy, mercapto, oxo, C₁₋₃ alkoxy, halogenated C₁₋₃ alkoxy, halogenated C₁₋₃ alkyl, C₁₋₆ alkylsulfonyl, bis(C₁₋₆ alkyl)phosphono, methanesulfonyloxy, trifluoromethanesulfonyloxy, p-toluenesulfonyloxy, optionally substituted 5- or 6-membered heterocyclyl, optionally substituted C₅-C₂₀ aryl, optionally substituted 5- or 6-membered heteroaryl, C₁₋₆ alkyl, C₁₋₃ alkoxycarbonyl, halogenated C₂₋₄ alkenyl, or any combination thereof, wherein the 5- or 6-membered heterocyclyl is optionally substituted with one or more (e.g., 1-6, 1-5, 1-4, 1-3, 1-2, or 1) substituents selected from the group consisting of halogen, cyano, amino, hydroxy, mercapto, oxo, C₁₋₆ alkyl, halogenated C₁₋₃ alkyl, or any combination thereof; and the C₅-C₂₀ aryl is optionally substituted with one or more (e.g., 1-6, 1-5, 1-4, 1-3, 1-2, or 1) substituents selected from the group consisting of halogen, cyano, amino, hydroxy, mercapto, C₁₋₆alkyl, halogenated C₁₋₃ alkyl, or any combination thereof; and the 5- or 6-membered heteroaryl is optionally substituted with one or more (e.g., 1-6, 1-5, 1-4, 1-3, 1-2, or 1) substituents selected from the group consisting of halogen, cyano, amino, hydroxy, mercapto, oxo, C₁₋₆ alkyl, halogenated C₁₋₃ alkyl, or any combination thereof.

In some embodiments of the compounds of Formula (I) and Formula (I-4) of the present disclosure, X₁ represents cyclopropyl, cyclobutyl, 3,3-difluorocyclobutyl, cyclopentyl, cyclopentenyl, cyclohexyl, cyclohexenyl, cycloheptyl, cyclooctyl, decalinyl, octahydropentalenyl, octahydro-1H-indenyl, 2,3-dihydro-1H-indenyl, 4-cyano-2,3-dihydro-1H-indenyl, spiro[3.3]heptanyl, spiro[2.5]octyl, spiro[3.5]nonyl, spiro[4.4]nonyl, spiro[4.5]decyl, spiro[5.5]undecyl, p-menthanyl, m-menthanyl, quinuclidinyl, adamantanyl, noradamantanyl, bornyl, 3,5-dimethyladamantan-1-yl, 1,7,7-trimethylbicyclo[2.2.1]heptan-2-yl, 7,7-dimethylbicyclo[2.2.1]heptan-1-yl, 7,7-dimethyl-2-oxobicyclo[2.2.1]heptan-1-yl, bicyclo[2.2.1]heptanyl, 2-oxobicyclo[2.2.1]heptyl, or bicyclo[2.2.1]heptenyl, with each group being optionally substituted with one or more substituents selected from the group consisting of halogen, cyano, amino, hydroxy, mercapto, oxo, C₁₋₃ alkoxy, halogenated C₁₋₃ alkoxy, halogenated C₁₋₃ alkyl, C₁₋₆ alkylsulfonyl, bis(C₁₋₆ alkyl)phosphono, methanesulfonyloxy, trifluoromethanesulfonyloxy, p-toluenesulfonyloxy, optionally substituted 5- or 6-membered heterocyclyl, optionally substituted C₅-C₂₀ aryl, optionally substituted 5- or 6-membered heteroaryl, C₁₋₆ alkyl, C₁₋₃ alkoxycarbonyl, halogenated C₂₋₄ alkenyl, or any combination thereof, wherein the 5- or 6-membered heterocyclyl is optionally substituted with one or more (e.g., 1-6, 1-5, 1-4, 1-3, 1-2, or 1) substituents selected from the group consisting of: halogen, cyano, amino, hydroxy, mercapto, oxo, C₁₋₆alkyl, halogenated C₁₋₃alkyl, or any combination thereof; and the C₅-C₂₀ aryl is optionally substituted with one or more (e.g., 1-6, 1-5, 1-4, 1-3, 1-2, or 1) substituents selected from the group consisting of: halogen, cyano, amino, hydroxy, mercapto, C₁₋₆ alkyl, halogenated C₁₋₃ alkyl, or any combination thereof; and the 5- or 6-membered heteroaryl is optionally substituted with one or more (e.g., 1-6, 1-5, 1-4, 1-3, 1-2, or 1) substituents selected from the group consisting of: halogen, cyano, amino, hydroxy, mercapto, oxo, C₁₋₆ alkyl, halogenated C₁₋₃ alkyl, or any combination thereof.

In some embodiments of the compounds of Formula (I) and Formula (I-4) of the present disclosure, X₁ represents adamantanyl, noradamantanyl, bornyl, or norbornyl, with each group being optionally substituted with one or more (e.g., 1-6, 1-5, 1-4, 1-3, 1-2, or 1) substituents selected from the group consisting of halogen, cyano, amino, hydroxy, mercapto, oxo, C₁₋₃ alkoxy, halogenated C₁₋₃ alkoxy, halogenated C₁₋₃alkyl, C₁₋₆ alkylsulfonyl, bis(C₁₋₆ alkyl)phosphono, methanesulfonyloxy, trifluoromethanesulfonyloxy, p-toluenesulfonyloxy, optionally substituted 5- or 6-membered heterocyclyl, optionally substituted C₅-C₂₀ aryl, optionally substituted 5- or 6-membered heteroaryl, C₁₋₆ alkyl, C₁₋₃ alkoxycarbonyl, halogenated C₂₋₄ alkenyl, or any combination thereof, wherein the 5- or 6-membered heterocyclyl is optionally substituted with one or more substituents selected from the group consisting of: halogen, cyano, amino, hydroxy, mercapto, oxo, C₁₋₆ alkyl, halogenated C₁₋₃ alkyl, or any combination thereof; and the C₅-C₂₀ aryl is optionally substituted with one or more (e.g., 1-6, 1-5, 1-4, 1-3, 1-2, or 1) substituents selected from the group consisting of halogen, cyano, amino, hydroxy, mercapto, C₁₋₆ alkyl, halogenated C₁₋₃ alkyl, or any combination thereof; and the 5- or 6-membered heteroaryl is optionally substituted with one or more (e.g., 1-6, 1-5, 1-4, 1-3, 1-2, or 1) substituents selected from the group consisting of halogen, cyano, amino, hydroxy, mercapto, oxo, C₁₋₆ alkyl, halogenated C₁₋₃ alkyl, or any combination thereof.

In some embodiments of the compounds of Formula (I) and Formula (I-4) of the present disclosure, X₁ represents C₅-C₁₄ aryl, which is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, C₁₋₃ alkoxy, halogenated C₁₋₃ alkoxy, halogenated C₁₋₃ alkyl, C₁₋₆ alkylsulfonyl, bis(C₁₋₆ alkyl)phosphono, methanesulfonyloxy, trifluoromethanesulfonyloxy, p-toluenesulfonyloxy, optionally substituted 5- or 6-membered heterocyclyl, optionally substituted C₅-C₂₀ aryl, optionally substituted 5- or 6-membered heteroaryl, C₁₋₆ alkyl, C₁₋₃ alkoxycarbonyl, halogenated C₂₋₄ alkenyl, or any combination thereof, wherein the 5- or 6-membered heterocyclyl is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, oxo, C₁₋₆ alkyl, halogenated C₁₋₃ alkyl, or any combination thereof; and the C₅-C₂₀ aryl is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, C₁₋₆alkyl, halogenated C₁₋₃ alkyl, or any combination thereof; and the 5- or 6-membered heteroaryl is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, oxo, C₁₋₆ alkyl, halogenated C₁₋₃ alkyl, or any combination thereof.

In some embodiments of the compounds of Formula (I) and Formula (I-4) of the present disclosure, X₁ represents phenyl or naphthyl, with each group being optionally substituted with one or more (e.g., 1-5, 1-4, 1-3, or 1-2, or 1) substituents selected from the group consisting of halogen, cyano, amino, hydroxy, mercapto, C₁₋₃ alkoxy, halogenated C₁₋₃ alkoxy, halogenated C₁₋₃ alkyl, C₁₋₆ alkylsulfonyl, bis(C₁₋₆ alkyl)phosphono, methanesulfonyloxy, trifluoromethanesulfonyloxy, p-toluenesulfonyloxy, optionally substituted 5- or 6-membered heterocyclyl, optionally substituted C₅-C₂₀ aryl, optionally substituted 5- or 6-membered heteroaryl, C₁₋₆ alkyl, C₁₋₃ alkoxycarbonyl, halogenated C₂₋₄ alkenyl, or any combination thereof, wherein the 5- or 6-membered heterocyclyl is optionally substituted with one or more (e.g., 1-10, 1-9, 1-8, 1-7, 1-6, 1-5, 1-4, 1-3 or 1-2, or 1) substituents selected from the group consisting of halogen, cyano, amino, hydroxy, mercapto, oxo, C₁₋₆ alkyl, halogenated C₁₋₃alkyl, or any combination thereof; and the C₅-C₂₀ aryl is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, C₁₋₆ alkyl, halogenated C₁₋₃ alkyl, or any combination thereof; and the 5- or 6-membered heteroaryl is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, oxo, C₁₋₆ alkyl, halogenated C₁₋₃ alkyl, or any combination thereof.

In some embodiments of the compounds of Formula (I) and Formula (I-4) of the present disclosure, X₁ represents phenyl, 4-fluorophenyl, 4-chlorophenyl, 3,5-difluorophenyl, 4-(bromomethyl)-2-fluorophenyl, 3,4,5-trifluorophenyl, or (2-bromovinyl)phenyl.

In some embodiments of the compounds of Formula (I) and Formula (I-4) of the present disclosure, X₁ represents 3- to 12-membered heterocyclyl, which is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, oxo, C₁₋₃ alkoxy, halogenated C₁₋₃ alkoxy, halogenated C₁₋₃alkyl, C₁₋₆ alkylsulfonyl, bis(C₁₋₆ alkyl)phosphono, methanesulfonyloxy, trifluoromethanesulfonyloxy, p-toluenesulfonyloxy, optionally substituted 5- or 6-membered heterocyclyl, optionally substituted C₅-C₂₀ aryl, optionally substituted 5- or 6-membered heteroaryl, C₁₋₆ alkyl, C₁₋₃ alkoxycarbonyl, halogenated C₂₋₄ alkenyl, or any combination thereof, wherein the 5- or 6-membered heterocyclyl is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, oxo, C₁₋₆ alkyl, halogenated C₁₋₃ alkyl, or any combination thereof; and the C₅-C₂₀ aryl is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, C₁₋₆ alkyl, halogenated C₁₋₃ alkyl, or any combination thereof; and the 5- or 6-membered heteroaryl is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, oxo, C₁₋₆alkyl, halogenated C₁₋₃ alkyl, or any combination thereof.

In some embodiments of the compounds of Formula (I) and Formula (I-4) of the present disclosure, X₁ represents azetidinyl, oxetanyl, pyrrolidinyl, imidazolidinyl, pyrazolidinyl, oxazolidinyl, thiazolidinyl, piperidinyl, piperazinyl, morpholinyl, thiomorpholinyl, azacycloheptyl, azacyclooctyl, diazacycloheptanyl, diazacyclooctyl, 3-azabicyclo[3.1.0]hexyl, 3-azabicyclo[3.1.1]heptanyl, 3-azabicyclo[4.1.0]heptanyl, 2-azabicyclo[2.2.1]heptanyl, 6-azabicyclo[3.1.1]heptanyl, 2-azabicyclo[2.2.2]octyl, 2,5-diazabicyclo[2.2.1]heptanyl, 3,6-diazabicyclo[3.1.1]heptanyl, 3-azabicyclo[3.2.1]octyl, 3,8-diazabicyclo[3.2.1]octyl, 2,5-diazabicyclo[2.2.2]octyl, 2,6-diazaspiro[3.3]heptanyl, 2,7-diazaspiro[3.5]nonyl, 3-azaspiro[5.5]undecyl, 5-azaspiro[2.4]heptanyl, 6-azaspiro[2.5]octyl, 2-oxa-7-azaspiro[3.5]nonyl, 7-azaspiro[3.5]nonyl, hexahydro-1H-isoindol-2(3H)-yl, (3aR,7aS)-octahydro-2H-isoindol-2-yl, octahydropyrrolo[3,4-c]pyrrolyl, or 5,7-dihydro-6H-pyrrolo[3,4-b]pyridyl, with each group being optionally substituted with one or more (e.g., 1-10, 1-9, 1-8, 1-7, 1-6, 1-5, 1-4, 1-3 or 1-2, or 1) substituents selected from the group consisting of halogen, cyano, amino, hydroxy, mercapto, oxo, C₁₋₃ alkoxy, halogenated C₁₋₃ alkoxy, halogenated C₁₋₃ alkyl, C₁₋₆ alkylsulfonyl, bis(C₁₋₆ alkyl)phosphono, methanesulfonyloxy, trifluoromethanesulfonyloxy, p-toluenesulfonyloxy, optionally substituted 5- or 6-membered heterocyclyl, optionally substituted C₅-C₂₀ aryl, optionally substituted 5- or 6-membered heteroaryl, C₁₋₆ alkyl, C₁₋₃ alkoxycarbonyl, halogenated C₂₋₄ alkenyl, or any combination thereof, wherein the 5- or 6-membered heterocyclyl is optionally substituted with one or more (e.g., 1-10, 1-9, 1-8, 1-7, 1-6, 1-5, 1-4, 1-3 or 1-2, or 1) substituents selected from the group consisting of halogen, cyano, amino, hydroxy, mercapto, oxo, C₁₋₆ alkyl, halogenated C₁₋₃ alkyl, or any combination thereof; and the C₅-C₂₀ aryl is optionally substituted with one or more (e.g., 1-10, 1-9, 1-8, 1-7, 1-6, 1-5, 1-4, 1-3 or 1-2, or 1) substituents selected from the group consisting of halogen, cyano, amino, hydroxy, mercapto, C₁₋₆ alkyl, halogenated C₁₋₃ alkyl, or any combination thereof; and the 5- or 6-membered heteroaryl is optionally substituted with one or more (e.g., 1-10, 1-9, 1-8, 1-7, 1-6, 1-5, 1-4, 1-3 or 1-2, or 1) substituents selected from the group consisting of halogen, cyano, amino, hydroxy, mercapto, oxo, C₁₋₆ alkyl, halogenated C₁₋₃ alkyl, or any combination thereof.

In some embodiments of the compounds of Formula (I) and Formula (I-4) of the present disclosure, X₁ represents morpholinyl, piperidinyl, difluoropiperidinyl, 4,4-dimethylpiperidin-1-yl, 3,5-dimethylpiperidin-1-yl, piperazinyl, 3-hydroxy-2-methylazetidin-1-yl, trifluoromethoxyazetidinyl, azetidinyl, cyanoazetidinyl, hydroxyazetidinyl, 3-hydroxy-3-methylazetidin-1-yl, 3-fluoro-4-hydroxypyrrolidin-1-yl, 3-methyl-4-(trifluoromethyl)pyrrolidin-1-yl, 7-fluoro-5-azaspiro[2.4]heptan-5-yl, 3-azabicyclo[3.1.0]hexan-3-yl, 6-(difluoromethyl)-3-azabicyclo[4.1.0]heptan-3-yl, 7-(3-fluoro-5,7-dihydro-6H-pyrrolo[3,4-b]pyridin-6-yl, 1,1-difluoro-5-azaspiro[2.4]heptan-5-yl, 1,1-difluoro-6-azaspiro[2.5]octan-6-yl, 2-oxa-7-azaspiro[3.5]nonan-7-yl, 4-(methylsulfonyl)piperazin-1-yl, 4-(ethylsulfonyl)piperazin-1-yl, 1,4-diazepan-1-yl, 3,8-diazabicyclo[3.2.1]octan-3-yl, 2-azabicyclo[3.2.1]octanyl, 1,4-diazabicyclo[3.2.1]octanyl, 2,5-diazabicyclo[2.2.2]octan-2-yl, or pyrrolidinyl.

In some embodiments of the compounds of Formula (I) and Formula (I-4) of the present disclosure, X₁ represents: morpholinyl, piperidinyl, 2-oxopiperidin-1-yl, piperazinyl, N-methylpiperazinyl, 4-(methylsulfonyl)piperazin-1-yl, 4-(ethylsulfonyl)piperazin-1-yl, (N-methylpiperazinyl)piperidinyl, 3,5-dimethylpiperazinyl, 1,4-diazepan-1-yl, 4-methyl-1,4-diazepan-1-yl, 1-methyl-1,4-diazepan-1-yl, 8-methyl-3,8-diazabicyclo[3.2.1]octan-3-yl, 1,4-diazabicyclo[3.2.1]octan-4-yl, 5-methyl-2,5-diazabicyclo[2.2.2]octan-2-yl, 8,8-difluoro-2-azabicyclo[3.2.1]octan-2-yl, 4-(4-fluorophenyl)piperazin-1-yl, 4-(3,4-difluorophenyl)piperazin-1-yl, 2-oxopiperidin-1-yl, 2-oxopyrrolidin-1-yl, 3-fluoro-4-hydroxypyrrolidin-1-yl, 3-methyl-4-(trifluoromethyl)pyrrolidin-1-yl, 2,6-dioxopiperidin-3-yl, 4,4-dimethylpiperidin-1-yl, 3,5-dimethylpiperidin-1-yl, 4,4-difluoropiperidin-1-yl, 4-methylpiperazin-1-yl, tetrahydro-2H-pyran-2-yl, pyrrolidinyl, pyrrolidin-1-yl, 3-cyanoazetidinyl, 3-hydroxyazetidinyl, 3-hydroxy-3-methylazetidinyl, 3-hydroxy-2-methylazetidin-1-yl, 3-(trifluoromethoxy)azetidin-1-yl, azepan-1-yl, azacyclooctan-1-yl, 7-fluoro-5-azaspiro[2.4]heptan-5-yl, 1,1-difluoro-5-azaspiro[2.4]heptan-5-yl, 1,1-difluoro-6-azaspiro[2.5]octan-6-yl, 6-azaspiro[2.5]octan-6-yl, 2-oxa-7-azaspiro[3.5]nonan-7-yl, 3-azabicyclo[3.1.0]hexan-3-yl, 6-(difluoromethyl)-3-azabicyclo[4.1.0]heptan-3-yl, thiomorpholino, [1,4′-dipiperidin]-1′-yl,

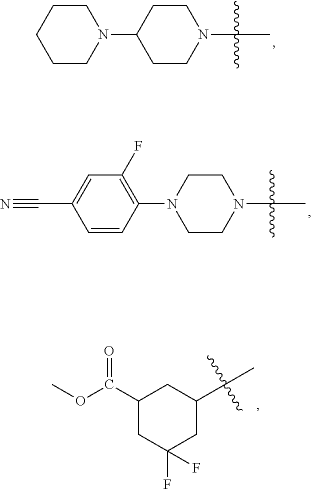 [SMILES: *N1CCC(N2CCCCC2)CC1 ; *N1CCN(c2ccc(C#N)cc2F)CC1 ; *C1CC(C(=O)OC)CC(F)(F)C1]

or 4,4-difluoropiperidinyl.

In some embodiments of the compounds of Formula (I) and Formula (I-4) of the present disclosure, X₁ represents 5- to 10-membered heteroaryl, which is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, C₁₋₃ alkoxy, halogenated C₁₋₃ alkoxy, halogenated C₁₋₃ alkyl, bis(C₁₋₆ alkyl)phosphono, C₁₋₆ alkylsulfonyl, methanesulfonyloxy, trifluoromethanesulfonyloxy, p-toluenesulfonyloxy, C₁₋₆ alkyl, optionally substituted 5- or 6-membered heterocyclyl, optionally substituted C₅-C₂₀ aryl, optionally substituted 5- or 6-membered heteroaryl, C₁₋₆ alkyl, C₁₋₃ alkoxycarbonyl, halogenated C₂₋₄ alkenyl, or any combination thereof, wherein the 5- or 6-membered heterocyclyl is optionally substituted with one or more (e.g., 1-10, 1-9, 1-8, 1-7, 1-6, 1-5, 1-4, 1-3, or 1-2, or 1) substituents selected from the group consisting of C₁₋₆ alkyl, cyano, halogenated C₁₋₃ alkyl, amino, hydroxy, mercapto, halogen, or any combination thereof; and the C₅-C₂₀ aryl is optionally substituted with one or more (e.g., 1-10, 1-9, 1-8, 1-7, 1-6, 1-5, 1-4, 1-3, or 1-2, or 1) substituents selected from the group consisting of C₁₋₆ alkyl, cyano, halogenated C₁₋₃ alkyl, amino, hydroxy, mercapto, halogen, or any combination thereof; and the 5- or 6-membered heteroaryl is optionally substituted with one or more (e.g., 1-10, 1-9, 1-8, 1-7, 1-6, 1-5, 1-4, 1-3, or 1-2, or 1) substituents selected from the group consisting of C₁₋₆ alkyl, halogenated C₁₋₃ alkyl, amino, hydroxy, mercapto, halogen, or any combination thereof.

In some embodiments of the compounds of Formula (I) and Formula (I-4) of the present disclosure, X₁ represents furanyl, oxazolyl, isoxazolyl, oxadiazolyl, thienyl, thiazolyl, isothiazolyl, thiadiazolyl, pyrrolyl, imidazolyl, pyrazolyl, triazolyl, pyridyl, pyrimidinyl, pyridazinyl, pyrazinyl, indolyl, isoindolyl, benzofuranyl, isobenzofuranyl, benzothienyl, indazolyl, benzimidazolyl, benzoxazolyl, benzisoxazolyl, benzothiazolyl, benzisothiazolyl, benzotriazolyl, benzo[2,1,3]oxadiazolyl, benzo[2,1,3]thiadiazolyl, benzo[1,2,3]thiadiazolyl, tetrahydro-2H-pyran-2-yl, quinolinyl, isoquinolinyl, naphthyridinyl, cinnolinyl, quinazolinyl, quinoxalinyl, phthalazinyl, pyrazolo[1,5-a]pyridyl, pyrazolo[1,5-a]pyrimidinyl, imidazo[1,2-a]pyridyl, 1H-pyrrolo[3,2-b]pyridyl, 1H-pyrrolo[2,3-b]pyridyl, 4H-fluoro[3,2-b]pyrrolyl, pyrrolo[2,1-b]thiazolyl, or imidazo[2,1-b]thiazolyl, with each group being optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, C₁₋₃ alkoxy, halogenated C₁₋₃ alkoxy, halogenated C₁₋₃ alkyl, bis(C₁₋₆ alkyl)phosphono, C₁₋₆ alkylsulfonyl, methanesulfonyloxy, trifluoromethanesulfonyloxy, p-toluenesulfonyloxy, C₁₋₆ alkyl, optionally substituted 5- or 6-membered heterocyclyl, optionally substituted C₅-C₂₀ aryl, optionally substituted 5- or 6-membered heteroaryl, C₁₋₆ alkyl, C₁₋₃ alkoxycarbonyl, halogenated C₂₋₄ alkenyl, or any combination thereof, wherein the 5- or 6-membered heterocyclyl is optionally substituted with one or more (e.g., 1-10, 1-9, 1-8, 1-7, 1-6, 1-5, 1-4, 1-3 or 1-2, or 1) substituents selected from the group consisting of C₁₋₆ alkyl, cyano, halogenated C₁₋₃ alkyl, amino, hydroxy, mercapto, halogen, or any combination thereof; and the C₅-C₂₀ aryl is optionally substituted with one or more (e.g., 1-10, 1-9, 1-8, 1-7, 1-6, 1-5, 1-4, 1-3 or 1-2, or 1) substituents selected from the group consisting of: C₁₋₆ alkyl, cyano, halogenated C₁₋₃alkyl, amino, hydroxy, mercapto, halogen, or any combination thereof; and the 5- or 6-membered heteroaryl is optionally substituted with one or more (e.g., 1-10, 1-9, 1-8, 1-7, 1-6, 1-5, 1-4, 1-3 or 1-2, or 1) substituents selected from the group consisting of: C₁₋₆ alkyl, halogenated C₁₋₃ alkyl, amino, hydroxy, mercapto, halogen, or any combination thereof.

In some embodiments of the compounds of Formula (I) and Formula (I-4) of the present disclosure, X₁ represents pyridyl, pyrimidinyl, thiazolyl, quinolyl, 6,7-difluoroquinazolin-4-yl, 3-cyano-quinolyl, indolyl, 1-methyl-1H-indol-7-yl, benzothienyl, benzofuranyl, quinazolinyl, 6,7-difluoroquinazolin-4-yl, 4-(pyridin-3-yl)pyrimidin-2-yl, 3-fluoro-5,7-dihydro-6H-pyrrolo[3,4-b]pyridin-6-yl, 3-(trifluoromethyl)-5,7-dihydro-6H-pyrrolo[3,4-b]pyridin-6-yl, or isoindol-2-yl.

In some embodiments of the compounds of Formula (I) and Formula (I-4) of the present disclosure, X₁ represents NR₂R₃, wherein R₂ and R₃ each independently represent H, optionally substituted linear or branched C₁₋₁₀ alkyl, optionally substituted cycloalkyl, optionally substituted aryl, optionally substituted heterocyclyl or optionally substituted heteroaryl, wherein R₂ and R₃ are not H at the same time.

In some embodiments of the compounds of Formula (I) and Formula (I-4) of the present disclosure, X₁ represents NR₂R₃, wherein
R₂ and R₃ each independently represent H, optionally substituted linear or branched C₁₋₁₀ alkyl, optionally substituted C₃-C₂₀ cycloalkyl, optionally substituted 3- to 20-membered heterocyclyl, optionally substituted C₅-C₁₄ aryl, or optionally substituted 5- to 20-membered heteroaryl, wherein R₂ and R₃ are not H at the same time,
   wherein the optionally substituted linear or branched C₁₋₁₀ alkyl is optionally substituted with one or more (e.g., 1-10, 1-9, 1-8, 1-7, 1-6, 1-5, 1-4, 1-3, or 1-2, or 1) substituents selected from the group consisting of halogen, cyano, hydroxy, amino, mercapto, C₁₋₃ alkyl, C₁₋₃ alkoxy, trifluoromethyl, optionally substituted C₅₋₁₀ aryl, optionally substituted 5- to 10-membered heteroaryl, optionally substituted C₃₋₁₂ cycloalkyl, optionally substituted 3- to 12-membered heterocyclyl, or any combination thereof, wherein the optionally substituted C₅₋₁₀ aryl, optionally substituted 5- to 10-membered heteroaryl, optionally substituted C₃₋₁₂ cycloalkyl and optionally substituted 3- to 12-membered heterocyclyl are each independently optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, C₁₋₆ alkyl, C₁₋₃ alkoxy, or any combination thereof;
   the optionally substituted C₃-C₂₀cycloalkyl and the optionally substituted 3- to 20-membered heterocyclyl are each independently optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, oxo, C₁₋₆ alkyl, C₁₋₃ alkoxy, C₁₋₃ alkoxycarbonyl, halogenated C₂₋₄ alkenyl, halogenated C₁₋₃ alkoxy, halogenated C₁₋₃ alkyl, C₁₋₆ alkylsulfonyl, bis(C₁₋₆ alkyl)phosphono, methanesulfonyloxy, trifluoromethanesulfonyloxy, p-toluenesulfonyloxy, optionally substituted 5- or 6-membered heterocyclyl, optionally substituted C₅-C₂₀ aryl, optionally substituted 5- or 6-membered heteroaryl, or any combination thereof, wherein the 5- or 6-membered heterocyclyl is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, oxo, mercapto, C₁₋₆ alkyl, halogenated C₁₋₃ alkyl, or any combination thereof; and the C₅-C₂₀ aryl is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, C₁₋₆ alkyl, halogenated C₁₋₃ alkyl, or any combination thereof; and the 5- or 6-membered heteroaryl is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, C₁₋₆ alkyl, halogenated C₁₋₃ alkyl, or any combination thereof; and
   wherein the optionally substituted C₅-C₁₄ aryl or optionally substituted 5- to 20-membered heteroaryl is each independently optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, C₁₋₃ alkoxy, halogenated C₁₋₃ alkoxy, halogenated C₁₋₃alkyl, C₁₋₆ alkylsulfonyl, bis(C₁₋₆ alkyl)phosphono, methanesulfonyloxy, trifluoromethanesulfonyloxy, p-toluenesulfonyloxy, optionally substituted 5- or 6-membered heterocyclyl, optionally substituted C₅-C₂₀ aryl, optionally substituted 5- or 6-membered heteroaryl, C₁₋₆ alkyl, C₁₋₃ alkoxycarbonyl, halogenated C₂₋₄ alkenyl, C(O)NHR_{d}, or any combination thereof, wherein Rd represents C₁₋₅ alkyl or optionally substituted phenyl, and the optionally substituted phenyl is optionally substituted with one or more (e.g., 1-5, 1-4, 1-3 or 1-2, or 1) substituents selected from the group consisting of halogen, cyano, amino, hydroxy, mercapto, oxo, and C₁₋₆ alkyl, or any combination thereof; and the 5- or 6-membered heterocyclyl is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, oxo, mercapto, C₁₋₆ alkyl, halogenated C₁₋₃ alkyl, or any combination thereof; and the C₅-C₂₀ aryl is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, C₁₋₆ alkyl, halogenated C₁₋₃ alkyl, or any combination thereof; and the 5- or 6-membered heteroaryl is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, oxo, mercapto, C₁₋₆ alkyl, halogenated C₁₋₃ alkyl, or any combination thereof.

In some embodiments of the compounds of Formula (I) and Formula (I-4) of the present disclosure, X₁ represents NR₂R₃, wherein
R₂ and R₃ each independently represent H or optionally substituted linear or branched C₁₋₁₀ alkyl, wherein R₂ and R₃ are not H at the same time, and the optionally substituted linear or branched C₁₋₁₀ alkyl is optionally substituted with one or more (e.g., 1-10, 1-9, 1-8, 1-7, 1-6, 1-5, 1-4, 1-3, or 1-2, or 1) substituents selected from the group consisting of halogen, cyano, hydroxy, amino, mercapto, C₁₋₃ alkyl, C₁₋₃ alkoxy, trifluoromethyl, optionally substituted C₅₋₁₀ aryl, optionally substituted 5- to 10-membered heteroaryl, optionally substituted C₃₋₁₂ cycloalkyl, optionally substituted 3- to 12-membered heterocyclyl, or any combination thereof, wherein the optionally substituted C₅₋₁₀ aryl, optionally substituted 5- to 10-membered heteroaryl, optionally substituted C₃₋₁₂ cycloalkyl and optionally substituted 3- to 12-membered heterocyclyl are each independently optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, C₁₋₆ alkyl, C₁₋₃ alkoxy, or any combination thereof.

In some embodiments of the compounds of Formula (I) and Formula (I-4) of the present disclosure, X₁ represents NR₂R₃, wherein
R₂ represents H or C₁₋₃ alkyl, and R₃ represents optionally substituted C₃-C₂₀ cycloalkyl, optionally substituted C₅-C₁₄ aryl, optionally substituted 3- to 20-membered heterocyclyl, or optionally substituted 5- to 20-membered heteroaryl, wherein the C₃-C₂₀ cycloalkyl and 3- to 20-membered heterocyclyl are each independently optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, oxo, C₁₋₆ alkyl, C₁₋₃ alkoxy, C₁₋₃ alkoxycarbonyl, halogenated C₂₋₄ alkenyl, halogenated C₁₋₃ alkoxy, halogenated C₁₋₃ alkyl, C₁₋₆ alkylsulfonyl, bis(C₁₋₆ alkyl)phosphono, methanesulfonyloxy, trifluoromethanesulfonyloxy, p-toluenesulfonyloxy, optionally substituted 5- or 6-membered heterocyclyl, optionally substituted C₅-C₂₀ aryl, optionally substituted 5- or 6-membered heteroaryl, or any combination thereof, wherein the 5- or 6-membered heterocyclyl is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, oxo, mercapto, C₁₋₆ alkyl, halogenated C₁₋₃ alkyl, or any combination thereof; and the C₅-C₂₀ aryl is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, C₁₋₆ alkyl, halogenated C₁₋₃ alkyl, or any combination thereof; and the 5- or 6-membered heteroaryl is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, C₁₋₆ alkyl, halogenated C₁₋₃ alkyl, or any combination thereof; and the C₅-C₁₄ aryl and 5- to 20-membered heteroaryl are each independently optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, C₁₋₃ alkoxy, halogenated C₁₋₃ alkoxy, halogenated C₁₋₃ alkyl, C₁₋₆ alkylsulfonyl, bis(C₁₋₆ alkyl)phosphono, methanesulfonyloxy, trifluoromethanesulfonyloxy, p-toluenesulfonyloxy, optionally substituted 5- or 6-membered heterocyclyl, optionally substituted C₅-C₂₀ aryl, optionally substituted 5- or 6-membered heteroaryl, C₁₋₆ alkyl, C₁₋₃ alkoxycarbonyl, halogenated C₂₋₄ alkenyl, —C(O)NHR_{d}, or any combination thereof, wherein Rd represents C₁₋₅ alkyl or optionally substituted phenyl, and the optionally substituted phenyl is optionally substituted with one or more (e.g., 1-5, 1-4, 1-3, or 1-2, or 1) substituents selected from the group consisting of halogen, cyano, amino, hydroxy, mercapto, oxo, and C₁₋₆ alkyl, or any combination thereof; and the 5- or 6-membered heterocyclyl is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, oxo, mercapto, C₁₋₆alkyl, halogenated C₁₋₃alkyl, or any combination thereof; and the C₅-C₂₀ aryl is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, C₁₋₆ alkyl, halogenated C₁₋₃ alkyl, or any combination thereof; and the 5- or 6-membered heteroaryl is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, oxo, mercapto, C₁₋₆ alkyl, halogenated C₁₋₃ alkyl, or any combination thereof.

In some embodiments of the compounds of Formula (I) and Formula (I-4) of the present disclosure, X₁ represents adamantanyl-NH—, noradamantanyl-NH—, cyclohexyl-NH—, adamantanyl-N(CH₃)—, noradamantanyl-N(CH₃)—, bornyl-NH—, norbornyl-NH—, spiro[3.3]heptyl-NH—, or N(CH₂CH₃)₂.

In some embodiments of the compounds of Formula (I) and Formula (I-4) of the present disclosure, X₁ represents NR₂R₃, wherein R₂ represents H, and R₃ represents ethyl substituted with 2,6-dichloro-3-fluorophenyl.

In some embodiments of the compounds of Formula (I) and Formula (I-4) of the present disclosure, X₁ represents NR₂R₃, wherein:
R₂ represents H, methyl, ethyl, isopropyl, or cyclohexyl, and
R₃ represents the following optionally substituted groups: adamantanyl, noradamantanyl, norbornyl, cyclohexyl, cyclopentyl, cyclopropyl, cyclobutyl, fluorocyclobutyl, difluorocyclobutyl, difluorocyclopentyl, hydroxycyclohexyl, ethyl, isopropyl, tert-butyl, methyl, 2,4-dimethylpent-3-yl, dicyclopropylmethyl, spiro[3.3]heptyl, 2-methoxycyclopropyl, hexahydro-1H-isoindol-2(3H)-yl, bicyclo[1.1.1]pentyl, bicyclo[2.2.2]octyl, 4-hydroxybicyclo[2.2.2]octan-1-yl, 4,4-dimethylcyclohexyl, oxetanyl, oxazolyl, 2,3-dihydro-1H-indenyl, quinuclidinyl, 1,7,7-trimethylbicyclo[2.2.1]heptyl, 7,7-dimethylbicyclo[2.2.1]heptyl, bicyclo[2.2.1]heptyl, 1-cyclopropylethyl,

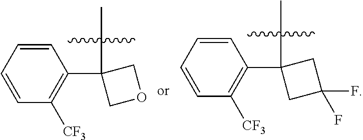 [SMILES: *C1(c2ccccc2C(F)(F)F)COC1 ; *C1(c2ccccc2C(F)(F)F)CC(F)(F)C1]

In some embodiments of the compounds of Formula (I) and Formula (I-4) of the present disclosure, X₁ represents NR₂R₃, wherein R₂ represents H, and R₃ represents-thiazolyl-C(O)NHR_{d}, where Rd represents C₁₅ alkyl or 2-chloro-6-methylphenyl.

In some embodiments of the compounds of Formula (I) and Formula (I-4) of the present disclosure, X₁ represents NR₂R₃, wherein R₂ represents H, and R₃ represents pyrimidin-4-yl, pyridin-4-yl, or quinazolin-4-yl.

In some embodiments of the compounds of Formula (I) and Formula (I-4) of the present disclosure, X₁ represents NR₂R₃, wherein R₂ and R₃ represents phenyl.

In some embodiments of the compounds of Formula (I) and Formula (I-4) of the present disclosure, X₁ represents NHCH₃, N(CH₃)₂, N(CH₂CH₃)₂ or N(CH(CH₃)₂)₂.

In some embodiments of the compounds of Formula (I) and Formula (I-4) of the present disclosure, X₁ represents NR₂R₃, wherein R₂ represents H or methyl, and R₃ represents pyrimidinyl, pyridyl, quinazolinyl, 6,7-difluoroquinazolin-4-yl, phenyl, 3-chloro-4-methylphenyl, 4-fluorophenyl, 3,5-difluorophenyl, 3,4,5-trifluorophenyl, 2-(dimethylphosphono)phenyl, 2-(isopropylsulfonyl)phenyl, 4-(pyridin-3-yl)pyrimidin-2-yl, indolyl, 1-methyl-1H-indol-7-yl, benzothienyl, benzo[b]thien-7-yl, benzofuran-7-yl, benzofuranyl, 3-cyano-quinol-4-yl, quinolyl, thiazolyl, adamantanyl, adamantan-1-yl, adamantan-2-yl, 3,5-dimethyladamantan-1-yl, 1,7,7-trimethylbicyclo[2.2.1]heptan-2-yl, 4-(4-methylpiperazin-1-yl)piperidin-1-yl, cyclohexyl, dimethylcyclohexyl, 4,4-dimethylcyclohexyl, spiro-cycloalkyl, spiro[5.5]undec-3-yl, spiro[3.3]heptyl, spiro[3.3]hept-2-yl, pyrrolidinyl, azepanyl, azacyclooctanyl, piperidinyl, dimethylpiperidinyl, or azaspirocycloalkyl.

In some embodiments of the compounds of Formula (I) and Formula (I-4) of the present disclosure, X₁ represents NR₂R₃, wherein
R₂ represents H, methyl, ethyl, isopropyl, or cyclohexyl, and
R₃ represents bicyclo[1.1.1]pent-1-yl, 4,4-dimethylcyclohexyl, 2-methoxycyclopropyl, 3-fluorocyclobutyl, 3,3-difluorocyclobutyl, 3,3-difluoro-1-(2-(trifluoromethyl)phenyl)cyclobutyl, 3-(2-(trifluoromethyl)phenyl)oxetan-3-yl, 3,3-difluorocyclopentyl, 3-hydroxycyclohexyl, 3-hydroxycyclohexyl, 3-cyano-bicyclo[1.1.1]pent-1-yl, bicyclo[2.2.2]octan-1-yl, 4-hydroxybicyclo[2.2.2]octan-1-yl, 2-isopropyl-5-methylcyclohexyl, 5-methyloxazol-2-yl, 4-cyano-2,3-dihydro-1H-inden-1-yl, 2,4-dimethylpentan-3-yl, dicyclopropylmethyl, quinuclidin-3-yl, (S)-quinuclidin-3-yl, (R)-quinuclidin-3-yl, adamantan-1-yl, 3-hydroxyadamantanyl, 3-hydroxyadamantan-1-yl, 3-chloroadamantan-1-yl, 4-chloroadamantan-1-yl, 2-chloroadamantan-1-yl, adamantan-2-yl, 1,7,7-trimethylbicyclo[2.2.1]heptan-2-yl, 7,7-dimethylbicyclo[2.2.1]heptan-1-yl, 7,7-dimethyl-2-oxobicyclo[2.2.1]heptan-1-yl, 3,5-dimethyladamantanyl, 3,5-dimethyladamantan-1-yl, hexahydro-2,5-methanopentalen-3a(1H)-yl, bicyclo[2.2.1]heptyl, bicyclo[2.2.1]heptan-2-yl, 1-cyclopropylethyl,

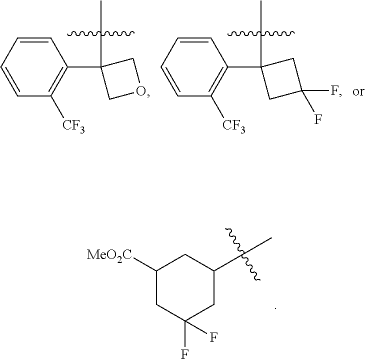

In some embodiments of the compounds of Formula (I) and Formula (I-4) of the present disclosure, X₁ represents C(O)NR₄R₅, wherein R₄ and R₅ each independently represent H, optionally substituted linear or branched C₁₋₁₀ alkyl, optionally substituted cycloalkyl, optionally substituted aryl, optionally substituted heterocyclyl, or optionally substituted heteroaryl; or wherein R₄ and R₅ together with the nitrogen atom to which they are attached form optionally substituted 5- to 8-membered heterocyclyl.

In some embodiments of the compounds of Formula (I) and Formula (I-4) of the present disclosure, X₁ represents C(O)NR₄R₅, wherein
R₄ and R₅ each independently represent H, optionally substituted linear or branched C₁₋₁₀ alkyl, optionally substituted C₃-C₂₀ cycloalkyl, optionally substituted C₅-C₁₄ aryl, optionally substituted 3- to 20-membered heterocyclyl, or optionally substituted 5- to 20-membered heteroaryl;
   wherein the optionally substituted linear or branched C₁₋₁₀ alkyl is optionally substituted with one or more (e.g., 1-10, 1-9, 1-8, 1-7, 1-6, 1-5, 1-4, 1-3, or 1-2, or 1) substituents selected from the group consisting of halogen, cyano, hydroxy, amino, mercapto, C₁₋₃ alkyl, C₁₋₃ alkoxy, trifluoromethyl, C₅₋₁₀ aryl, 5- to 10-membered heteroaryl, C₃₋₁₂ cycloalkyl, 3- to 12-membered heterocyclyl, or any combination thereof;
   the optionally substituted C₃-C₂₀ cycloalkyl and the optionally substituted 3- to 20-membered heterocyclyl are each independently optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, oxo, mercapto, C₁₋₆ alkyl, C₁₋₃ alkoxy, C₁₋₃ alkoxycarbonyl, halogenated C₂₋₄ alkenyl, halogenated C₁₋₃ alkoxy, halogenated C₁₋₃ alkyl, C₁₋₆ alkylsulfonyl, bis(C₁₋₆ alkyl)phosphono, methanesulfonyloxy, trifluoromethanesulfonyloxy, p-toluenesulfonyloxy, optionally substituted 5- or 6-membered heterocyclyl, optionally substituted C₅-C₂₀ aryl, optionally substituted 5- or 6-membered heteroaryl, or any combination thereof, wherein the 5- or 6-membered heterocyclyl is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, oxo, mercapto, C₁₋₆ alkyl, halogenated C₁₋₃ alkyl, or any combination thereof; and the C₅-C₂₀ aryl is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, C₁₋₆ alkyl, halogenated C₁₋₃ alkyl, or any combination thereof; and the 5- or 6-membered heteroaryl is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, C₁₋₆ alkyl, halogenated C₁₋₃ alkyl, or any combination thereof;
   the optionally substituted C₅-C₁₄ aryl or optionally substituted 5- to 20-membered heteroaryl is each independently optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, C₁₋₃ alkoxy, halogenated C₁₋₃ alkoxy, halogenated C₁₋₃ alkyl, C₁₋₆ alkylsulfonyl, bis(C₁₋₆ alkyl)phosphono, methanesulfonyloxy, trifluoromethanesulfonyloxy, p-toluenesulfonyloxy, optionally substituted 5- or 6-membered heterocyclyl, optionally substituted C₅-C₂₀ aryl, optionally substituted 5- or 6-membered heteroaryl, C₁₋₆ alkyl, C₁₋₃ alkoxycarbonyl, halogenated C₂₋₄ alkenyl, or any combination thereof, wherein the 5- or 6-membered heterocyclyl is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, oxo, mercapto, C₁₋₆ alkyl, halogenated C₁₋₃ alkyl, or any combination thereof; and the C₅-C₂₀ aryl is optionally substituted with one or more (e.g., 1-10, 1-9, 1-8, 1-7, 1-6, 1-5, 1-4, 1-3 or 1-2, or 1) substituents selected from the group consisting of halogen, cyano, amino, hydroxy, mercapto, C₁₋₆ alkyl, halogenated C₁₋₃ alkyl, or any combination thereof; and the 5- or 6-membered heteroaryl is optionally substituted with one or more (e.g., 1-10, 1-9, 1-8, 1-7, 1-6, 1-5, 1-4, 1-3 or 1-2, or 1) substituents selected from the group consisting of halogen, cyano, amino, hydroxy, oxo, mercapto, C₁₋₆ alkyl, halogenated C₁₋₃ alkyl, or any combination thereof.

In some embodiments of the compounds of Formula (I) and Formula (I-4) of the present disclosure, X₁ represents C(O)NR₄R₅, wherein R₄ and R₅ together with the nitrogen atom to which they are attached form optionally substituted 5- to 8-membered heterocyclyl;
the optionally substituted 5- to 8-membered heterocyclyl is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, C₁₋₃ alkoxy, halogenated C₁₋₃ alkoxy, halogenated C₁₋₃alkyl, C₁₋₆ alkylsulfonyl, bis(C₁₋₆ alkyl)phosphono, methanesulfonyloxy, trifluoromethanesulfonyloxy, p-toluenesulfonyloxy, optionally substituted 5- or 6-membered heterocyclyl, optionally substituted C₅-C₂₀ aryl, optionally substituted 5- or 6-membered heteroaryl, C₁₋₆alkyl, C₁₋₃ alkoxycarbonyl, halogenated C₂₋₄ alkenyl, or any combination thereof, wherein the 5- or 6-membered heterocyclyl is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, oxo, C₁₋₆alkyl, halogenated C₁₋₃alkyl, or any combination thereof; and the C₅-C₂₀ aryl is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, C₁₋₆alkyl, halogenated C₁₋₃alkyl, or any combination thereof; and the 5- or 6-membered heteroaryl is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, oxo, C₁₋₆alkyl, halogenated C₁₋₃ alkyl, or any combination thereof.

In some embodiments of the compounds of Formula (I) and Formula (I-4) of the present disclosure, X₁ represents piperidinyl-C(O)—, difluoropiperidinyl-C(O)—, —C(O)N(CH(CH₃)₂)₂, C(O)NH₂, N,N-diisopropylcarbamoyl, adamantanyl-NHC(O)—, 3,5-dimethyladamantan-1-yl-NHC(O)—, or adamantan-2-yl-NHC(O)—.

In some embodiments of the compounds of Formula (I) and Formula (I-4) of the present disclosure, X₁ represents NHC(O)R₆, wherein R₆ represents optionally substituted linear or branched C₁₋₆alkyl, optionally substituted cycloalkyl, optionally substituted aryl, optionally substituted heteroaryl or optionally substituted heterocyclyl.

In some embodiments of the compounds of Formula (I) and Formula (I-4) of the present disclosure, X₁ represents NHC(O)R₆, wherein R₆ represents optionally substituted linear or branched C₁₋₁₀alkyl, optionally substituted C₃-C₂₀ cycloalkyl, optionally substituted C₅-C₁₄ aryl, optionally substituted 5- to 20-membered heteroaryl, or optionally substituted 3- to 20-membered heterocyclyl; wherein the optionally substituted linear or branched C₁₋₁₀ alkyl is optionally substituted with one or more (e.g., 1-10, 1-9, 1-8, 1-7, 1-6, 1-5, 1-4, 1-3, or 1-2, or 1) substituents selected from the group consisting of halogen, cyano, hydroxy, amino, mercapto, C₁₋₃alkyl, C₁₋₃ alkoxy, trifluoromethyl, C₅₋₁₀ aryl, 5- to 10-membered heteroaryl, C₃₋₁₂ cycloalkyl, 3- to 12-membered heterocyclyl, or any combination thereof;
the optionally substituted C₃-C₂₀ cycloalkyl and the optionally substituted 3- to 20-membered heterocyclyl are each independently optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, oxo, C₁₋₆alkyl, C₁₋₃ alkoxy, C₁₋₃ alkoxycarbonyl, halogenated C₂₋₄ alkenyl, halogenated C₁₋₃ alkoxy, halogenated C₁₋₃alkyl, C₁₋₆ alkylsulfonyl, bis(C₁₋₆ alkyl)phosphono, methanesulfonyloxy, trifluoromethanesulfonyloxy, p-toluenesulfonyloxy, optionally substituted 5- or 6-membered heterocyclyl, optionally substituted C₅-C₂₀ aryl, optionally substituted 5- or 6-membered heteroaryl, or any combination thereof, wherein the 5- or 6-membered heterocyclyl is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, oxo, C₁₋₆alkyl, halogenated C₁₋₃alkyl, or any combination thereof; and the C₅-C₂₀ aryl is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, C₁₋₆ alkyl, halogenated C₁₋₃alkyl, or any combination thereof; and the 5- or 6-membered heteroaryl is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, C₁₋₆alkyl, halogenated C₁₋₃alkyl, or any combination thereof; and the optionally substituted C₅-C₁₄ aryl or optionally substituted 5- to 20-membered heteroaryl is each independently optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, C₁₋₃ alkoxy, halogenated C₁₋₃ alkoxy, halogenated C₁₋₃alkyl, C₁₋₆ alkylsulfonyl, bis(C₁₋₆ alkyl)phosphono, methanesulfonyloxy, trifluoromethanesulfonyloxy, p-toluenesulfonyloxy, optionally substituted 5- or 6-membered heterocyclyl, optionally substituted C₅-C₂₀ aryl, optionally substituted 5- or 6-membered heteroaryl, C₁₋₆ alkyl, C₁₋₃ alkoxycarbonyl, halogenated C₂₋₄ alkenyl, or any combination thereof, wherein the 5- or 6-membered heterocyclyl is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, oxo, C₁₋₆ alkyl, halogenated C₁₋₃ alkyl, or any combination thereof; and the C₅-C₂₀ aryl is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, C₁₋₆ alkyl, halogenated C₁₋₃ alkyl, or any combination thereof; and the 5- or 6-membered heteroaryl is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, oxo, C₁₋₆ alkyl, halogenated C₁₋₃ alkyl, or any combination thereof; or

In some embodiments of the compounds of Formula (I) and Formula (I-4) of the present disclosure, X₁ represents NHC(O)R₆, wherein R₆ represents adamantanyl-C(O)NH—, adamantan-1-yl-C(O)NH—, 3-hydroxyadamantanyl-C(O)NH—, 3-hydroxyadamantan-1-yl-C(O)NH—, 3-chloroadamantan-1-yl-C(O)NH—, 4-chloroadamantan-1-yl-C(O)NH—, 2-chloroadamantan-1-yl-C(O)NH—, adamantan-2-yl-C(O)NH— or —NHC(O)CH₃.

In some embodiments of the compounds of Formula (I) and Formula (I-4) of the present disclosure, X₁ represents NHC(O)NR₇R₈, wherein R₇ and R₈ each independently represent H, optionally substituted linear or branched C₁₋₁₀ alkyl, optionally substituted cycloalkyl, optionally substituted aryl, optionally substituted heterocyclyl or optionally substituted heteroaryl.

In some embodiments of the compounds of Formula (I) and Formula (I-4) of the present disclosure, X₁ represents NHC(O)NR₇R₈, wherein:
R₇ and R₈ each independently represent H, optionally substituted linear or branched C₁₋₁₀ alkyl, optionally substituted C₃-C₂₀ cycloalkyl, optionally substituted 3- to 20-membered heterocyclyl, optionally substituted C₅-C₁₄ aryl or optionally substituted 5- to 20-membered heteroaryl,
   wherein the optionally substituted linear or branched C₁₋₁₀ alkyl is optionally substituted with one or more (e.g., 1-10, 1-9, 1-8, 1-7, 1-6, 1-5, 1-4, 1-3, or 1-2, or 1) substituents selected from the group consisting of halogen, cyano, hydroxy, amino, mercapto, C₁₋₃ alkyl, C₁₋₃ alkoxy, trifluoromethyl, optionally substituted C₅₋₁₀ aryl, optionally substituted 5- to 10-membered heteroaryl, optionally substituted C₃₋₁₂ cycloalkyl, optionally substituted 3- to 12-membered heterocyclyl, or any combination thereof, wherein the optionally substituted C₅₋₁₀ aryl, optionally substituted 5- to 10-membered heteroaryl, optionally substituted C₃₋₁₂ cycloalkyl, and optionally substituted 3- to 12-membered heterocyclyl are each independently optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, C₁₋₆ alkyl, C₁₋₃ alkoxy, or any combination thereof;
   the optionally substituted C₃-C₂₀ cycloalkyl and the optionally substituted 3- to 20-membered heterocyclyl are each independently optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, oxo, C₁₋₆ alkyl, C₁₋₃ alkoxy, C₁₋₃ alkoxycarbonyl, halogenated C₂₋₄ alkenyl, halogenated C₁₋₃ alkoxy, halogenated C₁₋₃alkyl, C₁₋₆ alkylsulfonyl, bis(C₁₋₆ alkyl)phosphono, methanesulfonyloxy, trifluoromethanesulfonyloxy, p-toluenesulfonyloxy, optionally substituted 5- or 6-membered heterocyclyl, optionally substituted C₅-C₂₀ aryl, optionally substituted 5- or 6-membered heteroaryl, or any combination thereof, wherein the 5- or 6-membered heterocyclyl is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, oxo, mercapto, C₁₋₆ alkyl, halogenated C₁₋₃ alkyl, or any combination thereof; and the C₅-C₂₀ aryl is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, C₁₋₆ alkyl, halogenated C₁₋₃ alkyl, or any combination thereof; and the 5- or 6-membered heteroaryl is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, C₁₋₆ alkyl, halogenated C₁₋₃ alkyl, or any combination thereof; and wherein the optionally substituted C₅-C₁₄ aryl or optionally substituted 5- to 20-membered heteroaryl is each independently optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, C₁₋₃ alkoxy, halogenated C₁₋₃ alkoxy, halogenated C₁₋₃ alkyl, C₁₋₆ alkylsulfonyl, bis(C₁₋₆ alkyl)phosphono, methanesulfonyloxy, trifluoromethanesulfonyloxy, p-toluenesulfonyloxy, optionally substituted 5- or 6-membered heterocyclyl, optionally substituted C₅-C₂₀ aryl, optionally substituted 5- or 6-membered heteroaryl, C₁₋₆ alkyl, C₁₋₃ alkoxycarbonyl, halogenated C₂₋₄ alkenyl, C(O)NHR_{d}, or any combination thereof, wherein R_{d}represents C₁₋₅alkyl or optionally substituted phenyl, and the optionally substituted phenyl is optionally substituted with one or more (e.g., 1-5, 1-4, 1-3, or 1-2, or 1) substituents selected from the group consisting of halogen, cyano, amino, hydroxy, mercapto, oxo, and C₁₋₆ alkyl, or any combination thereof; and the 5- or 6-membered heterocyclyl is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, oxo, mercapto, C₁₋₆ alkyl, halogenated C₁₋₃ alkyl, or any combination thereof; and the C₅-C₂₀ aryl is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, C₁₋₆ alkyl, halogenated C₁₋₃ alkyl, or any combination thereof; and the 5- or 6-membered heteroaryl is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, oxo, mercapto, C₁₋₆ alkyl, halogenated C₁₋₃ alkyl, or any combination thereof.

In some embodiments of the compounds of Formula (I) and Formula (I-4) of the present disclosure, X₁ represents NHC(O)NR₇R₈, wherein R₇ represents H, and R₈ represents adamantanyl, adamantan-1-yl, adamantan-2-yl, 3,5-dimethyladamantan-1-yl, phenyl, or 3-chloro-4-methylphenyl.

In some embodiments of the compounds of Formula (I) and Formula (I-4) of the present disclosure, X₁ represents OR₉, wherein R₉ represents H, optionally substituted linear or branched C₁₋₁₀ alkyl, optionally substituted cycloalkyl, optionally substituted aryl, optionally substituted heterocyclyl or optionally substituted heteroaryl.

In some embodiments of the compounds of Formula (I) and Formula (I-4) of the present disclosure, X₁ represents OR₉, wherein R₉ represents H, optionally substituted linear or branched C₁₋₁₀ alkyl, optionally substituted C₃-C₂₀ cycloalkyl, optionally substituted 3- to 20-membered heterocyclyl, optionally substituted C₅-C₁₄ aryl or optionally substituted 5- to 20-membered heteroaryl, wherein the optionally substituted linear or branched C₁₋₁₀ alkyl is optionally substituted with one or more (e.g., 1-10, 1-9, 1-8, 1-7, 1-6, 1-5, 1-4, 1-3, or 1-2, or 1) substituents selected from the group consisting of halogen, cyano, hydroxy, amino, mercapto, C₁₋₃ alkyl, C₁₋₃ alkoxy, trifluoromethyl, optionally substituted C₅₋₁₀ aryl, optionally substituted 5- to 10-membered heteroaryl, optionally substituted C₃₋₁₂ cycloalkyl, optionally substituted 3- to 12-membered heterocyclyl, or any combination thereof, wherein the optionally substituted C₅₋₁₀ aryl, optionally substituted 5- to 10-membered heteroaryl, optionally substituted C₃₋₁₂ cycloalkyl and optionally substituted 3- to 12-membered heterocyclyl are each independently optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, C₁₋₆ alkyl, C₁₋₃ alkoxy, or any combination thereof;
the optionally substituted C₃-C₂₀ cycloalkyl and the optionally substituted 3- to 20-membered heterocyclyl are each independently optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, oxo, C₁₋₆ alkyl, C₁₋₃ alkoxy, C₁₋₃ alkoxycarbonyl, halogenated C₂₋₄ alkenyl, halogenated C₁₋₃ alkoxy, halogenated C₁₋₃ alkyl, C₁₋₆ alkylsulfonyl, bis(C₁₋₆ alkyl)phosphono, methanesulfonyloxy, trifluoromethanesulfonyloxy, p-toluenesulfonyloxy, optionally substituted 5- or 6-membered heterocyclyl, optionally substituted C₅-C₂₀ aryl, optionally substituted 5- or 6-membered heteroaryl, or any combination thereof, wherein the 5- or 6-membered heterocyclyl is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, oxo, mercapto, C₁₋₆ alkyl, halogenated C₁₋₃ alkyl, or any combination thereof; and the C₅-C₂₀ aryl is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, C₁₋₆ alkyl, halogenated C₁₋₃ alkyl, or any combination thereof; and the 5- or 6-membered heteroaryl is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, C₁₋₆ alkyl, halogenated C₁₋₃ alkyl, or any combination thereof;
wherein the optionally substituted C₅-C₁₄ aryl or optionally substituted 5- to 20-membered heteroaryl is each independently optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, C₁₋₃ alkoxy, halogenated C₁₋₃ alkoxy, halogenated C₁₋₃ alkyl, C₁₋₆ alkylsulfonyl, bis(C₁₋₆ alkyl)phosphono, methanesulfonyloxy, trifluoromethanesulfonyloxy, p-toluenesulfonyloxy, optionally substituted 5- or 6-membered heterocyclyl, optionally substituted C₅-C₂₀ aryl, optionally substituted 5- or 6-membered heteroaryl, C₁₋₆ alkyl, C₁₋₃ alkoxycarbonyl, halogenated C₂₋₄ alkenyl, C(O)NHR_{d}, or any combination thereof, wherein Rd represents C₁₋₅ alkyl or optionally substituted phenyl, and the optionally substituted phenyl is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, oxo, and C₁₋₆ alkyl, or any combination thereof; and the 5- or 6-membered heterocyclyl is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, oxo, mercapto, C₁₋₆ alkyl, halogenated C₁₋₃ alkyl, or any combination thereof; and the C₅-C₂₀ aryl is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, C₁₋₆ alkyl, halogenated C₁₋₃ alkyl, or any combination thereof; and the 5- or 6-membered heteroaryl is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, oxo, mercapto, C₁₋₆ alkyl, halogenated C₁₋₃ alkyl, or any combination thereof.

In some embodiments of the compounds of Formula (I) and Formula (I-4) of the present disclosure, X₁ represents OH, adamantanyl-O—, adamantan-1-yl-O—, adamantan-2-yl-O—, norbornyl-O—, 1,7,7-trimethyl-bicyclo[2.2.1]heptanyl-O—, or 2-isopropyl-5-methylcyclohexyl-O—.

In some embodiments of the compounds of Formula (I) and Formula (I-4) of the present disclosure, X₁ represents SR₁₀, wherein R₁₀ represents H, optionally substituted linear or branched C₁₋₁₀ alkyl, optionally substituted cycloalkyl, optionally substituted aryl, optionally substituted heterocyclyl or optionally substituted heteroaryl.

In some embodiments of the compounds of Formula (I) and Formula (I-4) of the present disclosure, X₁ represents SR₁₀, wherein R₁₀ represents H, optionally substituted linear or branched C₁₋₁₀ alkyl, optionally substituted C₃-C₂₀ cycloalkyl, optionally substituted 3- to 20-membered heterocyclyl, optionally substituted C₅-C₁₄ aryl or optionally substituted 5- to 20-membered heteroaryl,
wherein the optionally substituted linear or branched C₁₋₁₀ alkyl is optionally substituted with one or more (e.g., 1-10, 1-9, 1-8, 1-7, 1-6, 1-5, 1-4, 1-3, or 1-2, or 1) substituents selected from the group consisting of halogen, cyano, hydroxy, amino, mercapto, C₁₋₃ alkyl, C₁₋₃ alkoxy, trifluoromethyl, optionally substituted C₅₋₁₀aryl, optionally substituted 5- to 10-membered heteroaryl, optionally substituted C₃₋₁₂ cycloalkyl, optionally substituted 3- to 12-membered heterocyclyl, or any combination thereof, wherein the optionally substituted C₅₋₁₀aryl, optionally substituted 5- to 10-membered heteroaryl, optionally substituted C₃₋₁₂cycloalkyl and optionally substituted 3- to 12-membered heterocyclyl are each independently optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, C₁₋₆ alkyl, C₁₋₃ alkoxy, or any combination thereof;
the optionally substituted C₃-C₂₀ cycloalkyl and the optionally substituted 3- to 20-membered heterocyclyl are each independently optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, oxo, C₁₋₆ alkyl, C₁₋₃ alkoxy, C₁₋₃ alkoxycarbonyl, halogenated C₂₋₄ alkenyl, halogenated C₁₋₃ alkoxy, halogenated C₁₋₃ alkyl, C₁₋₆ alkylsulfonyl, bis(C₁₋₆ alkyl)phosphono, methanesulfonyloxy, trifluoromethanesulfonyloxy, p-toluenesulfonyloxy, optionally substituted 5- or 6-membered heterocyclyl, optionally substituted C₅-C₂₀ aryl, optionally substituted 5- or 6-membered heteroaryl, or any combination thereof, wherein the 5- or 6-membered heterocyclyl is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, oxo, mercapto, C₁₋₆ alkyl, halogenated C₁₋₃ alkyl, or any combination thereof; and the C₅-C₂₀ aryl is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, C₁₋₆ alkyl, halogenated C₁₋₃ alkyl, or any combination thereof; and the 5- or 6-membered heteroaryl is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, C₁₋₆ alkyl, halogenated C₁₋₃ alkyl, or any combination thereof;
wherein the optionally substituted C₅-C₁₄ aryl or optionally substituted 5- to 20-membered heteroaryl is each independently optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, C₁₋₃ alkoxy, halogenated C₁₋₃ alkoxy, halogenated C₁₋₃alkyl, C₁₋₆ alkylsulfonyl, bis(C₁₋₆ alkyl)phosphono, methanesulfonyloxy, trifluoromethanesulfonyloxy, p-toluenesulfonyloxy, optionally substituted 5- or 6-membered heterocyclyl, optionally substituted C₅-C₂₀ aryl, optionally substituted 5- or 6-membered heteroaryl, C₁₋₆ alkyl, C₁₋₃ alkoxycarbonyl, halogenated C₂₋₄ alkenyl, C(O)NHR_{d}, or any combination thereof, wherein Rd represents C₁₋₅ alkyl or optionally substituted phenyl, and the optionally substituted phenyl is optionally substituted with one or more (e.g., 1-5, 1-4, 1-3, or 1-2, or 1) substituents selected from the group consisting of halogen, cyano, amino, hydroxy, mercapto, oxo, and C₁₋₆ alkyl, or any combination thereof; and the 5- or 6-membered heterocyclyl is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, oxo, mercapto, C₁₋₆ alkyl, halogenated C₁₋₃ alkyl, or any combination thereof; and the C₅-C₂₀ aryl is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, C₁₋₆ alkyl, halogenated C₁₋₃ alkyl, or any combination thereof; and the 5- or 6-membered heteroaryl is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, oxo, mercapto, C₁₋₆ alkyl, halogenated C₁₋₃ alkyl, or any combination thereof.

In some embodiments of the compounds of Formula (I) and Formula (I-4) of the present disclosure, X₁ represents —SH, adamantanyl-S—, adamantan-1-yl-S—, 3-hydroxyadamantanyl-S—, 3-hydroxyadamantan-1-yl-S—, 3-chloroadamantan-1-yl-S—, 4-chloroadamantan-1-yl-S—, 2-chloroadamantan-1-yl-S—, or adamantan-2-yl-S—.

In some embodiments of the compounds of Formula (I) and Formula (I-4) of the present disclosure, X₁ represents a quaternary ammonium salt group.

In some embodiments of the compounds of Formula (I) and Formula (I-4) of the present disclosure, X₁ represents a quaternary ammonium salt group of Formula N⁺RₐR_{b}R_{c}X⁻, wherein Rₐ, R_{b}, R_{c} each independently represent C₁₋₂₀ alkyl, and X⁻ represents F⁻, Cl⁻, Br⁻, or I⁻.

In some embodiments of the compounds of Formula (I) and Formula (I-4) of the present disclosure, X₁ represents the quaternary ammonium salt group of Formula N⁺RₐR_{b}R_{c}X⁻, wherein Rₐ, R_{b}, R_{c} each independently represent C₁₋₁₀ alkyl, and X⁻ represents Cl⁻.

In some embodiments of the compounds of Formula (I) and Formula (I-4) of the present disclosure, X₁ represents the quaternary ammonium salt group of Formula N⁺RₐR_{b}R_{c}X⁻, wherein Rₐ and R_{b} each independently represent methyl, ethyl, propyl, isopropyl or butyl; R_{c} represents ethyl; and X⁻ represents Cl⁻.

In some embodiments of the compounds of Formula (I) and Formula (I-4) of the present disclosure, X₁ represents the quaternary ammonium salt group of Formula N⁺RₐR_{b}R_{c}X⁻, wherein Rₐ, R_{b}, and R_{c} represent ethyl, and X⁻ represents Cl⁻.

In some embodiments of the compounds of Formula (I) and Formula (I-4) of the present disclosure, X₁ represents the quaternary ammonium salt group of Formula N⁺RₐR_{b}R_{c}X⁻, wherein Rₐ, R_{b}, and R_{c} represent methyl, and X⁻ represents Cl⁻.

In some embodiments of the compounds of Formula (I) and Formula (I-4) of the present disclosure, X₁ represents the quaternary ammonium salt group of Formula N⁺RₐR_{b}R_{c}X⁻, wherein X-represents F⁻, Cl⁻, Br⁻, or I⁻; R_{c} represents C₁₋₂₀ alkyl or hydrogen; and Rₐ and R_{b} together with the nitrogen atom to which they are attached form a 5- to 8-membered heterocyclyl, wherein the ring atoms of the 5- to 8-membered heterocyclyl optionally further comprise a heteroatom selected from oxygen, nitrogen, and sulfur, and the 5- to 8-membered heterocyclyl is optionally substituted with one or more (e.g., 1-10, 1-9, 1-8, 1-7, 1-6, 1-5, 1-4, 1-3 or 1-2, or 1) substituents selected from the group consisting of: halogen, cyano, amino, hydroxy, mercapto, C₁₋₃ alkoxy, halogenated C₁₋₃ alkoxy, halogenated C₁₋₃ alkyl, C₁₋₆ alkylsulfonyl, bis(C₁₋₆ alkyl)phosphono, methanesulfonyloxy, trifluoromethanesulfonyloxy, p-toluenesulfonyloxy, optionally substituted 5- or 6-membered heterocyclyl, optionally substituted C₅-C₂₀ aryl, optionally substituted 5- or 6-membered heteroaryl, C₁₋₆alkyl, C₁₋₃ alkoxycarbonyl, halogenated C₂₋₄ alkenyl, or any combination thereof, wherein the 5- or 6-membered heterocyclyl is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, oxo, C₁-6 alkyl, halogenated C₁₋₃alkyl, or any combination thereof; and the C₅-C₂₀ aryl is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, C₁₋₆ alkyl, halogenated C₁₋₃ alkyl, or any combination thereof; and the 5- or 6-membered heteroaryl is optionally substituted with one or more (e.g., 1-10, 1-9, 1-8, 1-7, 1-6, 1-5, 1-4, 1-3, or 1-2, or 1) substituents selected from the group consisting of halogen, cyano, amino, hydroxy, mercapto, oxo, C₁₋₆ alkyl, halogenated C₁₋₃ alkyl, or any combination thereof.

In some embodiments of the compounds of Formula (I) and Formula (I-4) of the present disclosure, X₁ represents

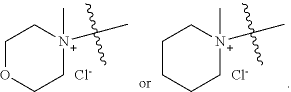

In some embodiments of the compounds of Formula (I) and Formula (I-4) of the present disclosure, wherein X₁ represents a group of Formula (G₁):

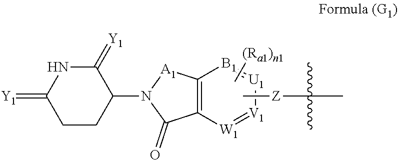

wherein A₁ represents CH₂ or C(O);
B₁, U₁, V₁, and W₁ are the same or different and each independently represent CH or N, wherein B₁, U₁, V₁, and W₁ are not N at the same time;
Y₁ represents O or S; and
Z represents S, S(O) or S(O)₂; and
(Rₐ₁)ₙ₁ denotes that the ring containing B₁, U₁, V₁, and W₁ is substituted with n1 Rₐ₁, where Rₐ₁ groups are the same or different and each independently represent deuterium, halogen, hydroxy, mercapto, nitro, amino, cyano, optionally deuterated C₁₋₆ alkyl, optionally deuterated C₁₋₆ alkoxy, halogenated C₁₋₆ alkyl, optionally substituted C₃₋₆ cycloalkyl, C₂₋₆ alkenyl, or C₂₋₆ alkynyl, and n represents an integer of 0, 1, 2, or 3, wherein the C₃₋₆ cycloalkyl is optionally substituted with one or more (e.g., 1-10, 1-9, 1-8, 1-7, 1-6, 1-5, 1-4, 1-3 or 1-2, or 1) substituents selected from the group consisting of C₁₋₆ alkyl, halogenated C₁₋₃ alkyl, amino, hydroxy, halogen, or any combination thereof.

In some embodiments of the compounds of Formula (I) and Formula (I-4) of the present disclosure, in Formula (G₁), B₁, U₁, V₁, and W₁ are identical, each being CH.

In some embodiments of the compounds of Formula (I) and Formula (I-4) of the present disclosure, in Formula (G₁), one of B₁, U₁, V₁, and W₁ is N, and the remaining are CH.

In some embodiments of the compounds of Formula (I) and Formula (I-4) of the present disclosure, in Formula (G₁), two of B₁, U₁, V₁, and W₁ are N, and the remaining are CH.

In some embodiments of the compounds of Formula (I) and Formula (I-4) of the present disclosure, in Formula (G₁), three of B₁, U₁, V₁, and W₁ are N, and the remaining is CH.

In some embodiments of the compounds of Formula (I) and Formula (I-4) of the present disclosure, wherein X₁ represents the group of Formula (G₂):

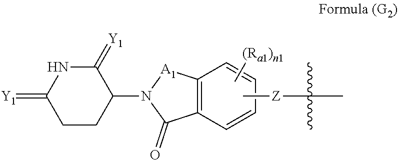

wherein A₁ represents CH₂ or C(O), Y₁ represents O or S, and Z represents S, S(O), S(O)₂, O, NH or CH₂, and (Rₐ₁)ₙ₁ denotes that the benzene ring is substituted with n1 Rₐ₁, where Rₐ₁ groups are the same or different and each independently represent deuterium, halogen, hydroxy, mercapto, nitro, amino, cyano, optionally deuterated C₁₋₆ alkyl, optionally deuterated C₁₋₆ alkoxy, halogenated C₁₋₆alkyl, optionally substituted C₃₋₆ cycloalkyl, C₂₋₆ alkenyl, or C₂₋₆ alkynyl, and n represents an integer of 0, 1, 2, or 3.

In some embodiments of the compounds of Formula (I) and Formula (I-4) of the present disclosure, wherein X₁ represents the group of Formula (G₃):

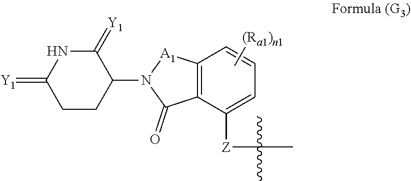

wherein A₁, Y₁, Z, and (Rₐ₁)ₙ₁ are as defined in the compounds of Formula (G₁) above and its various sub-embodiments described herein.

In some embodiments of the compounds of Formula (I) and Formula (I-4) of the present disclosure, wherein X₁ represents the group of Formula (G₄):

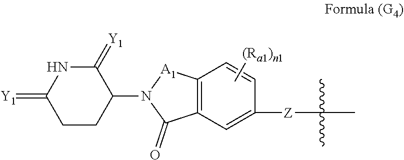

wherein A₁, Y₁, Z, and (Rₐ₁)ₙ₁ are as defined in the compounds of Formula (G₁) above and its various sub-embodiments described herein.

In some embodiments of the compounds of Formula (I) and Formula (I-4) of the present disclosure, wherein X₁ represents the group of Formula (G₅):

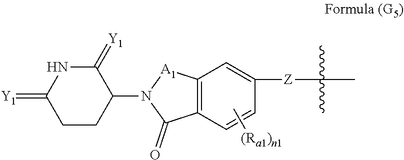

wherein A₁, Y₁, Z, and (Rₐ₁)ₙ₁ are as defined in the compounds of Formula (G₁) above and its various sub-embodiments described herein.

In some embodiments of the compounds of Formula (I) and Formula (I-4) of the present disclosure, wherein X₁ represents the group of Formula (G₆):

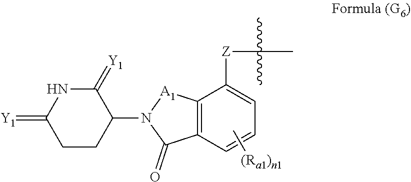

wherein A₁, Y₁, Z, and (Rₐ₁)ₙ₁ are as defined in the compounds of Formula (G₁) above and its various sub-embodiments described herein.

In some embodiments of the compounds of Formula (I) and Formula (I-4) of the present disclosure, wherein A₁ represents CH₂ or C(O), Y₁ represents O, and Z represents S, S(O) or S(O)₂.

In some embodiments of the present disclosure, the compounds of Formula (I), Formula (I-1), Formula (I-2), Formula (I-3), and Formula (I-4) are also of the compounds of Formula (Ia), Formula (Ib), Formula (Ic), Formula (Id), or Formula (Ie):

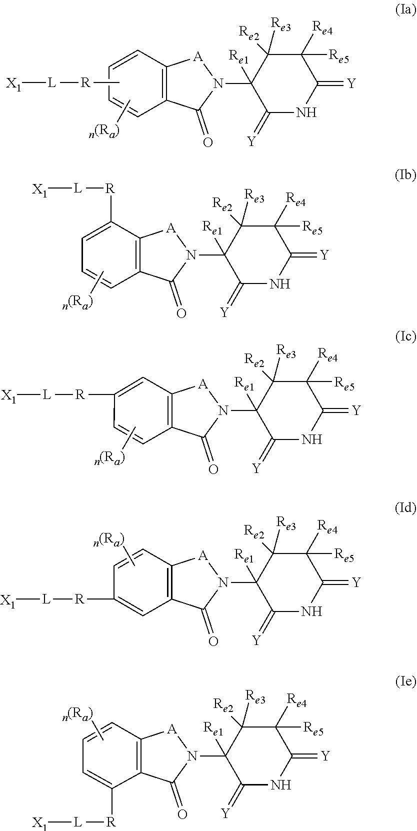

wherein A, (Rₐ)ₙ, Rₑ₁, Rₑ₂, Rₑ₃, Rₑ₄, Rₑ₅, R, L, X₁, and Y are as defined in the compounds of Formula (I), Formula (I-1), Formula (I-2), Formula (I-3), and Formula (I-4) and their various embodiments described herein.

Preferably the compounds of the present invention and their salts (especially pharmaceutically acceptable salts, such as hydrochloride, etc.), enantiomers, diastereomers, isotopically enriched analogs, solvates, prodrugs, or polymorphs thereof in Table 1 below are provided:

TABLE 1
The compounds of the present invention

| Com- | | |
|---|---|---|
| pound | | |
| No. | Structure of the compounds | The compounds' name |
| | 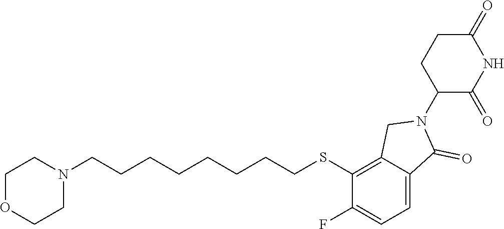 [SMILES: O=C1CCC(N2Cc3c(ccc(F)c3SCCCCCCCCN3CCOCC3)C2=O)C(=O)N1] | 3-(5-fluoro-4-((8-morpholinooctyl)thio)-1- oxoisoindolin-2-yl)piperidine-2,6-dione |
| GT- 05341 | 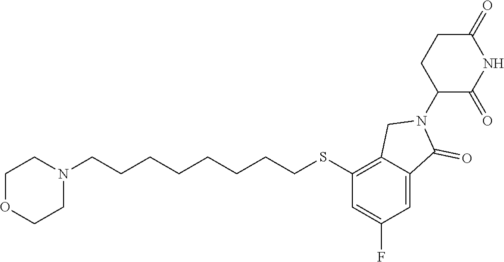 [SMILES: O=C1CCC(N2Cc3c(SCCCCCCCCN4CCOCC4)cc(F)cc3C2=O)C(=O)N1] | 3-(6-fluoro-4-((8-morpholinooctyl)thio)-1- oxoisoindolin-2-yl)piperidine-2,6-dione |
| | 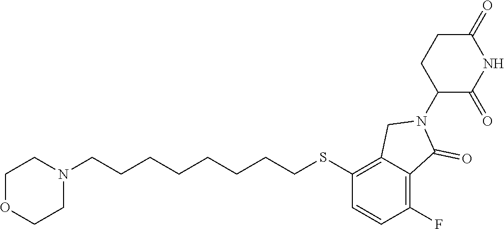 [SMILES: O=C1CCC(N2Cc3c(SCCCCCCCCN4CCOCC4)ccc(F)c3C2=O)C(=O)N1] | 3-(7-fluoro-4-((8-morpholinooctyl)thio)-1- oxoisoindolin-2-yl)piperidine-2,6-dione |
| | 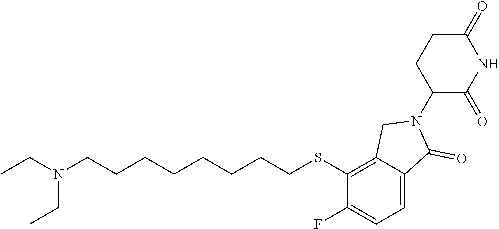 [SMILES: CCN(CC)CCCCCCCCSc1c(F)ccc2c1CN(C1CCC(=O)NC1=O)C2=O] | 3-(4-((8-(diethylamino)octyl)thio)-5-fluoro- 1-oxoisoindolin-2-yl)piperidine-2,6-dione |
| GT- 05342 | 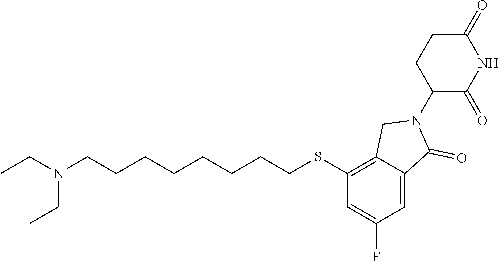 [SMILES: CCN(CC)CCCCCCCCSc1cc(F)cc2c1CN(C1CCC(=O)NC1=O)C2=O] | 3-(4-((8-(diethylamino)octyl)thio)-6-fluoro- 1-oxoisoindolin-2-yl)piperidine-2,6-dione |
| | 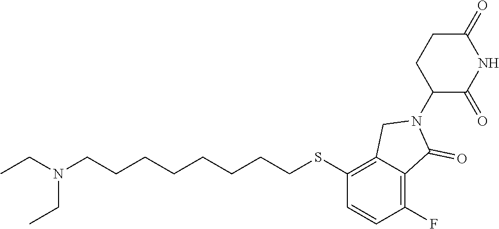 [SMILES: CCN(CC)CCCCCCCCSc1ccc(F)c2c1CN(C1CCC(=O)NC1=O)C2=O] | 3-(4-((8-(diethylamino)octyl)thio)-7-fluoro- 1-oxoisoindolin-2-yl)piperidine-2,6-dione |
| | 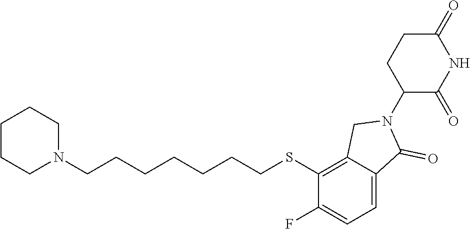 [SMILES: O=C1CCC(N2Cc3c(ccc(F)c3SCCCCCCCN3CCCCC3)C2=O)C(=O)N1] | 3-(5-fluoro-1-oxo-4-((7-(piperidin-1- yl)heptyl)thio)isoindolin-2-yl)piperidine-2,6- dione |
| GT- 05344 | 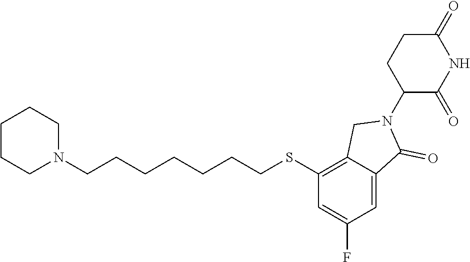 [SMILES: O=C1CCC(N2Cc3c(SCCCCCCCN4CCCCC4)cc(F)cc3C2=O)C(=O)N1] | 3-(6-fluoro-1-oxo-4-((7-(piperidin-1- yl)heptyl)thio)isoindolin-2-yl)piperidine-2,6- dione |
| | 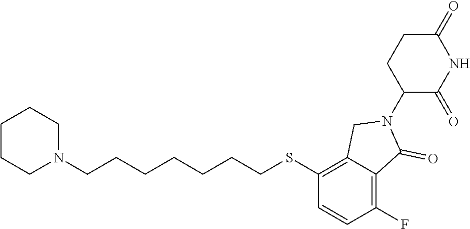 [SMILES: O=C1CCC(N2Cc3c(SCCCCCCCN4CCCCC4)ccc(F)c3C2=O)C(=O)N1] | 3-(7-fluoro-1-oxo-4-((7-(piperidin-1- yl)heptyl)thio)isoindolin-2-yl)piperidine-2,6- dione |
| | 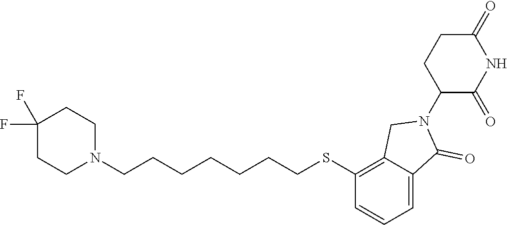 [SMILES: O=C1CCC(N2Cc3c(SCCCCCCCN4CCC(F)(F)CC4)cccc3C2=O)C(=O)N1] | 3-(4-((7-(4,4-difluoropiperidin-1- yl)heptyl)thio)-1-oxoisoindolin-2- yl)piperidine-2,6-dione |
| | 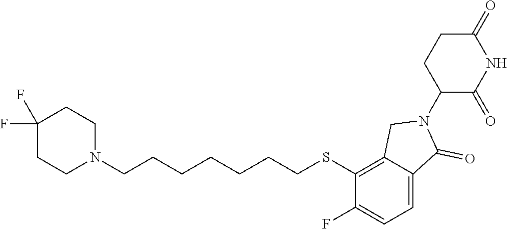 [SMILES: O=C1CCC(N2Cc3c(ccc(F)c3SCCCCCCCN3CCC(F)(F)CC3)C2=O)C(=O)N1] | 3-(4-((7-(4,4-difluoropiperidin-1- yl)heptyl)thio)-5-fluoro-1-oxoisoindolin-2- yl)piperidine-2,6-dione |
| GT- 05408 | 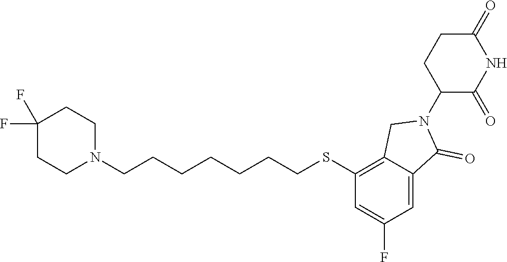 [SMILES: O=C1CCC(N2Cc3c(SCCCCCCCN4CCC(F)(F)CC4)cc(F)cc3C2=O)C(=O)N1] | 3-(4-((7-(4,4-difluoropiperidin-1- yl)heptyl)thio)-6-fluoro-1-oxoisoindolin-2- yl)piperidine-2,6-dione |
| | 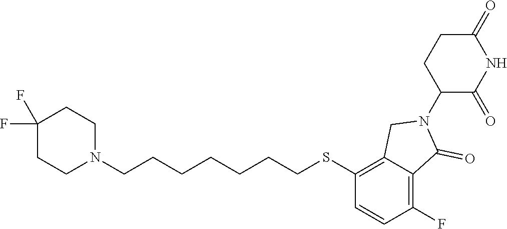 [SMILES: O=C1CCC(N2Cc3c(SCCCCCCCN4CCC(F)(F)CC4)ccc(F)c3C2=O)C(=O)N1] | 3-(4-((7-(4,4-difluoropiperidin-1- yl)heptyl)thio)-7-fluoro-1-oxoisoindolin-2- yl)piperidine-2,6-dione |
| | 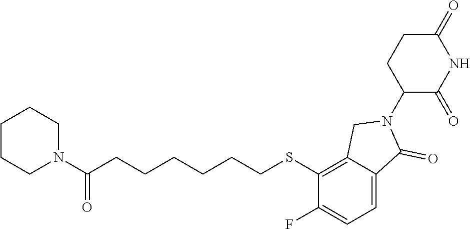 [SMILES: O=C1CCC(N2Cc3c(ccc(F)c3SCCCCCCC(=O)N3CCCCC3)C2=O)C(=O)N1] | 3-(5-fluoro-1-oxo-4-((7-oxo-7-(piperidin-1- yl)heptyl)thio)isoindolin-2-yl)piperidine-2,6- dione |
| GT- 05343 | 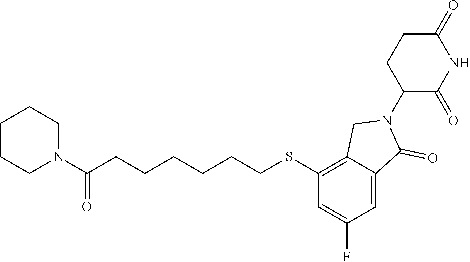 [SMILES: O=C1CCC(N2Cc3c(SCCCCCCC(=O)N4CCCCC4)cc(F)cc3C2=O)C(=O)N1] | 3-(6-fluoro-1-oxo-4-((7-oxo-7-(piperidin-1- yl)heptyl)thio)isoindolin-2-yl)piperidine-2,6- dione |
| | 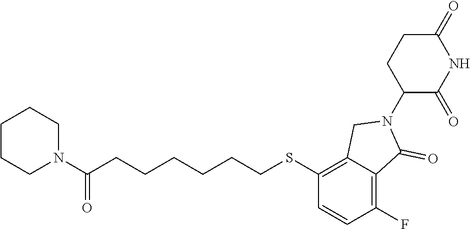 [SMILES: O=C1CCC(N2Cc3c(SCCCCCCC(=O)N4CCCCC4)ccc(F)c3C2=O)C(=O)N1] | 3-(7-fluoro-1-oxo-4-((7-oxo-7-(piperidin-1- yl)heptyl)thio)isoindolin-2-yl)piperidine-2,6- dione |
| | 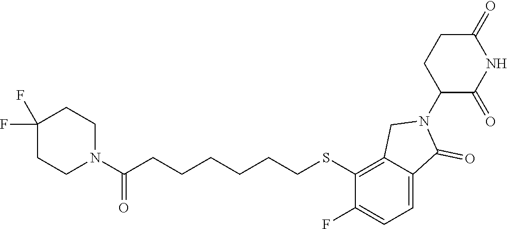 [SMILES: O=C1CCC(N2Cc3c(ccc(F)c3SCCCCCCC(=O)N3CCC(F)(F)CC3)C2=O)C(=O)N1] | 3-(4-((7-(4,4-difluoropiperidin-1-yl)-7- oxoheptyl)thio)-5-fluoro-1-oxoisoindolin-2- yl)piperidine-2,6-dione |
| GT- 05407 | 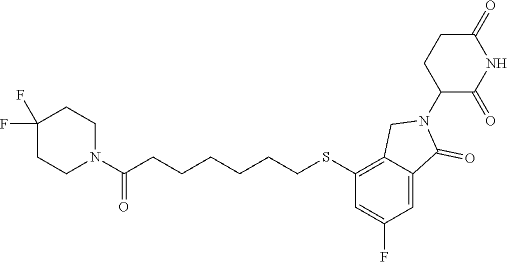 [SMILES: O=C1CCC(N2Cc3c(SCCCCCCC(=O)N4CCC(F)(F)CC4)cc(F)cc3C2=O)C(=O)N1] | 3-(4-((7-(4,4-difluoropiperidin-1-yl)-7- oxoheptyl)thio)-6-fluoro-1-oxoisoindolin-2- yl)piperidine-2,6-dione |
| | 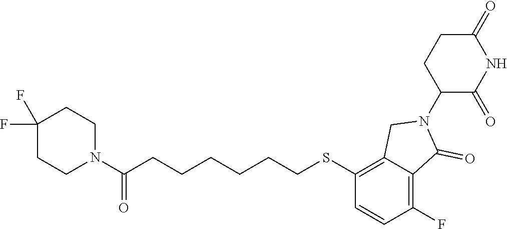 [SMILES: O=C1CCC(N2Cc3c(SCCCCCCC(=O)N4CCC(F)(F)CC4)ccc(F)c3C2=O)C(=O)N1] | 3-(4-((7-(4,4-difluoropiperidin-1-yl)-7- oxoheptyl)thio)-7-fluoro-1-oxoisoindolin-2- yl)piperidine-2,6-dione |
| | 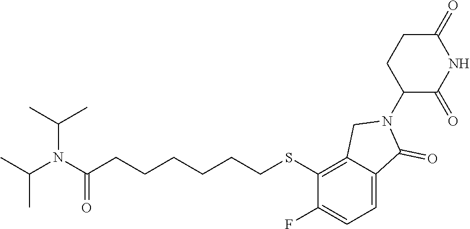 [SMILES: CC(C)N(C(=O)CCCCCCSc1c(F)ccc2c1CN(C1CCC(=O)NC1=O)C2=O)C(C)C] | 7-((2-(2,6-dioxopiperidin-3-yl)-5-fluoro-1- oxoisoindolin-4-yl)thio)-N,N- diisopropylheptanamide |
| | 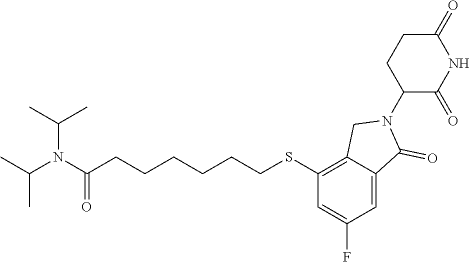 [SMILES: CC(C)N(C(=O)CCCCCCSc1cc(F)cc2c1CN(C1CCC(=O)NC1=O)C2=O)C(C)C] | 7-((2-(2,6-dioxopiperidin-3-yl)-6-fluoro-1- oxoisoindolin-4-yl)thio)-N,N- diisopropylheptanamide |
| | 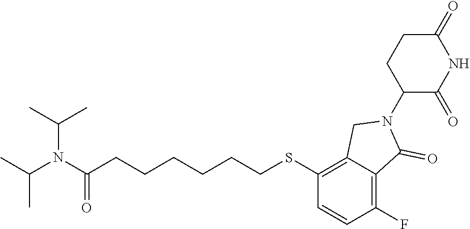 [SMILES: CC(C)N(C(=O)CCCCCCSc1ccc(F)c2c1CN(C1CCC(=O)NC1=O)C2=O)C(C)C] | 7-((2-(2,6-dioxopiperidin-3-yl)-7-fluoro-1- oxoisoindolin-4-yl)thio)-N,N- diisopropylheptanamide |
| | 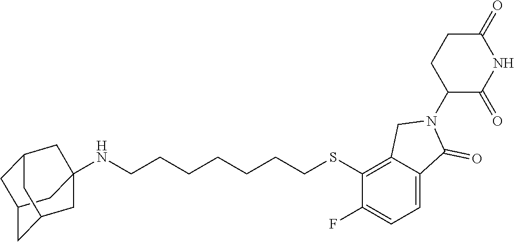 [SMILES: O=C1CCC(N2Cc3c(ccc(F)c3SCCCCCCCNC34CC5CC(CC(C5)C3)C4)C2=O)C(=O)N1] | 3-(4-((7-((adamantan-1- yl)amino)heptyl)thio)-5-fluoro-1- oxoisoindolin-2-yl)piperidine-2,6-dione |
| GT- 04243 | 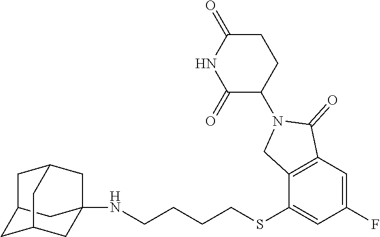 [SMILES: O=C1CCC(N2Cc3c(SCCCCNC45CC6CC(CC(C6)C4)C5)cc(F)cc3C2=O)C(=O)N1] | 3-(4-((4-((adamantan-1- yl)amino)butyl)thio)-6-fluoro-1- oxoisoindolin-2-yl)piperidine-2,6-dione |
| | 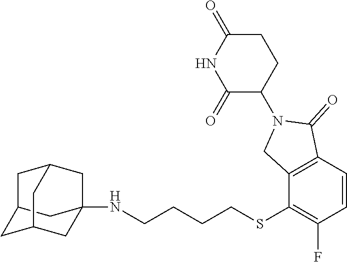 [SMILES: O=C1CCC(N2Cc3c(ccc(F)c3SCCCCNC34CC5CC(CC(C5)C3)C4)C2=O)C(=O)N1] | 3-(4-((4-((adamantan-1- yl)amino)butyl)thio)-5-fluoro-1- oxoisoindolin-2-yl)piperidine-2,6-dione |
| | 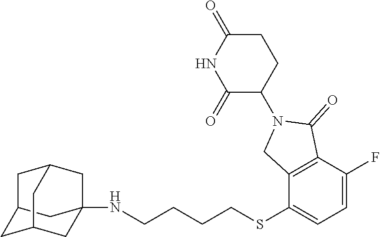 [SMILES: O=C1CCC(N2Cc3c(SCCCCNC45CC6CC(CC(C6)C4)C5)ccc(F)c3C2=O)C(=O)N1] | 3-(4-((4-((adamantan-1- yl)amino)butyl)thio)-7-fluoro-1- oxoisoindolin-2-yl)piperidine-2,6-dione |
| GT- 04333 | 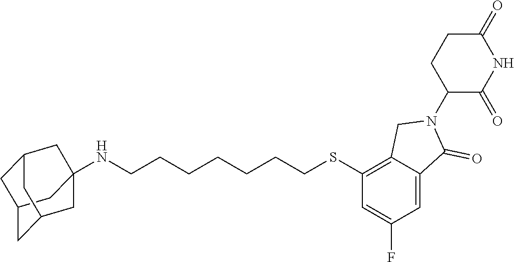 [SMILES: O=C1CCC(N2Cc3c(SCCCCCCCNC45CC6CC(CC(C6)C4)C5)cc(F)cc3C2=O)C(=O)N1] | 3-(4-((7-((adamantan-1- yl)amino)heptyl)thio)-6-fluoro-1- oxoisoindolin-2-yl)piperidine-2,6-dione |
| | 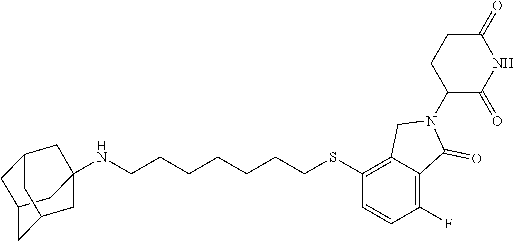 [SMILES: O=C1CCC(N2Cc3c(SCCCCCCCNC45CC6CC(CC(C6)C4)C5)ccc(F)c3C2=O)C(=O)N1] | 3-(4-((7-((adamantan-1- yl)amino)heptyl)thio)-7-fluoro-1- oxoisoindolin-2-yl)piperidine-2,6-dione |
| | 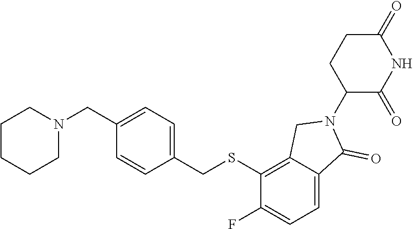 [SMILES: O=C1CCC(N2Cc3c(ccc(F)c3SCc3ccc(CN4CCCCC4)cc3)C2=O)C(=O)N1] | 3-(5-fluoro-1-oxo-4-((4-(piperidin-1- ylmethyl)benzyl)thio)isoindolin-2- yl)piperidine-2,6-dione |
| GT- 04117 | 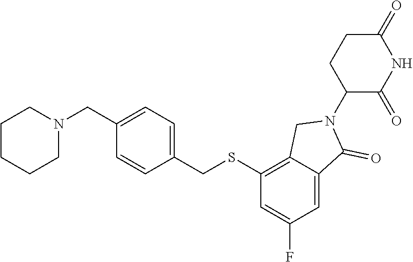 [SMILES: O=C1CCC(N2Cc3c(SCc4ccc(CN5CCCCC5)cc4)cc(F)cc3C2=O)C(=O)N1] | 3-(6-fluoro-1-oxo-4-((4-(piperidin-1- ylmethyl)benzyl)thio)isoindolin-2- yl)piperidine-2,6-dione |
| | 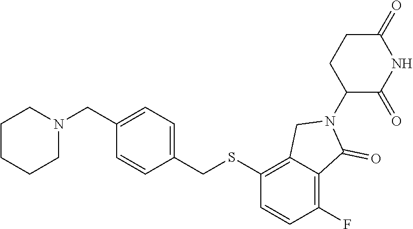 [SMILES: O=C1CCC(N2Cc3c(SCc4ccc(CN5CCCCC5)cc4)ccc(F)c3C2=O)C(=O)N1] | 3-(7-fluoro-1-oxo-4-((4-(piperidin-1- ylmethyl)benzyl)thio)isoindolin-2- yl)piperidine-2,6-dione |
| | 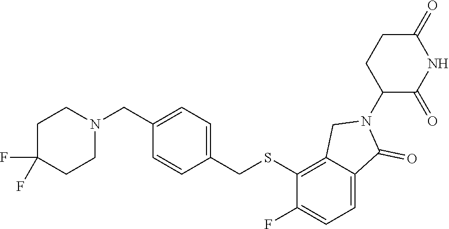 [SMILES: O=C1CCC(N2Cc3c(ccc(F)c3SCc3ccc(CN4CCC(F)(F)CC4)cc3)C2=O)C(=O)N1] | 3-(4-((4-((4,4-difluoropiperidin-1- yl)methyl)benzyl)thio)-5-fluoro-1- oxoisoindolin-2-yl)piperidine-2,6-dione |
| GT- 04024 | 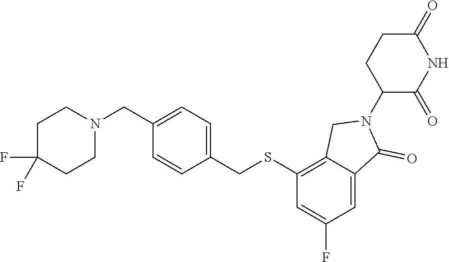 [SMILES: O=C1CCC(N2Cc3c(SCc4ccc(CN5CCC(F)(F)CC5)cc4)cc(F)cc3C2=O)C(=O)N1] | 3-(4-((4-((4,4-difluoropiperidin-1- yl)methyl)benzyl)thio)-6-fluoro-1- oxoisoindolin-2-yl)piperidine-2,6-dione |
| | 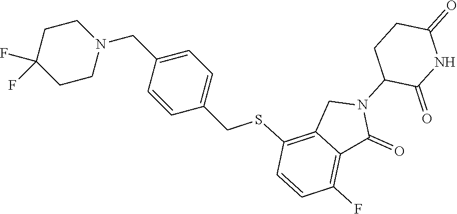 [SMILES: O=C1CCC(N2Cc3c(SCc4ccc(CN5CCC(F)(F)CC5)cc4)ccc(F)c3C2=O)C(=O)N1] | 3-(4-((4-((4,4-difluoropiperidin-1- yl)methyl)benzyl)thio)-7-fluoro-1- oxoisoindolin-2-yl)piperidine-2,6-dione |
| | 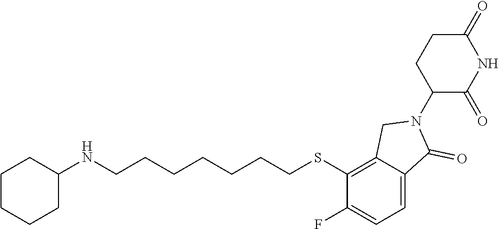 [SMILES: O=C1CCC(N2Cc3c(ccc(F)c3SCCCCCCCNC3CCCCC3)C2=O)C(=O)N1] | 3-(4-((7-(cyclohexylamino)heptyl)thio)-5- fluoro-1-oxoisoindolin-2-yl)piperidine-2,6- dione |
| GT- 05409 | 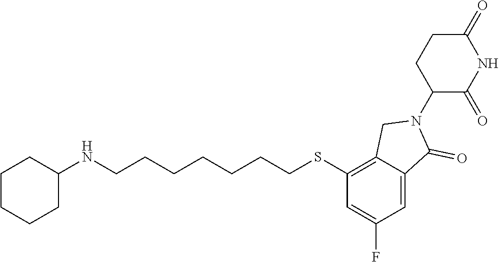 [SMILES: O=C1CCC(N2Cc3c(SCCCCCCCNC4CCCCC4)cc(F)cc3C2=O)C(=O)N1] | 3-(4-((7-(cyclohexylamino)heptyl)thio)-6- fluoro-1-oxoisoindolin-2-yl)piperidine-2,6- dione |
| | 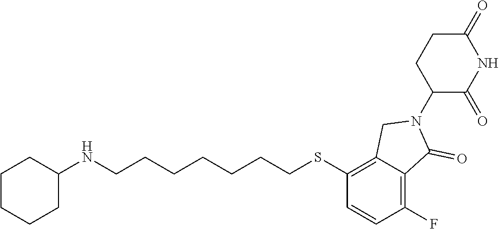 [SMILES: O=C1CCC(N2Cc3c(SCCCCCCCNC4CCCCC4)ccc(F)c3C2=O)C(=O)N1] | 3-(4-((7-(cyclohexylamino)heptyl)thio)-7- fluoro-1-oxoisoindolin-2-yl)piperidine-2,6- dione |
| | 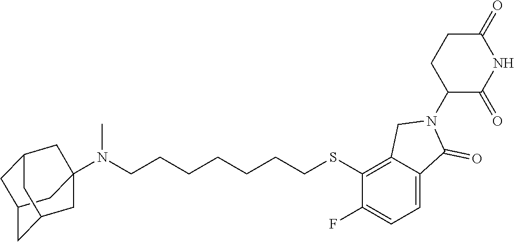 [SMILES: CN(CCCCCCCSc1c(F)ccc2c1CN(C1CCC(=O)NC1=O)C2=O)C12CC3CC(CC(C3)C1)C2] | 3-(4-((7-((adamantan-1- yl)(methyl)amino)heptyl)thio)-5-fluoro-1- oxoisoindolin-2-yl)piperidine-2,6-dione |
| GT- 05421 | 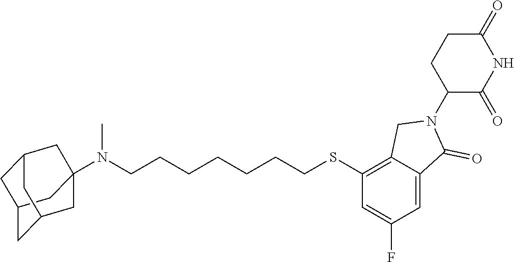 [SMILES: CN(CCCCCCCSc1cc(F)cc2c1CN(C1CCC(=O)NC1=O)C2=O)C12CC3CC(CC(C3)C1)C2] | 3-(4-((7-((adamantan-1- yl)(methyl)amino)heptyl)thio)-6-fluoro-1- oxoisoindolin-2-yl)piperidine-2,6-dione |
| | 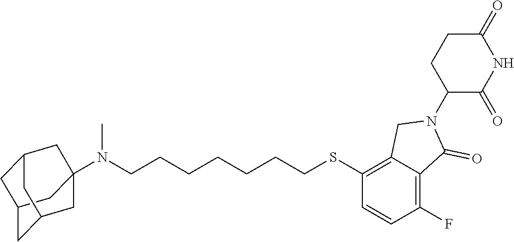 [SMILES: CN(CCCCCCCSc1ccc(F)c2c1CN(C1CCC(=O)NC1=O)C2=O)C12CC3CC(CC(C3)C1)C2] | 3-(4-((7-((adamantan-1- yl)(methyl)amino)heptyl)thio)-7-fluoro-1- oxoisoindolin-2-yl)piperidine-2,6-dione |
| | 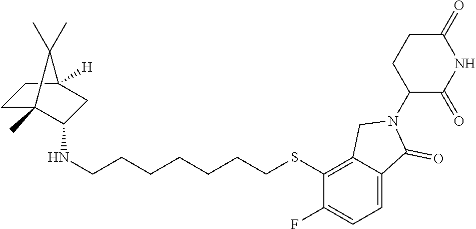 [SMILES: CC1(C)[C@@H]2CC[C@@]1(C)[C@@H](NCCCCCCCSc1c(F)ccc3c1CN(C1CCC(=O)NC1=O)C3=O)C2] | 3-(5-fluoro-1-oxo-4-((7-(((1R,2S,4R)-1,7,7- trimethylbicyclo[2.2.1]heptan-2- yl)amino)heptyl)thio)isoindolin-2- yl)piperidine-2,6-dione |
| GT- 05425 | 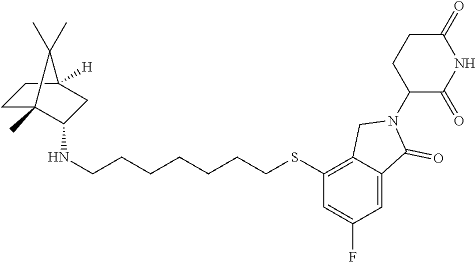 [SMILES: CC1(C)[C@@H]2CC[C@@]1(C)[C@@H](NCCCCCCCSc1cc(F)cc3c1CN(C1CCC(=O)NC1=O)C3=O)C2] | 3-(6-fluoro-1-oxo-4-((7-(((1R,2S,4R)-1,7,7- trimethylbicyclo[2.2.1]heptan-2- yl)amino)heptyl)thio)isoindolin-2- yl)piperidine-2,6-dione |
| | 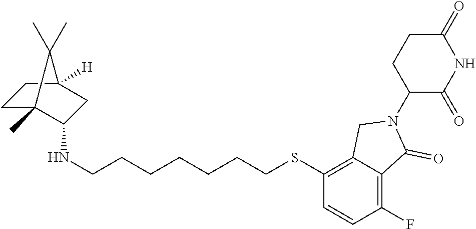 [SMILES: CC1(C)[C@@H]2CC[C@@]1(C)[C@@H](NCCCCCCCSc1ccc(F)c3c1CN(C1CCC(=O)NC1=O)C3=O)C2] | 3-(7-fluoro-1-oxo-4-((7-(((1R,2S,4R)-1,7,7- trimethylbicyclo[2.2.1]heptan-2- yl)amino)heptyl)thio)isoindolin-2- yl)piperidine-2,6-dione |
| | 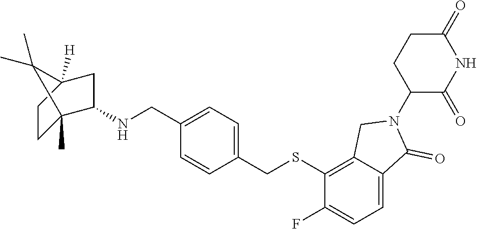 [SMILES: CC1(C)[C@@H]2CC[C@@]1(C)[C@@H](NCc1ccc(CSc3c(F)ccc4c3CN(C3CCC(=O)NC3=O)C4=O)cc1)C2] | 3-(5-fluoro-1-oxo-4-((4-((((1R,2S,4R)-1,7,7- trimethylbicyclo[2.2.1]heptan-2- yl)amino)methyl)benzyl)thio)isoindolin-2- yl)piperidine-2,6-dione |
| GT- 05424 | 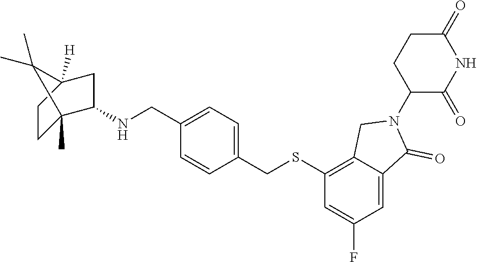 [SMILES: CC1(C)[C@@H]2CC[C@@]1(C)[C@@H](NCc1ccc(CSc3cc(F)cc4c3CN(C3CCC(=O)NC3=O)C4=O)cc1)C2] | 3-(6-fluoro-1-oxo-4-((4-((((1R,2S,4R)-1,7,7- trimethylbicyclo[2.2.1]heptan-2- yl)amino)methyl)benzyl)thio)isoindolin-2- yl)piperidine-2,6-dione |
| | 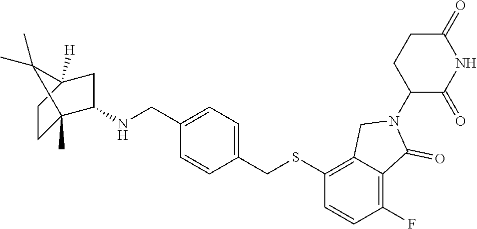 [SMILES: CC1(C)[C@@H]2CC[C@@]1(C)[C@@H](NCc1ccc(CSc3ccc(F)c4c3CN(C3CCC(=O)NC3=O)C4=O)cc1)C2] | 3-(7-fluoro-1-oxo-4-((4-((((1R,2S,4R)-1,7,7- trimethylbicyclo[2.2.1]heptan-2- yl)amino)methyl)benzyl)thio)isoindolin-2- yl)piperidine-2,6-dione |
| | 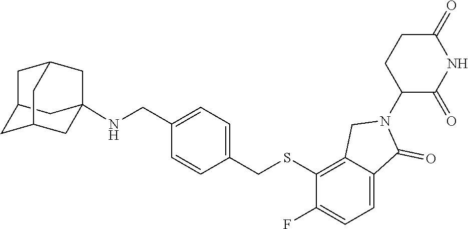 [SMILES: O=C1CCC(N2Cc3c(ccc(F)c3SCc3ccc(CNC45CC6CC(CC(C6)C4)C5)cc3)C2=O)C(=O)N1] | 3-(4-((4-(((adamantan-1- yl)amino)methyl)benzyl)thio)-5-fluoro-1- oxoisoindolin-2-yl)piperidine-2,6-dione |
| | 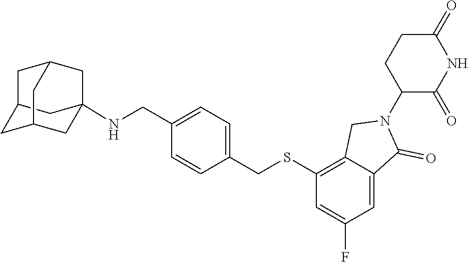 [SMILES: O=C1CCC(N2Cc3c(SCc4ccc(CNC56CC7CC(CC(C7)C5)C6)cc4)cc(F)cc3C2=O)C(=O)N1] | 3-(4-((4-(((adamantan-1- yl)amino)methyl)benzyl)thio)-6-fluoro-1- oxoisoindolin-2-yl)piperidine-2,6-dione |
| | 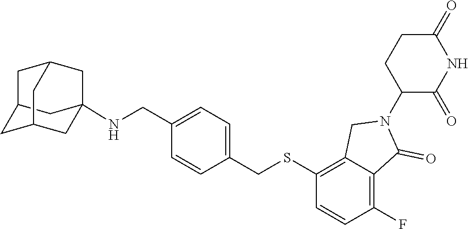 [SMILES: O=C1CCC(N2Cc3c(SCc4ccc(CNC56CC7CC(CC(C7)C5)C6)cc4)ccc(F)c3C2=O)C(=O)N1] | 3-(4-((4-(((adamantan-1- yl)amino)methyl)benzyl)thio)-7-fluoro-1- oxoisoindolin-2-yl)piperidine-2,6-dione |
| | 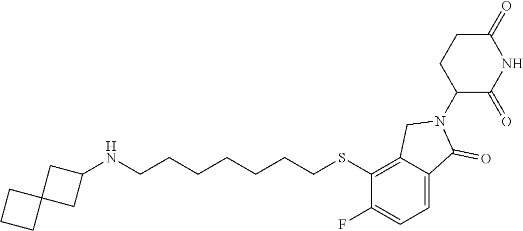 [SMILES: O=C1CCC(N2Cc3c(ccc(F)c3SCCCCCCCNC3CC4(CCC4)C3)C2=O)C(=O)N1] | 3-(5-fluoro-1-oxo-4-((7-(spiro[3.3]heptan-2- ylamino)heptyl)thio)isoindolin-2- yl)piperidine-2,6-dione |
| GT- 05422 | 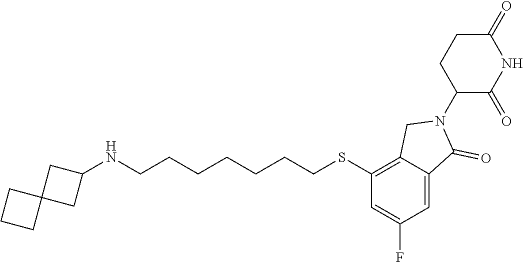 [SMILES: O=C1CCC(N2Cc3c(SCCCCCCCNC4CC5(CCC5)C4)cc(F)cc3C2=O)C(=O)N1] | 3-(6-fluoro-1-oxo-4-((7-(spiro[3.3]heptan-2- ylamino)heptyl)thio)isoindolin-2- yl)piperidine-2,6-dione |
| | 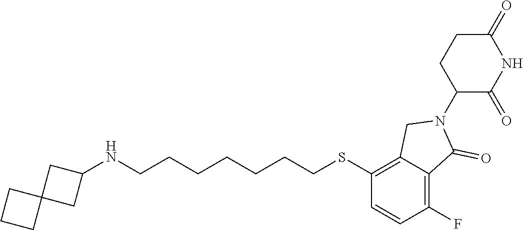 [SMILES: O=C1CCC(N2Cc3c(SCCCCCCCNC4CC5(CCC5)C4)ccc(F)c3C2=O)C(=O)N1] | 3-(7-fluoro-1-oxo-4-((7-(spiro[3.3]heptan-2- ylamino)heptyl)thio)isoindolin-2- yl)piperidine-2,6-dione |
| | 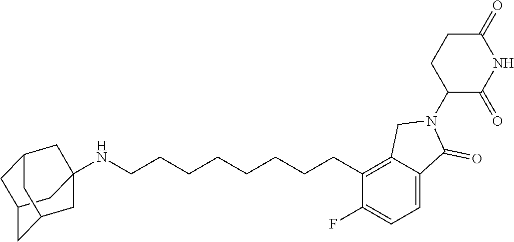 [SMILES: O=C1CCC(N2Cc3c(ccc(F)c3CCCCCCCCNC34CC5CC(CC(C5)C3)C4)C2=O)C(=O)N1] | 3-(4-(8-((adamantan-1-yl)amino)octyl)-5- fluoro-1-oxoisoindolin-2-yl)piperidine-2,6- dione |
| GT- 05617 | 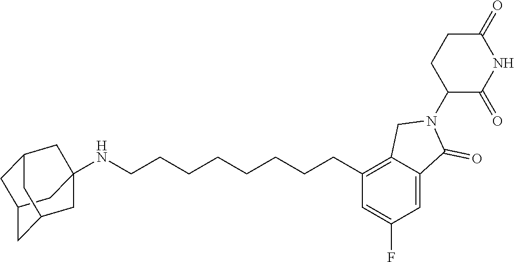 [SMILES: O=C1CCC(N2Cc3c(CCCCCCCCNC45CC6CC(CC(C6)C4)C5)cc(F)cc3C2=O)C(=O)N1] | 3-(4-(8-((adamantan-1-yl)amino)octyl)-6- fluoro-1-oxoisoindolin-2-yl)piperidine-2,6- dione |
| | 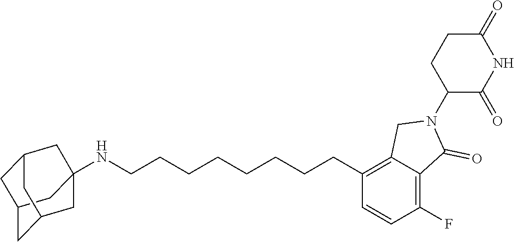 [SMILES: O=C1CCC(N2Cc3c(CCCCCCCCNC45CC6CC(CC(C6)C4)C5)ccc(F)c3C2=O)C(=O)N1] | 3-(4-(8-((adamantan-1-yl)amino)octyl)-7- fluoro-1-oxoisoindolin-2-yl)piperidine-2,6- dione |
| | 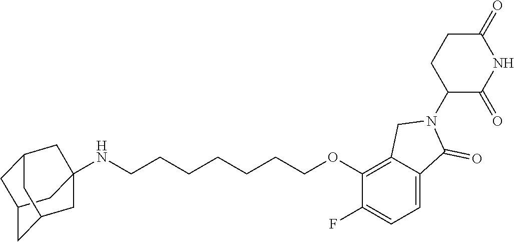 [SMILES: O=C1CCC(N2Cc3c(ccc(F)c3OCCCCCCCNC34CC5CC(CC(C5)C3)C4)C2=O)C(=O)N1] | 3-(4-((7-((yl)amino)heptyl)oxy)-5-fluoro-1- oxoisoindolin-2-yl)piperidine-2,6-dione |
| | 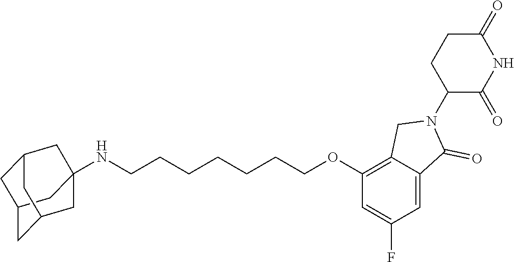 [SMILES: O=C1CCC(N2Cc3c(OCCCCCCCNC45CC6CC(CC(C6)C4)C5)cc(F)cc3C2=O)C(=O)N1] | 3-(4-((7-((adamantan-1- yl)amino)heptyl)oxy)-6-fluoro-1- oxoisoindolin-2-yl)piperidine-2,6-dione |
| | 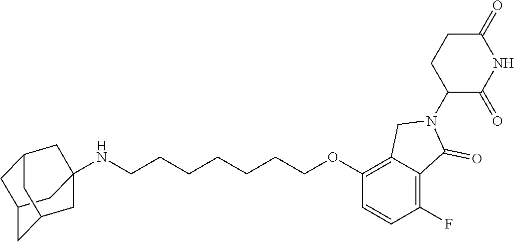 [SMILES: O=C1CCC(N2Cc3c(OCCCCCCCNC45CC6CC(CC(C6)C4)C5)ccc(F)c3C2=O)C(=O)N1] | 3-(4-((7-((adamantan-1- yl)amino)heptyl)oxy)-7-fluoro-1- oxoisoindolin-2-yl)piperidine-2,6-dione |
| | 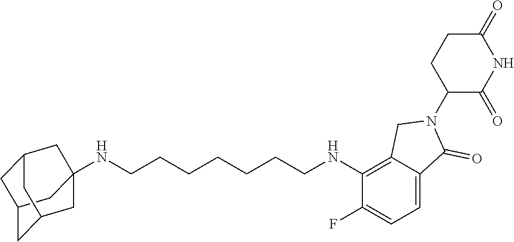 [SMILES: O=C1CCC(N2Cc3c(ccc(F)c3NCCCCCCCNC34CC5CC(CC(C5)C3)C4)C2=O)C(=O)N1] | 3-(4-((7-((adamantan-1- yl)amino)heptyl)amino)-5-fluoro-1- oxoisoindolin-2-yl)piperidine-2,6-dione |
| GT- 05879 | 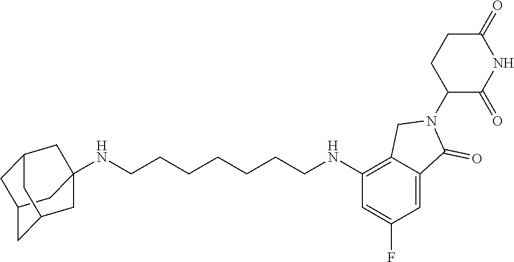 [SMILES: O=C1CCC(N2Cc3c(NCCCCCCCNC45CC6CC(CC(C6)C4)C5)cc(F)cc3C2=O)C(=O)N1] | 3-(4-((7-((adamantan-1- yl)amino)heptyl)amino)-6-fluoro-1- oxoisoindolin-2-yl)piperidine-2,6-dione |
| | 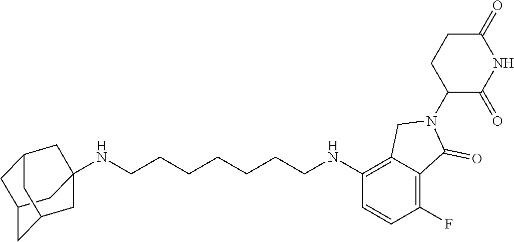 [SMILES: O=C1CCC(N2Cc3c(NCCCCCCCNC45CC6CC(CC(C6)C4)C5)ccc(F)c3C2=O)C(=O)N1] | 3-(4-((7-((adamantan-1- yl)amino)heptyl)amino)-7-fluoro-1- oxoisoindolin-2-yl)piperidine-2,6-dione |
| | 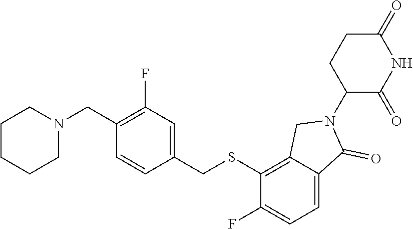 [SMILES: O=C1CCC(N2Cc3c(ccc(F)c3SCc3ccc(CN4CCCCC4)c(F)c3)C2=O)C(=O)N1] | 3-(5-fluoro-4-((3-fluoro-4-(piperidin-1- ylmethyl)benzyl)thio)-1-oxoisoindolin-2- yl)piperidine-2,6-dione |
| GT- 05443 | 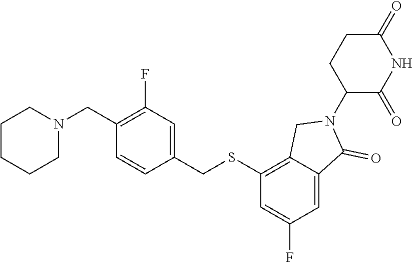 [SMILES: O=C1CCC(N2Cc3c(SCc4ccc(CN5CCCCC5)c(F)c4)cc(F)cc3C2=O)C(=O)N1] | 3-(6-fluoro-4-((3-fluoro-4-(piperidin-1- ylmethyl)benzyl)thio)-1-oxoisoindolin-2- yl)piperidine-2,6-dione |
| | 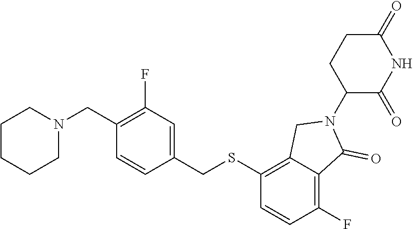 [SMILES: O=C1CCC(N2Cc3c(SCc4ccc(CN5CCCCC5)c(F)c4)ccc(F)c3C2=O)C(=O)N1] | 3-(7-fluoro-4-((3-fluoro-4-(piperidin-1- ylmethyl)benzyl)thio)-1-oxoisoindolin-2- yl)piperidine-2,6-dione |
| | 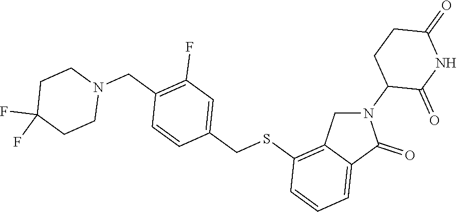 [SMILES: O=C1CCC(N2Cc3c(SCc4ccc(CN5CCC(F)(F)CC5)c(F)c4)cccc3C2=O)C(=O)N1] | 3-(4-((4-((4,4-difluoropiperidin-1- yl)methyl)-3-fluorobenzyl)thio)-1- oxoisoindolin-2-yl)piperidine-2,6-dione |
| | 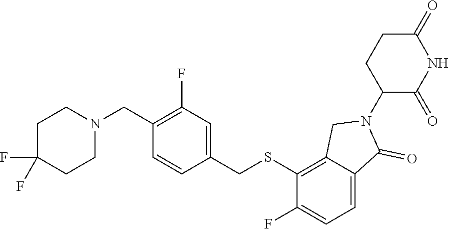 [SMILES: O=C1CCC(N2Cc3c(ccc(F)c3SCc3ccc(CN4CCC(F)(F)CC4)c(F)c3)C2=O)C(=O)N1] | 3-(4-((4-((4,4-difluoropiperidin-1- yl)methyl)-3-fluorobenzyl)thio)-5-fluoro-1- oxoisoindolin-2-yl)piperidine-2,6-dione |
| GT- 05444 | 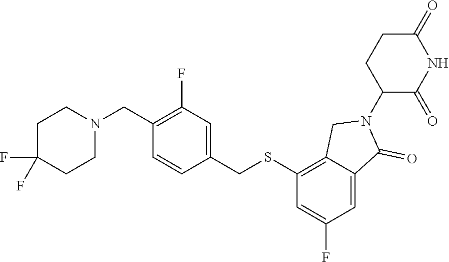 [SMILES: O=C1CCC(N2Cc3c(SCc4ccc(CN5CCC(F)(F)CC5)c(F)c4)cc(F)cc3C2=O)C(=O)N1] | 3-(4-((4-((4,4-difluoropiperidin-1- yl)methyl)-3-fluorobenzyl)thio)-6-fluoro-1- oxoisoindolin-2-yl)piperidine-2,6-dione |
| | 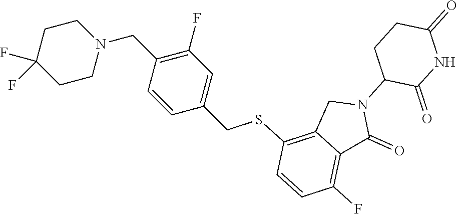 [SMILES: O=C1CCC(N2Cc3c(SCc4ccc(CN5CCC(F)(F)CC5)c(F)c4)ccc(F)c3C2=O)C(=O)N1] | 3-(4-((4-((4,4-difluoropiperidin-1- yl)methyl)-3-fluorobenzyl)thio)-7-fluoro-1- oxoisoindolin-2-yl)piperidine-2,6-dione |
| | 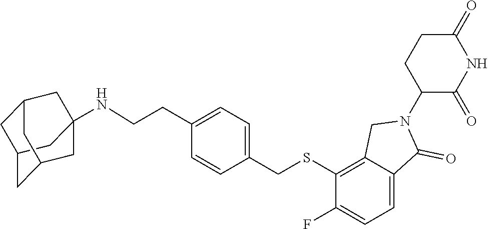 [SMILES: O=C1CCC(N2Cc3c(ccc(F)c3SCc3ccc(CCNC45CC6CC(CC(C6)C4)C5)cc3)C2=O)C(=O)N1] | 3-(4-((4-(2-((adamantan-1- yl)amino)ethyl)benzyl)thio)-5-fluoro-1- oxoisoindolin-2-yl)piperidine-2,6-dione |
| GT- 05431 | 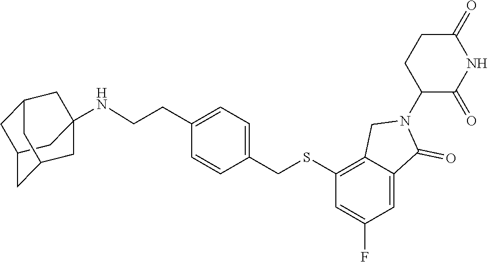 [SMILES: O=C1CCC(N2Cc3c(SCc4ccc(CCNC56CC7CC(CC(C7)C5)C6)cc4)cc(F)cc3C2=O)C(=O)N1] | 3-(4-((4-(2-((adamantan-1- yl)amino)ethyl)benzyl)thio)-6-fluoro-1- oxoisoindolin-2-yl)piperidine-2,6-dione |
| | 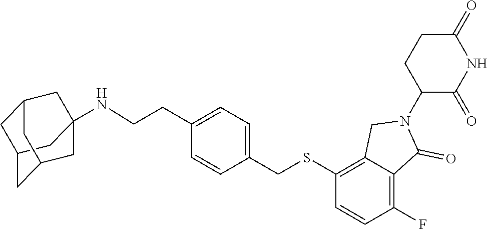 [SMILES: O=C1CCC(N2Cc3c(SCc4ccc(CCNC56CC7CC(CC(C7)C5)C6)cc4)ccc(F)c3C2=O)C(=O)N1] | 3-(4-((4-(2-((adamantan-1- yl)amino)ethyl)benzyl)thio)-7-fluoro-1- oxoisoindolin-2-yl)piperidine-2,6-dione |
| | 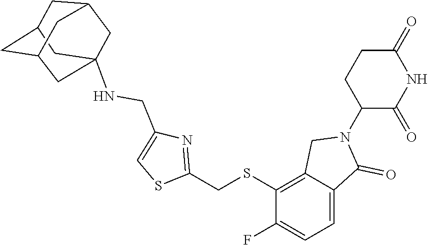 [SMILES: O=C1CCC(N2Cc3c(ccc(F)c3SCc3nc(CNC45CC6CC(CC(C6)C4)C5)cs3)C2=O)C(=O)N1] | 3-(4-(((4-(((adamantan-1- yl)amino)methyl)thiazol-2-yl)methyl)thio)- 5-fluoro-1-oxoisoindolin-2-yl)piperidine- 2,6-dione |
| | 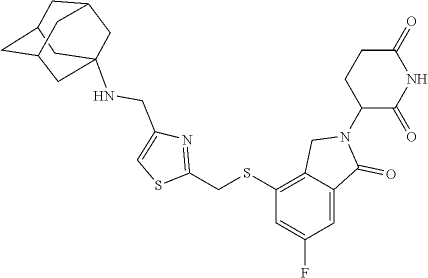 [SMILES: O=C1CCC(N2Cc3c(SCc4nc(CNC56CC7CC(CC(C7)C5)C6)cs4)cc(F)cc3C2=O)C(=O)N1] | 3-(4-(((4-(((adamantan-1- yl)amino)methyl)thiazol-2-yl)methyl)thio)- 6-fluoro-1-oxoisoindolin-2-yl)piperidine- 2,6-dione |
| | 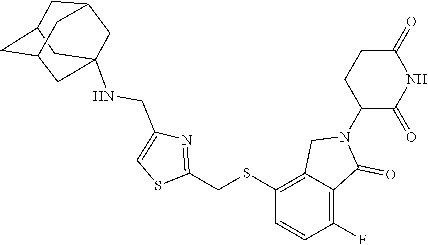 [SMILES: O=C1CCC(N2Cc3c(SCc4nc(CNC56CC7CC(CC(C7)C5)C6)cs4)ccc(F)c3C2=O)C(=O)N1] | 3-(4-(((4-(((adamantan-1- yl)amino)methyl)thiazol-2-yl)methyl)thio)- 7-fluoro-1-oxoisoindolin-2-yl)piperidine- 2,6-dione |
| | 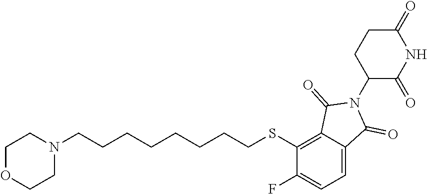 [SMILES: O=C1CCC(N2C(=O)c3ccc(F)c(SCCCCCCCCN4CCOCC4)c3C2=O)C(=O)N1] | 2-(2,6-dioxopiperidin-3-yl)-5-fluoro-4-((8- morpholinooctyl)thio)isoindoline-1,3-dione |
| | 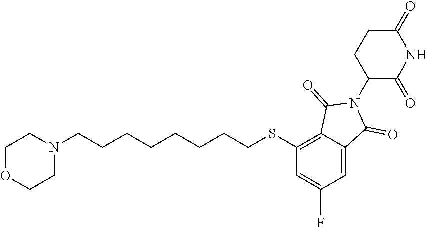 [SMILES: O=C1CCC(N2C(=O)c3cc(F)cc(SCCCCCCCCN4CCOCC4)c3C2=O)C(=O)N1] | 2-(2,6-dioxopiperidin-3-yl)-6-fluoro-4-((8- morpholinooctyl)thio)isoindoline-1,3-dione |
| | 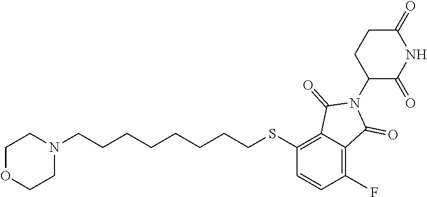 [SMILES: O=C1CCC(N2C(=O)c3c(F)ccc(SCCCCCCCCN4CCOCC4)c3C2=O)C(=O)N1] | 2-(2,6-dioxopiperidin-3-yl)-4-fluoro-7-((8- morpholinooctyl)thio)isoindoline-1,3-dione |
| | 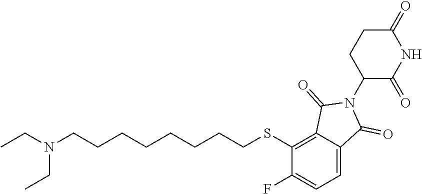 [SMILES: CCN(CC)CCCCCCCCSc1c(F)ccc2c1C(=O)N(C1CCC(=O)NC1=O)C2=O] | 4-((8-(diethylamino)octyl)thio)-2-(2,6- dioxopiperidin-3-yl)-5-fluoroisoindoline-1,3- dione |
| | 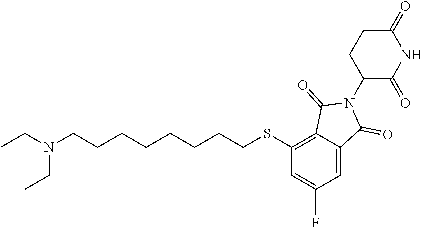 [SMILES: CCN(CC)CCCCCCCCSc1cc(F)cc2c1C(=O)N(C1CCC(=O)NC1=O)C2=O] | 4-((8-(diethylamino)octyl)thio)-2-(2,6- dioxopiperidin-3-yl)-6-fluoroisoindoline-1,3- dione |
| | 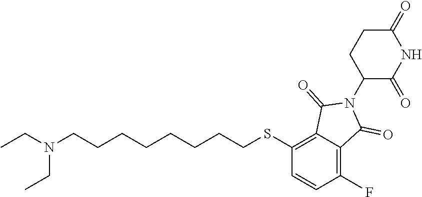 [SMILES: CCN(CC)CCCCCCCCSc1ccc(F)c2c1C(=O)N(C1CCC(=O)NC1=O)C2=O] | 4-((8-(diethylamino)octyl)thio)-2-(2,6- dioxopiperidin-3-yl)-7-fluoroisoindoline-1,3- dione |
| | 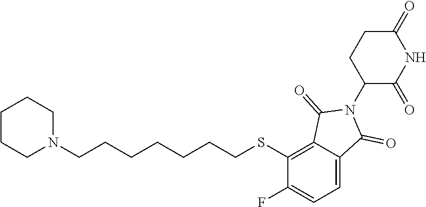 [SMILES: O=C1CCC(N2C(=O)c3ccc(F)c(SCCCCCCCN4CCCCC4)c3C2=O)C(=O)N1] | 2-(2,6-dioxopiperidin-3-yl)-5-fluoro-4-((7- (piperidin-1-yl)heptyl)thio)isoindoline-1,3- dione |
| | 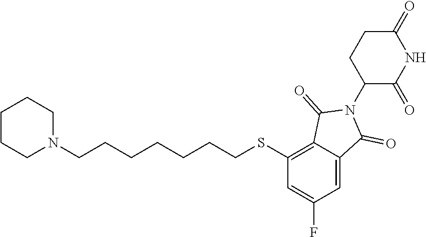 [SMILES: O=C1CCC(N2C(=O)c3cc(F)cc(SCCCCCCCN4CCCCC4)c3C2=O)C(=O)N1] | 2-(2,6-dioxopiperidin-3-yl)-6-fluoro-4-((7- (piperidin-1-yl)heptyl)thio)isoindoline-1,3- dione |
| | 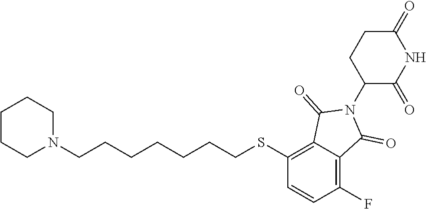 [SMILES: O=C1CCC(N2C(=O)c3c(F)ccc(SCCCCCCCN4CCCCC4)c3C2=O)C(=O)N1] | 2-(2,6-dioxopiperidin-3-yl)-4-fluoro-7-((7- (piperidin-1-yl)heptyl)thio)isoindoline-1,3- dione |
| | 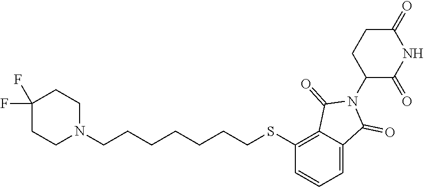 [SMILES: O=C1CCC(N2C(=O)c3cccc(SCCCCCCCN4CCC(F)(F)CC4)c3C2=O)C(=O)N1] | 4-((7-(4,4-difluoropiperidin-1- yl)heptyl)thio)-2-(2,6-dioxopiperidin-3- yl)isoindoline-1,3-dione |
| | 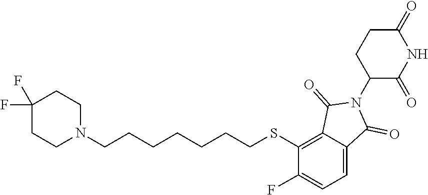 [SMILES: O=C1CCC(N2C(=O)c3ccc(F)c(SCCCCCCCN4CCC(F)(F)CC4)c3C2=O)C(=O)N1] | 4-((7-(4,4-difluoropiperidin-1- yl)heptyl)thio)-2-(2,6-dioxopiperidin-3-yl)- 5-fluoroisoindoline-1,3-dione |
| | 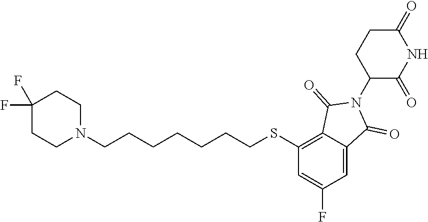 [SMILES: O=C1CCC(N2C(=O)c3cc(F)cc(SCCCCCCCN4CCC(F)(F)CC4)c3C2=O)C(=O)N1] | 4-((7-(4,4-difluoropiperidin-1- yl)heptyl)thio)-2-(2,6-dioxopiperidin-3-yl)- 6-fluoroisoindoline-1,3-dione |
| | 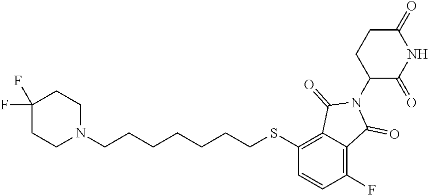 [SMILES: O=C1CCC(N2C(=O)c3c(F)ccc(SCCCCCCCN4CCC(F)(F)CC4)c3C2=O)C(=O)N1] | 4-((7-(4,4-difluoropiperidin-1- yl)heptyl)thio)-2-(2,6-dioxopiperidin-3-yl)- 7-fluoroisoindoline-1,3-dione |
| | 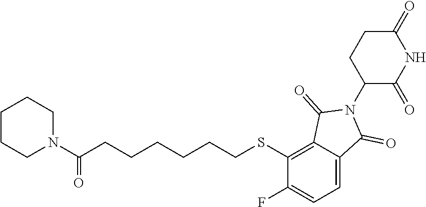 [SMILES: O=C1CCC(N2C(=O)c3ccc(F)c(SCCCCCCC(=O)N4CCCCC4)c3C2=O)C(=O)N1] | 2-(2,6-dioxopiperidin-3-yl)-5-fluoro-4-((7- oxo-7-(piperidin-1- yl)heptyl)thio)isoindoline-1,3-dione |
| | 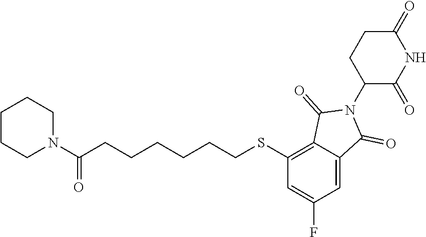 [SMILES: O=C1CCC(N2C(=O)c3cc(F)cc(SCCCCCCC(=O)N4CCCCC4)c3C2=O)C(=O)N1] | 2-(2,6-dioxopiperidin-3-yl)-6-fluoro-4-((7- oxo-7-(piperidin-1- yl)heptyl)thio)isoindoline-1,3-dione |
| | 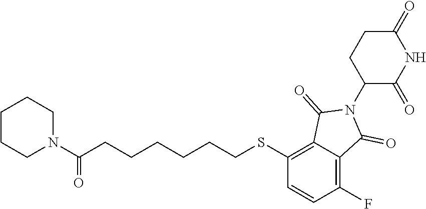 [SMILES: O=C1CCC(N2C(=O)c3c(F)ccc(SCCCCCCC(=O)N4CCCCC4)c3C2=O)C(=O)N1] | 2-(2,6-dioxopiperidin-3-yl)-4-fluoro-7-((7- oxo-7-(piperidin-1- yl)heptyl)thio)isoindoline-1,3-dione |
| | 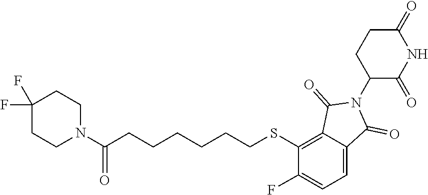 [SMILES: O=C1CCC(N2C(=O)c3ccc(F)c(SCCCCCCC(=O)N4CCC(F)(F)CC4)c3C2=O)C(=O)N1] | 4-((7-(4,4-difluoropiperidin-1-yl)-7- oxoheptyl)thio)-2-(2,6-dioxopiperidin-3-yl)- 5-fluoroisoindoline-1,3-dione |
| | 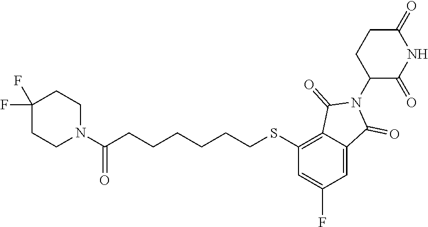 [SMILES: O=C1CCC(N2C(=O)c3cc(F)cc(SCCCCCCC(=O)N4CCC(F)(F)CC4)c3C2=O)C(=O)N1] | 4-((7-(4,4-difluoropiperidin-1-yl)-7- oxoheptyl)thio)-2-(2,6-dioxopiperidin-3-yl)- 6-fluoroisoindoline-1,3-dione |
| | 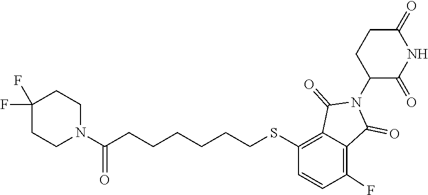 [SMILES: O=C1CCC(N2C(=O)c3c(F)ccc(SCCCCCCC(=O)N4CCC(F)(F)CC4)c3C2=O)C(=O)N1] | 4-((7-(4,4-difluoropiperidin-1-yl)-7- oxoheptyl)thio)-2-(2,6-dioxopiperidin-3-yl)- 7-fluoroisoindoline-1,3-dione |
| | 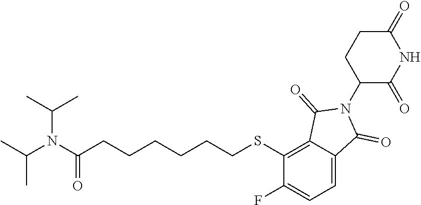 [SMILES: CC(C)N(C(=O)CCCCCCSc1c(F)ccc2c1C(=O)N(C1CCC(=O)NC1=O)C2=O)C(C)C] | 7-((2-(2,6-dioxopiperidin-3-yl)-5-fluoro-1,3- dioxoisoindolin-4-yl)thio)-N,N- diisopropylheptanamide |
| | 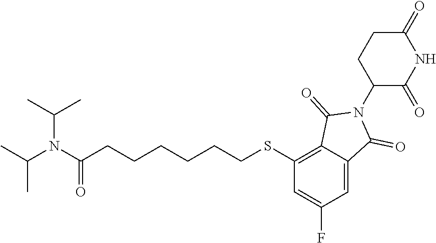 [SMILES: CC(C)N(C(=O)CCCCCCSc1cc(F)cc2c1C(=O)N(C1CCC(=O)NC1=O)C2=O)C(C)C] | 7-((2-(2,6-dioxopiperidin-3-yl)-6-fluoro-1,3- dioxoisoindolin-4-yl)thio)-N,N- diisopropylheptanamide |
| | 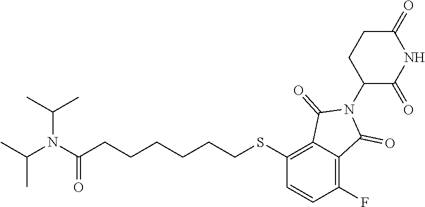 [SMILES: CC(C)N(C(=O)CCCCCCSc1ccc(F)c2c1C(=O)N(C1CCC(=O)NC1=O)C2=O)C(C)C] | 7-((2-(2,6-dioxopiperidin-3-yl)-7-fluoro-1,3- dioxoisoindolin-4-yl)thio)-N,N- diisopropylheptanamide |
| | 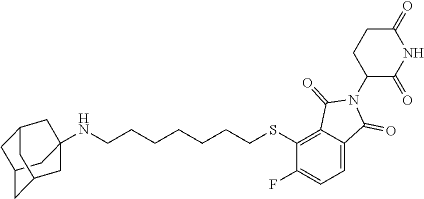 [SMILES: O=C1CCC(N2C(=O)c3ccc(F)c(SCCCCCCCNC45CC6CC(CC(C6)C4)C5)c3C2=O)C(=O)N1] | 4-((7-((adamantan-1-yl)amino)heptyl)thio)- 2-(2,6-dioxopiperidin-3-yl)-5- fluoroisoindoline-1,3-dione |
| | 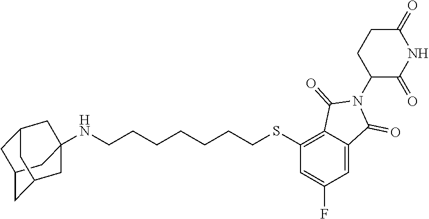 [SMILES: O=C1CCC(N2C(=O)c3cc(F)cc(SCCCCCCCNC45CC6CC(CC(C6)C4)C5)c3C2=O)C(=O)N1] | 4-((7-((adamantan-1-yl)amino)heptyl)thio)- 2-(2,6-dioxopiperidin-3-yl)-6- fluoroisoindoline-1,3-dione |
| | 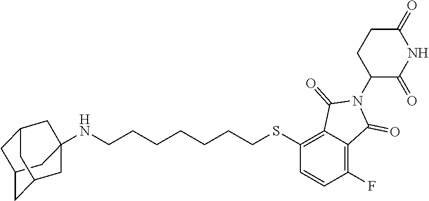 [SMILES: O=C1CCC(N2C(=O)c3c(F)ccc(SCCCCCCCNC45CC6CC(CC(C6)C4)C5)c3C2=O)C(=O)N1] | 4-((7-((adamantan-1-yl)amino)heptyl)thio)- 2-(2,6-dioxopiperidin-3-yl)-7- fluoroisoindoline-1,3-dione |
| | 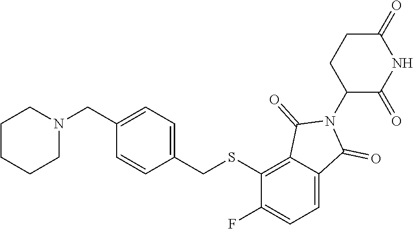 [SMILES: O=C1CCC(N2C(=O)c3ccc(F)c(SCc4ccc(CN5CCCCC5)cc4)c3C2=O)C(=O)N1] | 2-(2,6-dioxopiperidin-3-yl)-5-fluoro-4-((4- (piperidin-1- ylmethyl)benzyl)thio)isoindoline-1,3-dione |
| | 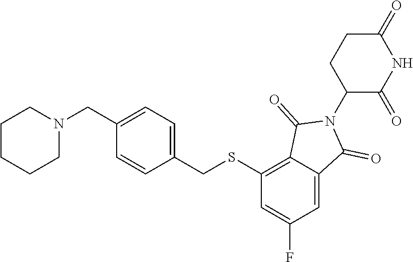 [SMILES: O=C1CCC(N2C(=O)c3cc(F)cc(SCc4ccc(CN5CCCCC5)cc4)c3C2=O)C(=O)N1] | 2-(2,6-dioxopiperidin-3-yl)-6-fluoro-4-((4- (piperidin-1- ylmethyl)benzyl)thio)isoindoline-1,3-dione |
| | 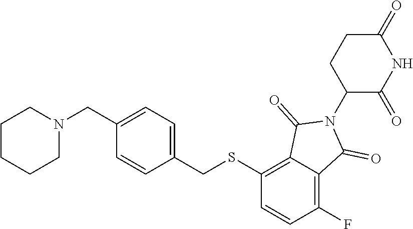 [SMILES: O=C1CCC(N2C(=O)c3c(F)ccc(SCc4ccc(CN5CCCCC5)cc4)c3C2=O)C(=O)N1] | 2-(2,6-dioxopiperidin-3-yl)-4-fluoro-7-((4- (piperidin-1- ylmethyl)benzyl)thio)isoindoline-1,3-dione |
| | 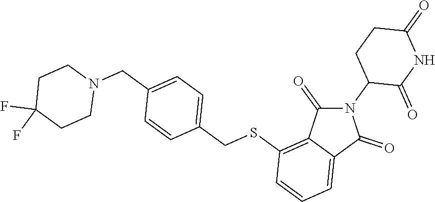 [SMILES: O=C1CCC(N2C(=O)c3cccc(SCc4ccc(CN5CCC(F)(F)CC5)cc4)c3C2=O)C(=O)N1] | 4-((4-((4,4-difluoropiperidin-1- yl)methyl)benzyl)thio)-2-(2,6- dioxopiperidin-3-yl)isoindoline-1,3-dione |
| | 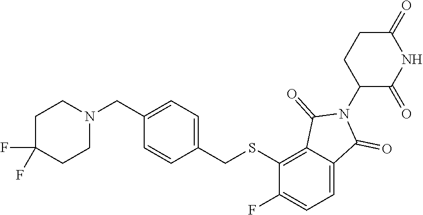 [SMILES: O=C1CCC(N2C(=O)c3ccc(F)c(SCc4ccc(CN5CCC(F)(F)CC5)cc4)c3C2=O)C(=O)N1] | 4-((4-((4,4-difluoropiperidin-1- yl)methyl)benzyl)thio)-2-(2,6- dioxopiperidin-3-yl)-5-fluoroisoindoline-1,3- dione |
| | 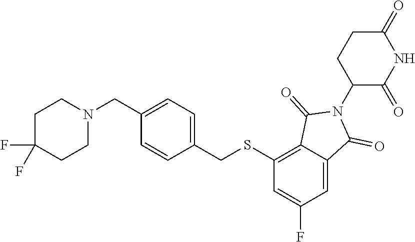 [SMILES: O=C1CCC(N2C(=O)c3cc(F)cc(SCc4ccc(CN5CCC(F)(F)CC5)cc4)c3C2=O)C(=O)N1] | 4-((4-((4,4-difluoropiperidin-1- yl)methyl)benzyl)thio)-2-(2,6- dioxopiperidin-3-yl)-6-fluoroisoindoline-1,3- dione |
| | 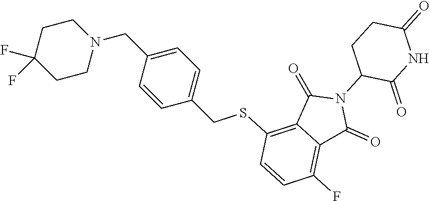 [SMILES: O=C1CCC(N2C(=O)c3c(F)ccc(SCc4ccc(CN5CCC(F)(F)CC5)cc4)c3C2=O)C(=O)N1] | 4-((4-((4,4-difluoropiperidin-1- yl)methyl)benzyl)thio)-2-(2,6- dioxopiperidin-3-yl)-7-fluoroisoindoline-1,3- dione |
| | 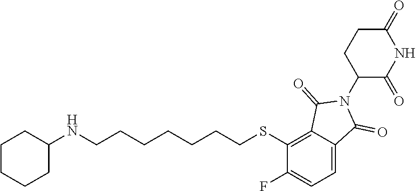 [SMILES: O=C1CCC(N2C(=O)c3ccc(F)c(SCCCCCCCNC4CCCCC4)c3C2=O)C(=O)N1] | 4-((7-(cyclohexylamino)heptyl)thio)-2-(2,6- dioxopiperidin-3-yl)-5-fluoroisoindoline-1,3- dione |
| | 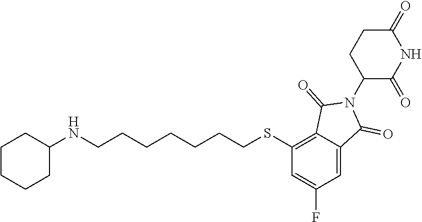 [SMILES: O=C1CCC(N2C(=O)c3cc(F)cc(SCCCCCCCNC4CCCCC4)c3C2=O)C(=O)N1] | 4-((7-(cyclohexylamino)heptyl)thio)-2-(2,6- dioxopiperidin-3-yl)-6-fluoroisoindoline-1,3- dione |
| | 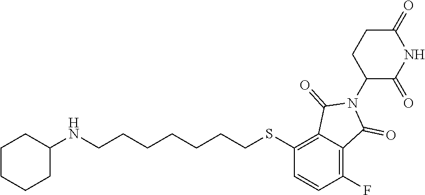 [SMILES: O=C1CCC(N2C(=O)c3c(F)ccc(SCCCCCCCNC4CCCCC4)c3C2=O)C(=O)N1] | 4-((7-(cyclohexylamino)heptyl)thio)-2-(2,6- dioxopiperidin-3-yl)-7-fluoroisoindoline-1,3- dione |
| | 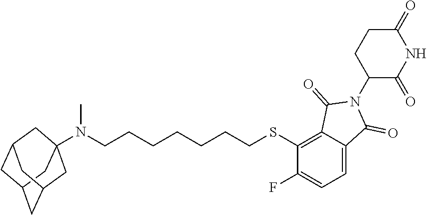 [SMILES: CN(CCCCCCCSc1c(F)ccc2c1C(=O)N(C1CCC(=O)NC1=O)C2=O)C12CC3CC(CC(C3)C1)C2] | 4-((7-((adamantan-1- yl)(methyl)amino)heptyl)thio)-2-(2,6- dioxopiperidin-3-yl)-5-fluoroisoindoline-1,3- dione |
| | 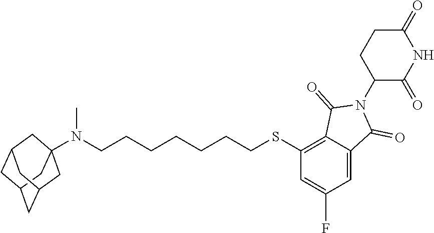 [SMILES: CN(CCCCCCCSc1cc(F)cc2c1C(=O)N(C1CCC(=O)NC1=O)C2=O)C12CC3CC(CC(C3)C1)C2] | 4-((7-((adamantan-1- yl)(methyl)amino)heptyl)thio)-2-(2,6- dioxopiperidin-3-yl)-6-fluoroisoindoline-1,3- dione |
| | 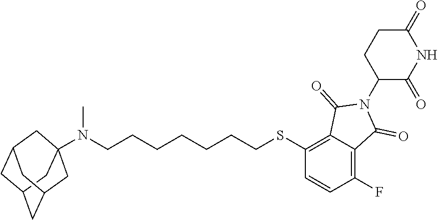 [SMILES: CN(CCCCCCCSc1ccc(F)c2c1C(=O)N(C1CCC(=O)NC1=O)C2=O)C12CC3CC(CC(C3)C1)C2] | 4-((7-((adamantan-1- yl)(methyl)amino)heptyl)thio)-2-(2,6- dioxopiperidin-3-yl)-7-fluoroisoindoline-1,3- dione |
| | 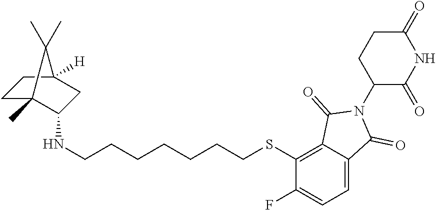 [SMILES: CC1(C)[C@@H]2CC[C@@]1(C)[C@@H](NCCCCCCCSc1c(F)ccc3c1C(=O)N(C1CCC(=O)NC1=O)C3=O)C2] | 2-(2,6-dioxopiperidin-3-yl)-5-fluoro-4-((7- (((1R,2S,4R)-1,7,7- trimethylbicyclo[2.2.1]heptan-2- yl)amino)heptyl)thio)isoindoline-1,3-dione |
| | 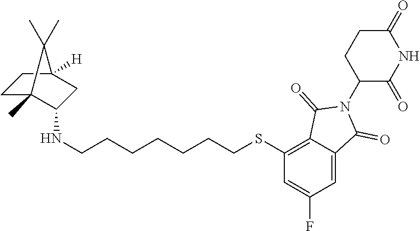 [SMILES: CC1(C)[C@@H]2CC[C@@]1(C)[C@@H](NCCCCCCCSc1cc(F)cc3c1C(=O)N(C1CCC(=O)NC1=O)C3=O)C2] | 2-(2,6-dioxopiperidin-3-yl)-6-fluoro-4-((7- (((1R,2S,4R)-1,7,7- trimethylbicyclo[2.2.1]heptan-2- yl)amino)heptyl)thio)isoindoline-1,3-dione |
| | 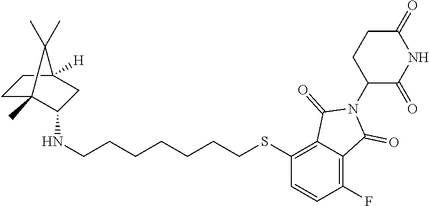 [SMILES: CC1(C)[C@@H]2CC[C@@]1(C)[C@@H](NCCCCCCCSc1ccc(F)c3c1C(=O)N(C1CCC(=O)NC1=O)C3=O)C2] | 2-(2,6-dioxopiperidin-3-yl)-4-fluoro-7-((7- (((1R,2S,4R)-1,7,7- trimethylbicyclo[2.2.1]heptan-2- yl)amino)heptyl)thio)isoindoline-1,3-dione |
| | 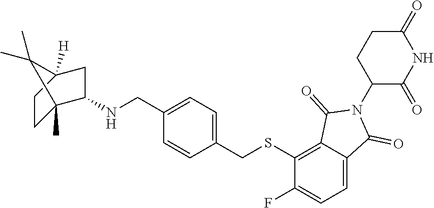 [SMILES: CC1(C)[C@@H]2CC[C@@]1(C)[C@@H](NCc1ccc(CSc3c(F)ccc4c3C(=O)N(C3CCC(=O)NC3=O)C4=O)cc1)C2] | 2-(2,6-dioxopiperidin-3-yl)-5-fluoro-4-((4- ((((1R,2S,4R)-1,7,7- trimethylbicyclo[2.2.1]heptan-2- yl)amino)methyl)benzyl)thio)isoindoline- 1,3-dione |
| | 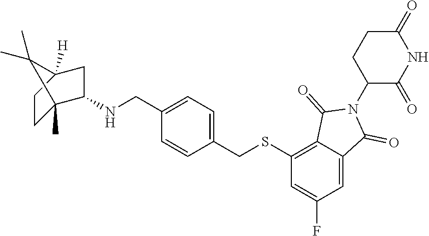 [SMILES: CC1(C)[C@@H]2CC[C@@]1(C)[C@@H](NCc1ccc(CSc3cc(F)cc4c3C(=O)N(C3CCC(=O)NC3=O)C4=O)cc1)C2] | 2-(2,6-dioxopiperidin-3-yl)-6-fluoro-4-((4- ((((1R,2S,4R)-1,7,7- trimethylbicyclo[2.2.1]heptan-2- yl)amino)methyl)benzyl)thio)isoindoline- 1,3-dione |
| | 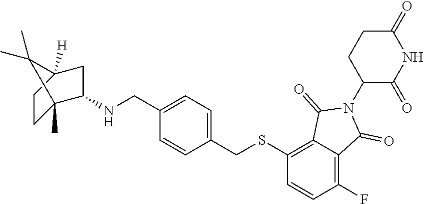 [SMILES: CC1(C)[C@@H]2CC[C@@]1(C)[C@@H](NCc1ccc(CSc3ccc(F)c4c3C(=O)N(C3CCC(=O)NC3=O)C4=O)cc1)C2] | 2-(2,6-dioxopiperidin-3-yl)-4-fluoro-7-((4- ((((1R,2S,4R)-1,7,7- trimethylbicyclo[2.2.1]heptan-2- yl)amino)methyl)benzyl)thio)isoindoline- 1,3-dione |
| | 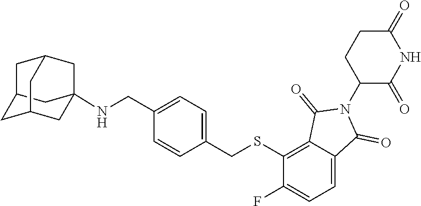 [SMILES: O=C1CCC(N2C(=O)c3ccc(F)c(SCc4ccc(CNC56CC7CC(CC(C7)C5)C6)cc4)c3C2=O)C(=O)N1] | 4-((4-(((adamantan-1- yl)amino)methyl)benzyl)thio)-2-(2,6- dioxopiperidin-3-yl)-5-fluoroisoindoline-1,3- dione |
| | 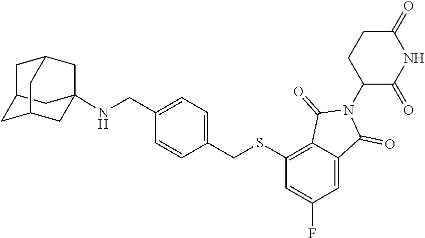 [SMILES: O=C1CCC(N2C(=O)c3cc(F)cc(SCc4ccc(CNC56CC7CC(CC(C7)C5)C6)cc4)c3C2=O)C(=O)N1] | 4-((4-(((adamantan-1- yl)amino)methyl)benzyl)thio)-2-(2,6- dioxopiperidin-3-yl)-6-fluoroisoindoline-1,3- dione |
| | 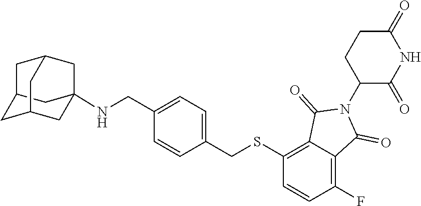 [SMILES: O=C1CCC(N2C(=O)c3c(F)ccc(SCc4ccc(CNC56CC7CC(CC(C7)C5)C6)cc4)c3C2=O)C(=O)N1] | 4-((4-(((adamantan-1- yl)amino)methyl)benzyl)thio)-2-(2,6- dioxopiperidin-3-yl)-7-fluoroisoindoline-1,3- dione |
| | 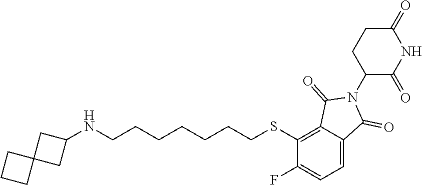 [SMILES: O=C1CCC(N2C(=O)c3ccc(F)c(SCCCCCCCNC4CC5(CCC5)C4)c3C2=O)C(=O)N1] | 2-(2,6-dioxopiperidin-3-yl)-5-fluoro-4-((7- (spiro[3.3]heptan-2- ylamino)heptyl)thio)isoindoline-1,3-dione |
| | 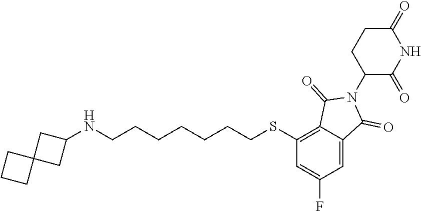 [SMILES: O=C1CCC(N2C(=O)c3cc(F)cc(SCCCCCCCNC4CC5(CCC5)C4)c3C2=O)C(=O)N1] | 2-(2,6-dioxopiperidin-3-yl)-6-fluoro-4-((7- (spiro[3.3]heptan-2- ylamino)heptyl)thio)isoindoline-1,3-dione |
| | 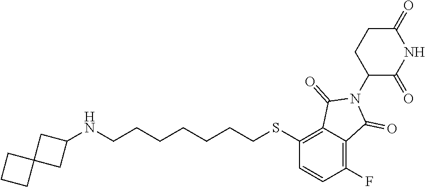 [SMILES: O=C1CCC(N2C(=O)c3c(F)ccc(SCCCCCCCNC4CC5(CCC5)C4)c3C2=O)C(=O)N1] | 2-(2,6-dioxopiperidin-3-yl)-4-fluoro-7-((7- (spiro[3.3]heptan-2- ylamino)heptyl)thio)isoindoline-1,3-dione |
| | 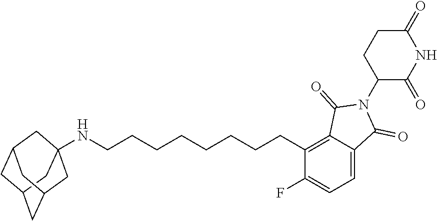 [SMILES: O=C1CCC(N2C(=O)c3ccc(F)c(CCCCCCCCNC45CC6CC(CC(C6)C4)C5)c3C2=O)C(=O)N1] | 4-(8-((adamantan-1-yl)amino)octyl)-2-(2,6- dioxopiperidin-3-yl)-5-fluoroisoindoline-1,3- dione |
| | 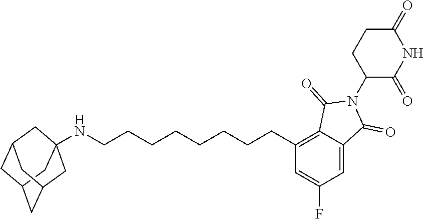 [SMILES: O=C1CCC(N2C(=O)c3cc(F)cc(CCCCCCCCNC45CC6CC(CC(C6)C4)C5)c3C2=O)C(=O)N1] | 4-(8-((adamantan-1-yl)amino)octyl)-2-(2,6- dioxopiperidin-3-yl)-6-fluoroisoindoline-1,3- dione |
| | 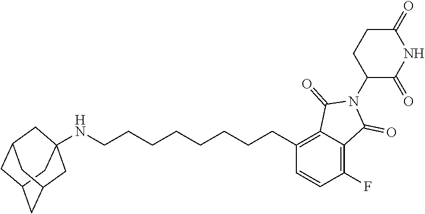 [SMILES: O=C1CCC(N2C(=O)c3c(F)ccc(CCCCCCCCNC45CC6CC(CC(C6)C4)C5)c3C2=O)C(=O)N1] | 4-(8-((adamantan-1-yl)amino)octyl)-2-(2,6- dioxopiperidin-3-yl)-7-fluoroisoindoline-1,3- dione |
| | 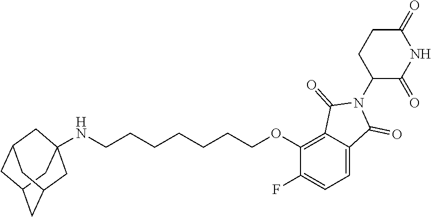 [SMILES: O=C1CCC(N2C(=O)c3ccc(F)c(OCCCCCCCNC45CC6CC(CC(C6)C4)C5)c3C2=O)C(=O)N1] | 4-((7-((adamantan-1-yl)amino)heptyl)oxy)- 2-(2,6-dioxopiperidin-3-yl)-5- fluoroisoindoline-1,3-dione |
| | 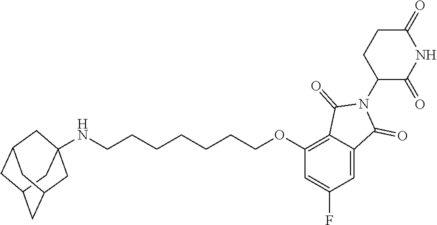 [SMILES: O=C1CCC(N2C(=O)c3cc(F)cc(OCCCCCCCNC45CC6CC(CC(C6)C4)C5)c3C2=O)C(=O)N1] | 4-((7-((adamantan-1-yl)amino)heptyl)oxy)- 2-(2,6-dioxopiperidin-3-yl)-6- fluoroisoindoline-1,3-dione |
| | 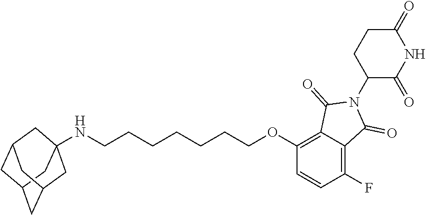 [SMILES: O=C1CCC(N2C(=O)c3c(F)ccc(OCCCCCCCNC45CC6CC(CC(C6)C4)C5)c3C2=O)C(=O)N1] | 4-((7-((adamantan-1-yl)amino)heptyl)oxy)- 2-(2,6-dioxopiperidin-3-yl)-7- fluoroisoindoline-1,3-dione |
| | 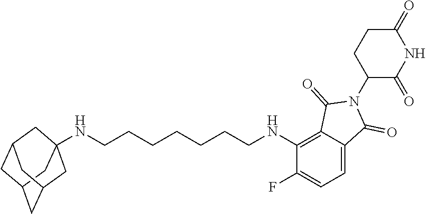 [SMILES: O=C1CCC(N2C(=O)c3ccc(F)c(NCCCCCCCNC45CC6CC(CC(C6)C4)C5)c3C2=O)C(=O)N1] | 4-((7-((adamantan-1- yl)amino)heptyl)amino)-2-(2,6- dioxopiperidin-3-yl)-5-fluoroisoindoline-1,3- dione |
| | 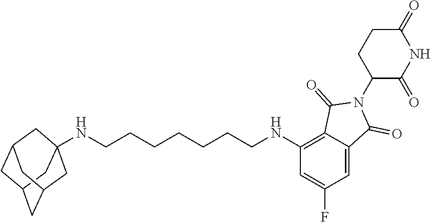 [SMILES: O=C1CCC(N2C(=O)c3cc(F)cc(NCCCCCCCNC45CC6CC(CC(C6)C4)C5)c3C2=O)C(=O)N1] | 4-((7-((adamantan-1- yl)amino)heptyl)amino)-2-(2,6- dioxopiperidin-3-yl)-6-fluoroisoindoline-1,3- dione |
| | 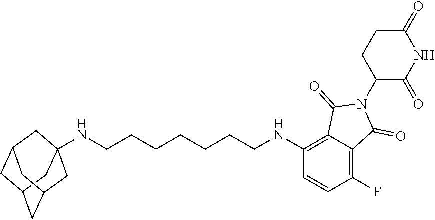 [SMILES: O=C1CCC(N2C(=O)c3c(F)ccc(NCCCCCCCNC45CC6CC(CC(C6)C4)C5)c3C2=O)C(=O)N1] | 4-((7-((adamantan-1- yl)amino)heptyl)amino)-2-(2,6- dioxopiperidin-3-yl)-7-fluoroisoindoline-1,3- dione |
| | 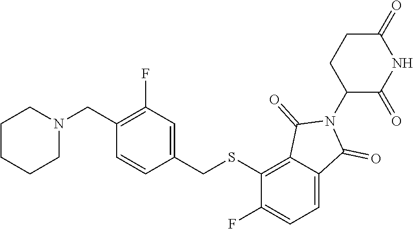 [SMILES: O=C1CCC(N2C(=O)c3ccc(F)c(SCc4ccc(CN5CCCCC5)c(F)c4)c3C2=O)C(=O)N1] | 2-(2,6-dioxopiperidin-3-yl)-5-fluoro-4-((3- fluoro-4-(piperidin-1- ylmethyl)benzyl)thio)isoindoline-1,3-dione |
| | 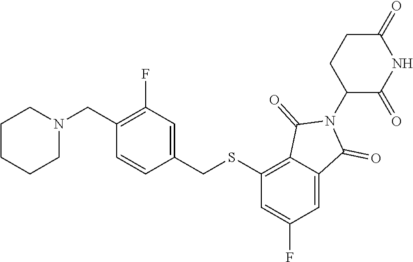 [SMILES: O=C1CCC(N2C(=O)c3cc(F)cc(SCc4ccc(CN5CCCCC5)c(F)c4)c3C2=O)C(=O)N1] | 2-(2,6-dioxopiperidin-3-yl)-6-fluoro-4-((3- fluoro-4-(piperidin-1- ylmethyl)benzyl)thio)isoindoline-1,3-dione |
| | 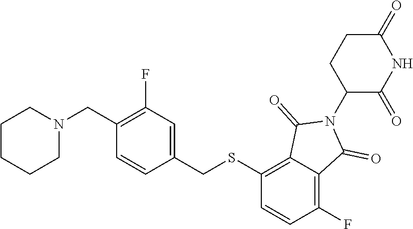 [SMILES: O=C1CCC(N2C(=O)c3c(F)ccc(SCc4ccc(CN5CCCCC5)c(F)c4)c3C2=O)C(=O)N1] | 2-(2,6-dioxopiperidin-3-yl)-4-fluoro-7-((3- fluoro-4-(piperidin-1- ylmethyl)benzyl)thio)isoindoline-1,3-dione |
| | 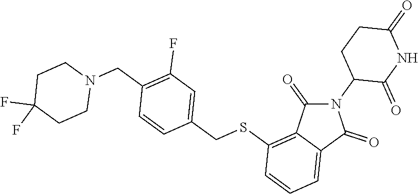 [SMILES: O=C1CCC(N2C(=O)c3cccc(SCc4ccc(CN5CCC(F)(F)CC5)c(F)c4)c3C2=O)C(=O)N1] | 4-((4-((4,4-difluoropiperidin-1-yl)methyl)-3- fluorobenzyl)thio)-2-(2,6-dioxopiperidin-3- yl)isoindoline-1,3-dione |
| | 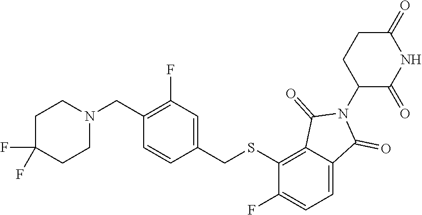 [SMILES: O=C1CCC(N2C(=O)c3ccc(F)c(SCc4ccc(CN5CCC(F)(F)CC5)c(F)c4)c3C2=O)C(=O)N1] | 4-((4-((4,4-difluoropiperidin-1-yl)methyl)-3- fluorobenzyl)thio)-2-(2,6-dioxopiperidin-3- yl)-5-fluoroisoindoline-1,3-dione |
| | 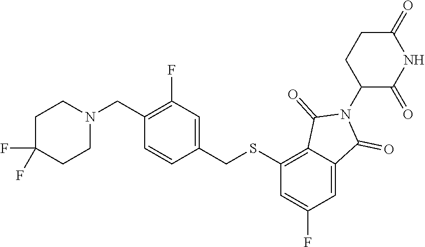 [SMILES: O=C1CCC(N2C(=O)c3cc(F)cc(SCc4ccc(CN5CCC(F)(F)CC5)c(F)c4)c3C2=O)C(=O)N1] | 4-((4-((4,4-difluoropiperidin-1-yl)methyl)-3- fluorobenzyl)thio)-2-(2,6-dioxopiperidin-3- yl)-6-fluoroisoindoline-1,3-dione |
| | 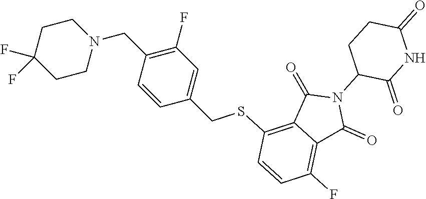 [SMILES: O=C1CCC(N2C(=O)c3c(F)ccc(SCc4ccc(CN5CCC(F)(F)CC5)c(F)c4)c3C2=O)C(=O)N1] | 4-((4-((4,4-difluoropiperidin-1-yl)methyl)-3- fluorobenzyl)thio)-2-(2,6-dioxopiperidin-3- yl)-7-fluoroisoindoline-1,3-dione |
| | 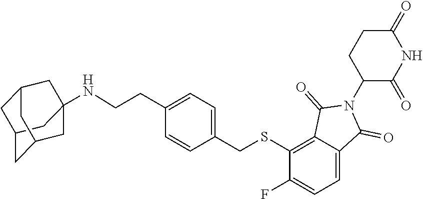 [SMILES: O=C1CCC(N2C(=O)c3ccc(F)c(SCc4ccc(CCNC56CC7CC(CC(C7)C5)C6)cc4)c3C2=O)C(=O)N1] | 4-((4-(2-((adamantan-1- yl)amino)ethyl)benzyl)thio)-2-(2,6- dioxopiperidin-3-yl)-5-fluoroisoindoline-1,3- dione |
| | 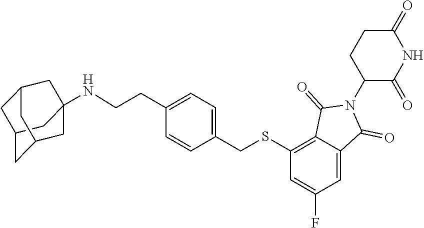 [SMILES: O=C1CCC(N2C(=O)c3cc(F)cc(SCc4ccc(CCNC56CC7CC(CC(C7)C5)C6)cc4)c3C2=O)C(=O)N1] | 4-((4-(2-((adamantan-1- yl)amino)ethyl)benzyl)thio)-2-(2,6- dioxopiperidin-3-yl)-6-fluoroisoindoline-1,3- dione |
| | 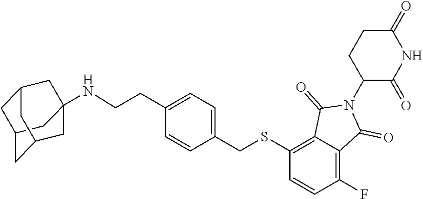 [SMILES: O=C1CCC(N2C(=O)c3c(F)ccc(SCc4ccc(CCNC56CC7CC(CC(C7)C5)C6)cc4)c3C2=O)C(=O)N1] | 4-((4-(2-((adamantan-1- yl)amino)ethyl)benzyl)thio)-2-(2,6- dioxopiperidin-3-yl)-7-fluoroisoindoline-1,3- dione |
| | 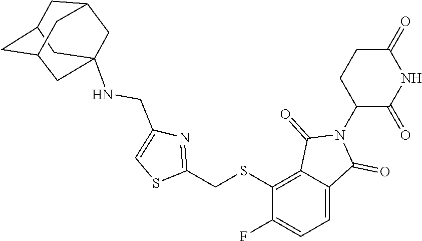 [SMILES: O=C1CCC(N2C(=O)c3ccc(F)c(SCc4nc(CNC56CC7CC(CC(C7)C5)C6)cs4)c3C2=O)C(=O)N1] | 4-(((4-(((adamantan-1- yl)amino)methyl)thiazol-2-yl)methyl)thio)- 2-(2,6-dioxopiperidin-3-yl)-5- fluoroisoindoline-1,3-dione |
| | 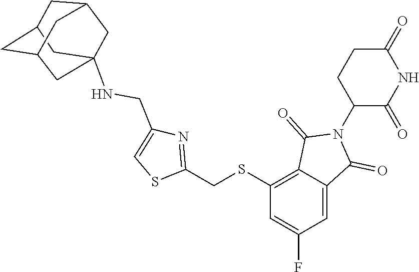 [SMILES: O=C1CCC(N2C(=O)c3cc(F)cc(SCc4nc(CNC56CC7CC(CC(C7)C5)C6)cs4)c3C2=O)C(=O)N1] | 4-(((4-(((adamantan-1- yl)amino)methyl)thiazol-2-yl)methyl)thio)- 2-(2,6-dioxopiperidin-3-yl)-6- fluoroisoindoline-1,3-dione |
| | 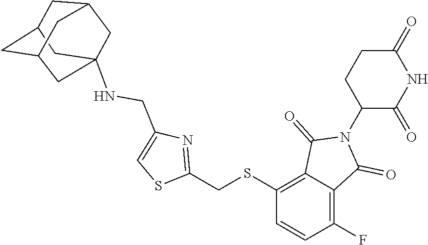 [SMILES: O=C1CCC(N2C(=O)c3c(F)ccc(SCc4nc(CNC56CC7CC(CC(C7)C5)C6)cs4)c3C2=O)C(=O)N1] | 4-(((4-(((adamantan-1- yl)amino)methyl)thiazol-2-yl)methyl)thio)- 2-(2,6-dioxopiperidin-3-yl)-7- fluoroisoindoline-1,3-dione |

### II. Other Forms of Compounds (Including Salts, Enantiomers, Stereoisomers, Solvates, Isotopically Enriched Analogs, Prodrugs, or Polymorphs of the Compounds)

The compounds of the present disclosure have the structures of any one of Formula (I), Formula (I-1), Formula (I-2), Formula (I-3), Formula (I-4), Formula (Ia), Formula (Ib), Formula (Ic), Formula (Id), or Formula (Ie). Unless otherwise specified, all references to the compounds of the present disclosure also include compounds of any one of Formula (I), Formula (I-1), Formula (I-2), Formula (I-3), Formula (I-4), Formula (Ia), Formula (Ib), Formula (Ic), Formula (Id), or Formula (Ie) and specific compounds within the scope of these general formulae.

It should be recognized that compounds of the present disclosure (including compounds of Formula (I), Formula (I-1), Formula (I-2), Formula (I-3), Formula (I-4), Formula (Ia), Formula (Ib), Formula (Ic), Formula (Id), or Formula (Ie)) may have a stereo-configuration and thus can exist in more than one stereoisomeric form. The present disclosure also relates to optically enriched compounds having a stereo-configuration, e.g., greater than about 90% enantiomeric/diastereomeric excess (“ee”), such as about 95% ee or 97% ee, or greater than 99% ee, and mixtures thereof, including racemic mixtures. As used herein, “optically enriched” means that a mixture of enantiomers consists of a significantly greater proportion of one enantiomer, and can be described by enantiomeric excess (ee %). Purification of isomers and separation of mixtures of isomers can be accomplished by standard techniques known in the art (e.g., column chromatography, preparative TLC, preparative HPLC, asymmetric synthesis (e.g., by using chiral intermediates) and/or or chiral resolution, etc.).

In some embodiments, polymorph forms or salts of the compounds of the present disclosure are also provided. Salts of the compounds of the present disclosure can be pharmaceutically acceptable salts including, but not limited to, hydrochlorides, sulfates, citrates, maleates, sulfonates, citrates, lactates, tartrates, fumarates, phosphates, dihydrophosphates, pyrophosphates, metaphosphates, oxalates, malonates, benzoates, mandelates, succinates, trifluoroacetates, hydroxyacetates, or p-toluenesulfonates, etc. The compounds of the present disclosure can exist as non-solvated or solvated forms in pharmaceutically acceptable solvents such as water, ethanol, and the like. In some embodiments, the compounds of the present disclosure can be prepared as prodrugs or precursor drugs.

Prodrugs can be converted into parent drugs in the body to play their role. In some embodiments, isotopically-labeled compounds of the present disclosure are also provided, examples of which include deuterium (D or ²H).

### III. Compositions/Formulations

In some embodiments, the present disclosure provides a pharmaceutical composition comprising as an active ingredient the compound of the present disclosure or a pharmaceutically acceptable salt, solvate, isotopically enriched analog, polymorph, prodrug, stereoisomer (including enantiomer), or mixture of stereoisomers thereof, and at least one pharmaceutically acceptable carrier.

In some embodiments, pharmaceutically acceptable carriers include, but are not limited to, fillers, stabilizers, dispersants, suspending agents, diluents, excipients, thickeners, colorants, solvents, or encapsulating materials. Each carrier must be “acceptable” in the sense of being compatible with the other ingredients of the formulation (including the compounds useful in the present disclosure) and not injurious to the patient. Some examples of materials that can be used as pharmaceutically acceptable carriers include: sugars such as lactose, glucose, and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository wax; oils such as peanut oil, cottonseed oil, safflower oil, sesame oil, olive oil, corn oil and soybean oil; glycols such as propylene glycol; polyols such as glycerol, sorbitol, mannitol, and polyethylene glycol; esters such as ethyl oleate and ethyl laurate; agar; buffers such as magnesium hydroxide and aluminum hydroxide; surfactant phosphate buffer solution; polyethylene oxide, polyvinylpyrrolidone, polyacrylamide, poloxamer; and other common non-toxic compatible substances used in pharmaceutical formulations.

The pharmaceutical composition of the present disclosure can further comprise at least one second therapeutic agent, e.g., an anticancer agent. The second therapeutic agent may be used in combination with the compounds of Formula (I) of the present disclosure to treat the diseases or disorders as disclosed herein. The second therapeutic agent includes, but is not limited to, chemotherapeutic agents, immunotherapeutic agents, gene therapeutic agents, anti-angiogenic agents, immunomodulators, and the like.

The pharmaceutical composition of the present disclosure comprising, as an active ingredient, the compounds of Formula (I) of the present disclosure or a pharmaceutically acceptable salt thereof can be formulated into any suitable formulations such as sprays, patches, tablets (such as conventional tablets, dispersible tablets, orally disintegrating tablets), capsules (such as soft capsules, hard capsules, enteric-coated capsules), dragees, troches, powders, granules, powder injections, suppositories, or liquid formulations (such as suspensions (e.g., aqueous or oily suspensions), solutions, emulsions, or syrups), or conventional injection dosage forms such as injectable solutions (e.g., sterile injectable solutions formulated according to methods known in the art using water, Ringer's solution, or isotonic sodium chloride solution or the like as a vehicle or solvent) or lyophilized injectable formulation and the like, depending upon a suitable route of administration (including, but not limited to, nasal administration, inhalation administration, topical administration, oral administration, oral mucosal administration, rectal administration, intrapleural administration, intraperitoneal administration, vaginal administration, intramuscular administration, subcutaneous administration, transdermal administration, epidural administration, intrathecal administration, and intravenous administration). Those skilled in the art can also formulate the compounds of Formula (I) of the present disclosure into conventional, dispersible, chewable, orally disintegrating or rapidly dissolving formulations, or sustained-release capsules or controlled-release capsules as needed.

The compounds of Formula (I) of the present disclosure, as an active ingredient, is contained in the pharmaceutically acceptable carrier or diluent in an amount sufficient to deliver to a subject a therapeutically effective amount for the indication to be treated, without causing serious toxic effects in the subject treated. A dosage of the active compound for all diseases or disorders mentioned herein ranges, for example, from about 5 ng/kg to 500 mg/kg, from about 10 ng/kg to 300 mg/kg per day, such as from 0.1 to 100 mg/kg, or from 0.5 to about 25 mg per kilogram body weight of the subject per day.

The compounds of Formula (I) of the present disclosure or pharmaceutically acceptable salts thereof can be conveniently administered in any suitable unit dosage form. Suitable unit dosage form specifications include, but are not limited to, less than 1 mg, 1 mg to 3000 mg, 5 mg to 1000 mg, for example, 5 to 500 mg, 25 to 250 mg of active ingredient per unit dosage form.

### IV. Kits/Packaged Products

The compounds of Formula (I) or a pharmaceutically acceptable salt, solvate, isotopically enriched analog, polymorph, prodrug, stereoisomer (including enantiomer), or mixture of stereoisomers thereof is used as a medicament. The medicament of the present disclosure or the pharmaceutical composition of the present disclosure may be presented in a kit/packaged product. The kit/packaged product may include a package or container including, but not limited to, ampoules, blister packs, pharmaceutical plastic bottles, vials, pharmaceutical glass bottles, containers, syringes, laminated flexible packaging, co-extruded film infusion containers, test tubes and dispensing devices, and the like. The kit/packaged product may contain instructions for use of the product.

### V. Methods and Uses

The compounds of Formula (I) or a pharmaceutically acceptable salt, solvate, isotopically enriched analog, polymorph, prodrug, stereoisomer (including enantiomer), or mixture of stereoisomers thereof can be used as a medicament. Especially the compounds of Formula (I) or a pharmaceutically acceptable salt, solvate, isotopically enriched analog, polymorph, prodrug, stereoisomer (including enantiomer), or mixture of stereoisomers thereof can be used for the manufacture of a medicament for the prevention and/or treatment of diseases or disorders associated with cereblon protein. The diseases or disorders associated with cereblon protein comprise: tumor, infectious disease, inflammatory disease, autoimmune disease, anemia, hemorrhagic shock, transplant rejection, multiple organ dysfunction syndrome (MODS), sarcoidosis, adult respiratory distress syndrome, cardiovascular disease, Richter syndrome (RS), acute liver failure, or diabetes. In some embodiments, the diseases or disorders associated with cereblon protein include, but are not limited to, tumor, infectious disease, inflammatory disease, autoimmune disease, anemia, hemorrhagic shock, transplant rejection, multiple organ dysfunction syndrome (MODS), sarcoidosis, adult respiratory distress syndrome, cardiovascular disease, Richter syndrome (RS), acute liver failure, or diabetes. In some embodiments, the diseases or disorders associated with cereblon protein include, but are not limited to, myeloma, including multiple myeloma, plasma cell myeloma, smoldering myeloma, smoldering multiple myeloma; myelofibrosis; bone marrow disease; myelodysplastic syndrome (MDS); previously treated myelodysplastic syndrome; transplantation-related cancer; neutropenia; leukemia, including acute myeloid leukemia, chronic myelogenous leukemia, B-cell chronic lymphocytic leukemia, leukemia-associated anemia, acute myeloid leukemia (AML); lymphoma, including diffuse large B-cell lymphoma, non-Hodgkin's lymphoma, Hodgkin's lymphoma, anaplastic lymphoma, anaplastic large cell lymphoma, CD20 positive lymphoma, mantle cell lymphoma, primary lymphoma, B-cell lymphoma, recurrent B-cell non-Hodgkin's lymphoma, recurrent diffuse large B-cell lymphoma, recurrent mediastinal (thymic) large B-cell lymphoma, primary mediastinal (thymic) large B-cell lymphoma, recurrent transformed non-Hodgkin's lymphoma, refractory B-cell non-Hodgkin's lymphoma, refractory diffuse large B-cell lymphoma, refractory primary mediastinal (thymic) large B-cell lymphoma, refractory transformed non-Hodgkin's lymphoma; Burkitt's lymphoma; thyroid cancer; melanoma; lung cancer, including lung adenocarcinoma, lung squamous cell carcinoma; inflammatory myofibroblastoma; colorectal cancer; intestinal cancer; brain glioma; astroblastoma; ovarian cancer; bronchial cancer; prostate cancer; breast cancer, including triple negative breast cancer, sporadic breast cancer and patients with Cowden syndrome; pancreatic cancer; central nervous system tumor; neuroblastoma; neuroglioma; Peripheral neuroepithelioma; extramedullary plasmacytoma; plasmacytoma; gastric cancer; gastrointestinal stromal tumors; esophageal cancer; colorectal adenocarcinoma; esophageal squamous cell carcinoma; liver cancer; renal cell carcinoma; bladder cancer; endometrial cancer; metrocarcinoma; head and neck cancer; brain cancer; oral cancer; sarcoma, including rhabdomyosarcoma, various lipogenic tumors, Ewing's sarcoma/primitive neuroectodermal tumors (Ewing/PNETs), and leiomyosarcoma; urothelial carcinoma; basal cell carcinoma; oral squamous cell carcinoma; cholangiocarcinoma; bone cancer; cervical cancer; skin cancer; Richter syndrome (RS); sepsis syndrome; autoimmune diseases, including rheumatoid arthritis, autoimmune encephalomyelitis, ankylosing spondylitis, psoriasis, systemic lupus erythematosus, multiple sclerosis, recurrent oral ulcers, Kawasaki disease, polymyositis/dermatomyositis, Sjogren's syndrome, and atopic dermatitis; keratoconjunctivitis; inflammatory diseases, including Crohn's disease and ulcerative colitis, pneumonia, osteoarthritis, synovitis, systemic inflammatory response syndrome, airway inflammation, bronchitis; cerebral malaria; infectious diseases, including viral pneumonia, Acquired immunodeficiency syndrome (AIDS), COVID-19 novel coronavirus infection, gram-negative bacteria infection, gram-positive bacteria infection, tuberculosis, etc.; septic shock; tuberculosis; bacterial meningitis; chronic obstructive pulmonary disease; asthma; hemorrhagic shock; organ (including kidney, heart, lung) or tissue transplantation rejection; diabetes; sarcoidosis; adult respiratory distress syndrome; anemia; pediatric aplastic anemia; cardiovascular diseases (e.g., coronary heart disease, congestive heart failure, myocardial infarction, atherosclerosis); multiple organ dysfunction caused by cachexia and septic shock; or acute liver failure.

The present disclosure provides a method for preventing and/or treating diseases or disorders associated with cereblon protein, comprising administering to a subject a therapeutically effective amount of the compounds of Formula (I) of the present disclosure or a pharmaceutically acceptable salt thereof, or the pharmaceutical composition of the present disclosure comprising as an active ingredient the compounds of Formula (I) of the present disclosure or a pharmaceutically acceptable salt thereof. In some embodiments, the diseases or disorders associated with cereblon protein comprise: tumor, infectious disease, inflammatory disease, autoimmune disease, anemia, hemorrhagic shock, transplant rejection, multiple organ dysfunction syndrome (MODS), sarcoidosis, adult respiratory distress syndrome, cardiovascular disease, Richter syndrome (RS), acute liver failure, or diabetes. In some embodiments, the diseases or disorders associated with cereblon protein include, but are not limited to, myeloma, including multiple myeloma, plasma cell myeloma, smoldering myeloma, smoldering multiple myeloma; myelofibrosis; bone marrow disease; myelodysplastic syndrome (MDS); previously treated myelodysplastic syndrome; transplantation-related cancer; neutropenia; leukemia, including acute myeloid leukemia, chronic myelogenous leukemia, B-cell chronic lymphocytic leukemia, leukemia-associated anemia, acute myeloid leukemia (AML); lymphoma, including diffuse large B-cell lymphoma, non-Hodgkin's lymphoma, Hodgkin's lymphoma, anaplastic lymphoma, anaplastic large cell lymphoma, CD20 positive lymphoma, mantle cell lymphoma, primary lymphoma, B-cell lymphoma, recurrent B-cell non-Hodgkin's lymphoma, recurrent diffuse large B-cell lymphoma, recurrent mediastinal (thymic) large B-cell lymphoma, primary mediastinal (thymic) large B-cell lymphoma, recurrent transformed non-Hodgkin's lymphoma, refractory B-cell non-Hodgkin's lymphoma, refractory diffuse large B-cell lymphoma, refractory primary mediastinal (thymic) large B-cell lymphoma, refractory transformed non-Hodgkin's lymphoma; Burkitt's lymphoma; thyroid cancer; melanoma; lung cancer, including lung adenocarcinoma, lung squamous cell carcinoma; inflammatory myofibroblastoma; colorectal cancer; intestinal cancer; brain glioma; astroblastoma; ovarian cancer; bronchial cancer; prostate cancer; breast cancer, including triple negative breast cancer, sporadic breast cancer and patients with Cowden syndrome; pancreatic cancer; central nervous system tumor; neuroblastoma; neuroglioma; Peripheral neuroepithelioma; extramedullary plasmacytoma; plasmacytoma; gastric cancer; gastrointestinal stromal tumors; esophageal cancer; colorectal adenocarcinoma; esophageal squamous cell carcinoma; liver cancer; renal cell carcinoma; bladder cancer; endometrial cancer; metrocarcinoma; head and neck cancer; brain cancer; oral cancer; sarcoma, including rhabdomyosarcoma, various lipogenic tumors, Ewing's sarcoma/primitive neuroectodermal tumors (Ewing/PNETs), and leiomyosarcoma; urothelial carcinoma; basal cell carcinoma; oral squamous cell carcinoma; cholangiocarcinoma; bone cancer; cervical cancer; skin cancer; Richter syndrome (RS); sepsis syndrome; autoimmune diseases, including rheumatoid arthritis, autoimmune encephalomyelitis, ankylosing spondylitis, psoriasis, systemic lupus erythematosus, multiple sclerosis, recurrent oral ulcers, Kawasaki disease, polymyositis/dermatomyositis, Sjogren's syndrome, and atopic dermatitis; keratoconjunctivitis; inflammatory diseases, including Crohn's disease and ulcerative colitis, pneumonia, osteoarthritis, synovitis, systemic inflammatory response syndrome, airway inflammation, bronchitis; cerebral malaria; infectious diseases, including viral pneumonia, Acquired immunodeficiency syndrome (AIDS), COVID-19 novel coronavirus infection, gram-negative bacteria infection, gram-positive bacteria infection, tuberculosis, etc.; septic shock; tuberculosis; bacterial meningitis; chronic obstructive pulmonary disease; asthma; hemorrhagic shock; organ (including kidney, heart, lung) or tissue transplantation rejection; diabetes; sarcoidosis; adult respiratory distress syndrome; anemia; pediatric aplastic anemia; cardiovascular diseases (e.g., coronary heart disease, congestive heart failure, myocardial infarction, atherosclerosis); multiple organ dysfunction caused by cachexia and septic shock; or acute liver failure.

In the method for preventing and/or treating diseases or disorders associated with cereblon protein, the compound of Formula (I) of the present disclosure or the pharmaceutical composition comprising as an active ingredient the compound of Formula (I) of the present disclosure is administered to the subject through at least one mode of administration selected from the group consisting of nasal administration, inhalation administration, topical administration, oral administration, oral mucosal administration, rectal administration, pleural cavity administration, peritoneal administration, vaginal administration, intramuscular administration, subcutaneous, transdermal, epidural, intrathecal, and intravenous administration.

The term “treatment” or “treating” refers to the administration of the compound of Formula (I) or a pharmaceutically acceptable salt thereof according to the present disclosure, or the pharmaceutical composition containing, as an active ingredient, the compound of Formula (I) or a pharmaceutically acceptable salt thereof, to a subject to mitigate (alleviate) undesirable diseases or conditions, such as the development of a cancer or tumor. The beneficial or desired clinical results of the present disclosure include, but are not limited to: alleviating symptoms, reducing the severity of the disease, stabilizing the state of the disease, slowing down or delaying the progression of the disease, improving or alleviating the condition, and alleviating the disease.

A “therapeutic effective amount” of the compound of the present disclosure depends on a variety of factors, including the activity of the specific compound used, the metabolic stability of the compound and the duration of its action, the age, sex and weight of the patient, the patient's current medical condition, the route and duration of administration, the excretion rate, the combined administration of additional drugs, and the progression of the diseases or conditions of the patient being treated. Those skilled in the art will be able to determine appropriate dosages based on these and other factors.

It is to be understood that the choice of using one or more active compounds and/or compositions and their dosage depends on the basic situations of the individual (which should generally render the individual situation to achieve the best effect). Dosing and dosing regimens should be within the ability of those skilled in the art, and the appropriate dosage depends on many factors including the knowledge and ability of the physicians, veterinarians or researchers (see e.g., Jun Li (chief editor), “Clinical Pharmacology”, 4th edition, People's Public Health Press, 2008).

As used herein, the term “patient” or “subject” to be treated refers to animal, for example mammal, including but not limited to primate (such as human being), cow, sheep, goat, horse, dog, cat, rabbit, guinea pig, rat, mice, etc.

### VI. Definitions

Unless otherwise specified, the following words, phrases and symbols used herein generally have the meanings as described below.

In general, the nomenclature used herein (including the IUPAC nomenclature) and the laboratory procedures described below (including those used in cell culture, organic chemistry, analytical chemistry, and pharmacology, etc.) are those well-known and commonly used in the art.

Unless otherwise defined, all scientific and technical terms used herein in connection with the present disclosure described herein have the same meaning as commonly understood by one skill in the art. In addition, the use of the word “a” or “an” when used in conjunction with the term “comprising” or a noun in the claims and/or the specification may mean “one”, but it is also consistent with the meaning of “one or more”, “at least one”, and “one or more than one”. Similarly, the terms “another” or “other” can mean at least a second or more.

It should be understood that whenever the term “comprise” or “include” is used herein to describe various aspects, other similar aspects described by “consisting of” and/or “consisting essentially of” are also provided.

As used herein, the term “about” used alone or in combination refers to approximately, roughly, nearly, or around. When the term “about” is used in conjunction with a numerical range, it modifies that range by extending the boundaries above and below the stated numerical value. In general, the term “about” can modify a numerical value above and below the stated value by an upward or downward (increasing or decreasing) variation, e.g., 10%, 5%, 2%, or 1%.

As used herein, the term “optionally substituted” used alone or in combination means that the referenced group may be unsubstituted or substituted with one or more substituents as defined here. Herein, the wording “optionally substituted with . . . ” and “unsubstituted or substituted” can be used interchangeably. The term “substituted” generally indicates that one or more hydrogens in the referenced structure are replaced by the same or different specific substituents. The number of substituents is not theoretically limited in any way, or is automatically limited by the size of the building units (i.e., the total number of replaceable hydrogen atoms in the building units), or as clearly defined herein.

As used herein, the expression “a hydrogen of one or more CH₂ of the linear or branched C_{x-y} alkylene is replaced with . . . ”, used alone or in combination, means that a hydrogen atom of any one or more CH₂ of the linear or branched C_{x-y} alkylene is replaced by a substituent(s) as defined herein. Herein, the term “one or more” of “a hydrogen atom of one or more CH₂ of groups —CH₂—, —(CH₂)₂—, —(CH₂)₃—, —(CH₂)₄—, —(CH₂)₅—, —(CH₂)₆—, —(CH₂)₇—, —(CH₂)₈—, —(CH₂)₉—, —(CH₂)₁₀—, —(CH₂)₁₁—, —(CH₂)₁₂—, —(CH₂)₁₃—, —(CH₂)₁₄—, —(CH₂)₁₅—, —(CH₂)₁₆—, —(CH₂)₁₇—, —(CH₂)₁₈—, —(CH₂)₁₉—, —(CH₂)₂₀—, —(CH₂)₂₁—, —(CH₂)₂₂—, —(CH₂)₂₅—, —(CH₂)₃₀—, —(CH₂)₃₅—, —(CH₂)₄₀—, —(CH₂)₄₅—, —(CH₂)₅₀—, —(CH₂)₅₅—, or —(CH₂)₆₀—” may refer to part or all hydrogen atoms of each referenced alkylene group, including but not limited to 1-60 hydrogens. In some embodiments, the expression “a hydrogen atom of one or more CH₂” may refer to part or all of the hydrogen atoms of the referenced alkylene group, including but not limited to 1-30, such as 1-25, 1-20, 1-15, 1-10, 1-5, 1-4, 1-3, 1-2 or 1 hydrogen atoms. In some embodiments, the expression “a hydrogen atom of one or more CH₂” may include 1-3 of the plurality of hydrogen atoms of the referenced alkylene group. The number of hydrogens to be replaced is in principle not limited in any way, or is automatically limited by the size of the building unit.

As used herein, the term “oxo”, used alone or in combination, refers to ═O.

As used herein, the term “C(O)” or “C(═O)” or “C═O”, used alone or in combination, refers to carbonyl.

As used herein, the term “interrupted” of the expression “one or more pairs of adjacent carbon atoms in the backbone carbon chain of the linear or branched alkylene is optionally interrupted by one or more groups selected from the group consisting of: R_{b}, R_{c}, and any combination thereof”, used alone or in combination, has a known definition in the art, which can mean that one or more groups R_{b}, one or more groups R_{c}, or any combination of one or more groups R_{b} and R_{c} are optionally inserted between one or more pairs of adjacent carbon atoms in the backbone carbon chain of the linear or branched alkylene group. Herein, examples of the above-mentioned expression “one or more pairs of adjacent carbon atoms in the backbone carbon chain . . . is optionally interrupted by one or more groups selected from the group consisting of . . . ” may include, but are not limited to, that one or more (e.g., 1-30, 1-20, or 1-15, 1-10, 1-9, 1-8, 1-7, 1-6, 1-5, 1-4, 1-3, or 1-2, or 1) groups R_{b} as defined herein and/or one or more (e.g., 1-30, 1-20, or 1-15, 1-10, 1-9, 1-8, 1-7, 1-6, 1-5, 1-4, 1-3, or 1-2, or 1) groups R_{c} as defined herein and/or one or more (e.g., 1-30, 1-20, or 1-15, 1-10, 1-9, 1-8, 1-7, 1-6, 1-5, 1-4, 1-3, or 1-2, or 1) combinations of R_{b} with R_{c} are inserted into the backbone carbon chain, and the resulting backbone chain group conforms to the covalent bond theory. When two or more groups R_{b} are inserted into the backbone carbon chain of the linear or branched alkylene group, the two or more groups R_{b}are not directly connected to each other. For example, the expression “one or more pairs of adjacent carbon atoms in the backbone carbon chain of the linear or branched C₂₋₄₀ alkylene is optionally interrupted by one or more groups selected from the group consisting of: R_{b}, R_{c}, and any combination thereof” refers to that one or more (e.g., 1-30, 1-20, or 1-15, 1-10, 1-9, 1-8, 1-7, 1-6, 1-5, 1-4, 1-3, or 1-2, or 1) groups R_{b} as defined herein and/or one or more (e.g., 1-30, 1-20, or 1-15, 1-10, 1-9, 1-8, 1-7, 1-6, 1-5, 1-4, 1-3, or 1-2, or 1) groups R_{c} as defined herein and/or one or more (e.g., 1-30, 1-20, or 1-15, 1-10, 1-9, 1-8, 1-7, 1-6, 1-5, 1-4, 1-3, or 1-2, or 1) combinations of R_{b} with R_{c} are inserted between one or more (e.g., 1-30, 1-20, 1-15, 1-10, 1-8, 1-7, 1-6, 1-5, 1-4, 1-3, 1-2 or 1) pairs of any two adjacent carbon atoms of the backbone carbon chain of the linear or branched C₂₋₄₀ alkylene group, resulting in the formation of a backbone chain group containing one or more (e.g., 1-30, 1-20, 1-15, 1-10, 1-8, 1-7, 1-6, 1-5, 1-4, 1-3, 1-2 or 1) fragments “—CH₂—R_{b}—CH₂—” and/or one or more (e.g., 1-30, 1-20, 1-15, 1-10, 1-8, 1-7, 1-6, 1-5, 1-4, 1-3, 1-2 or 1) fragments “—CH₂—(R_{c})˜₆—CH₂—” and/or one or more (e.g., 1-30, 1-20, 1-15, 1-10, 1-8, 1-7, 1-6, 1-5, 1-4, 1-3, 1-2 or 1) fragments “—CH₂—(R_{b}—(R_{c})ₙ₆)ₙ₇—CH₂—”, where each R_{b} group is the same or different, each R_{c} group is the same or different, and is as defined herein; and n6 represents e.g., an integer of 1, 2, 3, 4, 5, 6, 7, 8, 9, or 10, and n7 represents e.g., an integer of 1, 2, 3, 4, 5, 6, 7, 8, 9, or 10.

Herein, a bond interrupted by a wavy line shows the point of attachment of the depicted group to the rest of the molecule. For example, the group of Formula (G₁) represented by X₁ depicted below

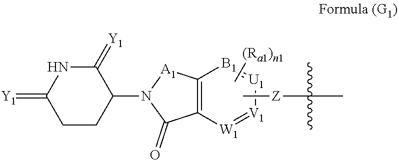

shows the point of attachment of Z in the group of Formula (G₁) to L of the compound of Formula (I).

Herein, the following structural diagram indicates that the group “—R-L-X₁” and n substituents “Rₐ” are connected to any position of the isoindoline ring, meaning that the connection sites of the group “—R-L-X₁” and n substituents “Rₐ” on the isoindoline ring are variable.

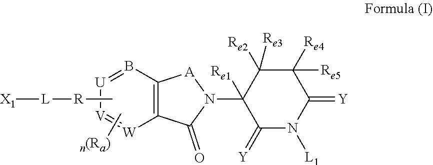

In other words, the structural diagram of the compound of Formula (I) indicates that the structure of the compound has multiple positional isomers. In the compound of Formula (I), the group “—R-L-X₁” and n substituents “Rₐ” are connected to at least two of B, U, V, and W, meaning that at least two of B, U, V, and W represent C, and the remaining represent CH or N. Here, n represents an integer of 1, 2, or 3.

Although the symbol “*” in the group L in the present disclosure indicates the point of attachment of the group L to the group R, the present invention also encompasses embodiments wherein the symbol “*” in group L indicates the point of attachment of the group L to the group X₁.

As used herein, the term “halogen atom” or “halogen”, used alone or in combination, refers to fluorine (F), chlorine (Cl), bromine (Br), or iodine (I).

As used herein, the term “alkyl”, used alone or in combination, refers to a linear or branched alkyl group. The term “Cₓ-C_{y} alkyl” or “C_{x-y} alkyl” (x and y each being an integer) refers to a linear or branched alkyl group containing from x to y carbon atoms. The term “C₁₋₆ alkyl” used alone or in combination in the present disclosure refers to a linear or branched alkyl group containing from 1 to 6 carbon atoms, examples of which include C₁-C₅ alkyl, C₁-C₄ alkyl, and C₁-C₃ alkyl and the like. The term “C₁₋₄ alkyl” used alone or in combination in the present disclosure refers to a linear or branched alkyl group containing from 1 to 4 carbon atoms. Examples of the C₁₋₆ alkyl of the present disclosure may include a C₁-C₅ alkyl, C₁-C₄ alkyl, C₁-C₃ alkyl, C₁-C₂ alkyl and methyl. Representative examples of the term “C₁₋₆ alkyl” include methyl, ethyl, n-propyl, isopropyl, n-butyl, isobutyl, sec-butyl, tert-butyl, pentyl, isopentyl, neopentyl, tert-amyl, and hexyl. The term “C₁₋₄ alkyl” used alone or in combination in the present disclosure refers to a linear or branched alkyl group containing from 1 to 4 carbon atoms. Examples of the C₁₋₄ alkyl of the present disclosure may include C₁-C₄ alkyl, C₁-C₃ alkyl, C₁-C₂ alkyl and methyl. Representative examples of the term “C₁₋₄ alkyl” include methyl, ethyl, n-propyl, isopropyl, n-butyl, isobutyl, sec-butyl, and tert-butyl. The term “C₁₋₃ alkyl” or “C₁-C₃ alkyl” in the present disclosure refers to an alkyl group containing from 1 to 3 carbon atoms, and representative examples of which include methyl, ethyl, n-propyl, and isopropyl. In the present disclosure, the “alkyl” is optionally substituted with one or more substituents optionally selected from the group consisting of D (i.e., deuterium), halogen, hydroxy, cyano, C₁₋₃ alkyl, C₁₋₃ alkoxy, trifluoromethyl, C₅₋₁₀aryl, C₅₋₁₀heteroaryl, C₃₋₁₂ cycloalkyl, heterocyclyl (e.g., 3- to 12-membered heterocyclyl), or a combination thereof.

As used herein, the term “halogenated alkyl” or “haloalkyl”, used alone or in combination, refers to a linear or branched alkyl group substituted with one or more halogens, wherein one or more hydrogen atom(s) of the alkyl group are replaced with one or more halogens. The term “halogenated Cₓ-C_{y} alkyl” or “halogenated C_{x-y} alkyl” (x and y are each an integer) refers to a linear or branched alkyl containing from x to y carbon atoms substituted with one or more halogens. The term “halogenated C₁₋₆ alkyl” used alone or in combination in the present invention refers to a linear or branched alkyl group containing from 1 to 6 carbon atoms substituted with one or more halogens. Examples of the halogenated C₁₋₆ alkyl group of the present disclosure include halogenated C₁₋₆alkyl, halogenated C₁₋₅ alkyl, halogenated C₁₋₄alkyl, halogenated C₁₋₃alkyl, halogenated C₁₋₂alkyl, and halogenated methyl. Representative examples of “halogenated C₁₋₆ alkyl” include halomethyl, haloethyl, halo-n-propyl, haloisopropyl, halo-n-butyl, haloisobutyl, halo-sec-butyl, halo-tert-butyl, halopentyl, haloisoamyl, haloneopentyl, halo-tert-amyl, and halohexyl, particularly including, for example, F₃C—, FCH₂—, F₂CH—, ClCH₂—, Cl₂CH—, CF₃CF₂—, CF₃CHF—, CHF₂CF₂—, CHF₂CHF—, CF₃CH₂— and CH₂ClCH₂—. The term “halogenated C₁₋₃alkyl” or “halo-C₁-C₃ alkyl” of the present disclosure refers to an alkyl group containing from 1 to 3 carbon atoms substituted with one or more halogens, and its representative examples include halomethyl, haloethyl, halo-n-propyl and haloisopropyl, particularly including, for example, F₃C—, FCH₂—, F₂CH—, ClCH₂—, Cl₂CH—, CF₃CF₂—, CF₃CHF—, CHF₂CF₂—, CHF₂CHF—, CF₃CH₂—, and CH₂ClCH₂—.

As used herein, the term “deuterated”, used alone or in combination, means that one or more hydrogen atom(s) of the referenced group are replaced with deuterium atoms (i.e., D).

As used herein, the term “deuterated C_{x-Cy} alkyl” or “deuterated C_{x-y} alkyl” (x and y are each an integer) refers to a linear or branched alkyl containing from x to y carbon atoms substituted with one or more deuterium atoms. The term “deuterated C₁₋₆ alkyl” used alone or in combination in the present disclosure refers to a linear or branched alkyl group containing from 1 to 6 carbon atoms substituted with one or more deuterium atoms. Examples of the deuterated C₁₋₆ alkyl group of the present disclosure include deuterated C₁₋₅ alkyl group, e.g., deuterated C₁₋₄ alkyl group, deuterated C₂₋₅ alkyl group, deuterated C₁₋₃ alkyl group, deuterated C₁₋₂ alkyl, and deuterated methyl. Representative examples of the deuterated C₁₋₆ alkyl group include perdeuterated methyl (CD₃), perdeuterated ethyl (CD₃CD₂), perdeuterated n-propyl, perdeuterated isopropyl, perdeuterated n-butyl, perdeuterated isobutyl, perdeuterated sec-butyl, perdeuterated tert-butyl, perdeuterated pentyl, perdeuterated isopentyl, perdeuterated neopentyl, perdeuterated tert-amyl, and perdeuterated hexyl. The term “deuterated C₁₋₃ alkyl” or “deuterated C₁-C₃ alkyl” of the present disclosure refers to an alkyl group containing from 1 to 3 carbon atoms substituted with one or more deuterium atoms, and its representative examples include perdeuterated methyl (CD₃) and perdeuterated ethyl (CD₃CD₂).

As used herein, the term “alkylene” (which is used interchangeably with “alkylene chain”), used alone or in combination, refers to a linear or branched divalent saturated hydrocarbon group composed of carbon and hydrogen atoms. The term “Cₓ-C_{y} alkylene” or “C_{x-y} alkylene” (x and y each being an integer) refers to a linear or branched alkylene group containing from x to y carbon atoms. The term “C₁-C₃₀ alkylene” or “C₁₋₃₀ alkylene” refers to a linear or branched alkylene group containing from 1 to 30 carbon atoms, examples of which include, but are not limited to C₁-C₂₉alkylene, C₁-C₂₈alkylene, C₁-C₂₇alkylene, C₁-C₂₆alkylene, C₁-C₂₅alkylene, C₁-C₂₄alkylene, C₁-C₂₃alkylene, C₁-C₂₂alkylene, C₁-C₂₁alkylene, C₁-C₂₀alkylene, C₁-C₁₉alkylene, C₁-C₁₈alkylene, C₁-C₁₇alkylene, C₁-C₁₆alkylene, C₁-C₁₅alkylene, C₁-C₁₄alkylene, C₁-C₁₃alkylene, C₁-C₁₂alkylene, C₁-C₁₁alkylene, C₁-C₁₀alkylene, C₁-C₉alkylene, C₁-C₈alkylene, C₁-C₇alkylene, C₁-C₆alkylene, C₁-C₅alkylene, C₁-C₄alkylene, C₁-C₃alkylene, or C₁-C₂alkylene. Representative examples include, but are not limited to, methylene, ethylene, propylene, isopropylene, butylene, isobutylene, sec-butylene, tert-butylene, n-pentylene, isopentylene, neopentylidene, tert-amylene, hexylene, heptylene, octylene, nonylene, decylene, undecylene, dodecylene, tridecylene, tetradecylene, pentadecylene, hexadecylene, heptadecylene, octadecylene, nonadecylene, eicosylene, heneicosylene, docosylene, tricosylene, tetracosylene, pentacosylene, hexacosylene, heptacosylene, octacosylene, nonacosylene, and triacontylene. In the present disclosure, the “alkylene” is optionally substituted with one or more substituents optionally selected from the group consisting of: halogen, cyano, C₁₋₃alkyl, C₁₋₃ alkoxy, trifluoromethyl, C₅₋₁₀ aryl, C₅₋₁₀ heteroaryl, C₃₋₁₂cycloalkyl, 3- to 12-membered heterocyclyl, or any combination thereof.

As used herein, the term “alkoxy”, used alone or in combination, refers to a linear or branched alkoxy group having structural formula of alkyl-O—. Optionally, the alkyl portion of the alkoxy group may contain 1-10 carbon atoms. Representative examples of “alkoxy” include, but are not limited to, methoxy, ethoxy, propoxy, isopropoxy, n-butoxy, isobutoxy, tert-butoxy, pentyloxy, 2-pentyloxy, isopentyloxy, neopentyloxy, hexyloxy, 2-hexyloxy, 3-hexyloxy, 3-methylpentyloxy, etc. The term “C₁-C₄ alkoxy” or “C₁₋₄ alkoxy” refers to a linear or branched alkoxy group containing from 1 to 4 carbon atoms. Representative examples of C₁₋₄ alkoxy include, but are not limited to, methoxy, ethoxy, n-propoxy, isopropoxy, n-butoxy, isobutoxy, and tert-butoxy.

As used herein, the term “halogenated alkoxy”, used alone or in combination, refers to a alkoxy group substituted with one or more halogen atoms. Optionally, the alkyl portion of the alkoxy group may contain 1-10 carbon atoms. Examples of “halogenated alkoxy” include, but are not limited to, halogenated C₁₋₆ alkoxy or halogenated C₁₋₄ alkoxy. Representative examples include, but are not limited to, F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, and CH₂ClCH₂—O—.

As used herein, the term “alkyl-NH—”, used alone or in combination, refers to a linear or branched alkyl-NH—, wherein alkyl is as defined above. Optionally, the alkyl portion of the alkyl-NH— group may contain 1-6 carbon atoms, i.e., C₁₋₆ alkyl-NH—. Representative examples of “alkyl-NH—” include, but are not limited to, methyl-NH—, ethyl-NH—, n-propyl-NH—, isopropyl-NH—, n-butyl-NH—, isobutyl-NH—, tert-butyl-NH—, pentyl-NH—, and hexyl-NH—, etc. The term “C₁-C₄ alkyl-NH—” or “C₁₋₄ alkyl-NH—” refers to a linear or branched alkyl-NH— group containing from 1 to 4 carbon atoms. Representative examples of C₁₋₄ alkyl-NH— include, but are not limited to, methyl-NH—, ethyl-NH—, n-propyl-NH—, isopropyl-NH—, n-butyl-NH—, isobutyl-NH—, and tert-butyl-NH—.

As used herein, the term “alkyl-NHC(O)—”, used alone or in combination, refers to a linear or branched alkyl-NHC(O)— group, wherein alkyl is as defined above. Optionally, the alkyl portion of the alkyl-NHC(O)— group may contain 1-6 carbon atoms. The term “C₁₋₄ alkyl-NHC(O)—” or “C₁-C₄ alkyl-NHC(O)—” refers to a linear or branched alkyl-NHC(O)— group containing from 1 to 4 carbon atoms. Representative examples of C₁₋₄ alkyl-NHC(O)— include, but are not limited to, CH₃—NHC(O)—, CH₃CH₂—NHC(O)—, and CH₃CH₂CH₂—NHC(O)—.

As used herein, the term “alkyl-C(O)NH—”, used alone or in combination, refers to a linear or branched alkyl-C(O)NH— group, wherein alkyl is as defined above. Optionally, the alkyl portion of the alkyl-C(O)NH— group may contain 1-6 carbon atoms. The term “C₁₋₄ alkyl-C(O)NH—” or “C₁-C₄ alkyl-C(O)NH—” refers to a linear or branched alkyl-C(O)NH— group containing from 1 to 4 carbon atoms. Representative examples of C₁₋₄ alkyl-C(O)NH— include, but are not limited to, CH₃—C(O)NH—, CH₃CH₂—C(O)NH—, and CH₃CH₂CH₂—C(O)NH—.

As used herein, the term “aryl”, used alone or in combination, refers to a monovalent aromatic hydrocarbon group containing from 5 to 14 carbon atoms and optionally one or more fused rings, such as phenyl group, naphthyl group, or fluorenyl group. As used herein, the “aryl” is optionally substituted. The aryl group is optionally substituted one or more times (e.g., 1-4, 1-3, or 1-2 times) with a substituent(s). For example, aryl is mono-, di-, or tri-substituted with a substituent(s) as defined above or optionally selected from e.g., optionally deuterated C₁-C₆alkyl, optionally deuterated C₃₋₆cycloalkyl, hydroxy, amino, mercapto, halogen, cyano, oxo, optionally deuterated C₁-C₆ alkoxy, C₁-C₄alkylamino, halogenated C₁-C₆alkyl, amino-substituted C₁₋₆alkylene, C₁₋₄alkyl-NHC(O)—, C₁₋₄alkyl-C(O)NH—, and any combination thereof.

As used herein, the term “arylene”, used alone or in combination, refers to a divalent aromatic hydrocarbon group containing from 5 to 14 carbon atoms and optionally one or more fused rings, such as phenylene, naphthylene, or fluorenylene. As used herein, the “arylene” is optionally substituted. The arylene group is optionally substituted one or more times (e.g., 1-4, 1-3, or 1-2 times) with a substituent(s). For example, arylene is mono-, di-, or tri-substituted with a substituent(s) as defined above or optionally selected from e.g., deuterium, optionally deuterated C₁-C₆ alkyl, optionally deuterated C₃₋₆ cycloalkyl, hydroxy, amino, mercapto, halogen, cyano, oxo, optionally deuterated C₁-C₆ alkoxy, C₁-C₄ alkylamino, halogenated C₁-C₆ alkyl, amino-substituted C₁₋₆ alkylene, C₁₋₄ alkyl-NHC(O)—, C₁₋₄ alkyl-C(O)NH— and any combination thereof.

As used herein, the term “heteroaryl”, used alone or in combination, refers to a 5- to 20-membered (e.g., 5- to 15-membered, 5- to 12-membered, 5- to 11-membered, 5- to 10-membered, 5- to 9-membered, 5- to 8-membered, 5- to 7-membered, 5- to 6-membered, 6- to 15-membered, or 6- to 9-membered) monocyclic, bicyclic, or polycyclic cyclic radical containing at least one aromatic ring having one or more (e.g., from 1 to 6, or from 1 to 5, or from 1 to 4, or from 1 to 3) heteroatoms independently selected from the group consisting of oxygen, nitrogen, and sulfur. Bicyclic or polycyclic heteroaryl groups include bicyclic, tricyclic or tetracyclic heteroaryl groups, which contain one aromatic ring having one or more heteroatoms independently selected from O, S and N, and the remaining rings which may be a saturated, partially unsaturated or aromatic ring and can be carbocyclic ring or contain one or more heteroatoms independently selected from O, S and N. Examples of monocyclic heteroaryl groups include, but are not limited to, furanyl, oxazolyl, isoxazolyl, oxadiazolyl, thienyl, thiazolyl, isothiazolyl, thiadiazolyl, pyrrolyl, imidazolyl, pyrazolyl, triazolyl, pyridyl, pyrimidinyl, pyridazinyl, pyrazinyl, tetrazolyl, and triazinyl. Examples of bicyclic heteroaryl groups include, but are not limited to, indolyl, isoindolyl, isoindolinyl, benzofuranyl, isobenzofuranyl, benzothienyl, indazolyl, benzimidazolyl, benzoxazolyl, benzisoxazolyl, benzothiazolyl, benzisothiazolyl, benzotriazolyl, benzo[2,1,3]oxadiazolyl, benzo[2,1,3]thiadiazolyl, benzo[1,2,3]thiadiazolyl, quinolinyl, isoquinolinyl, naphthyridinyl, cinnolinyl, quinazolinyl, quinoxalinyl, phthalazinyl, oxazolopyridyl, furopyridyl, pteridyl, purinyl, pyridopyridyl, pyrazolo[1,5-a]pyridyl, pyrazolo[1,5-a]pyrimidinyl, imidazo[1,2-a]pyridyl, 1H-pyrrolo[3,2-b]pyridyl, 1H-pyrrolo[2,3-b]pyridyl, pyrrolo[2,1-b]thiazolyl and imidazo[2,1-b]thiazolyl. Examples of tricyclic or polycyclic heteroaryl groups include, but are not limited to, acridinyl, benzindolyl, carbazolyl, dibenzofuranyl, and xanthyl. The heteroaryl group may be unsubstituted or substituted. A substituted heteroaryl refers to heteroaryl substituted one or more times (e.g., 1-4, 1-3, or 1-2 times) by a substituent(s) as defined above or optionally selected from the group consisting of C₁-C₃alkyl, C₃₋₆cycloalkyl, hydroxy, amino, mercapto, halogen, C₁-C₃ alkoxy, C₁-C₃alkylamino, halogenated C₁-C₃alkyl, amino C₁₋₃alkylene, C₁₋₃alkyl-NHC(O)—, C₁₋₃alkyl-C(O)NH—, cyano, or any combination thereof.

As used herein, the term “heteroarylene”, used alone or in combination, refers to a 5- to 20-membered (e.g., 5- to 15-membered, 5- to 12-membered, 5- to 11-membered, 5- to 10-membered, 5- to 9-membered, 5- to 8-membered, 5- to 7-membered, 5- to 6-membered, 6- to 15-membered, or 6- to 9-membered) bivalent monocyclic, bicyclic, or polycyclic cyclic aromatic ring radical containing at least one aromatic ring having one or more (e.g., from 1 to 6, or from 1 to 5, or from 1 to 4, or from 1 to 3) heteroatoms independently selected from the group consisting of oxygen, nitrogen, and sulfur. Bicyclic or polycyclic heteroarylene groups include bicyclic, tricyclic or tetracyclic heteroarylene groups, which contain one aromatic ring having one or more heteroatoms independently selected from O, S and N, and the remaining ring(s) which may be a saturated, partially unsaturated or aromatic ring and can be carbocyclic ring or contain one or more heteroatoms independently selected from O, S and N. Examples of monocyclic heteroarylene groups include, but are not limited to, furanylene, oxazolylene, isoxazolylene, oxadiazolylene, thienylene, thiazolylene, isothiazolylene, thiadiazolylene, pyrrolylene, imidazolylene, pyrazolylene, triazolylene, pyridylene, pyrimidinylene, pyridazinylene, pyrazinylene, tetrazolylene, and triazinylene. Examples of bicyclic heteroarylene groups include, but are not limited to, indolylene, isoindolylene, isoindolinylene, benzofuranylene, isobenzofuranylene, benzothienylene, indazolylene, benzimidazolylene, benzoxazolylene, benzisoxazolylene, benzothiazolylene, benzisothiazolylene, benzotriazolylene, benzo[2,1,3]oxadiazolylene, benzo[2,1,3]thiadiazolylene, benzo[1,2,3]thiadiazolylene, quinolinylene, isoquinolinylene, naphthyridinylene, cinnolinylene, quinazolinylene, quinoxalinylene, phthalazinylene, oxazolopyridylene, furopyridylene, pteridylene, purinylene, pyridopyridylene, pyrazolo[1,5-a]pyridylene, pyrazolo[1,5-a]pyrimidinylene, imidazo[1,2-a]pyridylene, 1H-pyrrolo[3,2-b]pyridylene, 1H-pyrrolo[2,3-b]pyridylene, pyrrolo[2,1-b]thiazolylene, and imidazo[2,1-b]thiazolylene. Examples of tricyclic or polycyclic heteroarylene groups include, but are not limited to, acridinylene, benzindolylene, carbazolylene, dibenzofuranylene, and xanthylene. The heteroarylene group may be unsubstituted or substituted. A substituted heteroarylene refers to heteroarylene substituted one or more times (e.g., 1-4, 1-3, or 1-2 times) by a substituent(s) as defined above or optionally selected from the group consisting of C₁-C₃alkyl, C₃₋₆cycloalkyl, hydroxy, amino, mercapto, halogen, C₁-C₃ alkoxy, C₁-C₃alkylamino, halogenated C₁-C₃alkyl, amino-substituted C₁₋₃alkylene, C₁₋₃alkyl-NHC(O)—, C₁₋₃alkyl-C(O)NH—, cyano, or any combination thereof.

As used herein, the term “cycloalkyl”, used alone or in combination, refers to a saturated or partially unsaturated (i.e., containing one or more double bonds, but not having a fully conjugated 7-electron system) monocyclic or bicyclic or polycyclic cyclic hydrocarbon radical, which in some embodiments contains from 3 to 20 carbon atoms (i.e., C₃₋₂₀ cycloalkyl), or from 3 to 15 carbon atoms (i.e., C₃₋₁₅ cycloalkyl), or from 3 to 12 carbon atoms (i.e., C₃₋₁₂ cycloalkyl), or from 3 to 11 carbon atoms (i.e., C₃₋₁₁ cycloalkyl), or from 3 to 10 carbon atoms (i.e., C₃₋₁₀ cycloalkyl), or from 3 to 8 carbon atoms (i.e., C₃₋₈ cycloalkyl), or from 3 to 7 carbon atoms (i.e., C₃₋₇ cycloalkyl), or from 3 to 6 carbon atoms (i.e., C₃₋₆ cycloalkyl). The term “cycloalkyl” includes monocyclic, bicyclic, tricyclic or polycyclic cyclic hydrocarbon radical having from 3 to 20 carbon atoms. Representative examples of monocyclic cycloalkyl groups include, but are not limited to, cyclopropyl, cyclobutyl, cyclopentyl, cyclopentenyl, cyclohexyl, cyclohexenyl, cycloheptyl, and cyclooctyl. Bicyclic, tricyclic and polycyclic cycloalkyl groups include bridged cycloalkyl (e.g., C₅₋₂₀ bridged cycloalkyl, C₅₋₁₅ bridged cycloalkyl and C₇₋₁₅ bridged cycloalkyl), fused cycloalkyl (e.g., C₅₋₂₀ fused cycloalkyl, C₅₋₁₅ fused cycloalkyl, C₆₋₂₀ fused cycloalkyl, C₆₋₁₅ fused cycloalkyl, C₇₋₂₀ fused cycloalkyl, C₇₋₁₅ fused cycloalkyl and C₈₋₁₅ fused cycloalkyl) and spiro-cycloalkyl groups (e.g., C₅₋₂₀ spiro-cycloalkyl, C₅₋₁₅ spiro-cycloalkyl, C₆₋₂₀ spiro-cycloalkyl, C₆₋₁₅ spiro-cycloalkyl, C₇₋₂₀ spiro-cycloalkyl, C₇₋₁₅ spiro-cycloalkyl and C₈₋₁₅ spiro-cycloalkyl), representative examples of which include, but not limited to, decalinyl, octahydropentalenyl, octahydro-1H-indenyl, C₅₋₂₀ spiro-cycloalkyl, C₅₋₁₅ spiro-cycloalkyl, adamantanyl, noradamantanyl, bornyl, norbornyl (also named as bicyclo[2.2.1]heptyl by the IUPAC system). As used herein, the “cycloalkyl” is optionally mono- or poly-substituted, such as, but not limited to, 2,2-, 2,3-, 2,4-, 2,5-, or 2,6-disubstituted cyclohexyl. The substituents of the substituted “cycloalkyl” can be optionally one or more (e.g., 1-5, 1-4, 1-3, 1-2, or 1) substituents selected from the group consisting of deuterium, optionally deuterated C₁-C₆ alkyl, optionally deuterated C₃₋₆ cycloalkyl, hydroxy, amino, mercapto, halogen, cyano, oxo, optionally deuterated C₁-C₆ alkoxy, C₁-C₄ alkylamino, halogenated C₁-C₆ alkyl, amino-substituted C₁₋₆ alkylene, C₁₋₄ alkyl-NHC(O)—, C₁₋₄ alkyl-C(O)NH— and any combination thereof. Examples of “C₃₋₆ cycloalkyl” include, but are not limited to, cyclopropyl, cyclobutyl, cyclopentyl, cyclopentenyl, cyclohexenyl, and cyclohexyl.

As used herein, the term “cycloalkylene”, used alone or in combination, refers to a saturated or partially unsaturated (i.e., containing one or more double bonds, but not having a fully conjugated π-electron system) monocyclic, bicyclic, or polycyclic divalent cyclic hydrocarbon radical, which in some embodiments contains from 3 to 20 carbon atoms (i.e., C₃₋₂₀ cycloalkylene), or from 3 to 15 carbon atoms (i.e., C₃₋₁₅ cycloalkylene), or from 3 to 12 carbon atoms (i.e., C₃₋₁₂ cycloalkylene), or from 3 to 11 carbon atoms (i.e., C₃₋₁₁ cycloalkylene), or from 3 to 10 carbon atoms (i.e., C₃₋₁₀ cycloalkylene), or from 3 to 8 carbon atoms (i.e., C₃₋₈ cycloalkylene), or from 3 to 7 carbon atoms (i.e., C₃₋₇ cycloalkylene), or from 3 to 6 carbon atoms (i.e., C₃₋₆ cycloalkylene). The term “cycloalkylene” includes monocyclic, bicyclic, tricyclic or polycyclic cycloalkylene having from 3 to 20 carbon atoms. Representative examples of monocyclic cycloalkylene groups include, but are not limited to, cyclopropylene, cyclobutylene, cyclopentylene, cyclopentenylene, cyclohexylene, cyclohexenylene, cycloheptylene, and cyclooctylene. Bicyclic, tricyclic and polycyclic cycloalkylene groups include bridged cycloalkylene (e.g., C₅₋₂₀ bridged cycloalkylene, C₅₋₁₅ bridged cycloalkylene and C₇₋₁₅ bridged cycloalkylene), fused cycloalkylene (e.g., C₅₋₂₀ fused cycloalkylene, C₅₋₁₅ fused cycloalkylene, C₆₋₂₀ fused cycloalkylene, C₆₋₁₅ fused cycloalkylene, C₇₋₂₀ fused cycloalkylene, C₇₋₁₅ fused cycloalkylene and C₈₋₁₅ fused cycloalkylene) and spiro-cycloalkylene groups (e.g., C₅₋₂₀ spiro-cycloalkylene, C₅₋₁₅ spiro-cycloalkylene, C₆₋₂₀ spiro-cycloalkylene, C₆₋₁₅ spiro-cycloalkylene, C₇₋₂₀ spiro-cycloalkylene, C₇₋₁₅ spiro-cycloalkylene and C₈₋₁₅ spiro-cycloalkylene), representative examples of which include, but not limited to, decalinylene, octahydropentalenylene, octahydro-1H-indenylene, 2,3-dihydro-1H-indenylene, C₅₋₂₀ spiro-cycloalkylene, C₅₋₁₅ spiro-cycloalkylene, adamantanylene, noradamantanylene, and norbornylene (also named as bicyclo[2.2.1]heptylene by the IUPAC system). As used herein, the “cycloalkylene” is optionally mono- or poly-substituted, such as, but not limited to, 2,2-, 2,3-, 2,4-, 2,5-, or 2,6-disubstituted cyclohexylene. The substituents of the substituted “cycloalkylene” can be optionally one or more substituents selected from the group consisting of deuterium, optionally deuterated C₁-C₆ alkyl, optionally deuterated C₃₋₆ cycloalkyl, hydroxy, amino, mercapto, halogen, cyano, oxo, optionally deuterated C₁-C₆ alkoxy, C₁-C₄ alkylamino, halogenated C₁-C₆ alkyl, amino-substituted C₁₋₆ alkylene, C₁₋₄ alkyl-NHC(O)—, C₁₋₄ alkyl-C(O)NH— and any combination thereof. Examples of “C₃₋₆ cycloalkylene” include, but are not limited to, cyclopropylene, cyclobutylene, cyclopentylene, cyclopentenylene, cyclohexenylene, and cyclohexylene.

As used herein, the term “C_{x-y} spiro-cycloalkyl” (x and y each being an integer), used alone or in combination, refers to a spiro-cycloalkyl group containing from x to y carbon atoms. As used herein, the term “C₇₋₁₂ spiro-cycloalkyl”, used alone or in combination, refers to a spiro-cycloalkyl group containing from 7 to 12 carbon atoms. The term “C₅₋₂₀ spiro-cycloalkyl” includes “C₅₋₁₅ spiro-cycloalkyl”, “C₇₋₁₅ spiro-cycloalkyl” and “C₇₋₂₀ spiro-cycloalkyl”. Representative examples of “C₇₋₁₁ spiro-cycloalkyl” include, but are not limited to, spiro[3.3]heptyl, spiro[2.5]octyl, spiro[3.5]nonyl, spiro[4.4]nonyl, spiro[4.5]decyl, or spiro[5.5]undecyl. The term “C₇₋₁₂ spiro-cycloalkyl” is optionally substituted with one or more substituents selected from the group consisting of deuterium, optionally deuterated C₁-C₆ alkyl, optionally deuterated C₃₋₆ cycloalkyl, hydroxy, amino, mercapto, halogen, cyano, oxo, optionally deuterated C₁-C₆ alkoxy, C₁-C₄ alkylamino, halogenated C₁-C₆ alkyl (e.g., trifluoromethyl), amino-substituted C₁₋₆ alkylene, C₁₋₄ alkyl-NHC(O)—, C₁₋₄ alkyl-C(O)NH— and any combination thereof.

As used herein, the term “C_{x-y} spiro-cycloalkylene” (x and y each being an integer), used alone or in combination, refers to a spiro-cycloalkylene group containing from x to y carbon atoms. As used herein, the term “C₇₋₁₂ spiro-cycloalkylene”, used alone or in combination, refers to a spiro-cycloalkylene group containing from 7 to 12 carbon atoms (e.g., 7-10, 7-9 carbon atoms). The term “C₅₋₂₀ spiro-cycloalkylene” includes “C₅₋₁₅ spiro-cycloalkylene”, “C₇₋₁₅ spiro-cycloalkylene” and “C₇. 20 spiro-cycloalkylene”. Representative examples of “C₇₋₁₂ spiro-cycloalkylene” include, but are not limited to, spiro[3.3]heptylene, spiro[2.5]octylene, spiro[3.5]nonylene, spiro[4.4]nonylene, spiro[4.5]decylene, or spiro[5.5]undecylene. The term “C₇₋₁₂ spiro-cycloalkylene” is optionally substituted with one or more substituents selected from the group consisting of deuterium, optionally deuterated C₁-C₆ alkyl, optionally deuterated C₃₋₆ cycloalkyl, hydroxy, amino, mercapto, halogen, cyano, oxo, optionally deuterated C₁-C₆ alkoxy, C₁-C₄ alkylamino, halogenated C₁-C₆ alkyl (e.g., trifluoromethyl), amino-substituted C₁₋₆ alkylene, C₁₋₄ alkyl-NHC(O)—, C₁₋₄ alkyl-C(O)NH— and any combination thereof.

As used herein, the term “heterocyclyl” or “heterocyclic group”, used alone or in combination, refers to a 3- to 20-membered saturated or partially unsaturated (i.e., containing one or more double bonds, but not having a fully conjugated π-electron system) monocyclic, bicyclic, tricyclic or polycyclic cyclic hydrocarbon group containing one or more (e.g., from 1 to 5, or from 1 to 4, from 1 to 3, from 1 to 2, or 1) heteroatoms independently selected from sulfur, oxygen, and nitrogen. In some embodiments, “heterocyclyl” may refer to a 3- to 15-membered (e.g., 3- to 14-membered, 3- to 12-membered, 3- to 11-membered, 3- to 10-membered, 3- to 9-membered, 3- to 8-membered, 3- to 7-membered, 3- to 6-membered, 3- to 5-membered, or 4- to 9-membered) saturated or partially unsaturated (i.e., containing one or more double bonds, but not having a fully conjugated R-electron system) monocyclic cyclic hydrocarbon group containing one or more (e.g., from 1 to 5, or from 1 to 4, from 1 to 3, from 1 to 2, or 1) heteroatoms independently selected from sulfur, oxygen, and nitrogen. Examples of the heterocyclyl groups include, but not limited to, monocyclic heterocyclyl (e.g., 4- to 20-membered monocyclic heterocyclyl, and 4- to 15-membered monocyclic heterocyclyl), bridged heterocyclyl (e.g., 5- to 20-membered bridged heterocyclyl, 5- to 15-membered bridged heterocyclyl, 7- to 20-membered bridged heterocyclyl, and 7- to 15-membered bridged heterocyclyl), fused heterocyclyl (e.g., 5- to 20-membered fused heterocyclyl, and 5- to 15-membered fused heterocyclyl), spiro-heterocyclyl groups (e.g., 5- to 20-membered spiro-heterocyclyl, and 5- to 15-membered spiro-heterocyclyl), cyclic ester group (i.e., (Lactone), such as 4- to 15-membered, 4- to 14-membered, 4- to 13-membered, 4- to 12-membered, 4- to 11-membered, 4- to 10-membered, 4- to 9-membered, 4- to 8-membered, 5- to 15-membered, 5- to 10-membered, 5- to 8-membered, 6- to 15-membered, or 6- to 10-membered cyclic ester group), and cyclic amide groups (i.e. lactams group, such as 4- to 15-membered, 4- to 14-membered, 4- to 13-membered, 4- to 12-membered, 4- to 11-membered, 4- to 10-membered, 4- to 9-membered, 4- to 8-membered, 5- to 15-membered, 5- to 10-membered, 5- to 8-membered, 6- to 15-membered, or 6- to 10-membered cyclic amide groups). Representative examples of the monocyclic heterocyclyl include, but are not limited to, azetidinyl, oxetanyl, pyrrolidinyl, imidazolidinyl, pyrazolidyl, tetrahydrofuranyl, tetrahydropyranyl, tetrahydrothienyl, tetrahydrothiopyranyl, oxazolidinyl, thiazolidinyl, piperidinyl, piperazinyl, morpholinyl, thiomorpholinyl, dioxacyclohexyl, azacycloheptyl, azacyclooctyl, diazacycloheptanyl (e.g., 1,4-diazacycloheptan-1-yl) and diazacyclooctyl. Bicyclic, tricyclic and polycyclic heterocyclyl groups include bridged heterocyclyl, fused heterocyclyl and spiro-heterocyclyl groups, representative examples of which include, but not limited to, 6-azabicyclo[3.1.1]heptan-3-yl, 2,5-diazabicyclo[2.2.1]heptan-2-yl, 3,6-diazabicyclo[3.1.1]heptan-3-yl, 3-azabicyclo[3.2.1]octan-8-yl, 3,8-diazabicyclo[3.2.1]octan-8-yl, 3,8-diazabicyclo[3.2.1]octan-3-yl, 2,5-diazabicyclo[2.2.2]octan-2-yl, and azaspirocycloalkyl (e.g., 5- to 20-membered azaspirocycloalkyl, such as 3-azaspiro[5.5]undecan-3-yl). The heterocyclyl may be unsubstituted or substituted as explicitly defined (e.g., mono-, di-, tri-, or poly-substituted) by a substituent(s) as defined above or optionally selected from the group consisting of deuterium, hydroxy, amino, mercapto, nitro, halogen, cyano, oxo, optionally deuterated C₁₋₆ alkyl, halogenated C₁₋₆ alkyl, optionally deuterated C₃₋₆ cycloalkyl, optionally deuterated C₁₋₆ alkoxy, C₁₋₄ alkyl-NH—, NH₂—C₁₋₆ alkylene, C₁₋₄ alkyl-NHC(O)—, C₁₋₄ alkyl-C(O)NH— and any combination thereof.

As used herein, the term “heterocyclylene”, used alone or in combination, refers to a 3- to 20-membered saturated or partially unsaturated (i.e., containing one or more double bonds, but not having a fully conjugated π-electron system) monocyclic, bicyclic, tricyclic or polycyclic bivalent cyclic hydrocarbon group containing one or more (e.g., from 1 to 5, or from 1 to 4, from 1 to 3, from 1 to 2, or 1) heteroatoms independently selected from sulfur, oxygen, and nitrogen. In some embodiments, “heterocyclylene” may refer to e.g., a 3- to 15-membered (optionally 3- to 14-membered, 3- to 12-membered, 3- to 11-membered, 3- to 10-membered, 3- to 9-membered, 3- to 8-membered, 3- to 7-membered, 3- to 6-membered, 3- to 5-membered, or 4- to 9-membered) saturated or partially unsaturated (i.e., containing one or more double bonds, but not having a fully conjugated 71-electron system) monocyclic bivalent cyclic hydrocarbon group containing one or more (e.g., from 1 to 5, or from 1 to 4, from 1 to 3, from 1 to 2, or 1) heteroatoms independently selected from sulfur, oxygen, and nitrogen. Examples of the heterocyclylene groups include, but not limited to, monocyclic heterocyclylene (e.g., 4- to 20-membered monocyclic heterocyclylene, and 4- to 15-membered monocyclic heterocyclylene), bridged heterocyclylene (e.g., 5- to 20-membered bridged heterocyclylene, 5- to 15-membered bridged heterocyclylene, 7- to 20-membered bridged heterocyclylene, and 7- to 15-membered bridged heterocyclylene), fused heterocyclylene (e.g., 5- to 20-membered fused heterocyclylene, and 5- to 15-membered fused heterocyclylene), spiro-heterocyclylene groups (e.g., 5- to 20-membered spiro-heterocyclylene, and 5- to 15-membered spiro-heterocyclylene), bivalent cyclic ester group (i.e., (Lactone), such as 4- to 15-membered, 4- to 14-membered, 4- to 13-membered, 4- to 12-membered, 4- to 11-membered, 4- to 10-membered, 4- to 9-membered, 4- to 8-membered, 5- to 15-membered, 5- to 10-membered, 5- to 8-membered, 6- to 15-membered, or 6- to 10-membered bivalent cyclic ester group), and bivalent cyclic amide groups (i.e. bivalent lactams group, such as 4- to 15-membered, 4- to 14-membered, 4- to 13-membered, 4- to 12-membered, 4- to 11-membered, 4- to 10-membered, 4- to 9-membered, 4- to 8-membered, 5- to 15-membered, 5- to 10-membered, 5- to 8-membered, 6- to 15-membered, or 6- to 10-membered bivalent cyclic amide groups). Representative examples of the monocyclic heterocyclylene include, but are not limited to, azetidinylene, oxetanylene, pyrrolidinylene, imidazolidinylene, pyrazolidylene, tetrahydrofuranylene, tetrahydropyranylene, tetrahydrothienylene, tetrahydrothiopyranylene, oxazolidinylene, thiazolidinylene, piperidinylene, piperazinylene, morpholinylene, thiomorpholinylene, dioxacyclohexylene, and diazacycloheptanylene (e.g., 1,4-diazacycloheptanylene, 4,5-diazacycloheptanylene, 1,3-diazacycloheptanylene). Bicyclic, tricyclic and polycyclic heterocyclylene groups include bridged heterocyclylene, fused heterocyclylene and spiro-heterocyclylene groups, representative examples of which include, but not limited to, 6-azabicyclo[3.1.1]heptanylene, 2,5-diazabicyclo[2.2.1]heptanylene, 3,6-diazabicyclo[3.1.1]heptanylene, 3-azabicyclo[3.2.1]octanylene, 3,8-diazabicyclo[3.2.1]octanylene, 3,8-diazabicyclo[3.2.1]octanylene, 2,5-diazabicyclo[2.2.2]octanylene, and azaspirocycloalkylene (e.g., 5- to 20-membered azaspirocycloalkylene, such as 3-azaspiro[5.5]undecanylene). The heterocyclylene may be unsubstituted or substituted as explicitly defined (e.g., mono-, di-, tri-, or poly-substituted) by a substituent(s) as defined above or optionally selected from the group consisting of deuterium, hydroxy, amino, mercapto, nitro, halogen, cyano, oxo, optionally deuterated C₁₋₆ alkyl, halogenated C₁₋₆ alkyl, optionally deuterated C₃₋₆ cycloalkyl, optionally deuterated C₁₋₆ alkoxy, C₁₋₄ alkyl-NH—, NH₂—C₁₋₆ alkylene, C₁₋₄ alkyl-NHC(O)—, C₁₋₄ alkyl-C(O)NH— and any combination thereof.

As used herein, the term “alkynyl”, used alone or in combination, refers to a linear or branched monovalent hydrocarbon group containing from 2 to 8 (e.g., from 2 to 6, from 2 to 5, from 2 to 4, or preferably 2) carbon atoms and having one or more (e.g., from 1 to 3, from 1 to 2, or 1) carbon-carbon triple bonds. Examples of alkynyl include, but are not limited to, ethynyl, 1-propynyl, 1-butynyl, and 1,3-diynyl.

As used herein, the term “alkenyl”, used alone or in combination, refers to a linear or branched monovalent hydrocarbon group containing from 2 to 8 (e.g., from 2 to 6, from 2 to 5, from 2 to 4, from 2 to 3, or 2) carbon atoms and having one or more (e.g., from 1 to 3, from 1 to 2, or 1) carbon-carbon double bonds. Examples of alkenyl group include, but are not limited to, vinyl (e.g., —CH═CH—), 1-propenyl, allyl, 1-butenyl, 2-butenyl, 3-butenyl, isobutenyl, pentenyl, n-pent-2,4-dienyl, 1-methyl-but-1-enyl, 2-methyl-but-1-enyl, 3-methyl-but-1-enyl, 1-methyl-but-2-enyl, 2-methyl-but-2-enyl, 3-methyl-but-2-enyl, 1-methyl-but-3-enyl, 2-methyl-but-3-enyl, 3-methyl-but-3-enyl, and hexenyl.

As used herein, the term “quaternary ammonium salt group” used alone or in combination refers to a quaternary ammonium salt group having the Formula NRₐR_{b}R_{c}X⁻, where Rₐ, R_{b}, and R_{c} each independently represent C₁₋₂₀ alkyl group, and X⁻ represents F⁻, Cl⁻, Br⁻, or I⁻; or where Rₐ and R_{b}together with the N atom to which they are attached form a 4- to 8-membered heterocyclyl (especially a 5- to 8-membered heterocyclyl, or a 5- to 6-membered heterocyclyl), and the ring atoms of the 5- to 8-membered heterocyclyl optionally further include a heteroatom selected from oxygen, nitrogen, and sulfur, and R_{c} represents a C₁₋₂₀ alkyl group, and X⁻ represents F⁻, Cl⁻, Br⁻, or I⁻. The heterocyclyl in the 5- to 8-membered heterocyclyl may be as defined above, and the heteroatom(s) of the heterocyclyl optionally further includes an oxygen atom, a nitrogen atom, and/or a sulfur atom. Exemplary heterocyclyl includes, but is not limited to, piperidinyl, morpholinyl, imidazolyl, triazolyl, piperazinyl.

Exemplary heterocyclyl quaternary ammonium salt groups include, but are not limited to

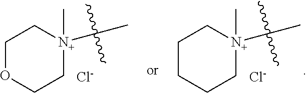

As used herein, “bornylane” or “bomane” (also known as 1,7,7-trimethylbicyclo[2.2.1]heptane; camphane; bornylane) has a definition known to those skilled in the art. As used herein, “camphanyl” or “bornyl” refers to a monovalent group of bornane, i.e., the group remaining after any one of the hydrogens in bornane is removed. Representative examples of “bornyl” include, but are not limited to, 1,7,7-trimethylbicyclo[2.2.1]heptan-2-yl, 1,7,7-trimethylbicyclo[2.2.1]heptan-3-yl, 1,7,7-trimethylbicyclo[2.2.1]heptan-4-yl, 1,7,7-trimethylbicyclo[2.2.1]heptan-5-yl, 1,7,7-trimethylbicyclo[2.2.1]heptan-6-yl,

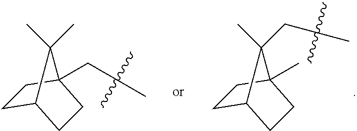 [SMILES: *CC12CCC(CC1)C2(C)C ; *CC1(C)C2CCC1(C)CC2]

As used herein, “bicyclo[2.2.1]heptane” also known as “norbornane”, has a definition known to those skilled in the art. As used herein, “bicyclo[2.2.1]heptyl” or “norbornyl” refers to a monovalent group of bicyclo[2.2.1]heptane, i.e., the group remaining after any hydrogen in bicyclo[2.2.1]heptane is removed. Representative examples of “bicyclo[2.2.1]heptyl” include, but are not limited to, bicyclo[2.2.1]heptan-2-yl, bicyclo[2.2.1]heptan-3-yl, bicyclo[2.2.1]heptan-4-yl, bicyclo[2.2.1]heptan-5-yl, or bicyclo[2.2.1]heptan-6-yl.

As used herein, the term “bicyclo[2.2.1]heptene” has a definition known to those skilled in the art. As used herein, “bicyclo[2.2.1]heptenyl” refers to a monovalent group of bicyclo[2.2.1]heptene, i.e., the group remaining after any hydrogen in bicyclo[2.2.1]heptene is removed. Representative examples of “bicyclo[2.2.1]heptenyl” include, but are not limited to, bicyclo[2.2.1]hept-5-en-2-yl, bicyclo[2.2.1]hept-5-en-3-yl, or bicyclo[2.2.1]hept-5-en-7-yl.

As used herein, “adamantane” (also known as tricyclo[3.3.1.1^{3,7}]decanet has a definition known to those skilled in the art, and its structural formula is e.g., as follows:

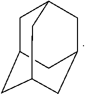

As used herein, “adamantanyl” refers to a monovalent group of adamantane, that is, the group remaining after any hydrogen in adamantane is removed. Representative examples of “adamantanyl” include, but are not limited to, 1-adamantanyl, 2-adamantanyl, 3-adamantanyl, 4-adamantanyl, 5-adamantanyl, 6-adamantanyl, 7-adamantanyl, 8-adamantanyl, 9-adamantanyl, or 10-adamantanyl.

As used herein, “noradamantane” (also known as octahydro-2,5-methanopentalen) has a definition known to those skilled in the art, and its structural formula is e.g., as follows:

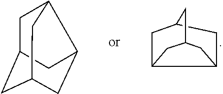 [SMILES: C1C2CC3CC1CC3C2 ; C1C2CC3CC1CC3C2]

As used herein, “noradamantanyl” refers to a monovalent group of noradamantane, that is, the group remaining after any hydrogen in noradamantane is removed. Representative examples of “noradamantanyl” include, but are not limited to, 1-noradamantanyl, 2-noradamantanyl, 3-noradamantanyl, 4-noradamantanyl, 5-noradamantanyl, 6-noradamantanyl, 7-noradamantanyl, 8-noradamantanyl or 9-noradamantanyl.

In all embodiments of the present disclosure, the salts or pharmaceutically acceptable salts of the compounds of Formula (I), Formula (II) or Formula (III) refer to non-toxic inorganic or organic acid and/or base addition salts. Examples include: sulfates, hydrohalides (including hydrochlorides and hydrobromates), maleates, sulfonates, citrates, lactates, lactobionates, L-tartrates, fumarates, L-malates, L-lactates, α-ketoglutarates, hippurates, D-glucuronates, D-gluconates, α-D-glucoheptates, glycolates, mucates, L-ascorbates, orotates, picrates, glycinates, alaninates, argininates, cinnamates, laurates, pamoates, sebacates, benzenesulfonates, methanesulfonates, ethanesulfonates, ethanedisulfonates, formates, acetates, 2,2-dichloroacetates, trimethylacetates, propionates, valerates, palmitates, triphenylacetates, 2-ethyl-butanedioates, iodates, nicotiniates, L-pyroglutamates, L-prolinates, ferulates, 2-hydroxyethanesulfonates, nitrates, gentisates, cholates, salicylate, terephthalates, glutarates, adipates, stearates, oleoates, undecylenates, camphorates, camphorsulfonates, dodecyl sulfonates, phosphates, thiocyanates, dihydrophosphates, pyrophosphates, metaphosphates, oxalates, carbonates, malonates, benzoates, mandelates, succinates, pyruvates, p-chlorobenzenesulfonates, 1,5-naphthalenedisulfonates, 3-hydroxy-2-naphthoates, 1-hydroxy-2-naphthoates, 2-naphthalenesulfonates, glycolates, or p-toluenesulfonates, etc.

“Pharmaceutically acceptable carrier” refers to a pharmaceutically acceptable material, such as a filler, stabilizer, dispersant, suspending agent, diluent, excipient, thickener, solvent, or encapsulating material, with which the useful compounds according to the present disclosure are carried or transported into or administered to a patient so that they can perform their intended function. Generally, such constructs are carried or transported from one organ or part of the body to another organ or part of the body. The carrier is compatible with the other ingredients of the formulation, including the compounds useful in the present disclosure, and is not harmful to the patient, and the carrier must be “acceptable”. Some examples of materials that can be used as pharmaceutically acceptable carriers include sugars such as lactose, glucose, and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository wax; oils such as peanut oil, cottonseed oil, safflower oil, sesame oil, olive oil, corn oil and soybean oil; glycols such as propylene glycol; polyols such as glycerol, sorbitol, mannitol, and polyethylene glycol; esters such as ethyl oleate and ethyl laurate; agar; buffers such as magnesium hydroxide and aluminum hydroxide; surfactant phosphate buffer solution; and other common non-toxic compatible substances used in pharmaceutical formulations.

As used herein, the term “room temperature” refers to the ambient temperature, such as 20-30° C.

As used herein, “stereoisomer” refers to a compound with the same chemical structural formula, but a different arrangement of atoms or groups in space. Stereoisomers include enantiomers, diastereomers, conformational isomers (rotational isomers), geometric isomers (cis/trans isomers), atropisomers, and so on.

As used herein, the term “solvate” refers to an association or complex formed by the interaction between one or more solvent molecules and compounds of the present invention. Examples of solvents include water, isopropanol, ethanol, methanol, DMSO, ethyl acetate, acetic acid and ethanolamine. The term “hydrate” means that a complex formed with water.

As used herein, the term “chiral” refers to a molecule that is nonsuperimposable on its mirror image; whereas “achiral” refers to a molecule that can be superimposed on its mirror image.

As used herein, the term “enantiomers” refers to two isomers of a compound that are non-superimposable mirror images.

As used herein, the term “diastereomers” refers to stereoisomers that have two or more chiral centers but are not mirror images of each other. The diastereomers have different physical properties, such as melting point, boiling point, spectral properties and reactivity. The diastereomeric mixture can be separated by high-resolution analytical operations such as electrophoresis and chromatography, such as HPLC.

As used herein, the term “oxo”, used alone or in combination, refers to =O.

As used herein, the term “C(O)” or “C(═O)” or “C═O”, used alone or in combination, refers to carbonyl.

As used herein, the term “C_{x-y} spiro-cycloalkyl” (x and y each being an integer), used alone or in combination, refers to a spiro-cycloalkyl group containing from x to y carbon atoms. As used herein, the term “C₇₋₁₁ spiro-cycloalkyl”, used alone or in combination, refers to a spiro-cycloalkyl group containing from 7 to 11 carbon atoms. The term “C₅₋₁₅ spiro-cycloalkyl” includes “C₇₋₁₁ spiro-cycloalkyl”, representative examples of which include, but are not limited to, spiro[3.3]heptyl, spiro[2.5]octyl, spiro[3.5]nonyl, spiro[4.4]nonyl, spiro[4.5]decyl, or spiro[5.5]undecyl. The term “C₇₋₁₁ spiro-cycloalkyl” is optionally substituted with one or more substituents selected from the group consisting of C₁-C₃ alkyl, halogenated C₁₋₃alkyl, C₁-C₃ alkoxy, deuterated C₁-C₃ alkoxy, C₁-C₃ alkylamino, amino, oxo, halogen, hydroxy, cyano, and any combination thereof.

As used herein, the term “C_{x-y} bridged cycloalkyl” (x and y each being an integer), used alone or in combination, refers to a bridged cycloalkyl group containing from x to y carbon atoms. As used herein, the term “C₇₋₁₁ bridged cycloalkyl”, used alone or in combination, refers to a bridged cycloalkyl group containing from 7 to 11 carbon atoms. Representative examples of “C₇₋₁₁ bridged cycloalkyl” include, but are not limited to, adamantanyl, noradamantanyl, norbornyl (also named as bicyclo[2.2.1]heptyl by the IUPAC system), and cubanyl. Herein, the term “bridged cycloalkyl” is optionally substituted with 1-10 substituents selected from the group consisting of C₁₋₃ alkyl, deuterated C₁₋₃ alkyl, C₁₋₃ alkoxy, halogen, halogenated C₁₋₃ alkyl, C₁₋₃ alkylamino, amino, hydroxy, cyano, oxo, or any combination thereof.

As used herein, “p-menthane” has the definitions known to those skilled in the art, and its structural formula is e.g., as follows:

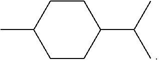

As used herein, “p-menthanyl” refers to a monovalent group of menthane, that is, the group remaining after any hydrogen in p-menthane is removed. Representative examples of “p-menthanyl” include, but are not limited to,

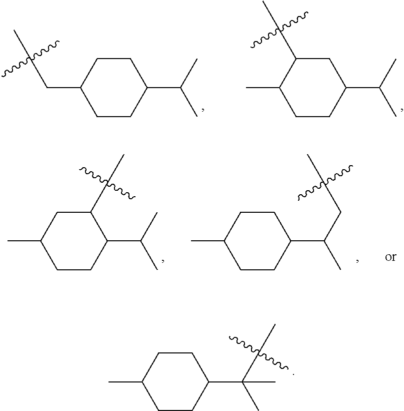

As used herein, “m-menthane” has the definitions known to those skilled in the art, and its structural formula is e.g., as follows:

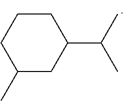

As used herein, “m-menthanyl” refers to a monovalent group of m-menthane, that is, the group remaining after any hydrogen in m-menthane is removed. Representative examples of “m-menthanyl” include, but are not limited to,

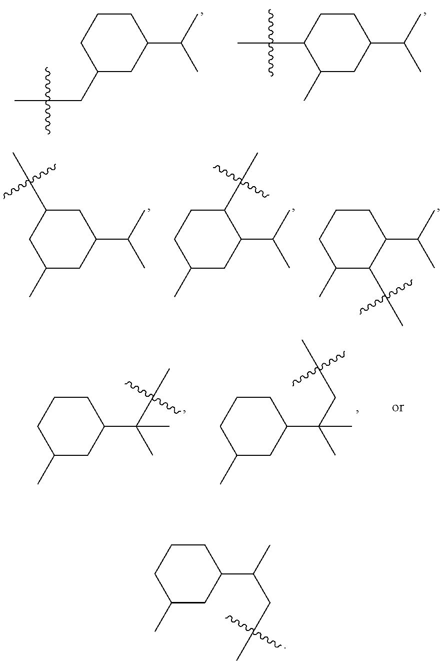

As used herein, “Quinuclidine” (also known as 1-azabicyclo[2.2.2]octane) has the definitions known to those skilled in the art, and its structural formula is e.g., as follows:

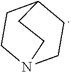

As used herein, “quinuclidinyl” refers to a monovalent group of Quinuclidine, that is, the group remaining after any hydrogen in Quinuclidine is removed. Representative examples of “quinuclidinyl” include, but are not

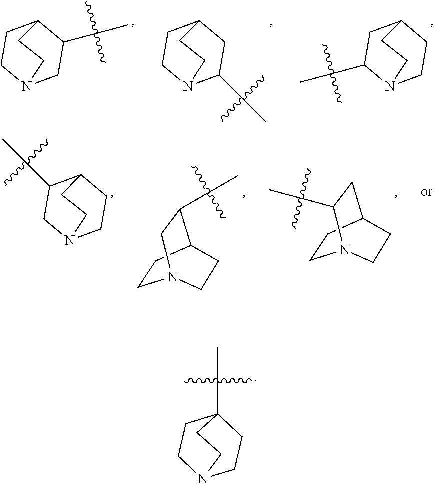

### EXAMPLES

In the following description, numerous specific details are set forth in order to provide a thorough understanding of the present disclosure. The present disclosure may be practiced without some or all of these specific details. In other cases, well-known process operations have not been described in detail in order not to unnecessarily obscure the present disclosure. Although the present disclosure will be described in conjunction with specific embodiments, it should be understood that this is not intended to limit the present disclosure to these embodiments.

The following abbreviations are used throughout the specification and examples:

| | |
|---|---|
| AcOH | acetic acid |
| BnCl | benzyl chloride |
| Bipy | bipyridine |
| BPO | benzoyl peroxide |
| t-BuONO | Tert-Butyl nitrite |
| DCE | 1,2-dichloroethane |
| DCM | dichloromethane |
| DIEA or DIPEA | N,N-diisopropylethylamine |
| DMF | N,N-dimethylformamide |
| DMSO | dimethyl sulfoxide |
| equiv | equivalent |
| ESI | electrospray ionization |
| EtOH | ethanol |
| HATU | 2-(7-Azabenzotriazol-1-yl)-N,N,N′,N′- |
| | tetramethyluronium hexafluorophosphate |
| HOAc or AcOH | acetic acid |
| HPLC | high performance liquid chromatography |
| HRMS | high resolution mass spectrometry |
| LC-MS | liquid chromatography-mass spectrometry |
| LRMS | low resolution mass spectrometry |
| LC | liquid chromatography |
| MeOH | Methanol |
| MsCl | Methanesulfonyl chloride |
| MsO- | Methanesulfonyloxy |
| ¹H NMR | Proton nuclear magnetic resonance |
| rt | room temperature |
| TEA | triethylamine |
| TFA | trifluoroacetic acid |
| THF | tetrahydrofuran |
| TLC | thin layer chromatography |
| TMS | tetramethylsilane |
| TsOH | p-toluenesulfonic acid |
| TsO- | Tosyloxy |

In the present disclosure, the ¹H NMR spectrum was recorded on a Bruker-500 MHz nuclear magnetic resonance instrument, by using, as a solvent, CD₃OD (6=3.31 ppm) containing 0.1% TMS (as an internal standard); or using, as a solvent, CDCl₃ (6=7.26 ppm) containing 0.1% TMS (as an internal standard); or using, as a solvent, DMSO-d₆ (6=2.50 ppm) containing 0.03% TMS (as an internal standard). LC-MS spectrum was recorded on a Sciex API 2000 mass spectrometer equipped with an Agilent 1100 binary pump and DAD, as well as an ELSD, or on an Agilent 1260-6125B single quadrupole liquid-mass spectrometer equipped with an Agilent 1260 quadrupole pump, DAD, and ELSD; HPLC preparation was measured on a SHIMADZU LC-20AP type instrument, and HPLC purity was measured on a SHIMADZU LC-30AP or Waters 1525 type instrument. Unless otherwise specified, all reactions were performed in the air atmosphere. The reactions were monitored via TLC or LC-MS. The intermediates were separated and purified by column chromatography using ISCO or Biotage systems, or used directly in the next step without further purification. The final products obtained from the synthesis methods were separated and purified using conventional techniques in the field, such as preparative liquid chromatography with a Waters 2767 system.

Solvents and reagents are processed as follows:

the solvents used in the reactions such as DCM, DMF, anhydrous EtOH, and anhydrous MeOH were all purchased from Sinopharm Group; HPLC preparation uses preparation grade CH₃CN and deionized water; Other reaction substrates, reagents and drugs can be commercially available without special instructions, or can be synthesized using or according to methods known in the art.

### General Synthesis Methods

Compounds and/or pharmaceutically acceptable salts thereof of the present disclosure can be synthesized using commercially available raw materials by synthetic techniques known in the art. The synthetic schemes described below illustrate the preparation of most compounds. The starting materials or reagents used in each scheme can be purchased from commercial sources or prepared by methods known to those skilled in the art. One skilled in the art can prepare the salts, racemates, enantiomers, phosphates, sulfates, hydrochlorides and prodrug forms of the compounds of Formula (I) of the present disclosure according to routine techniques in the art.

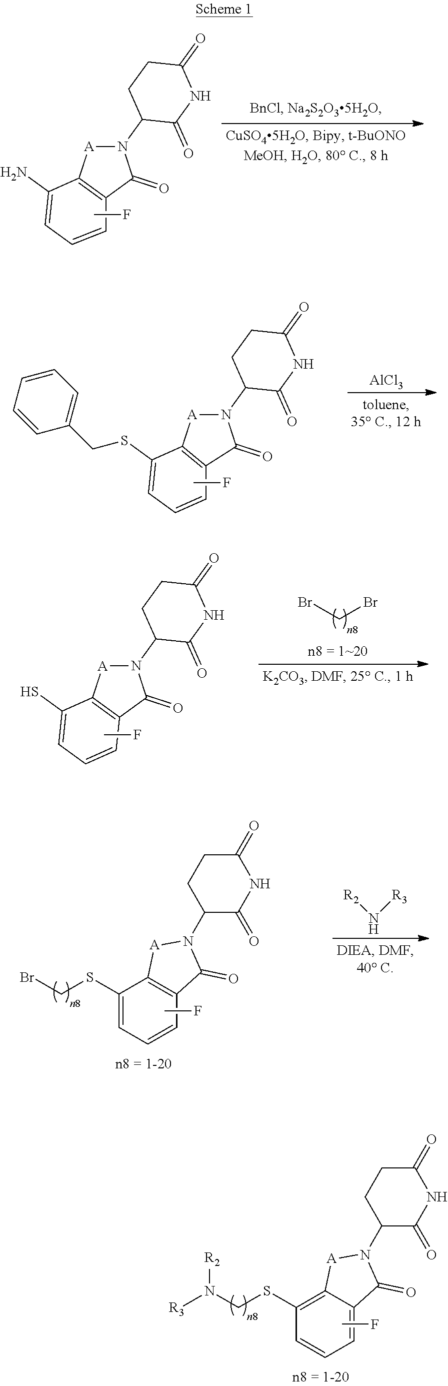

In Scheme 1, A represents CH₂ or C(O); n8 represents an integer of 1 to 20; and R₂ and R₃ are as defined in the compounds of Formula (I) above and its various sub-embodiments described herein.

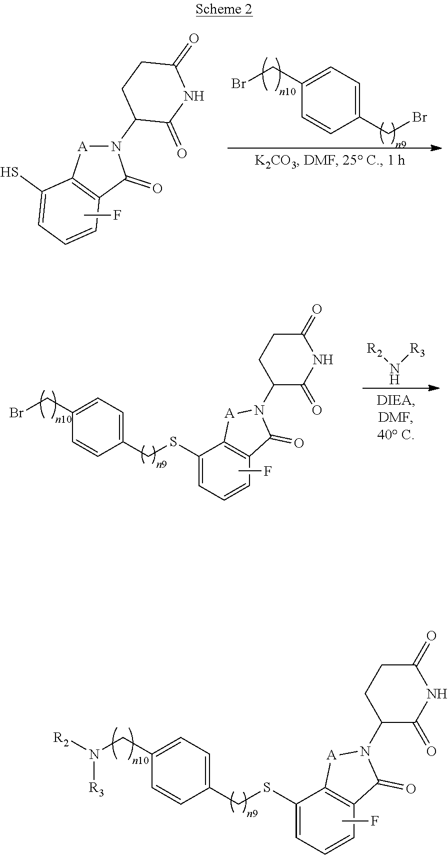

In Scheme 2, A represents CH₂ or C(O); n9 and n10 each independently represent an integer of 1 to 20; and R₂ and R₃ are as defined in the compounds of Formula (I) above and its various sub-embodiments described herein. In Step 1 of Scheme 2, the phenyl group of the dibromide reaction substrate can also be appropriately modified or replaced, depending on the target compounds, with options such as optionally substituted cycloalkylene, optionally substituted heterocyclylene, or optionally substituted heteroarylene, as defined in the compounds of Formula (I) and its various sub-embodiments in the present disclosure.

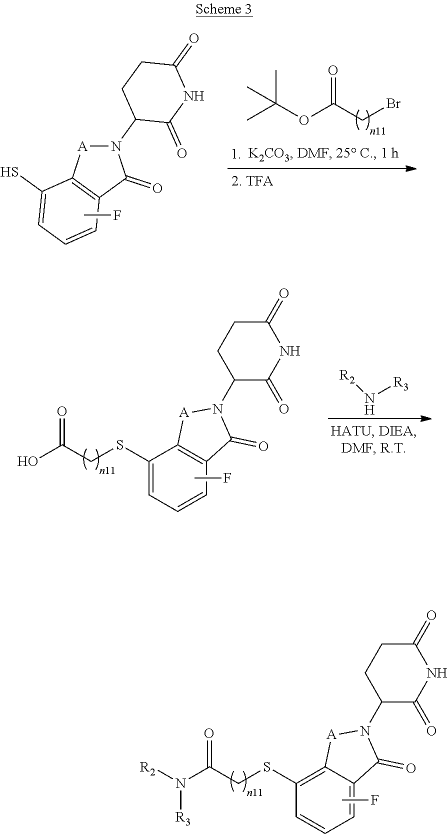

In Scheme 3, A represents CH₂ or C(O); n11 represents an integer of 1 to 20; and R₂ and R₃ are as defined in the compounds of Formula (I) above and its various sub-embodiments described herein.

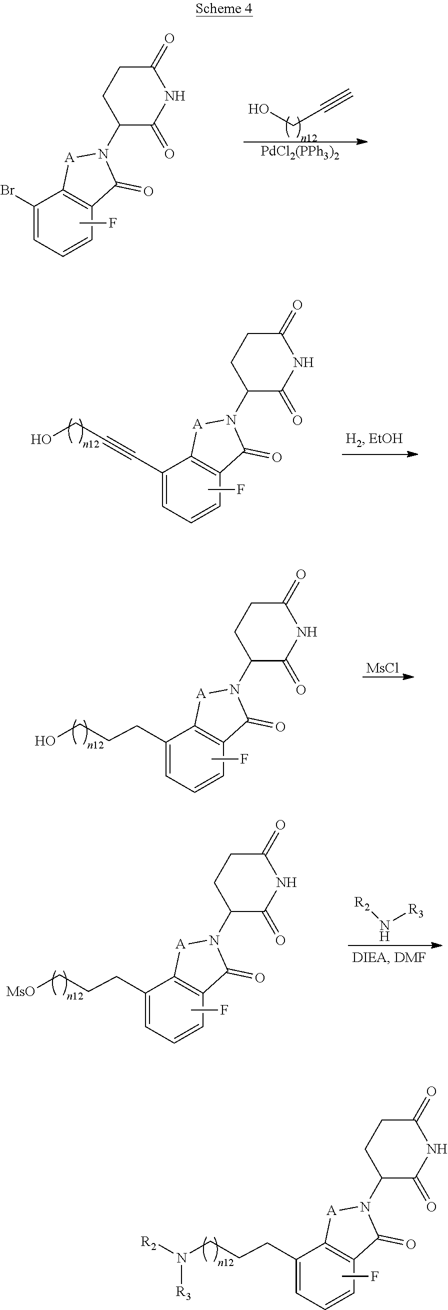

In Scheme 4, A represents CH₂ or C(O); n12 represents an integer of 1 to 20; and R₂ and R₃ are as defined in the compounds of Formula (I) above and its various sub-embodiments described herein.

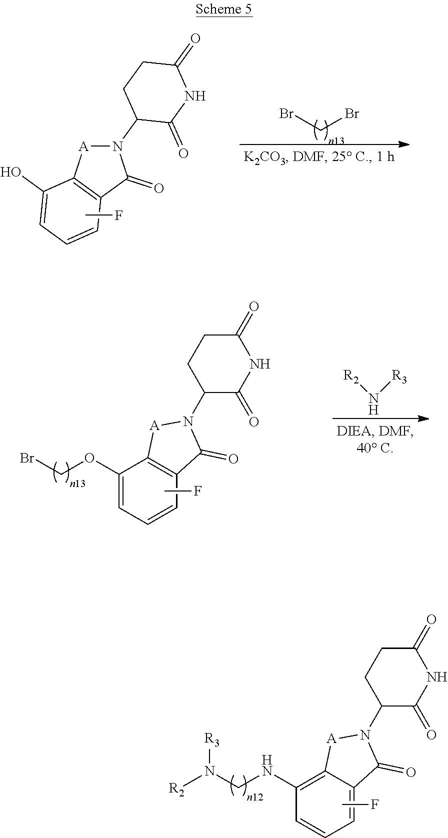

In Scheme 5, A represents CH₂ or C(O); n13 represents an integer of 1 to 20; and R₂ and R₃ are as defined in the compounds of Formula (I) above and its various sub-embodiments described herein.

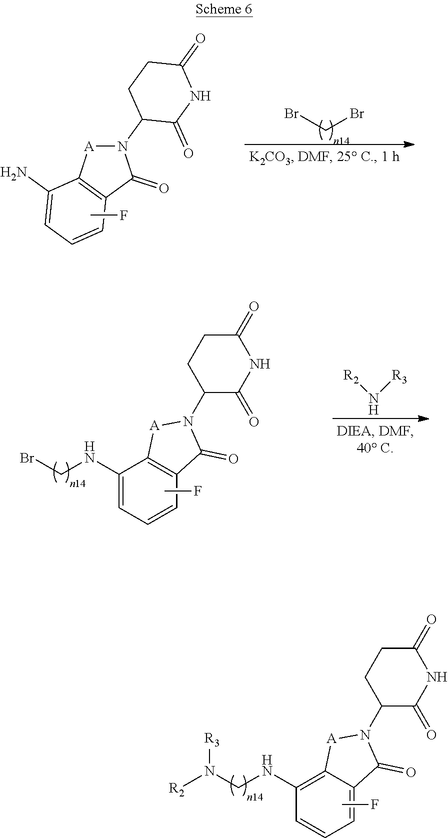

In Scheme 6, A represents CH₂ or C(O); n14 represents an integer of 1 to 20; and R₂ and R₃ are as defined in the compounds of Formula (I) above and its various sub-embodiments described herein.

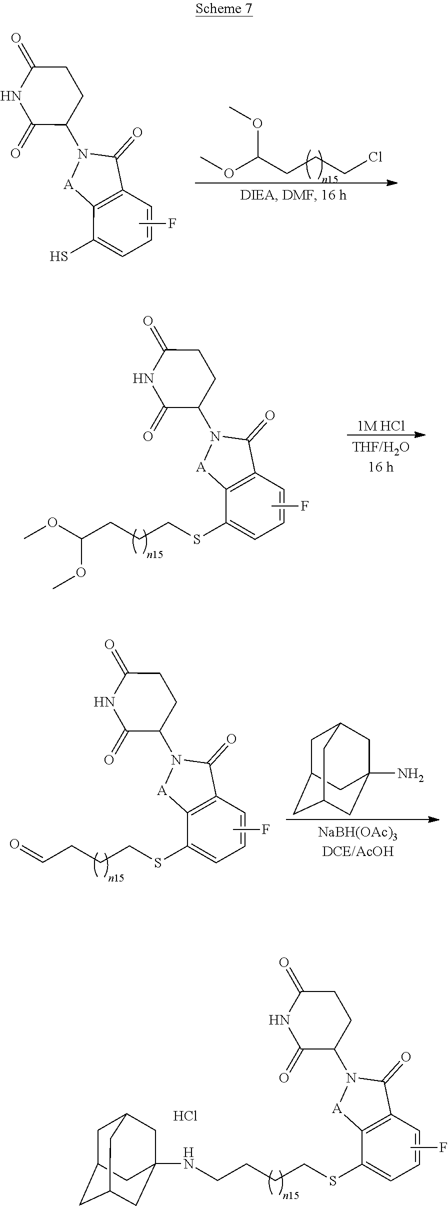

In Scheme 7, A represents CH₂ or C(O), and n15 represents an integer of 0 to 20.

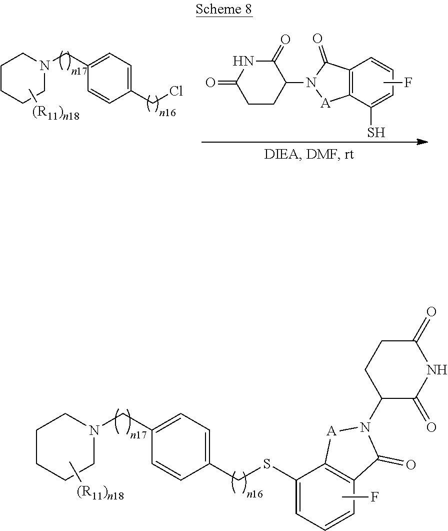

In Scheme 8, A represents CH₂ or C(O); n16 and n17 each independently represent an integer of 1 to 20; (R₁₁)ₙ₁₈ denotes that the piperidinyl is substituted with n18 R₁₁, where n18 represents an integer of 0, 1, 2, 3, 4, 5 or 6; and R₁₁ represents fluorine.

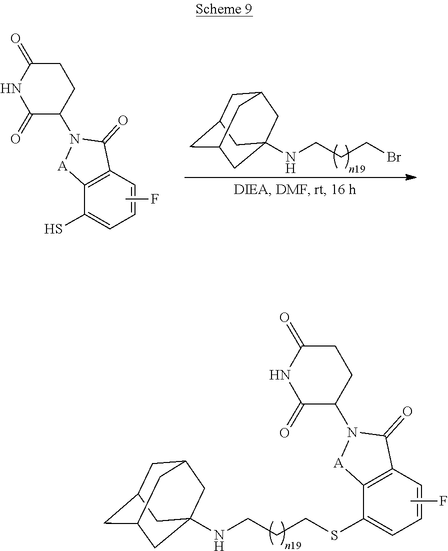

In Scheme 9, A represents CH₂ or C(O), and n19 represents an integer of 0 to 20.

Depending upon the target compounds, the above schemes and their reaction substrates, reaction conditions (including reaction dosage, temperature, duration, etc.), work up, etc. can be appropriately modified and adjusted by techniques and methods well known to those skilled in the art to obtain the desired target compounds. The obtained target compounds can be further modified by the substituents and the like to obtain subsequent target compounds through methods well known to those skilled in the art.

### EXAMPLES

### Example 1: Preparation of 3-(4-((4-((adamantan-1-yl)amino)butyl)thio)-6-fluoro-1-oxoisoindolin-2-yl)piperidine-2,6-dione (GT-04243)

The compound (GT-04243) was prepared by referring to the method of Scheme 7.

### Step 1:

At room temperature, to a 100 mL single-neck flask were sequentially added 3-(6-fluoro-4-mercapto-1-oxoisoindolin-2-yl)piperidine-2,6-dione (300 mg, 1.019 mmol), 4-chloro-1,1-dimethoxybutane (155.58 mg, 1.019 mmol), DMF (5 mL) and DIEA (0.505 mL, 3.058 mmol). The reaction mixture was stirred at 25° C. for 16 hours. After the reaction was completed as detected by TLC, the reaction was quenched with water. The reaction mixture was then extracted three times with ethyl acetate. The organic phases were combined, and washed three times with water and once with brine. The organic phase was concentrated under vacuum to yield 3-(4-((4,4-dimethoxybutyl)thio)-6-fluoro-1-oxoisoindolin-2-yl)piperidine-2,6-dione (yellow solid, 410 mg, yield 97.99%). ¹H NMR (400 MHz, d₆-DMSO) δ 11.06 (s, 1H), 7.68-7.49 (m, 1H), 7.52-7.20 (m, 1H), 5.18 (dd, J=13.2, 5.2 Hz, 1H), 4.59-4.18 (m, 3H), 3.76-3.57 (m, 1H), 3.31-3.18 (m, 6H), 2.96 (t, J=12.8 Hz, 1H), 2.77-2.37 (m, 6H), 2.12-1.99 (m, 1H), 1.77 (d, J=42.1 Hz, 4H). LCMS (ESI): calcd for C₁₉H₂₃FN₂O₅S; [M+Na+CH₃CN]⁺, 474.13; found, 474.2.

### Step 2:

At room temperature, to a 100 mL single-neck flask were added 3-(4-((4,4-dimethoxybutyl)thio)-6-fluoro-1-oxoisoindolin-2-yl)piperidine-2,6-dione (410 mg, 0.999 mmol), THF (20 mL) and 1M hydrochloric acid (20 mL). The reaction mixture was stirred at 25° C. for 2 hours. After the reaction was completed as detected by TLC, the reaction was quenched with water. The reaction mixture was then extracted four times with ethyl acetate. The organic phases were combined, and dried over anhydrous sodium sulfate, and concentrated under vacuum to give 4-((2-(2,6-dioxopiperidin-3-yl)-6-fluoro-1-oxoisoindolin-4-yl)thio)butanal (white solid, 320 mg, yield 87.92%). ¹H NMR (400 MHz, d₆-DMSO) δ 10.99 (s, 1H), 9.68 (s, 1H), 7.56 (dd, J=10.1, 1.9 Hz, 1H), 7.34 (dd, J=7.4, 1.9 Hz, 1H), 5.12 (dd, J=13.3, 5.2 Hz, 1H), 4.25 (dd, J=57.1, 17.3 Hz, 2H), 3.15 (t, J=7.4 Hz, 2H), 2.96-2.81 (m, 1H), 2.69-2.54 (m, 2H), 2.46 (dd, J=13.3, 4.3 Hz, 1H), 2.07-1.97 (m, 1H), 1.94-1.72 (m, 2H), 1.36 (s, 2H).

### Step 3:

At room temperature, to a 100 mL single-neck flask were added 4-((2-(2,6-dioxopiperidin-3-yl)-6-fluoro-1-oxoisoindolin-4-yl)thio)butanal (300 mg, 0.823 mmol), adamantan-1-amine (124.52 mg, 0.823 mmol), 1,2-dichloroethane (5 mL), sodium borohydride (436.22 mg, 2.058 mmol), and acetic acid (49.42 mg, 0.823 mmol). Under a nitrogen atmosphere, the reaction mixture was stirred at 25° C. for 16 hours. After the reaction was completed as detected by TLC, to the reaction mixture were added dichloromethane and water, followed by extraction three times with dichloromethane. The organic phases were combined, dried over anhydrous sodium sulfate, and concentrated under vacuum to give a crude product. The crude product was separated and purified by preparative HPLC, then treated with water and lyophilized to afford the target compound GT-04243 (white solid, 170 mg, yield 38.53%). ¹H NMR (400 MHz, d₆-DMSO) δ 11.01 (s, 1H), 8.74 (s, 2H), 7.55 (dt, J=13.2, 6.6 Hz, 1H), 7.43-7.33 (m, 1H), 5.13 (dd, J=13.3, 5.1 Hz, 1H), 4.38-4.15 (m, 2H), 3.21 (t, J=6.8 Hz, 2H), 2.98-2.82 (m, 3H), 2.65-2.55 (m, 1H), 2.49-2.42 (m, 1H), 2.11 (s, 3H), 2.01 (dd, J=10.4, 5.4 Hz, 1H), 1.86 (s, 6H), 1.79-1.54 (m, 10H). LCMS (ESI): calcd for C₇H₃₄FN₃O₃S; [M+H]⁺; 500.24; found, 500.3.

### Example 2: Preparation of 3-(6-fluoro-1-oxo-4-((4-(piperidin-1-ylmethyl)benzyl)thio)isoindolin-2-yl)piperidine-2,6-dione (GT-04117)

The compound (GT-04117) was prepared by referring to the method of Scheme 8.

At room temperature, to a 100 mL single-neck flask were sequentially added 1-(4-(chloromethyl)benzyl)piperidine (200 mg, 0.894 mmol), 3-(6-fluoro-4-mercapto-1-oxoisoindolin-2-yl)piperidine-2,6-dione (263.07 mg, 0.894 mmol), DMF (5 mL) and DIEA (0.443 mL, 2.682 mmol). The reaction mixture was stirred at 25° C. for 2 hours. After the reaction was completed as detected by TLC, the reaction was quenched with water. The reaction mixture was then extracted three times with ethyl acetate. The organic phases were combined, washed three times with water and once with brine, dried over anhydrous sodium sulfate, and concentrated under vacuum. The resulting residue was subjected to normal-phase column chromatography (eluent (v/v): ethyl acetate/petroleum ether=0-80%) for separation and purification, yielding the target compound GT-04117 (white solid, 371 mg, yield 86.18%). ¹H NMR (400 MHz, d₆-DMSO) δ 11.00 (s, 1H), 10.61 (s, 1H), 7.62-7.51 (m, 3H), 7.45 (d, J=8.1 Hz, 2H), 7.36 (dd, J=7.5, 2.2 Hz, 1H), 5.10 (dd, J=13.3, 5.1 Hz, 1H), 4.45 (s, 2H), 4.34-4.07 (m, 4H), 3.21 (d, J=11.0 Hz, 2H), 2.96-2.85 (m, 1H), 2.83-2.70 (m, 2H), 2.59 (d, J=16.9 Hz, 1H), 2.41 (dd, J=13.0, 4.5 Hz, 1H), 2.04-1.94 (m, 1H), 1.88-1.59 (m, 5H), 1.31 (d, J=7.3 Hz, 1H). LCMS (ESI): calcd for [M+H]⁺; 482.19; found, 482.50.

### Example 3: Preparation of 3-(4-((4-((4,4-difluoropiperidin-1-yl)methyl)benzyl)thio)-6-fluoro-1-oxoisoindolin-2-yl)piperidine-2,6-dione (GT-04024)

The compound (GT-04024) was prepared by referring to the method of Scheme 8.

At room temperature, to a 100 mL single-neck flask were sequentially added 1-(4-(chloromethyl)benzyl)-4,4-difluoropiperidine (150 mg, 0.578 mmol), 3-(6-fluoro-4-mercapto-1-oxoisoindolin-2-yl)piperidine-2,6-dione (169.97 mg, 0.578 mmol), DMF (5 mL) and DIEA (0.286 mL, 1.733 mmol). The reaction mixture was stirred at room temperature for 2 hours. After the reaction was completed as detected by TLC, the reaction was quenched with water. The reaction mixture was then extracted three times with ethyl acetate. The organic phases were combined, washed three times with water and once with brine, dried over anhydrous sodium sulfate, and concentrated under vacuum. The resulting residue was subjected to normal-phase column chromatography (eluent (v/v): ethyl acetate/petroleum ether=0-100%) for separation and purification, yielding the target compound GT-04024 (white solid, 220 mg, yield 68.75%). ¹H NMR (400 MHz, d₆-DMSO) δ 11.54 (s, 1H), 11.00 (s, 1H), 7.68-7.51 (m, 3H), 7.46 (d, J=7.9 Hz, 2H), 7.37 (dd, J=7.5, 2.1 Hz, 1H), 5.10 (dd, J=13.3, 5.1 Hz, 1H), 4.45 (s, 2H), 4.40-4.18 (m, 3H), 4.12 (d, J=17.3 Hz, 1H), 3.35-3.28 (m, 2H), 3.09 (s, 2H), 2.96-2.83 (m, 1H), 2.59 (d, J=17.5 Hz, 1H), 2.48-2.37 (m, 2H), 2.33 (d, J=18.2 Hz, 2H), 1.99 (dd, J=8.9, 3.7 Hz, 1H). LCMS (ESI): calcd for [M+H]⁺, 518.17, found, 518.30.

### Example 4: Preparation of 3-(4-((7-((adamantan-1-yl)amino)heptyl)thio)-6-fluoro-1-oxoisoindolin-2-yl)piperidine-2,6-dione (GT-04333)

The compound (GT-04333) was prepared by referring to the method of Scheme 9.

At room temperature, to a 100 mL single-neck flask were sequentially added 3-(6-fluoro-4-mercapto-1-oxoisoindolin-2-yl)piperidine-2,6-dione (200 mg, 0.680 mmol), N-(7-bromoheptyl)adamantan-1-amine (223.13 mg, 0.680 mmol), DMF (5 mL) and DIEA (0.337 mL, 2.039 mmol). The reaction mixture was stirred at 25° C. for 16 hours. TLC analysis indicated that the starting materials were completely consumed, with a significant amount of impurities generated. The reaction solution was separated by preparative HPLC, then lyophilized to afford the target compound GT-04333 (white solid, 40 mg, yield 40%). ¹H NMR (400 MHz, MeOD) δ 7.37 (d, J=8.2 Hz, 1H), 7.29 (d, J=7.3 Hz, 1H), 5.14 (dd, J=13.4, 5.1 Hz, 1H), 4.56 (s, 4H), 4.36 (d, J=9.2 Hz, 2H), 3.11 (dd, J=7.1, 3.9 Hz, 2H), 2.90 (ddd, J=36.5, 25.2, 18.7 Hz, 4H), 2.69 (s, 6H), 2.51 (dd, J=13.1, 4.6 Hz, 1H), 2.19 (s, 4H), 1.90 (s, 6H), 1.84-1.59 (m, 10H), 1.46 (d, J=41.5 Hz, 6H). LCMS (ESI): calcd for C₃₀H₄₀FN₃O₃S⁺, [M+H]⁺, 542.28; found, 542.3.

### Example 5: Preparation of 3-(6-fluoro-4-((8-morpholinooctyl)thio)-1-oxoisoindolin-2-yl)piperidine-2,6-dione (GT-05341)

Referring to the method of Scheme 1, the target product (GT-05341) was prepared as white solid (10 mg, yield 26.30%). ¹H NMR (400 MHz, DMSO) δ 11.00 (s, 1H), 10.54 (s, 1H), 7.51 (dd, J=10.2, 2.2 Hz, 1H), 7.34 (dd, J=7.5, 2.2 Hz, 1H), 5.13 (dd, J=13.3, 5.1 Hz, 1H), 4.32 (d, J=17.2 Hz, 1H), 4.17 (d, J=17.1 Hz, 1H), 3.94 (d, J=10.7 Hz, 2H), 3.75 (t, J=11.5 Hz, 2H), 3.39 (s, 2H), 3.15 (t, J=6.6 Hz, 2H), 3.06-2.98 (m, 3H), 2.97-2.87 (m, 1H), 2.62 (t, J=21.0 Hz, 2H), 2.48-2.39 (m, 1H), 2.04-1.96 (m, 1H), 1.62 (dt, J=14.7, 7.1 Hz, 4H), 1.41 (s, 2H), 1.28 (s, 6H). LCMS (ESI) calcd for C₂₅H₃₅FN₃O₄S⁺ [M+H]⁺: 492.23, found, 492.3.

### Example 6: Preparation of 3-(4-((8-(diethylamino)octyl)thio)-6-fluoro-1-oxoisoindolin-2-yl)piperidine-2,6-dione (GT-05342)

Referring to the method of Scheme 1, the target product (GT-05342) was prepared as white solid (20 mg, yield 37.77%). ¹H NMR (400 MHz, DMSO) δ 10.92 (s, 1H), 9.71 (s, 1H), 7.43 (dd, J=10.1, 2.1 Hz, 1H), 7.27 (dd, J=7.5, 2.0 Hz, 1H), 5.05 (dd, J=13.3, 5.1 Hz, 1H), 4.25 (d, J=17.3 Hz, 1H), 4.10 (d, J=17.3 Hz, 1H), 3.31 (d, J=8.6 Hz, 2H), 3.21 (s, 1H), 3.08 (t, J=7.0 Hz, 2H), 3.00 (dt, J=11.6, 5.8 Hz, 4H), 2.93-2.86 (m, 2H), 2.86-2.76 (m, 1H), 2.52 (d, J=17.7 Hz, 2H), 2.38 (dd, J=13.1, 4.3 Hz, 1H), 2.00-1.89 (m, 1H), 1.55 (dt, J=14.4, 7.2 Hz, 4H), 1.35 (s, 2H), 1.22 (s, 6H). LCMS (ESI) calcd for C₂₅H₃₇FN₃O₃S⁺ [M+H]⁺: 478.25, found, 478.2.

### Example 7: Preparation of 3-(6-fluoro-1-oxo-4-((7-oxo-7-(piperidin-1-yl)heptyl)thio)isoindolin-2-yl)piperidine-2,6-dione (GT-05343)

Referring to the method of Scheme 3, the target product (GT-05343) was prepared as white solid (28 mg, yield 48.46%). ¹H NMR (400 MHz, DMSO) δ 11.00 (s, 1H), 7.50 (dd, J=10.2, 2.2 Hz, 1H), 7.33 (dd, J=7.5, 2.1 Hz, 1H), 5.12 (dd, J=13.2, 5.1 Hz, 1H), 4.32 (d, J=17.3 Hz, 1H), 4.17 (d, J=17.2 Hz, 1H), 3.40 (d, J=5.6 Hz, 2H), 3.14 (t, J=7.2 Hz, 2H), 2.95-2.84 (m, 1H), 2.58 (d, J=17.8 Hz, 2H), 2.45 (dd, J=13.1, 4.5 Hz, 1H), 2.25 (t, J=7.4 Hz, 2H), 2.05-1.93 (m, 1H), 1.62 (dd, J=14.4, 7.2 Hz, 2H), 1.55 (dd, J=11.8, 6.6 Hz, 2H), 1.46 (s, 1H), 1.44 (d, J=4.8 Hz, 2H), 1.43-1.34 (m, 4H), 1.29 (dt, J=13.6, 6.8 Hz, 2H). LCMS (ESI) calcd for C₂₅H₃₃FN₃O₄S⁺ [M+H]⁺: 490.22, found, 490.2.

### Example 8: Preparation of 3-(6-fluoro-1-oxo-4-((7-(piperidin-1-yl)heptyl)thio)isoindolin-2-yl)piperidine-2,6-dione (GT-05344)

Referring to the method of Scheme 1, the target product (GT-05344) was prepared as white solid (5 mg, yield 13.15%). ¹H NMR (400 MHz, DMSO) δ 11.00 (s, 1H), 9.81 (s, 1H), 7.51 (dd, J=10.1, 2.1 Hz, 1H), 7.34 (dd, J=7.5, 2.1 Hz, 1H), 5.13 (dd, J=13.2, 5.0 Hz, 1H), 4.32 (d, J=17.1 Hz, 1H), 4.17 (d, J=17.3 Hz, 1H), 3.37 (s, 2H), 3.15 (t, J=7.0 Hz, 2H), 2.93 (ddd, J=21.2, 14.0, 6.9 Hz, 3H), 2.85-2.74 (m, 2H), 2.59 (d, J=17.4 Hz, 1H), 2.43 (dd, J=13.1, 8.9 Hz, 1H), 2.05-1.97 (m, 1H), 1.75 (d, J=10.5 Hz, 3H), 1.70 (s, 2H), 1.67-1.58 (m, 4H), 1.45-1.38 (m, 2H), 1.36-1.21 (m, 5H). LCMS (ESI) calcd for C₂₅H₃₅FN₃O₃S⁺ [M+H]⁺: 476.24, found, 476.3.

### Example 9: Preparation of 3-(4-((7-(4,4-difluoropiperidin-1-yl)-7-oxoheptyl)thio)-6-fluoro-1-oxoisoindolin-2-yl)piperidine-2,6-dione (GT-05407)

Referring to the method of Scheme 3, the target product (GT-05407) was prepared as white solid (26 mg, yield 41.80%). ¹H NMR (400 MHz, DMSO) δ 10.93 (s, 1H), 7.43 (dd, J=10.1, 2.0 Hz, 1H), 7.26 (dd, J=7.5, 1.9 Hz, 1H), 5.05 (dd, J=13.3, 5.0 Hz, 1H), 4.25 (d, J=17.3 Hz, 1H), 4.10 (d, J=17.4 Hz, 1H), 3.50-3.44 (m, 4H), 3.07 (t, J=7.2 Hz, 2H), 2.84 (dt, J=12.5, 6.6 Hz, 1H), 2.52 (d, J=17.6 Hz, 1H), 2.41-2.32 (m, 1H), 2.27 (t, J=7.4 Hz, 2H), 1.93 (dd, J=12.3, 5.3 Hz, 3H), 1.82 (dd, J=16.2, 10.0 Hz, 2H), 1.59-1.51 (m, 2H), 1.42 (dd, J=15.0, 7.4 Hz, 2H), 1.34 (dd, J=15.3, 7.6 Hz, 2H), 1.27-1.19 (m, 2H). LCMS (ESI) calcd for C₂₅H₃₁F₃N₃O₄S⁺ [M+H]⁺: 526.20, found, 526.2.

### Example 10: Preparation of 3-(4-((7-(4,4-difluoropiperidin-1-yl)heptyl)thio)-6-fluoro-1-oxoisoindolin-2-yl)piperidine-2,6-dione (GT-05408)

Referring to the method of Scheme 1, the target product (GT-05408) was prepared as white solid (7.5 mg, yield 16.10%). ¹H NMR (400 MHz, DMSO) δ 11.00 (s, 1H), 10.91 (s, 1H), 7.51 (dd, J=10.2, 2.2 Hz, 1H), 7.34 (dd, J=7.5, 2.2 Hz, 1H), 5.13 (dd, J=13.2, 5.1 Hz, 1H), 4.32 (d, J=17.1 Hz, 1H), 4.17 (d, J=17.1 Hz, 1H), 3.56 (s, 2H), 3.15 (t, J=7.1 Hz, 2H), 3.07 (s, 3H), 2.95-2.85 (m, 1H), 2.68-2.54 (m, 2H), 2.45 (dd, J=13.2, 4.3 Hz, 2H), 2.40-2.26 (m, 3H), 2.05-1.98 (m, 1H), 1.67 (d, J=8.9 Hz, 2H), 1.61 (dd, J=14.6, 7.5 Hz, 2H), 1.41 (dd, J=14.2, 6.4 Hz, 2H), 1.35-1.23 (m, 4H). LCMS (ESI) calcd for C₂₅H₃₃F₃N₃O₃S++[M+H]⁺: 512.22, found, 512.3.

### Example 11: Preparation of 3-(4-((7-(cyclohexylamino)heptyl)thio)-6-fluoro-1-oxoisoindolin-2-yl)piperidine-2,6-dione (GT-05409)

Referring to the method of Scheme 1, the target product (GT-05409) was prepared as white solid (7.3 mg, yield 16.32%). ¹H NMR (400 MHz, DMSO) δ 11.00 (s, 1H), 8.59 (s, 2H), 7.51 (dd, J=10.1, 2.2 Hz, 1H), 7.34 (dd, J=7.5, 2.1 Hz, 1H), 5.13 (dd, J=13.3, 5.1 Hz, 1H), 4.32 (d, J=17.2 Hz, 1H), 4.17 (d, J=17.2 Hz, 1H), 3.15 (t, J=7.2 Hz, 2H), 3.00-2.88 (m, 2H), 2.87 (d, J=5.5 Hz, 2H), 2.61 (s, 1H), 2.47-2.39 (m, 1H), 2.08-1.95 (m, 3H), 1.75 (d, J=12.1 Hz, 2H), 1.68-1.53 (m, 5H), 1.42 (s, 2H), 1.25 (dd, J=25.9, 13.8 Hz, 8H), 1.14-0.98 (m, 1H). LCMS (ESI) calcd for C₂₆H₃₇FN₃O₃S⁺ [M+H]⁺: 490.25, found, 490.3.

### Example 12: Preparation of 3-(4-((7-((adamantan-1-yl)(methyl)amino)heptyl)thio)-6-fluoro-1-oxoisoindolin-2-yl)piperidine-2,6-dione (GT-05421)

Referring to the method of Scheme 1, the target product (GT-05421) was prepared as white solid (8.5 mg, yield 16.89%). ¹H NMR (400 MHz, DMSO) δ 11.00 (s, 1H), 9.23 (s, 1H), 7.51 (dd, J=10.2, 2.1 Hz, 1H), 7.34 (dd, J=7.5, 2.1 Hz, 1H), 5.13 (dd, J=13.3, 5.1 Hz, 1H), 4.32 (d, J=17.3 Hz, 1H), 4.17 (d, J=17.4 Hz, 1H), 3.29-3.25 (m, 1H), 3.16 (t, J=6.7 Hz, 2H), 2.97-2.83 (m, 1H), 2.64 (t, J=9.9 Hz, 4H), 2.57 (s, 1H), 2.49-2.39 (m, 2H), 2.15 (s, 3H), 2.06-1.99 (m, 1H), 1.95 (d, J=11.3 Hz, 3H), 1.91-1.83 (m, 3H), 1.61 (d, J=12.9 Hz, 10H), 1.42 (s, 2H), 1.38-1.26 (m, 4H). LCMS (ESI) calcd for C₃₁H₄₃FN₃O₃S⁺ [M+H]⁺: 556.30, found, 556.3.

### Example 13: Preparation of 3-(6-fluoro-1-oxo-4-((7-(spiro[3.3]heptan-2-ylamino)heptyl)thio)isoindolin-2-yl)piperidine-2,6-dione (GT-05422)

Referring to the method of Scheme 1, the target product (GT-05422) was prepared as white solid (13 mg, yield 19.02%). ¹H NMR (400 MHz, DMSO) δ 10.99 (s, 1H), 8.83 (s, 2H), 7.50 (dd, J=10.1, 2.1 Hz, 1H), 7.34 (dd, J=7.5, 2.1 Hz, 1H), 5.12 (dd, J=13.3, 5.1 Hz, 1H), 4.31 (d, J=17.3 Hz, 1H), 4.17 (d, J=17.2 Hz, 1H), 3.49 (s, 1H), 3.15 (t, J=7.1 Hz, 2H), 2.96-2.86 (m, 1H), 2.70 (s, 2H), 2.59 (d, J=17.4 Hz, 1H), 2.43 (dd, J=13.0, 8.7 Hz, 1H), 2.28-2.23 (m, 2H), 2.15-2.08 (m, 2H), 2.00 (dd, J=14.0, 6.7 Hz, 4H), 1.91 (d, J=7.6 Hz, 2H), 1.81-1.76 (m, 2H), 1.66-1.58 (m, 2H), 1.54 (s, 2H), 1.41 (s, 2H), 1.29 (s, 3H). LCMS (ESI) calcd for C₂₇H₃₇FN₃O₃S⁺ [M+H]⁺: 502.25, found, 502.3.

### Example 14: Preparation of 3-(4-((4-(((adamantan-1-yl)amino)methyl)benzyl)thio)-6-fluoro-1-oxoisoindolin-2-yl)piperidine-2,6-dione (GT-05423)

Referring to the method of Scheme 2, the target product (GT-05423) was prepared as white solid (8.8 mg, yield 35.87%). ¹H NMR (400 MHz, DMSO) δ 11.00 (s, 1H), 8.82 (s, 2H), 7.60 (d, J=9.1 Hz, 1H), 7.48 (d, J=9.2 Hz, 4H), 7.35 (d, J=7.2 Hz, 1H), 5.10 (dd, J=13.1, 5.0 Hz, 1H), 4.51-4.35 (m, 2H), 4.28 (d, J=17.5 Hz, 1H), 4.13 (d, J=17.4 Hz, 1H), 4.06 (s, 2H), 2.93-2.85 (m, 1H), 2.59 (d, J=17.0 Hz, 2H), 2.46-2.35 (m, 1H), 2.14 (s, 3H), 2.01 (s, 1H), 1.94 (s, 5H), 1.64 (dd, J=31.0, 12.0 Hz, 6H). LCMS (ESI) calcd for C₃₁H₃₅FN₃O₃S⁺ [M+H]⁺: 548.24, found, 548.2.

### Example 15: Preparation of 3-(6-fluoro-1-oxo-4-((4-((((1R,2S,4R)-1,7,7-trimethylbicyclo[2.2.1]heptan-2-yl)amino)methyl)benzyl)thio)isoindolin-2-yl)piperidine-2,6-dione (GT-05424)

Referring to the method of Scheme 2, the target product (GT-05424) was prepared as white solid (13 mg, yield 52.80%). ¹H NMR (400 MHz, DMSO) δ 11.00 (s, 1H), 9.04 (s, 1H), 8.59 (s, 1H), 7.53 (d, J=8.4 Hz, 3H), 7.47 (d, J=8.1 Hz, 2H), 7.34 (d, J=7.5 Hz, 1H), 5.11 (dd, J=13.4, 4.8 Hz, 1H), 4.47 (s, 2H), 4.28 (d, J=17.3 Hz, 1H), 4.13 (dd, J=16.3, 9.4 Hz, 3H), 3.06 (s, 1H), 2.89 (d, J=12.6 Hz, 1H), 2.64 (d, J=25.3 Hz, 3H), 2.48-2.34 (m, 2H), 2.03-1.95 (m, 1H), 1.87 (s, 1H), 1.63 (d, J=14.7 Hz, 3H), 1.36 (d, J=7.8 Hz, 2H), 1.08 (d, J=13.3 Hz, 1H), 0.86-0.78 (m, 6H), 0.68 (s, 3H). LCMS (ESI) calcd for C₃₁H37FN₃O₃S⁺ [M+H]⁺: 550.25, found, 550.3.

### Example 16: Preparation of 3-(6-fluoro-1-oxo-4-((7-(((1R,2S,4R)-1,7,7-trimethylbicyclo[2.2.1]heptan-2-yl)amino)heptyl)thio)isoindolin-2-yl)piperidine-2,6-dione (GT-05425)

Referring to the method of Scheme 1, the target product (GT-05425) was prepared as white solid (16 mg, yield 32.44%). ¹H NMR (400 MHz, DMSO) δ 11.00 (s, 1H), 8.62 (s, 1H), 8.21 (s, 1H), 7.56-7.44 (m, 1H), 7.34 (d, J=7.5 Hz, 1H), 5.13 (dd, J=13.4, 5.0 Hz, 1H), 4.32 (d, J=17.3 Hz, 1H), 4.17 (d, J=17.3 Hz, 1H), 3.17 (dd, J=16.4, 9.2 Hz, 3H), 2.89 (dd, J=12.4, 4.3 Hz, 3H), 2.59 (d, J=17.1 Hz, 2H), 2.49-2.38 (m, 2H), 2.19-2.11 (m, 1H), 2.05-1.96 (m, 1H), 1.64 (dd, J=16.2, 9.1 Hz, 7H), 1.40 (d, J=9.4 Hz, 4H), 1.30 (s, 4H), 1.25-1.20 (m, 1H), 0.96 (s, 3H), 0.84 (s, 6H). LCMS (ESI) calcd for C₃₀H₄₃FN₃O₃S⁺ [M+H]⁺: 544.30, found, 544.3.

### Example 17: Preparation of 3-(4-((4-(2-((adamantan-1-yl)amino)ethyl)benzyl)thio)-6-fluoro-1-oxoisoindolin-2-yl)piperidine-2,6-dione (GT-05431)

Referring to the method of Scheme 2, the target product (GT-05431) was prepared as white solid (3.3 mg, yield 20.43%). ¹H NMR (400 MHz, DMSO-d6) δ 11.01 (s, 1H), 8.71 (s, 1H), 7.60 (dd, J=10.0, 2.2 Hz, 1H), 7.36 (dd, J=11.2, 5.1 Hz, 3H), 7.27-7.16 (m, 2H), 5.11 (dd, J=13.3, 5.1 Hz, 1H), 4.47-4.36 (m, 2H), 4.19 (dd, J=49.7, 17.4 Hz, 2H), 3.05 (s, 2H), 2.95-2.80 (m, 3H), 2.58 (d, J=15.8 Hz, 1H), 2.41 (dd, J=12.8, 4.2 Hz, 1H), 2.10 (s, 3H), 2.04-1.95 (m, 1H), 1.85 (s, 6H), 1.62 (dd, J=28.1, 11.9 Hz, 6H). LCMS (ESI) calcd for C₃₂H₃₇FN₃O₃S⁺ [M+H]⁺: 562.25, found, 562.3.

### Example 18: Preparation of 3-(6-fluoro-4-((3-fluoro-4-(piperidin-1-ylmethyl)benzyl)thio)-1-oxoisoindolin-2-yl)piperidine-2,6-dione (GT-05443)

Referring to the method of Scheme 2, the target product (GT-05443) was prepared as white solid (15 mg, yield 73.64%). ¹H NMR (400 MHz, DMSO-d6) δ 11.01 (s, 1H), 10.15 (d, J=88.6 Hz, 1H), 7.66-7.60 (m, 1H), 7.53-7.46 (m, 1H), 7.45-7.39 (m, 1H), 7.36-7.29 (m, 1H), 5.10 (dd, J=13.3, 5.0 Hz, 1H), 4.44 (s, 1H), 4.35-4.17 (m, 3H), 4.10 (dd, J=17.2, 10.0 Hz, 1H), 3.36 (s, 1H), 3.22 (t, J=17.7 Hz, 3H), 2.99-2.70 (m, 3H), 2.58 (d, J=17.3 Hz, 1H), 2.46-2.31 (m, 1H), 2.02-1.90 (m, 1H), 1.76 (s, 3H), 1.69-1.64 (m, 1H), 1.33 (d, J=8.3 Hz, 1H). LCMS (ESI) calcd for C₂₆H₂₈F₂N₃O₃S⁺ [M+H]⁺: 500.18, found, 500.2.

### Example 19: Preparation of 3-(4-((4-((4,4-difluoropiperidin-1-yl)methyl)-3-fluorobenzyl)thio)-6-fluoro-1-oxoisoindolin-2-yl)piperidine-2,6-dione (GT-05444)

Referring to the method of Scheme 2, the target product (GT-05444) was prepared as white solid (15 mg, yield 69.01%). ¹H NMR (400 MHz, DMSO-d6) δ 11.17 (s, 1H), 11.01 (s, 1H), 7.73-7.58 (m, 1H), 7.55-7.44 (m, 1H), 7.40 (ddd, J=12.0, 7.5, 2.1 Hz, 1H), 7.33 (d, J=7.1 Hz, 1H), 5.11 (dd, J=13.3, 5.0 Hz, 1H), 4.46 (d, J=12.7 Hz, 2H), 4.36 (s, 1H), 4.33-4.20 (m, 1H), 4.14-3.95 (m, 1H), 3.14 (d, J=19.1 Hz, 2H), 2.98-2.82 (m, 1H), 2.58 (d, J=16.4 Hz, 1H), 2.47-2.36 (m, 2H), 2.33 (s, 2H), 2.06-1.87 (m, 1H). LCMS (ESI) calcd for C₂₆H₂₆F₄N₃O₃S⁺ [M+H]⁺: 536.16, found, 536.2.

### Example 20: Preparation of 3-(4-(8-((adamantan-1-yl)amino)octyl)-6-fluoro-1-oxoisoindolin-2-yl)piperidine-2,6-dione (GT-05617)

Referring to the method of Scheme 4, the target product (GT-05617) was prepared as white solid (8.8 mg, yield 49.09%). ¹H NMR (400 MHz, DMSO-d6) δ 11.03 (s, 1H), 8.54 (s, 2H), 7.42-7.29 (m, 2H), 5.14 (dd, J=13.3, 5.0 Hz, 1H), 4.45 (d, J=17.1 Hz, 1H), 4.30 (t, J=14.1 Hz, 1H), 2.92 (dd, J=21.6, 9.4 Hz, 1H), 2.81 (s, 2H), 2.69-2.59 (m, 3H), 2.47-2.34 (m, 1H), 2.11 (s, 3H), 2.05-1.97 (m, 1H), 1.84 (s, 6H), 1.66 (d, J=12.5 Hz, 4H), 1.58 (d, J=11.4 Hz, 6H), 1.30 (s, 8H). LCMS (ESI) calcd for C₃₁H₄₃FN₃O₃⁺ [M+H]⁺: 524.33, found, 524.4.

### Example 21: Preparation of 3-(4-((7-((adamantan-1-yl)amino)heptyl)amino)-6-fluoro-1-oxoisoindolin-2-yl)piperidine-2,6-dione (GT-05879)

Referring to the method of Scheme 6, the target product (GT-05879) was prepared as white solid (3.0 mg, yield 13.35%). ¹H NMR (400 MHz, DMSO-d6) δ 8.21 (s, 2H), 6.56 (dd, J=7.7, 2.2 Hz, 1H), 6.51 (dd, J=11.8, 2.2 Hz, 1H), 6.07-5.42 (m, 1H), 5.06 (dd, J=13.3, 5.1 Hz, 1H), 4.14 (d, J=17.1 Hz, 1H), 3.99 (d, J=16.8 Hz, 1H), 3.57 (s, 2H), 2.99-2.87 (m, 2H), 2.72 (s, 3H), 2.27-2.19 (m, 1H), 2.02 (d, J=15.0 Hz, 4H), 1.74 (s, 6H), 1.59 (d, J=12.9 Hz, 3H), 1.51 (d, J=12.0 Hz, 4H), 1.40-1.33 (m, 2H), 1.23 (d, J=10.4 Hz, 4H), 1.19 (d, J=6.6 Hz, 3H). LCMS (ESI) calcd for C₃₀H₄₂FN₄O₃+[M+H]⁺: 525.32, found, 525.4.

### Biological Activity Assay

### Materials

| Reagents and materials | Suppliers or Source |
|---|---|
| Human diffuse large B-cell | Kyinno Biotechnology Co., Ltd |
| lymphoma cell line: TMD8 | |
| Human multiple myeloma cells: | ATCC (American Type Culture |
| MM.1S | Collection) |
| Human mantle cell lymphoma cells: | ATCC |
| Mino | |
| Human Burkitt's lymphoma cell line: | Dalian Meilun Biotech Co., Ltd |
| Daudi | |
| RPMI-1640 Medium | Dalian Meilun Biotech Co., Ltd |
| Meilun Cell Counting Kit-8 | Dalian Meilun Biotech Co., Ltd |
| Fetal bovine serum (FBS) | Sunrise Science Products Company |
| Penicillin-Streptomycin | Beijing TransGen Biotech Co., Ltd |
| Mycoplasma detection kit | Beijing TransGen Biotech Co., Ltd |
| STR genotype detection | Shanghai Biowing Applied |
| | Biotechnology Co. Ltd |

### Methods

### Cell Cultures

The tumor cells used herein were cultured in RPMI 1640 medium supplemented with 10% FBS, penicillin to a final concentration of 100 U/mL, and streptomycin to a final concentration of 100 μg/mL, at 37° C. in an incubator with 5% CO₂. Prior to experimentation, all cells used were correctly identified by STR profiling, and tested negative for Mycoplasma through routine screenings.

### I. Determination of Half Inhibitory Concentration (IC₅₀) of the Compounds Against Tumor Cells

IC₅₀ values of the compounds of the present disclosure (including the compounds in Table 1, and Examples compounds) were measured using the Meilun Cell Counting Kit-8 kit from Dalian Meilun Biotech Co., Ltd. Assay details are as follows: the tumor cells were seeded into RPMI-1640 medium containing serum at a certain seeding density, wherein the seeding densities of the tumor cells are as follows: TMD8 cells, MM.1S cells, or Mino cells were seeded at a density of 10,000 cells/100 μL/well, and Daudi cells were seeded at a density of 4,000 cells/100 μL/well. After 24 h, 100 μL of the inoculated cells were treated with 0.5 μL of the compounds of the present disclosure (including the compounds in Table 1, and Examples compounds) in DMSO at 10 different successively decreasing concentrations (starting at a maximum concentration of 10 μM; 5-fold serial dilutions). After the cells were treated with the compounds of the present disclosure for 96 hours, cell viability was measured according to the instructions provided with the CCK-8 kit. DMSO served as the negative control, and corresponding commercial immunomodulatory drugs (including lenalidomide (CAS No.: 191732-72-6) and pomalidomide (CAS No.: 19171-19-8)) were used as the positive controls. All controls were treated using the same protocol as the compounds of the present disclosure. The growth inhibition of the compounds of the present disclosure on the cells was plotted using Prism Graphpad software, and the IC₅₀ values of the compounds of the present disclosure were calculated. The results were shown in Table 2 below.

TABLE 2
IC₅₀ values (in nM) of the compounds of the present disclosure
for inhibiting the proliferation of tumor cells

| Comp. No. | MM.1S cell | Mino cell | Daudi cell | TMD8 cell |
|---|---|---|---|---|
| lenalidomide | D | C | F | D |
| pomalidomide | B | B | D | B |
| GT-04117 | — | A+++ | C | A+ |
| GT-04024 | — | — | — | A++ |
| GT-05408 | — | A | — | — |
| GT-05343 | — | A+ | — | — |
| GT-05407 | A | A+ | — | — |
| GT-05409 | — | A+++ | — | — |
| GT-05421 | A+++ | A+++ | — | — |
| GT-05425 | A | A+++ | C | — |
| GT-05424 | A+ | A+++ | B | — |
| GT-05423 | A+++ | A+++ | — | — |
| GT-05422 | A+++ | A+++ | — | — |
| GT-05617 | — | A | — | — |
| GT-05444 | A+++ | A+++ | A+ | — |
| GT-05431 | A+++ | A+++ | A+++ | — |
| GT-05443 | A+ | A+++ | B | — |

Note:
In Table 2, the symbols A+++, A++, A+, A, B, C, D, and E are defined as follows: 0 nM < A+++ ≤ 5 nM; 5 nM < A++ ≤ 10 nM; 10 nM < A+ ≤ 50 nM; 50 nM < A ≤ 100 nM; 100 nM < B ≤ 500 nM; 500 nM < C ≤ 1000 nM; 1000 nM < D ≤ 5000 nM; 5000 nM < E ≤ 10000 nM; 10000 nM < F. The symbol “—” indicates not tested.

The results indicated that the compounds of the present invention (including the compounds listed in Table 1 and the Examples compounds) can inhibit the proliferation of tumor cells (as shown in Table 2), with inhibitory effects superior to or comparable to those of the corresponding positive control drugs, and degradation effects superior to those of the corresponding positive control drugs. Furthermore, the results demonstrated that the compounds of the present invention (including the compounds listed in Table 1 and the Examples compounds) significantly inhibit the proliferation of Mino cells (human mantle cell lymphoma cells), MM.1S cells (human multiple myeloma cells), Daudi cells (human Burkitt's lymphoma cells), and TMD8 cells (human diffuse large B-cell lymphoma cells) (as shown in Table 2), with inhibitory effects superior to those of the corresponding positive control drugs.

### II. Determination of CRBN-Binding Affinity of Compounds

The CEREBLON BINDING kits (specification: 10,000 tests; product number: Catalog #64BDCRBNPEH; CISBIO company) was used to determine the CRBN binding ability of the compounds to be tested through a HTRF method (homogeneous time-resolved fluorescence). The specific methods are as follows:
1. According to the instructions of the CEREBLON BINDING kits, the compounds of the present invention to be tested and lenalidomide were serially diluted using diluent #9 (1×) solution to obtain a final concentration of 2 μM for both the tested compounds and lenalidomide solution.
2. 2.5 μL of the above 2 μM of the tested compounds and lenalidomide solution, as well as the same volume of diluent #9 (1×) solution (solvent control group, Std0) were added to each well of a 96-well plate, respectively. Then, 2.5 μL of human Cereblon WT GST-tagged protein solution was added to each well. Finally, 5 μL of the thoroughly mixed Thalidomide-Re reagent and GST Eu antibody working solution were added to each of the aforementioned wells. The final concentration of the tested compounds and lenalidomide in each well is 0.5 μM.
3. The blank control wells were sequentially added with 2.5 μL of diluent #9 (1×) solution, 2.5 μL of PROTAC binding buffer, and 5 μL of thoroughly mixed Thalidomide-Re reagent and GST Eu antibody working solution.
4. After sealing and incubating the solutions in the aforementioned wells at room temperature for 3 hours, the absorbance values at emission wavelengths of 620 nm and 665 nm were detected by the HTRF method using a Spark microplate reader (V3.1 SP1).

The corresponding CRBN binding inhibition rate was calculated using the following method:

$${R}_{compound} = O D 665 {nm}_{compound} / O D 620 {nm}_{compound} - O D 665 {nm}_{control} / O D 620 {nm}_{control}$$

$${R}_{Std 0} = O D 665 {nm}_{Std 0} / O D 620 {nm}_{Std 0} - O D 665 {nm}_{control} / O D 620 {nm}_{control}$$

$$inhibition rate ( \% ) = ( 1 - {R}_{compound} / {R}_{Std 0} ) * 100$$

Note: OD 665 nm_{compound} is the absorbance value of each well of the compounds of the present disclosure to be tested at an emission wavelength of 665 nm.
OD 620 nm_{compound} is the absorbance value of each well of the compounds of the present disclosure to be tested at an emission wavelength of 620 nm.
OD 665 nm_{control} is the absorbance value of the blank control well at an emission wavelength of 665 nm.
OD 620 nm_{control} is the absorbance value of the blank control well at an emission wavelength of 620 nm.
OD 665 nm_{Std0} is the absorbance value of the solvent control well at an emission wavelength of 665 nm.
OD 620 nm_{Std0} is the absorbance value of the solvent control well at an emission wavelength of 620 nm.

The test results were shown in Table 3 below.

TABLE 3
HTRF inhibitory activity (IC₅₀, μM) of the compounds
of the present disclosure (including the compounds
in Table 1 and the Examples compounds)

| Comp. No. | HTRF IC₅₀ (μM) | Comp. No. | HTRF IC₅₀ (μM) |
|---|---|---|---|
| lenalidomide | e | GT-05425 | a |
| GT-04117 | a | GT-05424 | a |
| GT-04024 | a | GT-05423 | a |
| GT-04243 | a | GT-05422 | a |
| GT-05341 | a | GT-05617 | a |
| GT-05342 | a | GT-05444 | a |
| GT-05344 | a | GT-05431 | a |
| GT-05408 | a | GT-05443 | a |
| GT-05343 | a | GT-05409 | a |
| GT-05407 | a | GT-05421 | a |
| GT-04333 | a | | |

Note:
In Table 3, the symbols a, b, c, d, e, f, g, and E are defined as follows: 0 μM < a ≤ 0.6 μM; 0.6 μM < b ≤ 1 μM; 1 μM < c ≤ 1.5 μM; 1.5 μM < d ≤ 2.0 μM; 2.0 μM < e ≤ 2.5 μM; 2.5 μM < f ≤ 3 μM; g > 3 μM.

The results showed that the compounds of the present disclosure (including the compounds listed in Table 1 and the Examples compounds) exhibited strong binding affinity to CRBN.

### Iii. Western-Blot Assay

The effect of the compounds of the present disclosure on the expression of target proteins in cells was assessed using conventional Western blot analysis.

After culturing 1.5×10⁶ hPBMC cells for 24 hours, the cells were treated with the compounds of the present disclosure (including the compounds listed in Table 1 and the Examples compounds) at concentrations of 0.1 nM, 0.5 nM, 10 nM, 50 nM, 500 nM and 1000 nM. DMSO was used as the negative control, and commercial immunomodulatory drugs (lenalidomide and pomalidomide) were used as positive controls. All controls were administered to the cells following the same protocol as the compounds of the present disclosure. After 24 hours of treatment with the compounds, the cells were harvested and lysed using RIPA protein lysate supplemented with phosphatase inhibitors. The lysate was incubated on ice for 30-60 minutes and then centrifuged. The supernatant, containing the total cellular protein, was aspirated. The protein concentration of the supernatant was quantified using the conventional BCA (Bicinchoninic Acid) assay. Subsequently, the supernatant was mixed with 4×SDS sample loading buffer, denatured at 95° C. for 5 minutes, and then subjected to SDS-PAGE electrophoresis on a polyacrylamide gel. The separated proteins were then transferred to a nitrocellulose membrane (NC membrane). The membrane was blocked with blocking buffer at room temperature for 1-2 hours, followed by antibody incubation and detection according to the manufacturer's instructions (Cell Signaling Technology).

Half-Maximal Degradation Concentration value (the drug concentration required for degrading 50% of the target protein, abbreviated as DC₅₀) was determined as follows: the grayscale values of the Western blotting bands from the drug-treated samples were compared with those from the DMSO control. The DC₅₀ value was identified as the drug concentration at which the grayscale value of the Western blotting bands from the drug-treated sample was equal to half of that from the DMSO control.

The DC₅₀ value can also be calculated as follows: using ImageJ software to quantify the grayscale values of the Western blotting bands from the drug-treated samples, fitting the concentration-response curve between drug concentrations and grayscale values, and calculating the drug concentration corresponding to half of the grayscale value of the Western blotting band for the DMSO control from the fitted curve.

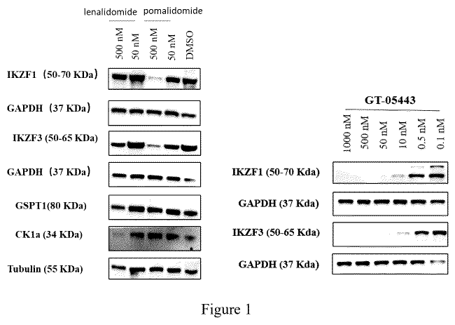

The results showed that the compounds of the present disclosure (including the compounds in Table 1 and Example compounds) can significantly degrade substrate proteins (e.g., IKZF1/3 proteins, GSPT1 proteins, etc.), particularly the compound GT-05443 of the present disclosure, which can significantly degrade IKZF3 protein at concentrations below 10 nM and IKZF1 protein at concentrations below 0.1 nM. In contrast, lenalidomide does not degrade IKZF1/3 proteins and GSPT1 protein at concentrations of 50 nM and 500 nM, and pomalidomide does not degrade IKZF1/3 proteins and GSPT1 protein at a concentration of 50 nM (FIG. 1). Western blotting results showed that, compared with lenalidomide and pomalidomide, the compounds of the present disclosure significantly improve activity in inhibiting tumor cells and inducing substrate protein degradation.

### Iv. Pharmacokinetic Study of the Compounds of the Present Disclosure

The pharmacokinetic properties of the compounds of the present disclosure were evaluated using conventional pharmacokinetic experimental methods.

The test compounds of the present disclosure (including the compounds listed in Table 1 and the Examples compounds) were orally administered to experimental animals to assess their pharmacokinetic properties. The experimental animals used can be those commonly used in pharmacokinetic experiments, e.g., adult and healthy rodents (e.g., mice, rats (such as Sprague-Dawley (SD) rats), guinea pigs) or non-rodents (rabbits, dogs, and monkeys). In the present disclosure, mice were used as the experimental animals.

The experimental animals were divided into two groups: a control group (for collecting blank plasma) and an orally administered group (dosed at 10 mg/kg or 30 mg/kg). Blood samples were collected at predetermined sampling time points after administration, such as 0.25 h, 0.5 h, 1 h, 2 h, 4 h, 8 h, and 24 h post-dosing. Blank plasma was collected from the control group.

Plasma samples were preprocessed before analysis: 10 μL of 50% acetonitrile and 200 μL of internal standard solution (containing 50 ng/mL dexamethasone in acetonitrile) were added to 10 μL of plasma samples. For the blank control group, the same volume of acetonitrile was added instead of the internal standard. The processed samples were vortexed, centrifuged, and the supernatant was taken for LC-MS/MS injection analysis.

After preparing standard curves and quality control (QC) samples, the concentrations of the compounds in the plasma samples were determined by LC-MS/MS analysis. Based on the plasma concentration data obtained from LC-MS/MS analysis, the pharmacokinetic parameters of the test compounds were calculated using the non-compartmental model in the pharmacokinetic calculation software WinNonlin v8.1. The results were listed in Table 4 below.

The results showed that the compounds of the present disclosure, administered orally, exhibited good pharmacokinetic (DMPK) properties and can be used as therapeutic agents for cancer patients.

### V. Effects of the Compounds of the Present Disclosure on TNF-α, IL-10, IL-6, IL-1β, IFN-γ, GM-CSF, IL-2, and IL-12p40 Release from PBMCs Induced by Lipopolysaccharide (LPS) or Phytohemagglutinin (PHA)

The following materials were used in this study:

### V.1 Reagents and Consumable Materials

| Name | Supplier or Source |
|---|---|
| Human PBMC cell | Shanghai Sai Li |
| Lipopolysaccharide (LPS) | Sigma |
| Phytohemagglutinin (PHA) | MCE |
| RPMI 1640 Medium | Gibco |
| Human TNF-alpha DuoSet ELISA | R&D Systems |
| Human IL-6 DuoSet ELISA | R&D Systems |
| Human IL-10 DuoSet ELISA | R&D Systems |
| Human IFN-gamma DuoSet ELISA | R&D Systems |
| Human IL-2 DuoSet ELISA | R&D Systems |
| Human IL-1 beta ELISA Kit | Abclonal |
| Human GM-CSF ELISA Kit | Abclonal |
| Human IL-12/IL-23 p40 ELISA Kit | Abclonal |

### V.2 Methods

### V.2.1 Cell Recovering and Plating

A complete medium was prepared and preheated to 37° C. Cells were retrieved from liquid nitrogen tank, and the frozen vial was held with forceps just below the cap-body junction, immersed in 37° C. warm water, and shaken occasionally to speed up thawing. The cell suspension was aspirated and added to a centrifuge tube containing 10 mL of the complete medium, and mixed well. The mixture was centrifuged at 400 g for 10 min at room temperature. The supernatant was discarded. The complete medium was added to the cells, followed by counting the cells and adjusting the cell density. The cells were seeded at 75 μL/well into a 96-well plate to achieve 1 X₁₀⁵ cells per well. The plate was incubated in a high-humidity, 37° C., 5% CO₂ incubator for 2 h.

### V.2.2 Preparation of Compounds and Addition to Cell Plates

1) 10 mM stock solutions of the test compounds in DMSO were prepared.
2) The test compounds were diluted stepwise 5-fold in DMSO, starting from 1 mM, to obtain nine concentration gradients.
3) The test compounds were diluted in medium to five times the final concentration intended for use.
4) The diluted compounds were added to the corresponding cell wells at 25 μL/well according to the following layout. The final compound concentrations start at 1 μM, with 5-fold dilutions, for a total of nine concentration points.
5) After 1 h, LPS or PHA was added to the cell wells at 25 μL/well to achieve final LPS concentrations of 1 μg/mL (for TNF-α, IL-10, IL-6, GM-CSF, and IL-12p40), 5 μg/mL (for IL-1β), 50 μg/mL (for IFN-γ), or a final PHA concentration of 1 μg/mL (for IL-2).
6) The cell plate was incubated in a high-humidity incubator at 37° C. with 5% CO₂ for 24 h.

Cell plate layout:

| | 1 | 2 | 3 | 4 | 5 | 6 | 7 | 8 | 9 | 10 | 11 | 12 |
|---|---|---|---|---|---|---|---|---|---|---|---|---|
| A | H₂O | H₂O | H₂O | H₂O | H₂O | H₂O | H₂O | H₂O | H₂O | H₂O | H₂O | H₂O |
| B | H₂O | Cmpd 1# (starting at a maximum concentration of 1 μM, | | | | | | | | | LPS/ | H₂O |
| C | H₂O | 5-fold serial dilutions, 9 points) | | | | | | | | | PHA | H₂O |
| D | H₂O | Cmpd 2# (starting at a maximum concentration of 1 μM, | | | | | | | | | | H₂O |
| E | H₂O | 5-fold serial dilutions, 9 points) | | | | | | | | | | H₂O |
| F | H₂O | Cmpd 3# (starting at a maximum concentration of 1 μM, | | | | | | | | | Blank | H₂O |
| G | H₂O | 5-fold serial dilutions, 9 points) | | | | | | | | | | H₂O |
| H | H₂O | H₂O | H₂O | H₂O | H₂O | H₂O | H₂O | H₂O | H₂O | H₂O | H₂O | H₂O |

### V.2.3 Detection

(1) The cell plate was centrifuged and the culture supermatant was aspirated. TNF-α, IL-10, IL-6, IL-1β, IFN-γ, GM-CSF, IL-2, and IL-12p40 cytokines in the culture supermatant were detected according to the ELISA kit instructions.
The concentrations of TNF-α, IL-10, IL-6, IL-1β, IFN-γ, GM-CSF, IL-2, and IL-12p40 in the samples were calculated based on the standard curve.

Inhibition rate %=[(Ac−As)/(Ac−Ab)]×10000
As: OA of the samples (cells+LPS/PHA+test compound)
Ac: OA of positive cell control (cells+LPS/PHA+DMS0)
Ab: OA of blank control (cells+culture medium+DMS0)
   (2) IC₅₀ value was calculated as follows: using GraphPad Prism 6 software and applying the calculation formula XY-analysis/Nonlinear regression (curve fit)/Dose response-Inhibition/log (inhibitor) vs. response-Variable slope (four parameters) to perform (C₅₀ curve fitting and determine the DC₅₀ values.

The experimental results indicated that the compounds of the present disclosure can modulate the production levels of cytokines associated with autoimmune diseases and may be used for the treatment of autoimmune diseases.

### Vi. Efficacy Experiment of the Compounds of the Present Disclosure on Psoriasis-Like Skin Lesions Induced by Imiquimod in Mice

The compounds of the present disclosure (test compounds) were administered orally via gavage once daily for 7 consecutive days to mice with psoriasis-like skin lesions induced by imiquimod, to investigate their efficacy on such lesions.

Ninety C₅₇BL/6J female mice (purchased from Shanghai SLAC Laboratory Animal Co., Ltd.) were divided into 9 groups based on their body weights, with 10 mice in each group:

| | | Number of | Dose | Volume | Concentration | Route and |
|---|---|---|---|---|---|---|
| Group | Drug | Animals | (mg/kg) | (mL/kg) | (mg/mL) | Duration |
| 1 | Control: Vehicle | 10 | NA | 10 | NA | ig. qd. 7 days |
| 2 | Model: Vehicle | 10 | NA | 10 | NA | ig. qd. 7 days |
| 3 | Positive Control | 10 | 3 | 10 | 0.3 | sc. qd. 7 days |
| 4 | Low-dose test | 10 | 3 | 10 | 0.3 | ig. qd. 7 days |
| | compounds group | | | | | |
| 5 | Medium-dose test | 10 | 10 | 10 | 1 | ig. qd. 7 days |
| | compounds group | | | | | |
| 6 | High-dose test | 10 | 30 | 10 | 3 | ig. qd. 7 days |
| | compounds group | | | | | |

Note:
ig. = oral gavage;
sc. = subcutaneous injection

After grouping the experimental animals, the backs of all mice were shaved. Except for the Control group, the remaining groups were topically treated with imiquimod cream once daily for 7 consecutive days. Simultaneously, each group was administered with a solvent control, a positive control drug, or the test compounds, according to the above table, once daily for 7 consecutive days.

All clinical symptoms of each animal were observed at the beginning and throughout the experiment. The animals' weights were measured and recorded every 2 days.

### Scoring

Gross Observation Scoring of Skin Lesions (PASI Method): during the last 3 days of modeling and drug administration, the changes in skin lesions of mice in each group were observed once daily. PASI Method: the erythema, scaling, and thickening/infiltration of the lesions were scored separately, and the scores were summed to obtain the total score. PASI Scoring Criteria: 0=none; 1=mild; 2=moderate; 3=severe; 4=very severe.

The skin lesions were photographed once daily during the last 3 days of modeling and drug administration.

After the experiment, the mice were euthanized by CO₂ inhalation. Three different areas of skin from the lesion site on the back were collected using a 6 mm punch, weighed, and recorded, and the average weight was calculated.

The skin tissue from the lesion site was homogenized and the levels of IL-23p19, IL-12p40, IL-17, and TNF-α were determined.

The local lesion tissue was fixed, stained with HE, and subjected to pathological scoring (based on epidermal thickness, degree of epidermal keratinization, thickness of the basal layer, and degree of dermal inflammatory cell infiltration). Scoring Criteria: 0=none; 1=mild; 2=moderate; 3=severe; 4=very severe.

Experimental data were presented as Mean±SD. Statistical analysis between two groups was performed using SPSS, with p<0.05 considered statistically significant.

The experimental results indicated that the compounds of the present disclosure improve psoriasis-like skin lesions on the back of mice induced by imiquimod and may be used for the treatment of psoriasis.

### VII. Efficacy Experiment of the Compounds of the Present Disclosure on the CIA Model in Rats Induced by Type II Collagen

The compounds of the present disclosure (test compounds) were administered orally via gavage once daily for 21 consecutive days to Wistar rats with collagen-induced arthritis (CIA) that was induced using bovine type II collagen (Chondrex, Inc.), in order to investigate their efficacy in the rat arthritis model.

Preparation of the Modeling Agent: 10 mg of CII (Immunization Grade Bovine Type II Collagen, purchased from Chondrex, Inc.) was dissolved in 5 mL of 0.05M acetic acid solution, allowing it to be fully dissolved, thereby preparing a solution with a concentration of 2 mg/mL, which was then stored at 4° C. in the dark overnight. On the day of the experiment, the 2 mg/mL CII solution was mixed with an equal volume of a 4 mg/mL solution of Complete Freund's Adjuvant (CFA, also purchased from Chondrex, Inc.) on ice, and was thoroughly emulsified into an emulsion.

On day 0, 10 Wistar rats (purchased from Beijing Vital River Laboratory Animal Technology Co., Ltd.) were randomly selected as the blank control group and were not immunized. The remaining 100 Wistar rats were intradermally injected at the base of the tail with an emulsion prepared by mixing equal volumes of CII (2 mg/mL) and CFA (4 mg/mL) for the first immunization. A second booster immunization was administered to them on day 7. Between days 10 and 14, i.e., 3 to 7 days after the second booster immunization, 80 rats with an arthritis index (AI) score ranging from 1 to 2 were selected from the modeled animals and were divided into 8 groups based on their body weight, foot volume, and AI score, with 10 rats per group:

| | | Number of | Dose | Volume | Concentration | Route and |
|---|---|---|---|---|---|---|
| Group | Drug | Animals | (mg/kg) | (mL/kg) | (mg/mL) | Duration |
| 1 | Control: Vehicle | 10 | NA | 10 | NA | ig. qd. 21 days |
| 2 | Model: Vehicle | 10 | NA | 10 | NA | ig. qd. 21 days |
| 3 | Positive Control | 10 | 0.1 | 10 | 0.01 | sc. qd. 21 days |
| 4 | Low-dose test | 10 | 3 | 10 | 0.3 | ig. qd. 21 days |
| | compounds group | | | | | |
| 5 | Medium-dose test | 10 | 10 | 10 | 1 | ig. qd. 21 days |
| | compounds group | | | | | |
| 6 | High-dose test | 10 | 30 | 10 | 3 | ig. qd. 21 days |
| | compounds group | | | | | |

Note:
ig. = oral gavage;
sc. = subcutaneous injection

Between 3 and 7 days after the second booster immunization, when the AI score of the affected feet reached 1 to 2, a solvent control, a positive control drug, or the test compounds were administered orally via gavage (or subcutaneously) to the animals in each group, according to the above table, once daily for 21 consecutive days.

Starting 3 days after the second booster immunization, the volume of both hind paws (or toes) was measured and recorded twice a week.

Foot Volume Measurement: Before the measurement, a line was marked on the rat's ankle joint with a marker pen to indicate the position. After clean water was added to the instrument, the instrument's reading was reset to zero in preparation for the measurement. The hind limb of the rat was placed in the water so that the marked line at the ankle joint was at the surface of the liquid. The reading obtained by pressing the foot pedal at this time was the foot volume of the rat. After the measurement was completed, the foot pedal was pressed again to reset the instrument to zero, preparing it for the measurement of the next rat.

### Scoring

Arthritis Index (AI): The foot volume was measured twice a week, and the swelling of the four toes was scored simultaneously. Each foot was scored on a scale from 0 to 4, with a maximum score of 16 for each rat.

Arthritis Index (AI) Scoring Criteria:

| Score | Degree |
|---|---|
| 0 | No swelling, normal appearance |
| 1 | Mild swelling or redness of the ankle, wrist, or finger joints |
| 2 | Moderate swelling or redness of the ankle, wrist, or finger joints |
| 3 | Severe swelling or redness of the ankle, wrist, or finger joints |
| 4 | Very severe swelling or redness of the ankle, wrist, or finger joints |

The day of starting drug administration is recorded as DO. Five days after starting drug administration, i.e., 2 hours after D5 administration, blood was collected from the jugular vein of the experimental animals, allowed to stand for more than 30 minutes, centrifuged to separate the serum, which was stored at −80° C. The levels of TNF-α, IL-1, and IL-6 in serum were measured by ELISA.

After the experiment, the animals were euthanized by CO₂ inhalation. The spleen and thymus of the animals were collected and weighed, and the organ coefficients were calculated.

Pathological Examination: One side of the foot joint tissue was fixed in 10% neutral formalin solution, followed by decalcification and paraffin embedding to prepare pathological sections. The sections were then stained with hematoxylin and eosin (HE) for pathological observation.

### Observation Indices:

1. Infiltration of synovial inflammatory cells (lymphocytes, plasma cells);
2. Synovial tissue hyperplasia;
3. Synovial pannus;
4. Fibrinoid necrosis on the synovial surface.
   Scoring and Counting Method: 0, 1, 2, 3, and 4 levels, where “0” indicates no observation; “1” is mild; “2” is moderate; “3” is severe; and “4” is extremely severe.

Experimental data were presented as Mean±SEM. Data between two groups were analyzed using Excel-T test, with p<0.05 considered statistically significant. Scoring data were analyzed using the non-parametric Mann-Whitney U test in SPSS, with p<0.05 considered statistically significant.

The experimental results indicated that the compounds of the present disclosure can regulate the serum levels of inflammatory cytokines in rats with collagen-induced arthritis (CIA) model that was induced using bovine type II collagen, and significantly improve the swelling of rat limbs, and thus might be used for the treatment of arthritis.

The basic principles, main features and advantages of the present disclosure are shown and described above. Those skilled in the art should understand that the present disclosure is not limited by the foregoing embodiments and they can make various changes, substitutions and alterations herein without departing from the spirit and scope of the present disclosure. These changes, substitutions and alterations fall within the scope of the present disclosure. The claimed scope of the present disclosure is defined by the appended claims and their equivalents.

1. A compound of Formula (I) 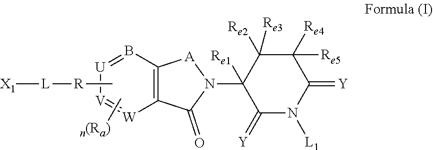
or salts, enantiomers, diastereoisomer, isotopically enriched analogs, solvates, prodrugs, or polymorphs thereof,
wherein
A represents CH₂ or C(O);
B, U, V, and W are the same or different and each independently represent CH or N, where B, U, V, and W are not N at the same time;
Y represents O or S;
L₁ represents hydrogen or C₁₋₃ alkyl;
Rₑ₁, Rₑ₂, Rₑ₃, Rₑ₄, and Rₑ₅ are the same or different and each independently represent hydrogen or halogen;
(Rₐ)ₙ denotes that the ring containing B, U, V, and W is substituted with n Rₐ, where Rₐ groups are the same or different and each independently represent deuterium, halogen, hydroxy, mercapto, nitro, amino, cyano, optionally deuterated C₁₋₆ alkyl, optionally deuterated C₁₋₆ alkoxy, halogenated C₁₋₆ alkyl, optionally substituted C₃₋₆ cycloalkyl, C₂₋₆ alkenyl, or C₂₋₆ alkynyl, and n represents an integer of 1, 2, or 3; and
R represents SH, and L and X₁ are absent; or
R represents S, L represents an optionally substituted linear or branched alkyl, and X₁ is absent; or
R represents S(O) or S(O)₂, L represents an optionally substituted linear or branched alkyl or amino, and X₁ is absent; or
R represents S, S(O), S(O)₂, O, NH or CH₂,
L represents optionally substituted linear or branched alkylene, wherein one or more pairs of adjacent carbon atoms in the backbone carbon chain of the linear or branched alkylene is optionally interrupted by one or more groups selected from the group consisting of: R_{b}, R_{c}, and any combination thereof, wherein R_{b} groups are each selected from the group consisting of O, C(O), OC(O), S, S(O), S(O)₂, S(O)₂N(R₁), N(R₁)S(O)₂, C(O)N(R₁), N(R₁)C(O), N(R₁), and N(R₁)C(O)N(R₁), where R₁ groups each independently represent H or C₁₋₃ alkyl, and when two or more groups R_{b} are inserted into the backbone carbon chain of the linear or branched alkylene group, the two or more groups R_{b} are not directly connected to each other; and wherein R_{c} groups are each selected from the group consisting of optionally substituted cycloalkylene, optionally substituted arylene, optionally substituted heterocyclylene, optionally substituted heteroarylene, and any combination thereof, and
X₁ represents NR₂R₃, C(O)NR₄R₅, NHC(O)R₆, NHC(O)NR₇R₈, OR₉, SR₁₀, quaternary ammonium salt group, linear or branched alkyl optionally substituted with one or more fluorine atoms, optionally substituted cycloalkyl, optionally substituted aryl, optionally substituted heterocyclyl or optionally substituted heteroaryl, where R₂, R₃, R₄, R₅, R₇, R₈, R₉, and R₁₀ each independently represent H, optionally substituted linear or branched alkyl, optionally substituted cycloalkyl, optionally substituted aryl, optionally substituted heterocyclyl or optionally substituted heteroaryl, wherein R₂ and R₃ are not H at the same time, and wherein R₆ represents optionally substituted linear or branched alkyl, optionally substituted cycloalkyl, optionally substituted aryl, optionally substituted heteroaryl or optionally substituted heterocyclyl, or wherein R₄ and R₅ together with the nitrogen atom to which they are attached form optionally substituted 5- to 8-membered heterocyclyl; or
X₁ represents a group of Formula (G₁): 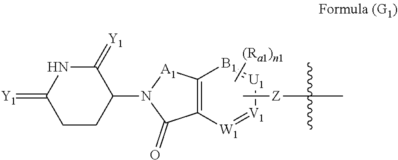
wherein A₁ represents CH₂ or C(O);
B₁, U₁, V₁, and W₁ are the same or different and each independently represent CH or N, wherein
B₁, U₁, V₁, and W₁ are not N at the same time;
Y₁ represents O or S; and
Z represents S, S(O), S(O)₂, O, NH or CH₂; and
(Rₐ₁)ₙ₁ denotes that the ring containing B₁, U₁, V₁, and W₁ is substituted with n1 Rₐ₁, where Rₐ₁ groups are the same or different and each independently represent deuterium, halogen, hydroxy, mercapto, nitro, amino, cyano, optionally deuterated C₁₋₆ alkyl, optionally deuterated C₁₋₆ alkoxy, halogenated C₁₋₆ alkyl, optionally substituted C₃₋₆ cycloalkyl, C₂₋₆ alkenyl, or C₂₋₆ alkynyl, and n represents an integer of 0, 1, 2, or 3.

2. The compound of Formula (I) or salts, enantiomers, diastereoisomer, isotopically enriched analogs, solvates, prodrugs, or polymorphs thereof of claim 1,
(i) wherein the compound of Formula (I) is also of Formula (Ia), Formula (Ib), Formula (Ic), Formula (Id), or Formula (Ie): 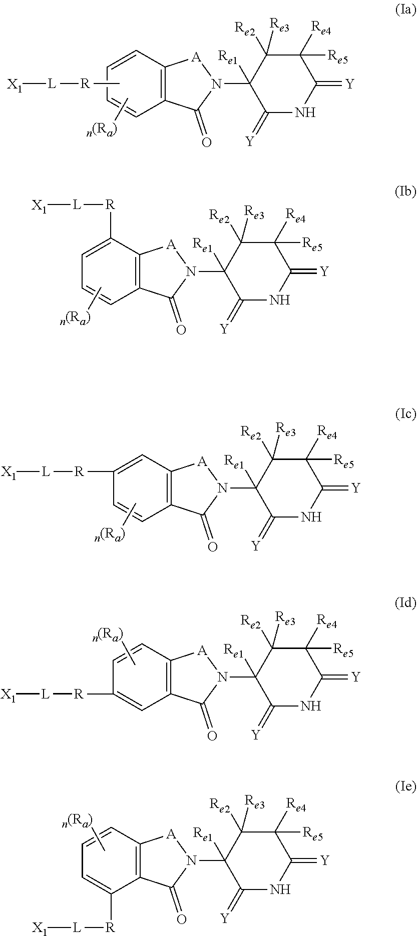
wherein A, (Rₐ)ₙ, Rₑ₁, Rₑ₂, Rₑ₃, Rₑ₄, Rₑ₅, R, L, X₁, and Y are as defined in claim 1; or
(ii) wherein Y represents 0;
(iii) wherein A represents C(O); or
(iv) wherein A represents CH₂.

3. The compound of Formula (I) or salts, enantiomers, diastereoisomer, isotopically enriched analogs, solvates, prodrugs, or polymorphs thereof of claim 1, wherein
R represents SH, and L and X₁ are absent; or
R represents S, L represents optionally substituted linear or branched C₁₋₄₀alkyl, and X₁ is absent; or
R represents S(O) or S(O)₂, L represents optionally substituted linear or branched C₁₋₄₀alkyl or amino, and X₁ is absent.

4. The compound of Formula (I) or salts, enantiomers, diastereoisomer, isotopically enriched analogs, solvates, prodrugs, or polymorphs thereof of claim 1, wherein
R represents S, L represents optionally substituted linear or branched C₁₋₃₀ alkyl, and X₁ is absent; or
R represents S(O) or S(O)₂, L represents optionally substituted linear or branched C₁₋₃₀ alkyl or amino, and X₁ is absent.

5. The compound of Formula (I) or salts, enantiomers, diastereoisomer, isotopically enriched analogs, solvates, prodrugs, or polymorphs thereof of claim 1, wherein
R represents S, L represents the following optionally substituted groups: methyl, ethyl, n-propyl, isopropyl, n-butyl, isobutyl, sec-butyl, tert-butyl, pentyl, isopentyl, neopentyl, tert-amyl, hexyl, heptyl, octyl, nonyl, decyl, undecyl, dodecyl, tridecyl, tetradecyl, or pentadecyl, and X₁ is absent; or
R represents S(O) or S(O)₂, L represents the following optionally substituted groups: methyl, ethyl, n-propyl, isopropyl, n-butyl, isobutyl, sec-butyl, tert-butyl, pentyl, isopentyl, neopentyl, tert-amyl, hexyl, heptyl, octyl, nonyl, decyl, undecyl, dodecyl, tridecyl, tetradecyl, pentadecyl, or amino, and X₁ is absent.

6. The compound of Formula (I) or salts, enantiomers, diastereoisomer, isotopically enriched analogs, solvates, prodrugs, or polymorphs thereof of claim 1, wherein
R represents S, S(O), S(O)₂, O, NH or CH₂;
L represents optionally substituted linear or branched C₁₋₄₀ alkylene, wherein one or more pairs of adjacent carbon atoms in the backbone carbon chain of the linear or branched C₁₋₄₀ alkylene is optionally interrupted by one or more groups selected from the group consisting of: R_{b}, R_{c}, and any combination thereof, wherein R_{b} groups are each selected from the group consisting of O, C(O), OC(O), S, S(O), S(O)₂, S(O)₂N(R₁), N(R₁)S(O)₂, C(O)N(R₁), N(R₁)C(O), N(R₁), and N(R₁)C(O)N(R₁), wherein R₁ groups each independently represent H or C₁₋₃ alkyl, and when two or more groups R_{b} are inserted into the backbone carbon chain of the linear or branched C₁₋₄₀ alkylene group, the two or more groups R_{b} are not directly connected to each other; and wherein R_{c} groups are each selected from the group consisting of optionally substituted cycloalkylene, optionally substituted arylene, optionally substituted heterocyclylene, optionally substituted heteroarylene, and any combination thereof; and
X₁ represents NR₂R₃, C(O)NR₄R₅, NHC(O)R₆, NHC(O)NR₇R₈, OR₉, SR₁₀, quaternary ammonium salt group, linear or branched C₁₋₁₀ alkyl optionally substituted with one or more fluorine, optionally substituted cycloalkyl, optionally substituted aryl, optionally substituted heterocyclyl or optionally substituted heteroaryl, where R₂, R₃, R₄, R₅, R₇, R₈, R₉, and R₁₀ each independently represent H, optionally substituted linear or branched C₁₋₁₀alkyl, optionally substituted cycloalkyl, optionally substituted aryl, optionally substituted heterocyclyl or optionally substituted heteroaryl, wherein R₂ and R₃ are not H at the same time, and wherein R₆ represents optionally substituted linear or branched C₁₋₈ alkyl, optionally substituted cycloalkyl, optionally substituted aryl, optionally substituted heteroaryl or optionally substituted heterocyclyl, or wherein R₄ and R₅ together with the nitrogen atom to which they are attached form optionally substituted 5- to 8-membered heterocyclyl; or
X₁ represents a group of Formula (G₁): 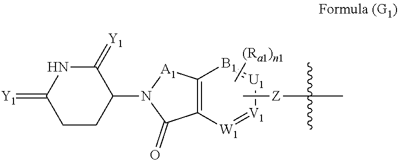
wherein A₁ represents CH₂ or C(O);
B₁, U₁, V₁, and W₁ are the same or different and each independently represent CH or N, wherein B₁, U₁, V₁, and W₁ are not N at the same time;
Y₁ represents O or S; and
Z represents S, S(O) or S(O)₂; and
(Rₐ₁)ₙ₁ denotes that the ring containing B₁, U₁, V₁, and W₁ is substituted with n1 Rₐ₁, where Rₐ₁ groups are the same or different and each independently represent deuterium, halogen, hydroxy, mercapto, nitro, amino, cyano, optionally deuterated C₁₋₆ alkyl, optionally deuterated C₁₋₆ alkoxy, halogenated C₁₋₆ alkyl, optionally substituted C₃₋₆ cycloalkyl, C₂₋₆ alkenyl, or C₂₋₆ alkynyl, and n represents an integer of 0, 1, 2, or 3.

7. The compound of Formula (I) or salts, enantiomers, diastereoisomer, isotopically enriched analogs, solvates, prodrugs, or polymorphs thereof of claim 1, wherein
R represents S, S(O), S(O)₂, O, NH or CH₂;
L represents linear or branched C₁₋₄₀ alkylene group optionally substituted with one or more substituents selected from the group consisting of halogen, cyano, hydroxy, amino, mercapto, C₁₋₃ alkyl, C₁₋₃ alkoxy, halogenated C₁₋₃ alkyl (e.g., trifluoromethyl), C₅₋₁₀ aryl, C₅₋₁₀ heteroaryl, C₃₋₁₂ cycloalkyl, 3- to 12-membered heterocyclyl, or any combination thereof, wherein one or more pairs of adjacent carbon atoms in the backbone carbon chain of the linear or branched C₁₋₄₀ alkylene is optionally interrupted by one or more groups selected from the group consisting of: R_{b}, R_{c}, and any combination thereof, wherein R_{b} groups are each selected from the group consisting of O, C(O), OC(O), S, S(O), S(O)₂, S(O)₂N(R₁), N(R₁)S(O)₂, C(O)N(R₁), N(R₁)C(O), N(R₁), and N(R₁)C(O)N(R₁), where R₁ groups each independently represent H or C₁₋₃ alkyl, and when two or more groups R_{b} are inserted into the backbone carbon chain of the linear or branched alkylene group, the two or more groups R_{b} are not directly connected to each other; and wherein R_{c} groups are each selected from the group consisting of cycloalkylene (e.g., C₃₋₂₀ cycloalkylene), arylene (e.g., C₅₋₂₀ arylene), heterocyclylene (e.g., 4- to 20-membered heterocyclylene), heteroarylene (e.g., 5- to 20-membered heteroarylene), and any combination thereof, wherein the cycloalkylene and heterocyclylene are each independently optionally substituted with one or more substituents selected from the group consisting of: halogen, cyano, hydroxy, amino, mercapto, oxo, C₁₋₃ alkoxy, halogenated C₁₋₃ alkyl, methanesulfonyloxy, trifluoromethanesulfonyloxy, p-toluenesulfonyloxy, optionally deuterated C₁₋₆ alkyl, optionally deuterated C₃₋₆ cycloalkyl, or any combination thereof; and the arylene and heteroarylene are each independently optionally substituted with one or more substituents selected from the group consisting of: halogen, cyano, hydroxy, amino, mercapto, C₁₋₃ alkoxy, halogenated C₁₋₃ alkyl, methanesulfonyloxy, trifluoromethanesulfonyloxy, p-toluenesulfonyloxy, optionally deuterated C₁₋₆ alkyl, optionally deuterated C₃₋₆ cycloalkyl, or any combination thereof; and
X₁ represents:
NR₂R₃, C(O)NR₄R₅, NHC(O)R₆, NHC(O)NR₇R₈, OR₉, SR₁₀, or quaternary ammonium salt group, where R₂, R₃, R₄, R₅, R₇, R₈, R₉, and R₁₀ each independently represent H, optionally substituted linear or branched C₁₋₁₀ alkyl, optionally substituted cycloalkyl, optionally substituted aryl, optionally substituted heterocyclyl or optionally substituted heteroaryl, wherein R₂ and R₃ are not H at the same time, and wherein R₆ represents optionally substituted linear or branched C₁₋₆ alkyl, optionally substituted cycloalkyl, optionally substituted aryl, optionally substituted heteroaryl or optionally substituted heterocyclyl, or wherein R₄ and R₅ together with the nitrogen atom to which they are attached form optionally substituted 5- to 8-membered heterocyclyl; or
linear or branched C₁₋₁₀ alkyl optionally substituted with one or more fluorine; or
C₃-C₁₂ cycloalkyl, optionally substituted with one or more substituents selected from the group consisting of: halogen, cyano, amino, hydroxy, mercapto, oxo, C₁₋₃ alkoxy, halogenated C₁₋₃ alkoxy, halogenated C₁₋₃alkyl, C₁₋₆ alkylsulfonyl, bis(C₁₋₆ alkyl)phosphono, methanesulfonyloxy, trifluoromethanesulfonyloxy, p-toluenesulfonyloxy, optionally substituted 5- or 6-membered heterocyclyl, optionally substituted C₅-C₂₀ aryl, optionally substituted 5- or 6-membered heteroaryl, C₁₋₆ alkyl, C₁₋₃ alkoxycarbonyl, halogenated C₂₋₄ alkenyl, or any combination thereof, wherein the 5- or 6-membered heterocyclyl is optionally substituted with one or more substituents selected from the group consisting of: halogen, cyano, amino, hydroxy, mercapto, oxo, C₁₋₆ alkyl, halogenated C₁₋₃alkyl, or any combination thereof; and the C₅-C₂₀ aryl is optionally substituted with one or more substituents selected from the group consisting of: halogen, cyano, amino, hydroxy, mercapto, C₁₋₆ alkyl, halogenated C₁₋₃ alkyl, or any combination thereof; and the 5- or 6-membered heteroaryl is optionally substituted with one or more substituents selected from the group consisting of: halogen, cyano, amino, hydroxy, mercapto, oxo, C₁₋₆ alkyl, halogenated C₁₋₃ alkyl, or any combination thereof; or
C₅-C₁₄ aryl, optionally substituted with one or more substituents selected from the group consisting of: halogen, cyano, amino, hydroxy, mercapto, C₁₋₃ alkoxy, halogenated C₁₋₃ alkoxy, halogenated C₁₋₃alkyl, C₁₋₆ alkylsulfonyl, bis(C₁₋₆ alkyl)phosphono, methanesulfonyloxy, trifluoromethanesulfonyloxy, p-toluenesulfonyloxy, optionally substituted 5- or 6-membered heterocyclyl, optionally substituted C₅-C₂₀ aryl, optionally substituted 5- or 6-membered heteroaryl, C₁₋₆ alkyl, C₁₋₃ alkoxycarbonyl, halogenated C₂₋₄ alkenyl, or any combination thereof, wherein the 5- or 6-membered heterocyclyl is optionally substituted with one or more substituents selected from the group consisting of: halogen, cyano, amino, hydroxy, mercapto, oxo, C₁₋₆ alkyl, halogenated C₁₋₃ alkyl, or any combination thereof; and the C₅-C₂₀ aryl is optionally substituted with one or more substituents selected from the group consisting of: halogen, cyano, amino, hydroxy, mercapto, C₁₋₆ alkyl, halogenated C₁₋₃ alkyl, or any combination thereof; and the 5- or 6-membered heteroaryl is optionally substituted with one or more substituents selected from the group consisting of: halogen, cyano, amino, hydroxy, mercapto, oxo, C₁₋₆ alkyl, halogenated C₁₋₃ alkyl, or any combination thereof; or
3- to 12-membered heterocyclyl, optionally substituted with one or more substituents selected from the group consisting of: halogen, cyano, amino, hydroxy, mercapto, oxo, C₁₋₃ alkoxy, halogenated C₁₋₃ alkoxy, halogenated C₁₋₃ alkyl, C₁₋₆ alkylsulfonyl, bis(C₁₋₆ alkyl)phosphon, methanesulfonyloxy, trifluoromethanesulfonyloxy, p-toluenesulfonyloxy, optionally substituted 5- or 6-membered heterocyclyl, optionally substituted C₅-C₂₀ aryl, optionally substituted 5- or 6-membered heteroaryl, C₁₋₆ alkyl, C₁₋₃ alkoxycarbonyl, halogenated C₂₋₄ alkenyl, or any combination thereof, wherein the 5- or 6-membered heterocyclyl is optionally substituted with one or more substituents selected from the group consisting of: halogen, cyano, amino, hydroxy, mercapto, oxo, C₁₋₆ alkyl, halogenated C₁₋₃ alkyl, or any combination thereof; and the C₅-C₂₀ aryl is optionally substituted with one or more substituents selected from the group consisting of: halogen, cyano, amino, hydroxy, mercapto, C₁₋₆ alkyl, halogenated C₁₋₃ alkyl, or any combination thereof; and the 5- or 6-membered heteroaryl is optionally substituted with one or more substituents selected from the group consisting of: halogen, cyano, amino, hydroxy, mercapto, oxo, C₁₋₆ alkyl, halogenated C₁₋₃ alkyl, or any combination thereof; or
5- to 10-membered heteroaryl, optionally substituted with one or more substituents selected from the group consisting of: halogen, cyano, amino, hydroxy, mercapto, C₁₋₃ alkoxy, halogenated C₁₋₃ alkoxy, halogenated C₁₋₃ alkyl, bis(C₁₋₆ alkyl)phosphon, C₁₋₆ alkylsulfonyl, methanesulfonyloxy, trifluoromethanesulfonyloxy, p-toluenesulfonyloxy, C₁₋₆ alkyl, optionally substituted 5- or 6-membered heterocyclyl, optionally substituted C₅-C₂₀ aryl, optionally substituted 5- or 6-membered heteroaryl, C₁₋₆ alkyl, C₁₋₃ alkoxycarbonyl, halogenated C₂₋₄ alkenyl, or any combination thereof, wherein the 5- or 6-membered heterocyclyl is optionally substituted with one or more substituents selected from the group consisting of: C₁₋₆ alkyl, cyano, halogenated C₁₋₃ alkyl, amino, hydroxy, mercapto, halogen, or any combination thereof; and the C₅-C₂₀ aryl is optionally substituted with one or more substituents selected from the group consisting of: C₁₋₆ alkyl, cyano, halogenated C₁₋₃alkyl, amino, hydroxy, mercapto, halogen, or any combination thereof; and the 5- or 6-membered heteroaryl is optionally substituted with one or more substituents selected from the group consisting of: C₁₋₆ alkyl, halogenated C₁₋₃ alkyl, amino, hydroxy, mercapto, halogen, or any combination thereof; or
X₁ represents a group of Formula (G₁): 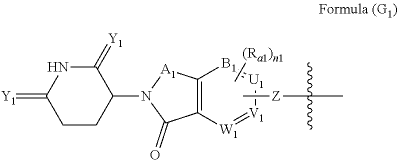
wherein A₁ represents CH₂ or C(O);
B₁, U₁, V₁, and W₁ are the same or different and each independently represent CH or N, wherein B₁, U₁, V₁, and W₁ are not N at the same time;
Y₁ represents O or S; and
Z represents S, S(O) or S(O)₂; and
(Rₐ₁)ₙ₁ denotes that the ring containing B₁, U₁, V₁, and W₁ is substituted with n1 Rₐ₁, where Rₐ₁ groups are the same or different and each independently represent deuterium, halogen, hydroxy, mercapto, nitro, amino, cyano, optionally deuterated C₁₋₆ alkyl, optionally deuterated C₁₋₆ alkoxy, halogenated C₁₋₆ alkyl, optionally substituted C₃₋₆ cycloalkyl, C₂₋₆ alkenyl, or C₂₋₆ alkynyl, and n represents an integer of 0, 1, 2, or 3, wherein the C₃₋₆ cycloalkyl is optionally substituted with a substituent selected from the group consisting of: C₁₋₆ alkyl, halogenated C₁₋₃ alkyl, amino, hydroxy, halogen, or any combination thereof.

8. The compound of Formula (I) or salts, enantiomers, diastereoisomer, isotopically enriched analogs, solvates, prodrugs, or polymorphs thereof of claim 1,
(i) wherein X₁ represents:
NR₂R₃, NHC(O)NR₇R₈, OR₉ or SR₁₀, wherein R₂, R₃, R₇, R₈, R₉, and R₁₀ each independently represent H, optionally substituted linear or branched C₁₋₁₀ alkyl, optionally substituted C₃-C₂₀ cycloalkyl, optionally substituted 3- to 20-membered heterocyclyl, optionally substituted C₅-C₁₄ aryl or optionally substituted 5- to 20-membered heteroaryl, wherein R₂ and R₃ are not H at the same time,
wherein the optionally substituted linear or branched C₁₋₁₀ alkyl is optionally substituted with one or more substituents selected from the group consisting of: halogen, cyano, hydroxy, amino, mercapto, C₁₋₃alkyl, C₁₋₃ alkoxy, trifluoromethyl, optionally substituted C₅₋₁₀ aryl, optionally substituted 5- to 10-membered heteroaryl, optionally substituted C₃₋₁₂ cycloalkyl, optionally substituted 3- to 12-membered heterocyclyl, or any combination thereof, wherein the optionally substituted C₅₋₁₀ aryl, optionally substituted 5- to 10-membered heteroaryl, optionally substituted C₃₋₁₂ cycloalkyl and optionally substituted 3- to 12-membered heterocyclyl are each independently optionally substituted with one or more substituents selected from the group consisting of: halogen, cyano, amino, hydroxy, mercapto, C₁₋₆ alkyl, C₁₋₃ alkoxy, or any combination thereof;
the optionally substituted C₃-C₂₀ cycloalkyl and the optionally substituted 3- to 20-membered heterocyclyl are each independently optionally substituted with one or more substituents selected from the group consisting of: halogen, cyano, amino, hydroxy, mercapto, oxo, C₁₋₆ alkyl, C₁₋₃ alkoxy, C₁₋₃ alkoxycarbonyl, halogenated C₂₋₄ alkenyl, halogenated C₁₋₃ alkoxy, halogenated C₁₋₃alkyl, C₁₋₆ alkylsulfonyl, bis(C₁₋₆ alkyl)phosphono, methanesulfonyloxy, trifluoromethanesulfonyloxy, p-toluenesulfonyloxy, optionally substituted 5- or 6-membered heterocyclyl, optionally substituted C₅-C₂₀ aryl, optionally substituted 5- or 6-membered heteroaryl, or any combination thereof, wherein the 5- or 6-membered heterocyclyl is optionally substituted with a substituent selected from the group consisting of: halogen, cyano, amino, hydroxy, oxo, mercapto, C₁₋₆alkyl, halogenated C₁₋₃ alkyl, or any combination thereof; and the C₅-C₂₀ aryl is optionally substituted with a substituent selected from the group consisting of: halogen, cyano, amino, hydroxy, mercapto, C₁₋₆alkyl, halogenated C₁₋₃alkyl, or any combination thereof; and the 5- or 6-membered heteroaryl is optionally substituted with a substituent selected from the group consisting of: halogen, cyano, amino, hydroxy, mercapto, C₁₋₆ alkyl, halogenated C₁₋₃ alkyl, or any combination thereof;
wherein the optionally substituted C₅-C₁₄ aryl or optionally substituted 5- to 20-membered heteroaryl is each independently optionally substituted with one or more substituents selected from the group consisting of: halogen, cyano, amino, hydroxy, mercapto, C₁₋₃ alkoxy, halogenated C₁₋₃ alkoxy, halogenated C₁₋₃alkyl, C₁₋₆ alkylsulfonyl, bis(C₁₋₆ alkyl)phosphono, methanesulfonyloxy, trifluoromethanesulfonyloxy, p-toluenesulfonyloxy, optionally substituted 5- or 6-membered heterocyclyl, optionally substituted C₅-C₂₀ aryl, optionally substituted 5- or 6-membered heteroaryl, C₁₋₆alkyl, C₁₋₃ alkoxycarbonyl, halogenated C₂₄ alkenyl, C(O)NHR_{d}, or any combination thereof, wherein R_{d} represents C₁₋₅alkyl or optionally substituted phenyl, and the optionally substituted phenyl is optionally substituted with one or more substituents selected from the group consisting of: halogen, cyano, amino, hydroxy, mercapto, oxo, C₁₋₆alkyl, and any combination thereof; and the 5- or 6-membered heterocyclyl is optionally substituted with a substituent selected from the group consisting of: halogen, cyano, amino, hydroxy, oxo, mercapto, C₁₋₆alkyl, halogenated C₁₋₃alkyl, or any combination thereof; and the C₅-C₂₀ aryl is optionally substituted with a substituent selected from the group consisting of: halogen, cyano, amino, hydroxy, mercapto, C₁₋₆alkyl, halogenated C₁₋₃alkyl, or any combination thereof; and the 5- or 6-membered heteroaryl is optionally substituted with a substituent selected from the group consisting of: halogen, cyano, amino, hydroxy, oxo, mercapto, C₁₋₆alkyl, halogenated C₁₋₃alkyl, or any combination thereof; or
X₁ represents:
C(O)NR₄R₅, wherein R₄ and R₅ each independently represent H, optionally substituted linear or branched C₁₋₁₀alkyl, optionally substituted C₃-C₂₀cycloalkyl, optionally substituted C₅-C₁₄ aryl, optionally substituted 3- to 20-membered heterocyclyl or optionally substituted 5- to 20-membered heteroaryl, or wherein R₄ and R₅ together with the nitrogen atom to which they are attached form optionally substituted 5- to 8-membered heterocyclyl;
wherein the optionally substituted linear or branched C₁₋₁₀alkyl is optionally substituted with one or more substituents selected from the group consisting of: halogen, cyano, hydroxy, amino, mercapto, C₁₋₃alkyl, C₁₋₃ alkoxy, trifluoromethyl, C₅₋₁₀ aryl, 5- to 10-membered heteroaryl, C₃₋₁₂cycloalkyl, 3- to 12-membered heterocyclyl, or any combination thereof;
the optionally substituted C₃-C₂₀cycloalkyl and the optionally substituted 3- to 20-membered heterocyclyl are each independently optionally substituted with one or more substituents selected from the group consisting of: halogen, cyano, amino, hydroxy, oxo, mercapto, C₁₋₆alkyl, C₁₋₃ alkoxy, C₁₋₃ alkoxycarbonyl, halogenated C₂₋₄ alkenyl, halogenated C₁₋₃ alkoxy, halogenated C₁₋₃alkyl, C₁₋₆ alkylsulfonyl, bis(C₁₋₆ alkyl)phosphono, methanesulfonyloxy, trifluoromethanesulfonyloxy, p-toluenesulfonyloxy, optionally substituted 5- or 6-membered heterocyclyl, optionally substituted C₅-C₂₀ aryl, optionally substituted 5- or 6-membered heteroaryl, or any combination thereof, wherein the 5- or 6-membered heterocyclyl is optionally substituted with a substituent selected from the group consisting of: halogen, cyano, amino, hydroxy, oxo, mercapto, C₁₋₆alkyl, halogenated C₁₋₃ alkyl, or any combination thereof; and the C₅-C₂₀ aryl is optionally substituted with a substituent selected from the group consisting of: halogen, cyano, amino, hydroxy, mercapto, C₁₋₆alkyl, halogenated C₁₋₃alkyl, or any combination thereof; and the 5- or 6-membered heteroaryl is optionally substituted with a substituent selected from the group consisting of: halogen, cyano, amino, hydroxy, mercapto, C₁₋₆alkyl, halogenated C₁₋₃alkyl, or any combination thereof;
the optionally substituted C₅-C₁₄ aryl or optionally substituted 5- to 20-membered heteroaryl is each independently optionally substituted with one or more substituents selected from the group consisting of: halogen, cyano, amino, hydroxy, mercapto, C₁₋₃ alkoxy, halogenated C₁₋₃ alkoxy, halogenated C₁₋₃alkyl, C₁₋₆ alkylsulfonyl, bis(C₁₋₆ alkyl)phosphono, methanesulfonyloxy, trifluoromethanesulfonyloxy, p-toluenesulfonyloxy, optionally substituted 5- or 6-membered heterocyclyl, optionally substituted C₅-C₂₀ aryl, optionally substituted 5- or 6-membered heteroaryl, C₁₋₆alkyl, C₁₋₃ alkoxycarbonyl, halogenated C₂₋₄ alkenyl, or any combination thereof, wherein the 5- or 6-membered heterocyclyl is optionally substituted with a substituent selected from the group consisting of: halogen, cyano, amino, hydroxy, oxo, mercapto, C₁₋₆alkyl, halogenated C₁₋₃alkyl, or any combination thereof; and the C₅-C₂₀ aryl is optionally substituted with a substituent selected from the group consisting of: halogen, cyano, amino, hydroxy, mercapto, C₁₋₆alkyl, halogenated C₁₋₃alkyl, or any combination thereof; and the 5- or 6-membered heteroaryl is optionally substituted with a substituent selected from the group consisting of: halogen, cyano, amino, hydroxy, oxo, mercapto, C₁₋₆alkyl, halogenated C₁₋₃alkyl, or any combination thereof; and
the optionally substituted 5- to 8-membered heterocyclyl is optionally substituted with one or more substituents selected from the group consisting of: halogen, cyano, amino, hydroxy, mercapto, C₁₋₃ alkoxy, halogenated C₁₋₃ alkoxy, halogenated C₁₋₃alkyl, C₁₋₆ alkylsulfonyl, bis(C₁₋₆ alkyl)phosphono, methanesulfonyloxy, trifluoromethanesulfonyloxy, p-toluenesulfonyloxy, optionally substituted 5- or 6-membered heterocyclyl, optionally substituted C₅-C₂₀ aryl, optionally substituted 5- or 6-membered heteroaryl, C₁₋₆alkyl, C₁₋₃ alkoxycarbonyl, halogenated C₂₋₄ alkenyl, or any combination thereof, wherein the 5- or 6-membered heterocyclyl is optionally substituted with a substituent selected from the group consisting of: halogen, cyano, amino, hydroxy, mercapto, oxo, C₁₋₆alkyl, halogenated C₁₋₃ alkyl, or any combination thereof; and the C₅-C₂₀ aryl is optionally substituted with a substituent selected from the group consisting of: halogen, cyano, amino, hydroxy, mercapto, C₁₋₆alkyl, halogenated C₁₋₃alkyl, or any combination thereof; and the 5- or 6-membered heteroaryl is optionally substituted with a substituent selected from the group consisting of: halogen, cyano, amino, hydroxy, mercapto, oxo, C₁₋₆alkyl, halogenated C₁₋₃alkyl, or any combination thereof; or
X₁ represents:
NHC(O)R₆, wherein R₆ represents optionally substituted linear or branched C₁₋₁₀alkyl, optionally substituted C₃-C₂₀cycloalkyl, optionally substituted C₅-C₁₄ aryl, optionally substituted 5- to 20-membered heteroaryl or optionally substituted 3- to 20-membered heterocyclyl;
wherein the optionally substituted linear or branched C₁₋₁₀alkyl is optionally substituted with one or more substituents selected from the group consisting of: halogen, cyano, hydroxy, amino, mercapto, C₁₋₃alkyl, C₁₋₃ alkoxy, trifluoromethyl, C₅₋₁₀ aryl, 5- to 10-membered heteroaryl, C₃₋₁₂cycloalkyl, 3- to 12-membered heterocyclyl, or any combination thereof;
the optionally substituted C₃-C₂₀cycloalkyl and the optionally substituted 3- to 20-membered heterocyclyl are each independently optionally substituted with one or more substituents selected from the group consisting of: halogen, cyano, amino, hydroxy, mercapto, oxo, C₁₋₆alkyl, C₁₋₃ alkoxy, C₁₋₃ alkoxycarbonyl, halogenated C₂₋₄ alkenyl, halogenated C₁₋₃ alkoxy, halogenated C₁₋₃alkyl, C₁₋₆ alkylsulfonyl, bis(C₁₋₆ alkyl)phosphono, methanesulfonyloxy, trifluoromethanesulfonyloxy, p-toluenesulfonyloxy, optionally substituted 5- or 6-membered heterocyclyl, optionally substituted C₅-C₂₀ aryl, optionally substituted 5- or 6-membered heteroaryl, or any combination thereof, wherein the 5- or 6-membered heterocyclyl is optionally substituted with one or more substituents selected from the group consisting of: halogen, cyano, amino, hydroxy, mercapto, oxo, C₁₋₆alkyl, halogenated C₁₋₃alkyl, or any combination thereof; and the C₅-C₂₀ aryl is optionally substituted with one or more substituents selected from the group consisting of: halogen, cyano, amino, hydroxy, mercapto, C₁₋₆alkyl, halogenated C₁₋₃alkyl, or any combination thereof; and the 5- or 6-membered heteroaryl is optionally substituted with one or more substituents selected from the group consisting of: halogen, cyano, amino, hydroxy, mercapto, C₁₋₆alkyl, halogenated C₁₋₃alkyl, or any combination thereof; and
the optionally substituted C₅-C₁₄ aryl or optionally substituted 5- to 20-membered heteroaryl is each independently optionally substituted with one or more substituents selected from the group consisting of: halogen, cyano, amino, hydroxy, mercapto, C₁₋₃ alkoxy, halogenated C₁₋₃ alkoxy, halogenated C₁₋₃alkyl, C₁₋₆ alkylsulfonyl, bis(C₁₋₆ alkyl)phosphono, methanesulfonyloxy, trifluoromethanesulfonyloxy, p-toluenesulfonyloxy, optionally substituted 5- or 6-membered heterocyclyl, optionally substituted C₅-C₂₀ aryl, optionally substituted 5- or 6-membered heteroaryl, C₁₋₆alkyl, C₁₋₃ alkoxycarbonyl, halogenated C₂₋₄ alkenyl, or any combination thereof, wherein the 5- or 6-membered heterocyclyl is optionally substituted with one or more substituents selected from the group consisting of: halogen, cyano, amino, hydroxy, mercapto, oxo, C₁₋₆alkyl, halogenated C₁₋₃alkyl, or any combination thereof; and the C₅-C₂₀ aryl is optionally substituted with one or more substituents selected from the group consisting of: halogen, cyano, amino, hydroxy, mercapto, C₁₋₆alkyl, halogenated C₁₋₃alkyl, or any combination thereof; and the 5- or 6-membered heteroaryl is optionally substituted with one or more substituents selected from the group consisting of: halogen, cyano, amino, hydroxy, mercapto, oxo, C₁₋₆ alkyl, halogenated C₁₋₃ alkyl, or any combination thereof; or
X₁ represents:
a quaternary ammonium salt group of Formula N⁺RₐR_{b}R_{c}X⁻,
wherein Rₐ, R_{b}, and R_{c} each independently represent C₁₋₂₀alkyl, and X⁻ represents F⁻, Cl⁻, Br⁻, or I⁻; or
wherein X⁻ represents F⁻, Cl⁻, Br⁻, or I⁻, R_{c} represents C₁₋₂₀alkyl or hydrogen, and Rₐ and R_{b} together with the nitrogen atom to which they are attached form a 5- to 8-membered heterocyclyl, wherein the ring atoms of the 5- to 8-membered heterocyclyl optionally further comprise a heteroatom selected from oxygen, nitrogen, and sulfur, and the 5- to 8-membered heterocyclyl is optionally substituted with one or more substituents selected from the group consisting of: halogen, cyano, amino, hydroxy, mercapto, C₁₋₃ alkoxy, halogenated C₁₋₃ alkoxy, halogenated C₁₋₃alkyl, C₁₋₆ alkylsulfonyl, bis(C₁₋₆ alkyl)phosphono, methanesulfonyloxy, trifluoromethanesulfonyloxy, p-toluenesulfonyloxy, optionally substituted 5- or 6-membered heterocyclyl, optionally substituted C₅-C₂₀ aryl, optionally substituted 5- or 6-membered heteroaryl, C₁₋₆alkyl, C₁₋₃ alkoxycarbonyl, halogenated C₂₋₄ alkenyl, or any combination thereof, wherein the 5- or 6-membered heterocyclyl is optionally substituted with a substituent selected from the group consisting of: halogen, cyano, amino, hydroxy, mercapto, oxo, C₁₋₆alkyl, halogenated C₁₋₃alkyl, or any combination thereof; and the C₅-C₂₀ aryl is optionally substituted with a substituent selected from the group consisting of: halogen, cyano, amino, hydroxy, mercapto, C₁₋₆alkyl, halogenated C₁₋₃ alkyl, or any combination thereof; and the 5- or 6-membered heteroaryl is optionally substituted with a substituent selected from the group consisting of: halogen, cyano, amino, hydroxy, mercapto, oxo, C₁₋₆alkyl, halogenated C₁₋₃alkyl, or any combination thereof; or
(ii) wherein L represents the structure of the following formula:
linear or branched C₁₋₄₀ alkylene;
*—(C(Rₐ₂)(Rₐ₃))ₙ₂—(R_{b}—(C(Rₐ₄)(Rₐ₅))ₙ₃)ₘ₁;
*—(C(Rₐ₂)(Rₐ₃))ₙ₂—(R_{b}—(C(Rₐ₄)(Rₐ₅))ₙ₃)ₘ₁—(R_{b}—(C(Rₐ₆)(Rₐ₇))ₙ₄)ₘ₂—;
*—(C(Rₐ₂)(Rₐ₃))ₙ₂—(R_{b}—(C(Rₐ₄)(Rₐ₅))ₙ₃)ₘ₁—(R_{b}—(C(Rₐ₆)(Rₐ₇))ₙ₄)ₘ₂—(R_{b}—(C(Rₐ₈)(Rₐ₉))ₙ₅)ₘ₃—;
*—(C(Rₐ₂)(Rₐ₃))ₙ₂—(R_{c}—(C(Rₐ₄)(Rₐ₅))ₙ₃)ₘ₁—;
*—(C(Rₐ₂)(Rₐ₃))ₙ₂—(R_{c}—(C(Rₐ₄)(Rₐ₅))ₙ₃)ₘ₁—(R_{c}—(C(Rₐ₆)(Rₐ₇))ₙ₄)ₘ₂—;
*—(C(Rₐ₂)(Rₐ₃))ₙ₂—(R_{c}—(C(Rₐ₄)(Rₐ₅))ₙ₃)ₘ₁—(R_{c}—(C(Rₐ₆)(Rₐ₇))ₙ₄)ₘ₂—(R_{c}—(C(Rₐ₈)(Rₐ₉))ₙ₅)ₘ₃—;
*—(C(Rₐ₂)(Rₐ₃))ₙ₂—(R_{b}—R_{c}—(C(Rₐ₄)(Rₐ₅))ₙ₃)ₘ₁—;
*—(C(Rₐ₂)(Rₐ₃))ₙ₂—(R_{b}—(C(Rₐ₄)(Rₐ₅))ₙ₃)ₘ₁—(R_{c}—(C(Rₐ₆)(Rₐ₇))ₙ₄)ₘ₂—;
*—(C(Rₐ₂)(Rₐ₃))ₙ₂—(R_{c}—R_{b}—(C(Rₐ₄)(Rₐ₅))ₙ₃)ₘ₁—; or
*—(C(Rₐ₂)(Rₐ₃))ₙ₂—(R_{c}—(C(Rₐ₄)(Rₐ₅))ₙ₃)ₘ₁—(R_{b}—(C(Rₐ₆)(Rₐ₇))ₙ₄)ₘ₂—;
wherein * indicates the point of attachment to R;
a hydrogen of one or more CH₂ groups of the linear or branched C₁₋₄₀ alkylene is optionally replaced with a substituent selected from the group consisting of: halogen, cyano, C₁₋₃alkyl, C₁₋₃ alkoxy, trifluoromethyl, C₅₋₁₀ aryl, C₅₋₁₀ heteroaryl, C₃₋₁₂cycloalkyl, 3- to 12-membered heterocyclyl, or any combination thereof;
R_{b} groups are each independently selected from the group consisting of O, C(O), OC(O), S, S(O), S(O)₂, S(O)₂N(R₁), N(R₁)S(O)₂, C(O)N(R₁), N(R₁)C(O), N(R₁) and N(R₁)C(O)N(R₁), wherein R₁ groups each independently represent H or C₁₋₃alkyl;
R_{c} groups are each independently selected from the group consisting of optionally substituted cycloalkylene (e.g., C₃₋₂₀ cycloalkylene), optionally substituted arylene (e.g., C₅₋₂₀ arylene), optionally substituted heterocyclylene (e.g., 4- to 20-membered heterocyclylene), optionally substituted heteroarylene (e.g., 5- to 20-membered heteroarylene), and any combination thereof, wherein the cycloalkylene and heterocyclylene are each independently optionally substituted with a substituent selected from the group consisting of halogen, cyano, hydroxy, amino, mercapto, oxo, C₁₋₃ alkoxy, halogenated C₁₋₃alkyl, methanesulfonyloxy, trifluoromethanesulfonyloxy, p-toluenesulfonyloxy, optionally deuterated C₁₋₆ alkyl, optionally deuterated C₃₋₆ cycloalkyl, or any combination thereof; and the arylene and heteroarylene are each independently optionally substituted with a substituent selected from the group consisting of halogen, cyano, hydroxy, amino, mercapto, C₁₋₃ alkoxy, halogenated C₁₋₃ alkyl, methanesulfonyloxy, trifluoromethanesulfonyloxy, p-toluenesulfonyloxy, optionally deuterated C₁₋₆ alkyl, optionally deuterated C₃₋₆ cycloalkyl, or any combination thereof;
Rₐ₂, Rₐ₃, Rₐ₄, Rₐ₅, Rₐ₆, Rₐ₇, Rₐ₈, and Rₐ₉ each independently represent H, halogen, cyano, hydroxy, amino, mercapto, C₁₋₃alkyl, C₁₋₃ alkoxy, trifluoromethyl, C₅₋₁₀ aryl, C₅₋₁₀ heteroaryl, C₃₋₁₂cycloalkyl or 3- to 12-membered heterocyclyl; and
n2, n3, n4, n5, m1, m2, and m3 each independently represent an integer of 1, 2, 3, 4, 5, 6, 7, 8, 9, 10, 11, 12, 13, 14, or 15; or
(iii) wherein X₁ represents:
(iii-1) linear or branched C₁₋₁₀ alkyl optionally substituted with one or more fluorine; or
(iii-2) cyclopropyl, cyclobutyl, 3,3-difluorocyclobutyl, cyclopentyl, cyclopentenyl, cyclohexyl, cyclohexenyl, cycloheptyl, cyclooctyl, decalinyl, octahydropentalenyl, octahydro-1H-indenyl, 2,3-dihydro-1H-indenyl, 4-cyano-2,3-dihydro-1H-indenyl, spiro[3.3]heptanyl, spiro[2.5]octyl, spiro[3.5]nonyl, spiro[4.4]nonyl, spiro[4.5]decyl, spiro[5.5]undecyl, p-menthanyl, m-menthanyl, quinuclidinyl, adamantanyl, noradamantanyl, bornyl, 3,5-dimethyladamantan-1-yl, 1,7,7-trimethylbicyclo[2.2.1]heptan-2-yl, 7,7-dimethylbicyclo[2.2.1]heptan-1-yl, 7,7-dimethyl-2-oxobicyclo[2.2.1]heptan-1-yl, bicyclo[2.2.1]heptanyl, 2-oxobicyclo[2.2.1]heptyl, or bicyclo[2.2.1]heptenyl, with each group being optionally substituted with one or more substituents selected from the group consisting of: halogen, cyano, amino, hydroxy, mercapto, oxo, C₁₋₃ alkoxy, halogenated C₁₋₃ alkoxy, halogenated C₁₋₃alkyl, C₁₋₆ alkylsulfonyl, bis(C₁₋₆ alkyl)phosphono, methanesulfonyloxy, trifluoromethanesulfonyloxy, p-toluenesulfonyloxy, optionally substituted 5- or 6-membered heterocyclyl, optionally substituted C₅-C₂₀ aryl, optionally substituted 5- or 6-membered heteroaryl, C₁₋₆alkyl, C₁₋₃ alkoxycarbonyl, halogenated C₂₋₄ alkenyl, or any combination thereof, wherein the 5- or 6-membered heterocyclyl is optionally substituted with one or more substituents selected from the group consisting of: halogen, cyano, amino, hydroxy, mercapto, oxo, C₁₋₆alkyl, halogenated C₁₋₃ alkyl, or any combination thereof; and the C₅-C₂₀ aryl is optionally substituted with one or more substituents selected from the group consisting of: halogen, cyano, amino, hydroxy, mercapto, C₁₋₆alkyl, halogenated C₁₋₃alkyl, or any combination thereof; and the 5- or 6-membered heteroaryl is optionally substituted with one or more substituents selected from the group consisting of: halogen, cyano, amino, hydroxy, mercapto, oxo, C₁₋₆alkyl, halogenated C₁₋₃alkyl, or any combination thereof; or
(iii-3) phenyl or naphthyl, with each group being optionally substituted with one or more substituents selected from the group consisting of halogen, cyano, amino, hydroxy, mercapto, C₁₋₃ alkoxy, halogenated C₁₋₃ alkoxy, halogenated C₁₋₃ alkyl, C₁₋₆ alkylsulfonyl, bis(C₁₋₆ alkyl)phosphono, methanesulfonyloxy, trifluoromethanesulfonyloxy, p-toluenesulfonyloxy, optionally substituted 5- or 6-membered heterocyclyl, optionally substituted C₅-C₂₀ aryl, optionally substituted 5- or 6-membered heteroaryl, C₁₋₆alkyl, C₁₋₃ alkoxycarbonyl, halogenated C₂₋₄ alkenyl, or any combination thereof, wherein the 5- or 6-membered heterocyclyl is optionally substituted with one or more substituents selected from the group consisting of: halogen, cyano, amino, hydroxy, mercapto, oxo, C₁₋₆alkyl, halogenated C₁₋₃alkyl, or any combination thereof; and the C₅-C₂₀ aryl is optionally substituted with one or more substituents selected from the group consisting of: halogen, cyano, amino, hydroxy, mercapto, C₁₋₆alkyl, halogenated C₁₋₃alkyl, or any combination thereof; and the 5- or 6-membered heteroaryl is optionally substituted with one or more substituents selected from the group consisting of: halogen, cyano, amino, hydroxy, mercapto, oxo, C₁₋₆alkyl, halogenated C₁₋₃alkyl, or any combination thereof; or
(iii-4) azetidinyl, oxetanyl, pyrrolidinyl, imidazolidinyl, pyrazolidinyl, oxazolidinyl, thiazolidinyl, piperidinyl, piperazinyl, morpholinyl, thiomorpholinyl, azacycloheptyl, azacyclooctyl, diazacycloheptanyl, diazacyclooctyl, 3-azabicyclo[3.1.0]hexyl, 3-azabicyclo[3.1.1]heptanyl, 3-azabicyclo[4.1.0]heptanyl, 2-azabicyclo[2.2.1]heptanyl, 6-azabicyclo[3.1.1]heptanyl, 2-azabicyclo[2.2.2]octyl, 2,5-diazabicyclo[2.2.1]heptanyl, 3,6-diazabicyclo[3.1.1]heptanyl, 3-azabicyclo[3.2.1]octyl, 3,8-diazabicyclo[3.2.1]octyl, 2,5-diazabicyclo[2.2.2]octyl, 2,6-diazaspiro[3.3]heptanyl, 2,7-diazaspiro[3.5]nonyl, 3-azaspiro[5.5]undecyl, 5-azaspiro[2.4]heptanyl, 6-azaspiro[2.5]octyl, 2-oxa-7-azaspiro[3.5]nonyl, 7-azaspiro[3.5]nonyl, hexahydro-1H-isoindol-2(3H)-yl, (3aR,7aS)-octahydro-2H-isoindol-2-yl, octahydropyrrolo[3,4-c]pyrrolyl, or 5,7-dihydro-6H-pyrrolo[3,4-b]pyridyl, with each group being optionally substituted with one or more substituents selected from the group consisting of halogen, cyano, amino, hydroxy, mercapto, oxo, C₁₋₃ alkoxy, halogenated C₁₋₃ alkoxy, halogenated C₁₋₃alkyl, C₁₋₆ alkylsulfonyl, bis(C₁₋₆ alkyl)phosphono, methanesulfonyloxy, trifluoromethanesulfonyloxy, p-toluenesulfonyloxy, optionally substituted 5- or 6-membered heterocyclyl, optionally substituted C₅-C₂₀ aryl, optionally substituted 5- or 6-membered heteroaryl, C₁₋₆alkyl, C₁₋₃ alkoxycarbonyl, halogenated C₂₋₄ alkenyl, or any combination thereof, wherein the 5- or 6-membered heterocyclyl is optionally substituted with one or more substituents selected from the group consisting of: halogen, cyano, amino, hydroxy, mercapto, oxo, C₁₋₆alkyl, halogenated C₁₋₃alkyl, or any combination thereof; and the C₅-C₂₀ aryl is optionally substituted with one or more substituents selected from the group consisting of: halogen, cyano, amino, hydroxy, mercapto, C₁₋₆alkyl, halogenated C₁₋₃alkyl, or any combination thereof; and the 5- or 6-membered heteroaryl is optionally substituted with one or more substituents selected from the group consisting of: halogen, cyano, amino, hydroxy, mercapto, oxo, C₁₋₆alkyl, halogenated C₁₋₃alkyl, or any combination thereof; or
(iii-5) furanyl, oxazolyl, isoxazolyl, oxadiazolyl, thienyl, thiazolyl, isothiazolyl, thiadiazolyl, pyrrolyl, imidazolyl, pyrazolyl, triazolyl, pyridyl, pyrimidinyl, pyridazinyl, pyrazinyl, indolyl, isoindolyl, benzofuranyl, isobenzofuranyl, benzothienyl, indazolyl, benzimidazolyl, benzoxazolyl, benzisoxazolyl, benzothiazolyl, benzisothiazolyl, benzotriazolyl, benzo[2,1,3]oxadiazolyl, benzo[2,1,3]thiadiazolyl, benzo[1,2,3]thiadiazolyl, tetrahydro-2H-pyran-2-yl, quinolinyl, isoquinolinyl, naphthyridinyl, cinnolinyl, quinazolinyl, quinoxalinyl, phthalazinyl, pyrazolo[1,5-a]pyridyl, pyrazolo[1,5-a]pyrimidinyl, imidazo[1,2-a]pyridyl, 1H-pyrrolo[3,2-b]pyridyl, 1H-pyrrolo[2,3-b]pyridyl, 4H-fluoro[3,2-b]pyrrolyl, pyrrolo[2,1-b]thiazolyl, or imidazo[2,1-b]thiazolyl, with each group being optionally substituted with one or more substituents selected from the group consisting of halogen, cyano, amino, hydroxy, mercapto, C₁₋₃ alkoxy, halogenated C₁₋₃ alkoxy, halogenated C₁₋₃alkyl, bis(C₁₋₆ alkyl)phosphono, C₁₋₆ alkylsulfonyl, methanesulfonyloxy, trifluoromethanesulfonyloxy, p-toluenesulfonyloxy, C₁₋₆ alkyl, optionally substituted 5- or 6-membered heterocyclyl, optionally substituted C₅-C₂₀ aryl, optionally substituted 5- or 6-membered heteroaryl, C₁₋₆alkyl, C₁₋₃ alkoxycarbonyl, halogenated C₂₋₄ alkenyl, or any combination thereof, wherein the 5- or 6-membered heterocyclyl is optionally substituted with one or more substituents selected from the group consisting of: C₁₋₆ alkyl, cyano, halogenated C₁₋₃alkyl, amino, hydroxy, mercapto, halogen, or any combination thereof; and the C₅-C₂₀ aryl is optionally substituted with one or more substituents selected from the group consisting of: C₁₋₆ alkyl, cyano, halogenated C₁₋₃alkyl, amino, hydroxy, mercapto, halogen, or any combination thereof; and the 5- or 6-membered heteroaryl is optionally substituted with one or more substituents selected from the group consisting of: C₁₋₆alkyl, halogenated C₁₋₃alkyl, amino, hydroxy, mercapto, halogen, or any combination thereof; or
(iii-6) X₁ represents the group of Formula (G₂): 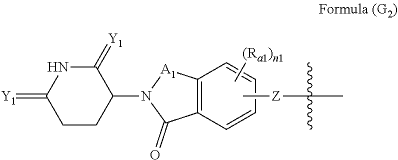
wherein A₁ represents CH₂ or C(O), Y₁ represents O or S, and Z represents S, S(O), S(O)₂, O, NH or CH₂, and (Rₐ₁)ₙ₁ denotes that the benzene ring is substituted with n1 Rₐ₁, where Rₐ₁ groups are the same or different and each independently represent deuterium, halogen, hydroxy, mercapto, nitro, amino, cyano, optionally deuterated C₁₋₆ alkyl, optionally deuterated C₁₋₆ alkoxy, halogenated C₁₋₆ alkyl, optionally substituted C₃₋₆ cycloalkyl, C₂₋₆ alkenyl, or C₂₋₆ alkynyl, and n represents an integer of 0, 1, 2, or 3.

9. The compound of Formula (I) or salts, enantiomers, diastereoisomer, isotopically enriched analogs, solvates, prodrugs, or polymorphs thereof of claim 1,
(i) wherein L represents the structure of the following formula:
*—(C(Rₐ₂)(Rₐ₃))ₙ₂—(C₅₋₂₀ arylene-(C(Rₐ₄)(Rₐ₅))ₙ₃)ₘ₁—;
*—(C(Rₐ₂)(Rₐ₃))ₙ₂—(C₅₋₂₀ arylene-(C(Rₐ₄)(Rₐ₅))ₙ₃)ₘ₁—(C₅₋₂₀ arylene-(C(Rₐ₆)(Rₐ₇))ₙ₄)ₘ₂—;
*—(C(Rₐ₂)(Rₐ₃))ₙ₂—(C₅₋₂₀arylene-(C(Rₐ₄)(Rₐ₅))ₙ₃)ₘ₁—(C₅₋₂₀ arylene-(C(Rₐ₆)(Rₐ₇))ₙ₄)ₘ₂—(C₅₋₂₀ arylene-(C(Rₐ₈)(Rₐ₉))ₙ₅)ₘ₃—;
*—(C(Rₐ₂)(Rₐ₃))ₙ₂-(5- to 20-membered heteroarylene-(C(Rₐ₄)(Rₐ₅))ₙ₃)ₘ₁—;
*—(C(Rₐ₂)(Rₐ₃))ₙ₂-(5- to 20-membered heteroarylene-(C(Rₐ₄)(Rₐ₅))ₙ₃)ₘ₁-(5- to 20-membered heteroarylene-(C(Rₐ₆)(Rₐ₇))ₙ₄)ₘ₂—;
*—(C(R₂)(R₃))ₙ₂-(5- to 20-membered heteroarylene-(C(Rₐ₄)(Rₐ₅))ₙ₃)ₘ₁-(5- to 20-membered heteroarylene-(C(Rₐ₆)(Rₐ₇))ₙ₄)ₘ₂-(5- to 20-membered heteroarylene-(C(Rₐ₈)(Rₐ₉))ₙ₅)ₘ₃—;
*—(C(Rₐ₂)(Rₐ₃))ₙ₂-(4- to 20-membered heterocyclylene-(C(Rₐ₄)(Rₐ₅))ₙ₃)ₘ₁—;
*—(C(Rₐ₂)(Rₐ₃))ₙ₂-(4- to 20-membered heterocyclylene-(C(Rₐ₄)(Rₐ₅))ₙ₃)ₘ₁-(4- to 20-membered heterocyclylene-(C(Rₐ₆)(Rₐ₇))ₙ₄)ₘ₂—;
*—(C(Rₐ₂)(Rₐ₃))ₙ₂—(O—(C(Rₐ₄)(Rₐ₅))ₙ₃)ₘ₁—;
*—(C(Rₐ₂)(Rₐ₃))ₙ₂—(O—(C(Rₐ₄)(Rₐ₅))ₙ₃)ₘ₁—(O—(C(Rₐ₆)(Rₐ₇))ₙ₄)ₘ₂—;
*—(C(Rₐ₂)(Rₐ₃))ₙ₂—(O—(C(Rₐ₄)(Rₐ₅))ₙ₃)ₘ₁—(O—(C(Rₐ₆)(Rₐ₇))ₙ₄)ₘ₂—(O—(C(Rₐ₈)(Rₐ₉))ₙ₅)ₘ₃—;
*—(C(Rₐ₂)(Rₐ₃))ₙ₂—(S—(C(Rₐ₄)(Rₐ₅))ₙ₃)ₘ₁—;
*—(C(Rₐ₂)(Rₐ₃))ₙ₂—(S—(C(Rₐ₄)(Rₐ₅))ₙ₃)ₘ₁—(S—(C(Rₐ₆)(Rₐ₇))ₙ₄)ₘ₂—;
*—(C(Rₐ₂)(Rₐ₃))ₙ₂—(S—(C(Rₐ₄)(Rₐ₅))ₙ₃)ₘ₁—(S—(C(Rₐ₆)(Rₐ₇))ₙ₄)ₘ₂—(S—(C(Rₐ₈)(Rₐ₉))ₙ₅)ₘ₃—;
*—(C(Rₐ₂)(Rₐ₃))ₙ₂—(S(O)—(C(Rₐ₄)(Rₐ₅))ₙ₃)ₘ₁—;
*—(C(Rₐ₂)(Rₐ₃))ₙ₂—(S(O)₂—(C(Rₐ₄)(Rₐ₅))ₙ₃)ₘ₁—;
*—(C(Rₐ₂)(Rₐ₃))ₙ₂—(S—(C(Rₐ₄)(Rₐ₅))ₙ₃)ₘ₁—;
*—(C(Rₐ₂)(Rₐ₃))ₙ₂—(S(O)₂N(R₁)—(C(Rₐ₄)(Rₐ₅))ₙ₃)ₘ₁—;
*—(C(Rₐ₂)(Rₐ₃))ₙ₂—(N(R₁)S(O)₂—(C(Rₐ₄)(Rₐ₅))ₙ₃)ₘ₁—;
*—(C(Rₐ₂)(Rₐ₃))ₙ₂—(C(O)N(R₁)—(C(Rₐ₄)(Rₐ₅))ₙ₃)ₘ₁—;
*—(C(Rₐ₂)(Rₐ₃))ₙ₂—(N(R₁)C(O)—(C(Rₐ₄)(Rₐ₅))ₙ₃)ₘ₁—;
*—(C(Rₐ₂)(Rₐ₃))ₙ₂—(N(R₁)—(C(Rₐ₄)(Rₐ₅))ₙ₃)ₘ₁—;
*—(C(Rₐ₂)(Rₐ₃))ₙ₂—(N(R₁)C(O)N(R₁)—(C(Rₐ₄)(Rₐ₅))ₙ₃)ₘ₁—;
wherein * indicates the point of attachment to R;
R₁ groups each independently represent H or C₁₋₃alkyl;
Rₐ₂, Rₐ₃, Rₐ₄, Rₐ₅, Rₐ₆, Rₐ₇, Rₐ₈, and Rₐ₉ each independently represent H, halogen, cyano, hydroxy, amino, mercapto, C₁₋₃alkyl, C₁₋₃ alkoxy, trifluoromethyl, C₅₋₁₀ aryl, C₅₋₁₀ heteroaryl, C₃₋₁₂cycloalkyl, or 3- to 12-membered heterocyclyl;
n2, n3, n4, n5, m1, m2, and m3 each independently represent an integer of 1, 2, 3, 4, 5, 6, 7, 8, 9, 10, 11, 12, 13, 14, or 15; and
the 4- to 20-membered heterocyclylene is optionally substituted with a substituent selected from the group consisting of halogen, cyano, hydroxy, amino, mercapto, oxo, C₁₋₃ alkoxy, halogenated C₁₋₃alkyl, methanesulfonyloxy, trifluoromethanesulfonyloxy, p-toluenesulfonyloxy, optionally deuterated C₁₋₆ alkyl, optionally deuterated C₃₋₆ cycloalkyl, or any combination thereof; and the C₅₋₂₀ arylene and the 5- to 20-membered heteroarylene are each independently optionally substituted with a substituent selected from the group consisting of halogen, cyano, hydroxy, amino, mercapto, C₁₋₃ alkoxy, halogenated C₁₋₃alkyl, methanesulfonyloxy, trifluoromethanesulfonyloxy, p-toluenesulfonyloxy, optionally deuterated C₁₋₆ alkyl, optionally deuterated C₃₋₆ cycloalkyl, or any combination thereof; or
(ii) wherein X₁ represents: morpholinyl, piperidinyl, difluoropiperidinyl, 4,4-dimethylpiperidin-1-yl, 3,5-dimethylpiperidin-1-yl, piperazinyl, 3-hydroxy-2-methylazetidin-1-yl, trifluoromethoxyazetidinyl, azetidinyl, cyanoazetidinyl, hydroxyazetidinyl, 3-hydroxy-3-methylazetidin-1-yl, 3-fluoro-4-hydroxypyrrolidin-1-yl, 3-methyl-4-(trifluoromethyl)pyrrolidin-1-yl, 7-fluoro-5-azaspiro[2.4]heptan-5-yl, 3-azabicyclo[3.1.0]hexan-3-yl, 6-(difluoromethyl)-3-azabicyclo[4.1.0]heptan-3-yl, 7-(3-fluoro-5,7-dihydro-6H-pyrrolo[3,4-b]pyridin-6-yl, 1,1-difluoro-5-azaspiro[2.4]heptan-5-yl, 1,1-difluoro-6-azaspiro[2.5]octan-6-yl, 2-oxa-7-azaspiro[3.5]nonan-7-yl, 4-(methylsulfonyl)piperazin-1-yl, 4-(ethylsulfonyl)piperazin-1-yl, 1,4-diazepan-1-yl, 3,8-diazabicyclo[3.2.1]octan-3-yl, 2-azabicyclo[3.2.1]octanyl, 1,4-diazabicyclo[3.2.1]octanyl, 2,5-diazabicyclo[2.2.2]octan-2-yl, pyrrolidinyl, adamantanyl, noradamantanyl, bornyl, or norbornyl; or
(iii) wherein X₁ represents: piperidinyl-C(O)—, difluoropiperidinyl-C(O)—, —C(O)N(CH(CH₃)₂)₂, C(O)NH₂, N,N-diisopropylcarbamoyl, adamantanyl-NHC(O)—, 3,5-dimethyladamantan-1-yl-NHC(O)—, adamantan-2-yl-NHC(O)—, adamantanyl-C(O)NH—, adamantan-1-yl-C(O)NH—, 3-hydroxyadamantanyl-C(O)NH—, 3-hydroxyadamantan-1-yl-C(O)NH—, 3-chloroadamantan-1-yl-C(O)NH—, 4-chloroadamantan-1-yl-C(O)NH—, 2-chloroadamantan-1-yl-C(O)NH—, adamantan-2-yl-C(O)NH—, —NHC(O)CH₃, OH, adamantanyl-O—, adamantan-1-yl-O—, adamantan-2-yl-O—, norbornyl-O-1,7,7-trimethyl-bicyclo[2.2.1]heptanyl-O—, —SH, adamantanyl-S—, adamantan-1-yl-S—, 3-hydroxyadamantanyl-S—, 3-hydroxyadamantan-1-yl-S—, 3-chloroadamantan-1-yl-S—, 4-chloroadamantan-1-yl-S—, 2-chloroadamantan-1-yl-S—, adamantan-2-yl-S—, or 2-isopropyl-5-methylcyclohexyl-O—; or
(iv) wherein X₁ represents NR₂R₃, where R₂ and R₃ each independently represent H or optionally substituted linear or branched C₁₋₁₀ alkyl, wherein R₂ and R₃ are not H at the same time, and the optionally substituted linear or branched C₁₋₁₀ alkyl is optionally substituted with one or more substituents selected from the group consisting of: halogen, cyano, hydroxy, amino, mercapto, C₁₋₃ alkyl, C₁₋₃ alkoxy, trifluoromethyl, optionally substituted C₅₋₁₀ aryl, optionally substituted 5- to 10-membered heteroaryl, optionally substituted C₃₋₁₂cycloalkyl, optionally substituted 3- to 12-membered heterocyclyl, or any combination thereof, wherein the optionally substituted C₅₋₁₀ aryl, optionally substituted 5- to 10-membered heteroaryl, optionally substituted C₃₋₁₂ cycloalkyl and optionally substituted 3- to 12-membered heterocyclyl are each independently optionally substituted with one or more substituents selected from the group consisting of: halogen, cyano, amino, hydroxy, mercapto, C₁₋₆ alkyl, C₁₋₃ alkoxy, or any combination thereof; or
(v) X₁ represents NR₂R₃, where R₂ represents H or C₁₋₃alkyl, and R₃ represents optionally substituted C₃-C₂₀ cycloalkyl, optionally substituted C₅-C₁₄ aryl, optionally substituted 3- to 20-membered heterocyclyl, or optionally substituted 5- to 20-membered heteroaryl, wherein the C₃-C₂₀ cycloalkyl and 3- to 20-membered heterocyclyl are each independently optionally substituted with one or more substituents selected from the group consisting of: halogen, cyano, amino, hydroxy, mercapto, oxo, C₁₋₆ alkyl, C₁₋₃ alkoxy, C₁₋₃ alkoxycarbonyl, halogenated C₂₋₄ alkenyl, halogenated C₁₋₃ alkoxy, halogenated C₁₋₃ alkyl, C₁₋₆ alkylsulfonyl, bis(C₁₋₆ alkyl)phosphono, methanesulfonyloxy, trifluoromethanesulfonyloxy, p-toluenesulfonyloxy, optionally substituted 5- or 6-membered heterocyclyl, optionally substituted C₅-C₂₀ aryl, optionally substituted 5- or 6-membered heteroaryl, or any combination thereof, wherein the 5- or 6-membered heterocyclyl is optionally substituted with a substituent selected from the group consisting of: halogen, cyano, amino, hydroxy, oxo, mercapto, C₁₋₆ alkyl, halogenated C₁₋₃ alkyl, or any combination thereof; and the C₅-C₂₀ aryl is optionally substituted with a substituent selected from the group consisting of: halogen, cyano, amino, hydroxy, mercapto, C₁₋₆ alkyl, halogenated C₁₋₃ alkyl, or any combination thereof; and the 5- or 6-membered heteroaryl is optionally substituted with a substituent selected from the group consisting of: halogen, cyano, amino, hydroxy, mercapto, C₁₋₆ alkyl, halogenated C₁₋₃ alkyl, or any combination thereof; and
the C₅-C₁₄ aryl and 5- to 20-membered heteroaryl are each independently optionally substituted with one or more substituents selected from the group consisting of: halogen, cyano, amino, hydroxy, mercapto, C₁₋₃ alkoxy, halogenated C₁₋₃ alkoxy, halogenated C₁₋₃ alkyl, C₁₋₆ alkylsulfonyl, bis(C₁₋₆ alkyl)phosphono, methanesulfonyloxy, trifluoromethanesulfonyloxy, p-toluenesulfonyloxy, optionally substituted 5- or 6-membered heterocyclyl, optionally substituted C₅-C₂₀ aryl, optionally substituted 5- or 6-membered heteroaryl, C₁₋₆ alkyl, C₁₋₃ alkoxycarbonyl, halogenated C₂₋₄ alkenyl, —C(O)NHR_{d}, or any combination thereof, wherein R_{d} represents C₁₋₅ alkyl or optionally substituted phenyl, and the optionally substituted phenyl is optionally substituted with one or more substituents selected from the group consisting of: halogen, cyano, amino, hydroxy, mercapto, oxo, and C₁₋₆ alkyl, or any combination thereof; and the 5- or 6-membered heterocyclyl is optionally substituted with a substituent selected from the group consisting of: halogen, cyano, amino, hydroxy, oxo, mercapto, C₁₋₆ alkyl, halogenated C₁₋₃ alkyl, or any combination thereof; and the C₅-C₂₀ aryl is optionally substituted with a substituent selected from the group consisting of: halogen, cyano, amino, hydroxy, mercapto, C₁₋₆ alkyl, halogenated C₁₋₃ alkyl, or any combination thereof; and the 5- or 6-membered heteroaryl is optionally substituted with a substituent selected from the group consisting of: halogen, cyano, amino, hydroxy, oxo, mercapto, C₁₋₆ alkyl, halogenated C₁₋₃ alkyl, or any combination thereof; or
(vi) wherein X₁ represents CF₂CF₃, CF₃, adamantanyl-NH—, noradamantanyl-NH—, cyclohexyl-NH—, adamantanyl-N(CH₃)—, noradamantanyl-N(CH₃)—, bornyl-NH—, norbornyl-NH—, spiro[3.3]heptyl-NH—, N(CH₂CH₃)₂, phenyl, 4-fluorophenyl, 4-chlorophenyl, 3,5-difluorophenyl, 4-(bromomethyl)-2-fluorophenyl, or 3,4,5-trifluorophenyl; or
(vii) wherein X₁ represents NR₂R₃, wherein R₂ represents H, and R₃ represents ethyl substituted with 2,6-dichloro-3-fluorophenyl; or
(viii) wherein X₁ represents NR₂R₃, wherein
R₂ represents H, methyl, ethyl, isopropyl, or cyclohexyl, and
R₃ represents the following optionally substituted groups: adamantanyl, noradamantanyl, norbornyl, cyclohexyl, cyclopentyl, cyclopropyl, cyclobutyl, fluorocyclobutyl, difluorocyclobutyl, difluorocyclopentyl, hydroxycyclohexyl, ethyl, isopropyl, tert-butyl, methyl, 2,4-dimethylpent-3-yl, dicyclopropylmethyl, spiro[3.3]heptyl, 2-methoxycyclopropyl, hexahydro-1H-isoindol-2(3H)-yl, bicyclo[1.1.1]pentyl, bicyclo[2.2.2]octyl, 4-hydroxybicyclo[2.2.2]octan-1-yl, 4,4-dimethylcyclohexyl, oxetanyl, oxazolyl, 2,3-dihydro-1H-indenyl, quinuclidinyl, 1,7,7-trimethylbicyclo[2.2.1]heptyl, 7,7-dimethylbicyclo[2.2.1]heptyl, bicyclo[2.2.1]heptyl, 1-cyclopropylethyl, 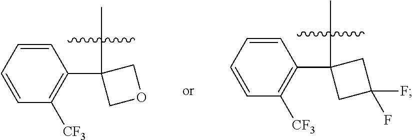 [SMILES: *C1(c2ccccc2C(F)(F)F)COC1 ; *C1(c2ccccc2C(F)(F)F)CC(F)(F)C1]
or
(ix) wherein X₁ represents NR₂R₃, wherein R₂ represents H, and R₃ represents -thiazolyl-C(O)NHR_{d}, wherein R_{d} represents C₁₋₅ alkyl or 2-chloro-6-methylphenyl; or
(x) wherein X₁ represents NR₂R₃, wherein R₂ represents H, and R₃ represents pyrimidin-4-yl, pyridin-4-yl, or quinazolin-4-yl; or
(xi) wherein X₁ represents NR₂R₃, wherein R₂ and R₃ represents phenyl; or
(xii) wherein X₁ represents NHCH₃, N(CH₃)₂, N(CH₂CH₃)₂ or N(CH(CH₃)₂)₂; or
(xiii) wherein X₁ represents NHC(O)NR₇R₈, wherein R₇ represents H, and R₈ represents adamantanyl, adamantan-1-yl, adamantan-2-yl, 3,5-dimethyladamantan-1-yl, phenyl, or 3-chloro-4-methylphenyl; or
(xiv) wherein X₁ represents NR₂R₃, wherein R₂ represents H or methyl, and R₃ represents pyrimidinyl, pyridyl, quinazolinyl, 6,7-difluoroquinazolin-4-yl, phenyl, 3-chloro-4-methylphenyl, 4-fluorophenyl, 3,5-difluorophenyl, 3,4,5-trifluorophenyl, 2-(dimethylphosphono)phenyl, 2-(isopropylsulfonyl)phenyl, 4-(pyridin-3-yl)pyrimidin-2-yl, indolyl, 1-methyl-1H-indol-7-yl, benzothienyl, benzo[b]thien-7-yl, benzofuran-7-yl, benzofuranyl, 3-cyano-quinol-4-yl, quinolyl, thiazolyl, adamantanyl, adamantan-1-yl, adamantan-2-yl, 3,5-dimethyladamantan-1-yl, 1,7,7-trimethylbicyclo[2.2.1]heptan-2-yl, 4-(4-methylpiperazin-1-yl)piperidin-1-yl, cyclohexyl, dimethylcyclohexyl, 4,4-dimethylcyclohexyl, spiro-cycloalkyl, spiro[5.5]undec-3-yl, spiro[3.3]heptyl, spiro[3.3]hept-2-yl, pyrrolidinyl, azepanyl, azacyclooctanyl, piperidinyl, dimethylpiperidinyl, or azaspirocycloalkyl; or
(xv) wherein X₁ represents a quaternary ammonium salt group of Formula N⁺RₐR_{b}R_{c}X⁻, wherein Rₐ, R_{b}, and R_{c} each independently represent C₁₋₁₀ alkyl, and X⁻ represents Cl⁻; or
Rₐ and R_{b} each independently represent methyl, ethyl, propyl, isopropyl or butyl, R_{c} represents ethyl, and X⁻ represents Cl⁻; or
Rₐ, R_{b}, and R_{c} represent ethyl, and X⁻ represents Cl⁻; or
Rₐ, R_{b}, and R_{c} represent methyl, and X⁻ represents Cl⁻; or
(xvi) wherein X₁ represents 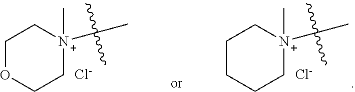
(xvii) wherein X₁ represents the group of Formula (G₂): 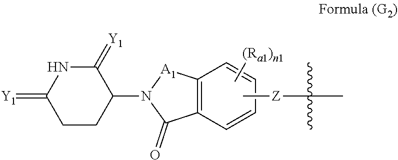
wherein A₁ represents CH₂ or C(O), Y₁ represents O, and Z represents S, S(O) or S(O)₂, and (Rₐ₁)ₙ₁ represents denotes that the benzene ring is substituted with n1 Rₐ, where R₁ groups are the same or different and each independently represent deuterium, halogen, hydroxy, mercapto, nitro, amino, cyano, optionally deuterated C₁₋₆ alkyl, optionally deuterated C₁₋₆ alkoxy, halogenated C₁₋₆ alkyl, optionally substituted C₃₋₆ cycloalkyl, C₂₋₆ alkenyl, or C₂₋₆ alkynyl, and n represents an integer of 0, 1, 2, or 3.

10. The compound of Formula (I) or salts, enantiomers, diastereoisomer, isotopically enriched analogs, solvates, prodrugs, or polymorphs thereof of claim 1,
(i) wherein L represents the structure of the following formula:
linear or branched C₁-C₄₀alkylene, *—(CH₂)ₙ₂—(O—(CH₂)ₙ₃)ₘ₁—, *—(CH₂)ₙ₂—(O—(CH₂)ₙ₃)ₘ₁—(O—(CH₂)ₙ₄)ₘ₂—, *—(CH₂)ₙ₂—(O—(CH₂)ₙ₃)ₘ₁—(O—(CH₂)ₙ₄)ₘ₂—(O—(CH₂)ₙ₅)ₘ₃—, *—(CH₂)ₙ₂—S—(CH₂)ₙ₃—, *—(CH₂)ₙ₂—S(O)—(CH₂)ₙ₃—, *—(CH₂)ₙ₂—S(O)₂—(CH₂)ₙ₃—, *—(CH₂)ₙ₂—N(R₁)S(O)₂—(CH₂)ₙ₃—, *—(CH₂)ₙ₂—S(O)₂N(R₁)—(CH₂)ₙ₃—, *—(CH₂)ₙ₂—N(R₁)—(CH₂)ₙ₃—, *—(CH₂)ₙ₂—N(R₁)C(O)—(CH₂)ₙ₃—, *—(CH₂)ₙ₂—C(O)N(R₁)—(CH₂)ₙ₃—, *—(CH₂)ₙ₂—N(R₁)C(O)N(R₁)—(CH₂)ₙ₃—, *—(CH₂)ₙ₂—(N(R₁)C(O)—(CH₂)ₙ₃)ₘ₁—, *—(CH₂)ₙ₂-piperazinylene-(CH₂)ₙ₃—, *—(CH₂)ₙ₂-phenylene-(CH₂)ₙ₃—, *—(CH₂)ₙ₂-phenylene-(CH₂)ₙ₃—N(R₁)—(CH₂)ₙ₄—, *—(CH₂)ₙ₂-furanylene-(CH₂)ₙ₃—, *—(CH₂)ₙ₂-furanylene-(CH₂)ₙ₃—N(R₁)—(CH₂)ₙ₄—, *—(CH₂)ₙ₂-thiazolylene-(CH₂)ₙ₃—, *—(CH₂)ₙ₂-thiazolylene-C(O)N(R₁)—(CH₂)ₙ₃—, *—(CH₂)ₙ₂-thiazolylene-(CH₂)ₙ₃—N(R₁)—(CH₂)ₙ₄—;
wherein a hydrogen of one or more CH₂ groups of the linear or branched C₁₋₄₀ alkylene is optionally replaced with a substituent selected from the group consisting of: halogen, cyano, C₁₋₃alkyl, C₁₋₃ alkoxy, trifluoromethyl, C₅₋₁₀ aryl, 5- to 10-membered heteroaryl, C₃₋₁₂cycloalkyl, 3- to 12-membered heterocyclyl, or any combination thereof;
symbol * indicates the point of attachment to R;
R₁ groups each independently represent H or C₁₋₃alkyl;
n2, n3, n4, n5, m1, m2, and m3 each independently represent an integer of 1, 2, 3, 4, 5, 6, 7, 8, 9, 10, 11, 12, 13, 14, or 15;
the piperazinylene is optionally substituted with a substituent selected from the group consisting of halogen, cyano, hydroxy, amino, mercapto, oxo, C₁₋₃ alkoxy, halogenated C₁₋₃alkyl, methanesulfonyloxy, trifluoromethanesulfonyloxy, p-toluenesulfonyloxy, optionally deuterated C₁₋₆ alkyl, optionally deuterated C₃₋₆ cycloalkyl, or any combination thereof; and the phenylene, the furanylene, and the thiazolylene are each independently optionally substituted with a substituent selected from the group consisting of halogen, cyano, hydroxy, amino, mercapto, C₁₋₃ alkoxy, halogenated C₁₋₃alkyl, methanesulfonyloxy, trifluoromethanesulfonyloxy, p-toluenesulfonyloxy, optionally deuterated C₁₋₆ alkyl, optionally deuterated C₃₋₆ cycloalkyl, or any combination thereof; or
(ii) wherein X₁ represents:
morpholinyl, piperidinyl, 2-oxopiperidin-1-yl, piperazinyl, N-methylpiperazinyl, 4-(methylsulfonyl)piperazin-1-yl, 4-(ethylsulfonyl)piperazin-1-yl, (N-methylpiperazinyl)piperidinyl, 3,5-dimethylpiperazinyl, 1,4-diazepan-1-yl, 4-methyl-1,4-diazepan-1-yl, 1-methyl-1,4-diazepan-1-yl, 8-methyl-3,8-diazabicyclo[3.2.1]octan-3-yl, 1,4-diazabicyclo[3.2.1]octan-4-yl, 5-methyl-2,5-diazabicyclo[2.2.2]octan-2-yl, 8,8-difluoro-2-azabicyclo[3.2.1]octan-2-yl, 4-(4-fluorophenyl)piperazin-1-yl, 4-(3,4-difluorophenyl)piperazin-1-yl, 2-oxopiperidin-1-yl, 2-oxopyrrolidin-1-yl, 3-fluoro-4-hydroxypyrrolidin-1-yl, 3-methyl-4-(trifluoromethyl)pyrrolidin-1-yl, 2,6-dioxopiperidin-3-yl, 4,4-dimethylpiperidin-1-yl, 3,5-dimethylpiperidin-1-yl, 4,4-difluoropiperidin-1-yl, 4-methylpiperazin-1-yl, tetrahydro-2H-pyran-2-yl, pyridyl, pyrimidinyl, thiazolyl, quinolyl, 6,7-difluoroquinazolin-4-yl, 3-cyano-quinolyl, indolyl, 1-methyl-1H-indol-7-yl, benzothienyl, benzofuranyl, quinazolinyl, 6,7-difluoroquinazolin-4-yl, pyrrolidinyl, pyrrolidin-1-yl, 4-(pyridin-3-yl)pyrimidin-2-yl, 3-cyanoazetidinyl, 3-hydroxyazetidinyl, 3-hydroxy-3-methylazetidinyl, 3-hydroxy-2-methylazetidin-1-yl, 3-(trifluoromethoxy)azetidin-1-yl, azepan-1-yl, azacyclooctan-1-yl, 7-fluoro-5-azaspiro[2.4]heptan-5-yl, 1,1-difluoro-5-azaspiro[2.4]heptan-5-yl, 1,1-difluoro-6-azaspiro[2.5]octan-6-yl, 6-azaspiro[2.5]octan-6-yl, 2-oxa-7-azaspiro[3.5]nonan-7-yl, 3-azabicyclo[3.1.0]hexan-3-yl, 6-(difluoromethyl)-3-azabicyclo[4.1.0]heptan-3-yl, 3-fluoro-5,7-dihydro-6H-pyrrolo[3,4-b]pyridin-6-yl, 3-(trifluoromethyl)-5,7-dihydro-6H-pyrrolo[3,4-b]pyridin-6-yl, isoindol-2-yl, thiomorpholino, [1,4′-dipiperidin]-1′-yl, (2-bromovinyl)phenyl, 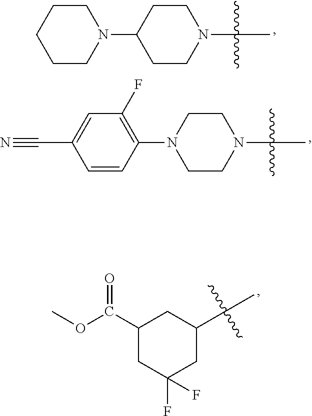
or 4,4-difluoropiperidinyl; or
(iii) wherein X₁ represents NR₂R₃, wherein
R₂ represents H, methyl, ethyl, isopropyl, or cyclohexyl, and
R₃ represents bicyclo[1.1.1]pent-1-yl, 4,4-dimethylcyclohexyl, 2-methoxycyclopropyl, 3-fluorocyclobutyl, 3,3-difluorocyclobutyl, 3,3-difluoro-1-(2-(trifluoromethyl)phenyl)cyclobutyl, 3-(2-(trifluoromethyl)phenyl)oxetan-3-yl, 3,3-difluorocyclopentyl, 3-hydroxycyclohexyl, 3-hydroxycyclohexyl, 3-cyano-bicyclo[1.1.1]pent-1-yl, bicyclo[2.2.2]octan-1-yl, 4-hydroxybicyclo[2.2.2]octan-1-yl, 2-isopropyl-5-methylcyclohexyl, 5-methyloxazol-2-yl, 4-cyano-2,3-dihydro-1H-inden-1-yl, 2,4-dimethylpentan-3-yl, dicyclopropylmethyl, quinuclidin-3-yl, (S)-quinuclidin-3-yl, (R)-quinuclidin-3-yl, adamantan-1-yl, 3-hydroxyadamantanyl, 3-hydroxyadamantan-1-yl, 3-chloroadamantan-1-yl, 4-chloroadamantan-1-yl, 2-chloroadamantan-1-yl, adamantan-2-yl, 1,7,7-trimethylbicyclo[2.2.1]heptan-2-yl, 7,7-dimethylbicyclo[2.2.1]heptan-1-yl, 7,7-dimethyl-2-oxobicyclo[2.2.1]heptan-1-yl, 3,5-dimethyladamantanyl, 3,5-dimethyladamantan-1-yl, hexahydro-2,5-methanopentalen-3a(1H)-yl, bicyclo[2.2.1]heptyl, bicyclo[2.2.1]heptan-2-yl, 1-cyclopropylethyl,

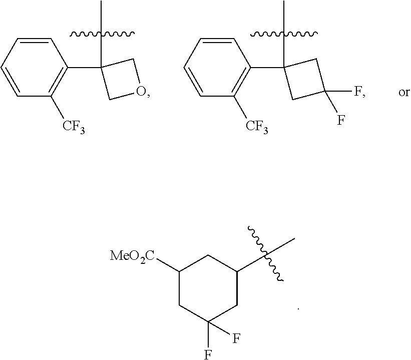 [SMILES: *C1(c2ccccc2C(F)(F)F)COC1 ; *C1(c2ccccc2C(F)(F)F)CC(F)(F)C1 ; *C1CC(C(=O)OC)CC(F)(F)C1]

11. The compound of Formula (I) or salts, enantiomers, diastereoisomer, isotopically enriched analogs, solvates, prodrugs, or polymorphs thereof of claim 1, wherein L represents the following groups:
—CH₂—; —(CH₂)₂—; —(CH₂)₃—; —(CH₂)₄—; —(CH₂)₅—; —(CH₂)₆—; —(CH₂)₇—; —(CH₂)₈—; —(CH₂)₉—; —(CH₂)₁₀—; —(CH₂)₁₁—; —(CH₂)₁₂—; —(CH₂)₁₃—; —(CH₂)₁₄—; —(CH₂)₁₅—; —(CH₂)₁₆—; —(CH₂)₁₇—; —(CH₂)₁₈—; —(CH₂)₁₉—; or —(CH₂)₂₀—;
*—CH₂CH₂O(CH₂)₂—; *—(CH₂CH₂O)₂—(CH₂)₂—; *—(CH₂CH₂O)₃—(CH₂)₂—; *—(CH₂CH₂O)₄—(CH₂)₂—; *—(CH₂CH₂O)₅—(CH₂)₂—; *—(CH₂CH₂O)₆—(CH₂)₂—; *—(CH₂CH₂O)₇—(CH₂)₂—; *—(CH₂CH₂O)₈—(CH₂)₂—; *—(CH₂CH₂O)₉(CH₂)₂—; *—(CH₂CH₂O)₁₀(CH₂)₂—; *—CH₂CH₂OCH₂—; *—(CH₂CH₂O)₂—CH₂—; *—(CH₂CH₂O)₃—CH₂—; *—(CH₂CH₂O)₄—CH₂—; *—(CH₂CH₂O)₅—CH₂—; *—(CH₂CH₂O)₆—CH₂—; *—(CH₂CH₂O)₇—CH₂—; *—(CH₂CH₂O)₈—CH₂—; *—(CH₂CH₂O)₉—CH₂—; *—(CH₂CH₂O)₁₀—CH₂—; *—CH₂CH₂O(CH₂)₃—; *—(CH₂CH₂O)₂—(CH₂)₃—; *—(CH₂CH₂O)₃—(CH₂)₃—; *—(CH₂CH₂O)₄—(CH₂)₃—; *—(CH₂CH₂O)₅—(CH₂)₃—; *—(CH₂CH₂O)₆—(CH₂)₃—; *—(CH₂CH₂O)₇—(CH₂)₃—; *—(CH₂CH₂O)₈—(CH₂)₃—; *—(CH₂CH₂O)₉(CH₂)₃—; *—(CH₂CH₂O)₁₀(CH₂)₃—; *—CH₂CH₂OCH₂CH₂CH₂OCH₂—; *—CH₂CH₂OCH₂CH₂CH₂O—(CH₂)₂—; *—CH₂CH₂OCH₂CH₂CH₂O—(CH₂)₃—; *—(CH₂CH₂O)₂(CH₂CH₂CH₂O)(CH₂)₃—; *—(CH₂CH₂O)₂(CH₂CH₂CH₂O)₂(CH₂)₃—; *—(CH₂)₁O(CH₂)₁—; *—(CH₂)₁O(CH₂)₂—; *—(CH₂)₂O(CH₂)₂—; *—(CH₂)₂O(CH₂)₁—; *—(CH₂)₂O(CH₂)₃—; *—(CH₂)₂O(CH₂)₄—; *—(CH₂)₂O(CH₂)₅—; *—(CH₂)₂O(CH₂)₆—; *—(CH₂)₃O(CH₂)₁—; *—(CH₂)₃O(CH₂)₂—; *—(CH₂)₃O(CH₂)₃—; *—(CH₂)₄O(CH₂)₁—; *—(CH₂)₄O(CH₂)₂—; *—(CH₂)₄O(CH₂)₃—; *—(CH₂)₅O(CH₂)₁—; *—(CH₂)₅O(CH₂)₂—; *—(CH₂)₅O(CH₂)₃—; *—(CH₂)₅O(CH₂)₄—; or *—(CH₂)₅O(CH₂)₅—;
*—(CH₂)₁—NH—(CH₂)₁—; *—(CH₂)₂—NH—(CH₂)₁—; *—(CH₂)₂—NH—(CH₂)₂—; *—(CH₂)₂—NH—(CH₂)₃—; *—(CH₂)₂—NH—(CH₂)₄—; *—(CH₂)₂—NH—(CH₂)₅—; *—(CH₂)₂—NH—(CH₂)₆—; *—(CH₂)₂—NH—(CH₂)₇—; *—(CH₂)₂—NH—(CH₂)₈—; *—(CH₂)₂—NH—(CH₂)₉—; *—(CH₂)₂—NH—(CH₂)₁₀—; *—(CH₂)₂—NH—(CH₂)₁₁—; *—(CH₂)₂—NH—(CH₂)₁₂—; *—(CH₂)₃—NH—(CH₂)₁—; *—(CH₂)₃—NH—(CH₂)₂—; *—(CH₂)₃—NH—(CH₂)₃—; *—(CH₂)₄—NH—(CH₂)₁—; *—(CH₂)₄—NH—(CH₂)₂—; *—(CH₂)₅—NH—(CH₂)₃—; *—(CH₂)₅—NH—(CH₂)₁—; *—(CH₂)₅—NH—(CH₂)₂—; *—(CH₂)₈—NH—(CH₂)₂—; *—(CH₂)₅—NH—(CH₂)₃—; *—(CH₂)₅—NH—(CH₂)₄—; *—(CH₂)₅—NH—(CH₂)₅—; *—(CH₂)₁—N(CH₃)—(CH₂)₈—; *—(CH₂)₂—N(CH₃)—(CH₂)₁—; *—(CH₂)₃—N(CH₃)—(CH₂)₁—; *—(CH₂)₄—N(CH₃)—(CH₂)₁—; *—(CH₂)₅—N(CH₃)—(CH₂)₁—; *—(CH₂)₆—N(CH₃)—(CH₂)₁—; *—(CH₂)₂—N(CH₃)—(CH₂)₂—; *—(CH₂)₂—N(CH₃)—(CH₂)₃—; *—(CH₂)₂—N(CH₃)—(CH₂)₄—; *—(CH₂)₂—N(CH₃)—(CH₂)₅—; *—(CH₂)₂—N(CH₃)—(CH₂)₆—; *—(CH₂)₂—N(CH₃)—(CH₂)₇—; *—(CH₂)₂—N(CH₃)—(CH₂)₈—; *—(CH₂)₂—N(CH₃)—(CH₂)₉—; *—(CH₂)₂—N(CH₃)—(CH₂)₁₀—; *—(CH₂)₂—N(CH₃)—(CH₂)₁₁—; *—(CH₂)₂—N(CH₃)—(CH₂)₁₂—; *—(CH₂)₂—NHC(O)—CH₂—; *—(CH₂)₂—NHC(O)—(CH₂)₂—; *—(CH₂)₂—NHC(O)—(CH₂)₃—; *—(CH₂)₂—NHC(O)—(CH₂)₄—; *—(CH₂)₂—NHC(O)—(CH₂)₅—; *—(CH₂)₂—NHC(O)—(CH₂)₆—; *—(CH₂)₂—NHC(O)—(CH₂)₇—; *—(CH₂)₂—NHC(O)—(CH₂)₈—; *—(CH₂)₂—NHC(O)—(CH₂)₉—; *—(CH₂)₂—NHC(O)—(CH₂)₁₀—; *—(CH₂)₂—NHC(O)—(CH₂)₁₁—; *—(CH₂)₂—NHC(O)—(CH₂)₁₂—; *—(CH₂)₂—NHC(O)—(CH₂)₁₃—; *—(CH₂)₂—NHC(O)—(CH₂)₁₄—; *—(CH₂)₂—NHC(O)—(CH₂)₁₅—; *—(CH₂)₃—NHC(O)—CH₂—; *—(CH₂)₃—NHC(O)—(CH₂)₂—; *—(CH₂)₃—NHC(O)—(CH₂)₃—; *—(CH₂)₃—NHC(O)—(CH₂)₄—; *—(CH₂)₃—NHC(O)—(CH₂)₅—; *—(CH₂)₃—NHC(O)—(CH₂)₆—; *—(CH₂)₃—NHC(O)—(CH₂)₇—; *—(CH₂)₃—NHC(O)—(CH₂)₈—; *—(CH₂)₃—NHC(O)—(CH₂)₉—; *—(CH₂)₃—NHC(O)—(CH₂)₁₀—; *—(CH₂)₃—NHC(O)—(CH₂)₁—; *—(CH₂)₃—NHC(O)—(CH₂)₁₂—; *—(CH₂)₃—NHC(O)—(CH₂)₁₃—; *—(CH₂)₃—NHC(O)—(CH₂)₁₄—; *—(CH₂)₃—NHC(O)—(CH₂)₁₅—; *—(CH₂)₄NHC(O)(CH₂)₁—; *—(CH₂)₄NHC(O)(CH₂)₂—; *—(CH₂)₄NHC(O)(CH₂)₃—; *—(CH₂)₄NHC(O)(CH₂)₄—; *—(CH₂)₄NHC(O)(CH₂)₅—; *—(CH₂)₄NHC(O)(CH₂)₆—; *—(CH₂)₄NHC(O)(CH₂)₇—; *—(CH₂)₄NHC(O)(CH₂)₈—; *—(CH₂)₄NHC(O)(CH₂)₉—; *—(CH₂)₄NHC(O)(CH₂)₁₀—; *—(CH₂)₅NHC(O)(CH₂)₁—; *—(CH₂)₈NHC(O)(CH₂)₂—; *—(CH₂)₂—N(CH₃)C(O)—CH₂—; *—(CH₂)₂—N(CH₃)C(O)—(CH₂)₂—; *—(CH₂)₂—N(CH₃)C(O)—(CH₂)₃—; *—(CH₂)₂—N(CH₃)C(O)—(CH₂)₄—*—(CH₂)₂—N(CH₃)C(O)—(CH₂)₅—; *—(CH₂)₂—N(CH₃)C(O)—(CH₂)₆—; *—(CH₂)₂—N(CH₃)C(O)—(CH₂)₇—; *—(CH₂)₂—N(CH₃)C(O)—(CH₂)₈—; *—(CH₂)₂—N(CH₃)C(O)—(CH₂)₉—; *—(CH₂)₂—N(CH₃)C(O)—(CH₂)₁₀—; *—(CH₂)₂—N(CH₃)C(O)—(CH₂)₁₁—; *—(CH₂)₂—N(CH₃)C(O)—(CH₂)₁₂—; *—(CH₂)₂—N(CH₃)C(O)—(CH₂)₁₃—; *—(CH₂)₂—N(CH₃)C(O)—(CH₂)₁₄—; *—(CH₂)₂—N(CH₃)C(O)—(CH₂)₁₅—; *—(CH₂)₂—C(O)NH—CH₂—; *—(CH₂)₂—C(O)NH—(CH₂)₂—; *—(CH₂)₂—C(O)NH—(CH₂)₃—; *—(CH₂)₂—C(O)NH—(CH₂)₄—; *—(CH₂)₂—C(O)NH—(CH₂)₅—; *—(CH₂)₂—C(O)NH—(CH₂)₆—; *—(CH₂)₂—C(O)NH—(CH₂)₇—; *—(CH₂)₂—C(O)NH—(CH₂)₈—; *—(CH₂)₂—C(O)NH—(CH₂)₉—; *—(CH₂)₂—C(O)NH—(CH₂)₁₀—; *—(CH₂)₂—C(O)NH—(CH₂)₁₁—; *—(CH₂)₂—C(O)NH—(CH₂)₁₂—; *—(CH₂)₂—C(O)NH—(CH₂)₁₃—; *—(CH₂)₂—C(O)NH—(CH₂)₁₄—; *—(CH₂)₂—C(O)NH—(CH₂)₁₅—; *—(CH₂)₃—C(O)NH—CH₂—; *—(CH₂)₃—C(O)NH—(CH₂)₂—; *—(CH₂)₃—C(O)NH—(CH₂)₃—; *—(CH₂)₃—C(O)NH—(CH₂)₄—; *—(CH₂)₃—C(O)NH—(CH₂)₅—; *—(CH₂)₃—C(O)NH—(CH₂)₆—; *—(CH₂)₃—C(O)NH—(CH₂)₇—; *—(CH₂)₃—C(O)NH—(CH₂)₈—; *—(CH₂)₃—C(O)NH—(CH₂)₉—; *—(CH₂)₃—C(O)NH—(CH₂)₁₀—; *—(CH₂)₃—C(O)NH—(CH₂)₁₁—; *—(CH₂)₃—C(O)NH—(CH₂)₁₂—; *—(CH₂)₃—C(O)NH—(CH₂)₁₃—; *—(CH₂)₃—C(O)NH—(CH₂)₁₄—; *—(CH₂)₃—C(O)NH—(CH₂)₁₅—; *—(CH₂)₄C(O)NH(CH₂)₁—; *—(CH₂)₄C(O)NH(CH₂)₂—; *—(CH₂)₄C(O)NH(CH₂)₃—; *—(CH₂)₄C(O)NH(CH₂)₄—; *—(CH₂)₄C(O)NH(CH₂)₅—; *—(CH₂)₄C(O)NH(CH₂)₆—; *—(CH₂)₄C(O)NH(CH₂)₇—; *—(CH₂)₄C(O)NH(CH₂)₈—; *—(CH₂)₄C(O)NH(CH₂)₉—; *—(CH₂)₄C(O)NH(CH₂)₁₀—; *—(CH₂)₂—C(O)N(CH₃)—CH₂—; *—(CH₂)₂—C(O)N(CH₃)—(CH₂)₂—; *—(CH₂)₂—C(O)N(CH₃)—(CH₂)₃—; *—(CH₂)₂—C(O)N(CH₃)—(CH₂)₄—; *—(CH₂)₂—C(O)N(CH₃)—(CH₂)₅—; *—(CH₂)₂—C(O)N(CH₃)—(CH₂)₆—; *—(CH₂)₂—C(O)N(CH₃)—(CH₂)₇—; *—(CH₂)₂—C(O)N(CH₃)—(CH₂)₈—; *—(CH₂)₂—C(O)N(CH₃)—(CH₂)₉—; *—(CH₂)₂—C(O)N(CH₃)—(CH₂)₁₀—; *—(CH₂)₂—C(O)N(CH₃)—(CH₂)₁—; *—(CH₂)₂—C(O)N(CH₃)—(CH₂)₁₂—; *—(CH₂)₂—C(O)N(CH₃)—(CH₂)₁₃—; *—(CH₂)₂—C(O)N(CH₃)—(CH₂)₁₄—; *—(CH₂)₂—C(O)N(CH₃)—(CH₂)₁₅—; *—(CH₂)₂—NHC(O)NH—(CH₂)₄—; *—(CH₂)₄—NHC(O)NH—(CH₂)₂—; *—CH₂—NHC(O)NH—(CH₂)₂—; *—(CH₂)₂—NHC(O)NH—CH₂—; *—(CH₂)₂—NHC(O)NH—(CH₂)₂—; *—(CH₂)₂—NHC(O)NH—(CH₂)₃—; *—(CH₂)₃—NHC(O)NH—(CH₂)₂—; *—CH₂-piperazinylene-CH₂—; *—(CH₂)₂-piperazinylene-(CH₂)₂—; *—(CH₂)₂-piperazinylene-(CH₂)₃—; *—(CH₂)₂-piperazinylene-(CH₂)₄—; *—(CH₂)₂-piperazinylene-(CH₂)₅—; *—(CH₂)₃-piperazinylene-CH₂—; *—(CH₂)₃-piperazinylene-(CH₂)₂—; *—(CH₂)₃-piperazinylene-(CH₂)₃—; *—(CH₂)₄-piperazinylene-CH₂—; *—(CH₂)₄-piperazinylene-(CH₂)₂—; *—(CH₂)₄-piperazinylene-(CH₂)₃—; *—(CH₂)₈-piperazinylene-CH₂—; *—(CH₂)₈-piperazinylene-(CH₂)₂—; *—(CH₂)₈-piperazinylene-(CH₂)₃—; *—(CH₂)₈-piperazinylene-(CH₂)₄—; *—(CH₂)₈-piperazinylene-(CH₂)₅—; *—(CH₂)₈-piperazinylene-(CH₂)₆—; *—(CH₂)₈-piperazinylene-(CH₂)₇—; *—(CH₂)₈-piperazinylene-(CH₂)₈—; *—CH₂-piperazinylene-(CH₂)₈—; *—(CH₂)₂-piperazinylene-(CH₂)₈—; *—(CH₂)₃-piperazinylene-(CH₂)₈—; *—(CH₂)₄-piperazinylene-(CH₂)₈—; *—(CH₂)₅-piperazinylene-(CH₂)₈—; *—(CH₂)₆-piperazinylene-(CH₂)₈—; *—(CH₂)₇-piperazinylene-(CH₂)₈—; *—CH₂-phenylene-CH₂—; *—(CH₂)₂-phenylene-(CH₂)₂—; *—(CH₂)₂-phenylene-(CH₂)₃—; *—(CH₂)₂-phenylene-(CH₂)₄—; *—(CH₂)₂-phenylene-(CH₂)₅—; *—(CH₂)₃-phenylene-CH₂—; *—(CH₂)₃-phenylene-(CH₂)₂—; *—(CH₂)₃-phenylene-(CH₂)₃—; *—(CH₂)₄-phenylene-CH₂—; *—(CH₂)₄-phenylene-(CH₂)₂—; *—(CH₂)₄-phenylene-(CH₂)₃—; *—(CH₂)₅-phenylene-(CH₂)₃—; *—(CH₂)₆-phenylene-(CH₂)₃—; *—(CH₂)₇-phenylene-(CH₂)₃—; *—(CH₂)₈-phenylene-CH₂—; *—(CH₂)₈-phenylene-(CH₂)₂—; *—(CH₂)₈-phenylene-(CH₂)₃—; *—(CH₂)₈-phenylene-(CH₂)₄—; *—(CH₂)₈-phenylene-(CH₂)₅—; *—(CH₂)₈-phenylene-(CH₂)₆—; *—(CH₂)₈-phenylene-(CH₂)₇—; *—(CH₂)₈-phenylene-(CH₂)₈—; *—CH₂-phenylene-(CH₂)₈—; *—(CH₂)₂-phenylene-(CH₂)₈—; *—(CH₂)₃-phenylene-(CH₂)₈—; *—(CH₂)₄-phenylene-(CH₂)₈—; *—(CH₂)₅-phenylene-(CH₂)₈—; *—(CH₂)₆-phenylene-(CH₂)₈—; *—(CH₂)₇-phenylene-(CH₂)₈—; *—(CH₂)₁S(CH₂)₁—; *—(CH₂)₂S(CH₂)₂—; *—(CH₂)₂S(CH₂)₁—; *—(CH₂)₁S(CH₂)₂—; *—(CH₂)₁S(CH₂)₃—; *—(CH₂)₁S(CH₂)₄—; *—(CH₂)₂S(CH₂)₃—; *—(CH₂)₂S(CH₂)₄—; *—(CH₂)₂S(CH₂)₅—; *—(CH₂)₃S(CH₂)₁—; *—(CH₂)₃S(CH₂)₂—; *—(CH₂)₃S(CH₂)₃—; *—(CH₂)₄S(CH₂)₁—; *—(CH₂)₄S(CH₂)₂—; *—(CH₂)₄S(CH₂)₃—; *—(CH₂)₅S(CH₂)₁—; *—(CH₂)₅S(CH₂)₂—; *—(CH₂)₅S(CH₂)₃—; *—(CH₂)₆S(CH₂)₁—; *—(CH₂)₆S(CH₂)₂—; *—(CH₂)₆S(CH₂)₃—; *—(CH₂)₇S(CH₂)₁—; *—(CH₂)₇S(CH₂)₂—; *—(CH₂)₇S(CH₂)₃—; *—(CH₂)₈S(CH₂)₁—; *—(CH₂)₈S(CH₂)₂—; *—(CH₂)₈S(CH₂)₃—; *—(CH₂)₉S(CH₂)₁—; *—(CH₂)₉S(CH₂)₂—; *—(CH₂)₉S(CH₂)₃—; *—(CH₂)₁S(O) (CH₂)₁—; *—(CH₂)₂S(O)(CH₂)₂—; *—(CH₂)₂S(O)(CH₂)₁—; *—(CH₂)₁S(O)(CH₂)₂—; *—(CH₂)₁S(O)(CH₂)₃—; *—(CH₂)₁S(O)(CH₂)₄—; *—(CH₂)₂S(O)(CH₂)₃—; *—(CH₂)₂S(O)(CH₂)₄—; *—(CH₂)₂S(O)(CH₂)₅—; *—(CH₂)₃S(O)(CH₂)₁—; *—(CH₂)₃S(O)(CH₂)₂—; *—(CH₂)₃S(O)(CH₂)₃—; *—(CH₂)₄S(O)(CH₂)₁—; *—(CH₂)₄S(O)(CH₂)₂—; *—(CH₂)₄S(O)(CH₂)₃—; *—(CH₂)₅S(O)(CH₂)₁—; *—(CH₂)₅S(O)(CH₂)₂; *—(CH₂)₅S(O)(CH₂)₃; *—(CH₂)₆S(O)(CH₂)₁—; *—(CH₂)₆S(O)(CH₂)₂—; *—(CH₂)₆S(O)(CH₂)₃—; *—(CH₂)₇S(O)(CH₂)₁—; *—(CH₂)₇S(O)(CH₂)₂—; *—(CH₂)₇S(O)(CH₂)₃—; *—(CH₂)₈S(O)(CH₂)₁—; *—(CH₂)₈S(O)(CH₂)₂—; *—(CH₂)₈S(O)(CH₂)₃—; *—(CH₂)₉S(O)(CH₂)₁—; *—(CH₂)₉S(O)(CH₂)₂—; *—(CH₂)₉S(O)(CH₂)₃—; *—(CH₂)₁S(O)₂(CH₂)₁—; *—(CH₂)₂S(O)₂(CH₂)₂—; *—(CH₂)₂S(O)₂(CH₂)₁—; *—(CH₂)₁S(O)₂(CH₂)₂—; *—(CH₂)₁S(O)₂(CH₂)₃—; *—(CH₂)₁S(O)₂(CH₂)₄—; *—(CH₂)₂S(O)₂(CH₂)₃—; *—(CH₂)₂S(O)₂(CH₂)₄—; *—(CH₂)₂S(O)₂(CH₂)₅—; *—(CH₂)₃S(O)₂(CH₂)₁—; *—(CH₂)₃S(O)₂(CH₂)₂—; *—(CH₂)₃S(O)₂(CH₂)₃—; *—(CH₂)₄S(O)₂(CH₂)₁—; *—(CH₂)₄S(O)₂(CH₂)₂—; *—(CH₂)₄S(O)₂(CH₂)₃—; *—(CH₂)₅S(O)₂(CH₂)₁—; *—(CH₂)₅S(O)₂(CH₂)₂—; *—(CH₂)₅S(O)(CH₂)₃—; *—(CH₂)₆S(O)₂(CH₂)₁—; *—(CH₂)₆S(O)₂(CH₂)₂—; *—(CH₂)₆S(O)₂(CH₂)₃—; *—(CH₂)₇S(O)₂(CH₂)₁—; *—(CH₂)₇S(O)₂(CH₂)₂—; *—(CH₂)₇S(O)₂(CH₂)₃—; *—(CH₂)₈S(O)₂(CH₂)₁—; *—(CH₂)₈S(O)₂(CH₂)₂—; *—(CH₂)₈S(O)₂(CH₂)₃—; *—(CH₂)₉S(O)₂(CH₂)₁—; *—(CH₂)₉S(O)₂(CH₂)₂—; or *—(CH₂)₉S(O)₂(CH₂)₃—;
*—(CH₂)₆—NH—(CH₂)₁—; *—(CH₂)₆—NH—(CH₂)₃—; *—(CH₂)₆—NH—(CH₂)₄—; *—(CH₂)₆—NH—(CH₂)₅—; *—(CH₂)₇—NH—(CH₂)₁—; *—(CH₂)₇—NH—(CH₂)₂—; *—(CH₂)₇—NH—(CH₂)₃—; *—(CH₂)₇—NH—(CH₂)₄—; *—(CH₂)₆—NH—CH(CH₃)—; *—(CH₂)₅—NH—CH(CH₃)—; *—(CH₂)₄—NH—CH(CH₃)—; *—(CH₂)₃—NH—CH(CH₃)—; *—(CH₂)₂—NH—CH(CH₃)—; *—(CH₂)₇—NH—CH(CH₃)—; *—(CH₂)₈—NH—CH(CH₃)—; *—(CH₂)₇—NH—CH(CF₃)—; *—(CH₂)₆—NH—CH(CF₃)—; *—(CH₂)₅—NH—CH(CF₃)—; *—(CH₂)₄—NH—CH(CF₃)—; *—(CH₂)₃—NH—CH(CF₃)—; *—(CH₂)₂—NH—CH(CF₃)—; *—(CH₂)₈—NH—CH(CF₃)—; *—CH₂—C(O)NH—(CH₂)₄—; *—CH₂—C(O)NH—(CH₂)₂—; *—CH₂—C(O)NH—(CH₂)₃—; *—CH₂—C(O)NH—(CH₂)₅—; *—(CH₂)₂-phenylene-(CH₂)₁—; *—(CH₂)₁-phenylene-(CH₂)₂—; *—(CH₂)₁-phenylene-(CH₂)₃—; *—(CH₂)₃-phenylene-(CH₂)₁—; *—(CH₂)₄-phenylene-(CH₂)₁—; *—(CH₂)₁-phenylene-(CH₂)₄—; *—(CH₂)₂-phenylene-(CH₂)₂—; *—CH₂-phenylene-CH₂—NH—CH(CH₃)—; *—CH₂-phenylene-(CH₂)₂—NH—CH(CH₃)—; *—CH₂-phenylene-CH₂—NH—CH₂—; *—CH₂-phenylene-(CH₂)₂—NH—CH₂—; *—(CH₂)₁—C(O)NH—(CH₂)₄—; *—(CH₂)₁-furanylene-(CH₂)₁—; *—(CH₂)₁-furanylene-(CH₂)₂—; *—(CH₂)₁-furanylene-(CH₂)₃—; *—(CH₂)₂-furanylene-(CH₂)₁—; *—(CH₂)₂-furanylene-(CH₂)₂—; *—(CH₂)₃-furanylene-(CH₂)₁—; *—(CH₂)₃-furanylene-(CH₂)₂—; *—(CH₂)₁-thiazolylene-(CH₂)₁—; *—(CH₂)₁-thiazolylene-(CH₂)₂—; *—(CH₂)₁-thiazolylene-(CH₂)₃—; *—(CH₂)₂-thiazolylene-(CH₂)₁—; *—(CH₂)₂-thiazolylene-(CH₂)₂—; *—(CH₂)₃-thiazolylene-(CH₂)₁—; *—(CH₂)₃-thiazolylene-(CH₂)₂—; or *—CH₂-thiazolylene-CH₂—NH—CH₂—;
wherein a hydrogen of one or more CH₂ of the groups is optionally replaced with a substituent selected from the group consisting of: halogen, cyano, hydroxy, amino, mercapto, C₁₋₃alkyl, C₁₋₃ alkoxy, trifluoromethyl, C₅₋₁₀ aryl, 5- to 10-membered heteroaryl, C₃₋₁₂cycloalkyl, 3- to 12-membered heterocyclyl, or any combination thereof;
the piperazinylene is optionally substituted with a substituent selected from the group consisting of halogen, cyano, hydroxy, amino, mercapto, oxo, C₁₋₃ alkoxy, halogenated C₁₋₃alkyl, methanesulfonyloxy, trifluoromethanesulfonyloxy, p-toluenesulfonyloxy, optionally deuterated C₁₋₆ alkyl, optionally deuterated C₃₋₆ cycloalkyl, or any combination thereof,
the phenylene, the furanylene, and the thiazolylene are each independently optionally substituted with a substituent selected from the group consisting of halogen, cyano, hydroxy, amino, mercapto, C₁₋₃ alkoxy, halogenated C₁₋₃alkyl, methanesulfonyloxy, trifluoromethanesulfonyloxy, p-toluenesulfonyloxy, optionally deuterated C₁₋₆ alkyl, optionally deuterated C₃₋₆ cycloalkyl, or any combination thereof; and
symbol * indicates the point of attachment to R.

12. The compound of Formula (I) or salts, enantiomers, diastereoisomer, isotopically enriched analogs, solvates, prodrugs, or polymorphs thereof of claim 10, wherein L represents the following optionally substituted groups: 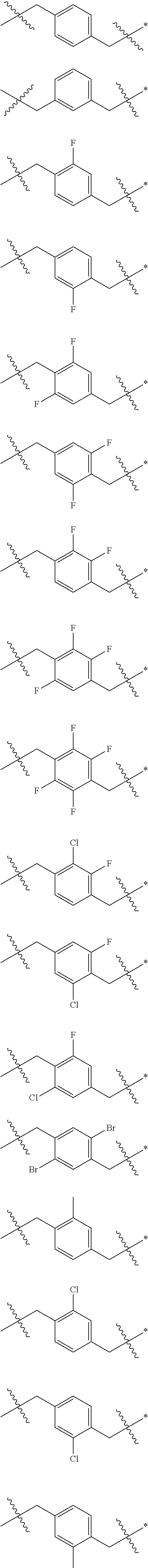 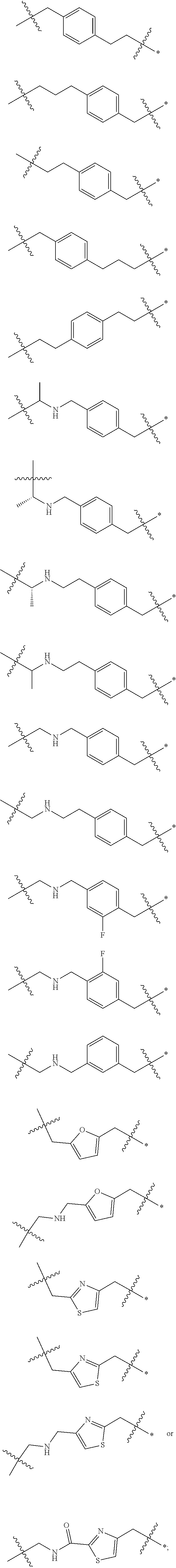
wherein * indicates the point of attachment to R.

13. The compound of Formula (I) or salts, enantiomers, diastereoisomer, isotopically enriched analogs, solvates, prodrugs, or polymorphs thereof of claim 1, which is selected from:
3-(5-fluoro-4-((8-morpholinooctyl)thio)-1-oxoisoindolin-2-yl)piperidine-2,6-dione;
3-(6-fluoro-4-((8-morpholinooctyl)thio)-1-oxoisoindolin-2-yl)piperidine-2,6-dione;
3-(7-fluoro-4-((8-morpholinooctyl)thio)-1-oxoisoindolin-2-yl)piperidine-2,6-dione;
3-(4-((8-(diethylamino)octyl)thio)-5-fluoro-1-oxoisoindolin-2-yl)piperidine-2,6-dione;
3-(4-((8-(diethylamino)octyl)thio)-6-fluoro-1-oxoisoindolin-2-yl)piperidine-2,6-dione;
3-(4-((8-(diethylamino)octyl)thio)-7-fluoro-1-oxoisoindolin-2-yl)piperidine-2,6-dione;
3-(5-fluoro-1-oxo-4-((7-(piperidin-1-yl)heptyl)thio)isoindolin-2-yl)piperidine-2,6-dione;
3-(6-fluoro-1-oxo-4-((7-(piperidin-1-yl)heptyl)thio)isoindolin-2-yl)piperidine-2,6-dione;
3-(7-fluoro-1-oxo-4-((7-(piperidin-1-yl)heptyl)thio)isoindolin-2-yl)piperidine-2,6-dione;
3-(4-((7-(4,4-difluoropiperidin-1-yl)heptyl)thio)-1-oxoisoindolin-2-yl)piperidine-2,6-dione;
3-(4-((7-(4,4-difluoropiperidin-1-yl)heptyl)thio)-5-fluoro-1-oxoisoindolin-2-yl)piperidine-2,6-dione;
3-(4-((7-(4,4-difluoropiperidin-1-yl)heptyl)thio)-6-fluoro-1-oxoisoindolin-2-yl)piperidine-2,6-dione;
3-(4-((7-(4,4-difluoropiperidin-1-yl)heptyl)thio)-7-fluoro-1-oxoisoindolin-2-yl)piperidine-2,6-dione;
3-(5-fluoro-1-oxo-4-((7-oxo-7-(piperidin-1-yl)heptyl)thio)isoindolin-2-yl)piperidine-2,6-dione;
3-(6-fluoro-1-oxo-4-((7-oxo-7-(piperidin-1-yl)heptyl)thio)isoindolin-2-yl)piperidine-2,6-dione;
3-(7-fluoro-1-oxo-4-((7-oxo-7-(piperidin-1-yl)heptyl)thio)isoindolin-2-yl)piperidine-2,6-dione;
3-(4-((7-(4,4-difluoropiperidin-1-yl)-7-oxoheptyl)thio)-5-fluoro-1-oxoisoindolin-2-yl)piperidine-2,6-dione;
3-(4-((7-(4,4-difluoropiperidin-1-yl)-7-oxoheptyl)thio)-6-fluoro-1-oxoisoindolin-2-yl)piperidine-2,6-dione;
3-(4-((7-(4,4-difluoropiperidin-1-yl)-7-oxoheptyl)thio)-7-fluoro-1-oxoisoindolin-2-yl)piperidine-2,6-dione;
7-((2-(2,6-dioxopiperidin-3-yl)-5-fluoro-1-oxoisoindolin-4-yl)thio)-N,N-diisopropylheptanamide;
7-((2-(2,6-dioxopiperidin-3-yl)-6-fluoro-1-oxoisoindolin-4-yl)thio)-N,N-diisopropylheptanamide;
7-((2-(2,6-dioxopiperidin-3-yl)-7-fluoro-1-oxoisoindolin-4-yl)thio)-N,N-diisopropylheptanamide;
3-(4-((7-((adamantan-1-yl)amino)heptyl)thio)-5-fluoro-1-oxoisoindolin-2-yl)piperidine-2,6-dione;
3-(4-((4-((adamantan-1-yl)amino)butyl)thio)-6-fluoro-1-oxoisoindolin-2-yl)piperidine-2,6-dione;
3-(4-((4-((adamantan-1-yl)amino)butyl)thio)-5-fluoro-1-oxoisoindolin-2-yl)piperidine-2,6-dione;
3-(4-((4-((adamantan-1-yl)amino)butyl)thio)-7-fluoro-1-oxoisoindolin-2-yl)piperidine-2,6-dione;
3-(4-((7-((adamantan-1-yl)amino)heptyl)thio)-6-fluoro-1-oxoisoindolin-2-yl)piperidine-2,6-dione;
3-(4-((7-((adamantan-1-yl)amino)heptyl)thio)-7-fluoro-1-oxoisoindolin-2-yl)piperidine-2,6-dione;
3-(5-fluoro-1-oxo-4-((4-(piperidin-1-ylmethyl)benzyl)thio)isoindolin-2-yl)piperidine-2,6-dione;
3-(6-fluoro-1-oxo-4-((4-(piperidin-1-ylmethyl)benzyl)thio)isoindolin-2-yl)piperidine-2,6-dione;
3-(7-fluoro-1-oxo-4-((4-(piperidin-1-ylmethyl)benzyl)thio)isoindolin-2-yl)piperidine-2,6-dione;
3-(4-((4-((4,4-difluoropiperidin-1-yl)methyl)benzyl)thio)-5-fluoro-1-oxoisoindolin-2-yl)piperidine-2,6-dione;
3-(4-((4-((4,4-difluoropiperidin-1-yl)methyl)benzyl)thio)-6-fluoro-1-oxoisoindolin-2-yl)piperidine-2,6-dione;
3-(4-((4-((4,4-difluoropiperidin-1-yl)methyl)benzyl)thio)-7-fluoro-1-oxoisoindolin-2-yl)piperidine-2,6-dione;
3-(4-((7-(cyclohexylamino)heptyl)thio)-5-fluoro-1-oxoisoindolin-2-yl)piperidine-2,6-dione;
3-(4-((7-(cyclohexylamino)heptyl)thio)-6-fluoro-1-oxoisoindolin-2-yl)piperidine-2,6-dione;
3-(4-((7-(cyclohexylamino)heptyl)thio)-7-fluoro-1-oxoisoindolin-2-yl)piperidine-2,6-dione;
3-(4-((7-((adamantan-1-yl)(methyl)amino)heptyl)thio)-5-fluoro-1-oxoisoindolin-2-yl)piperidine-2,6-dione;
3-(4-((7-((adamantan-1-yl)(methyl)amino)heptyl)thio)-6-fluoro-1-oxoisoindolin-2-yl)piperidine-2,6-dione;
3-(4-((7-((adamantan-1-yl)(methyl)amino)heptyl)thio)-7-fluoro-1-oxoisoindolin-2-yl)piperidine-2,6-dione;
3-(5-fluoro-1-oxo-4-((7-(((1R,2S,4R)-1,7,7-trimethylbicyclo[2.2.1]heptan-2-yl)amino)heptyl)thio)isoindolin-2-yl)piperidine-2,6-dione;
3-(6-fluoro-1-oxo-4-((7-(((1R,2S,4R)-1,7,7-trimethylbicyclo[2.2.1]heptan-2-yl)amino)heptyl)thio)isoindolin-2-yl)piperidine-2,6-dione;
3-(7-fluoro-1-oxo-4-((7-(((1R,2S,4R)-1,7,7-trimethylbicyclo[2.2.1]heptan-2-yl)amino)heptyl)thio)isoindolin-2-yl)piperidine-2,6-dione;
3-(5-fluoro-1-oxo-4-((4-((((1R,2S,4R)-1,7,7-trimethylbicyclo[2.2.1]heptan-2-yl)amino)methyl)benzyl)thio)isoindolin-2-yl)piperidine-2,6-dione;
3-(6-fluoro-1-oxo-4-((4-((((1R,2S,4R)-1,7,7-trimethylbicyclo[2.2.1]heptan-2-yl)amino)methyl)benzyl)thio)isoindolin-2-yl)piperidine-2,6-dione;
3-(7-fluoro-1-oxo-4-((4-((((1R,2S,4R)-1,7,7-trimethylbicyclo[2.2.1]heptan-2-yl)amino)methyl)benzyl)thio)isoindolin-2-yl)piperidine-2,6-dione;
3-(4-((4-(((adamantan-1-yl)amino)methyl)benzyl)thio)-5-fluoro-1-oxoisoindolin-2-yl)piperidine-2,6-dione;
3-(4-((4-(((adamantan-1-yl)amino)methyl)benzyl)thio)-6-fluoro-1-oxoisoindolin-2-yl)piperidine-2,6-dione;
3-(4-((4-(((adamantan-1-yl)amino)methyl)benzyl)thio)-7-fluoro-1-oxoisoindolin-2-yl)piperidine-2,6-dione;
3-(5-fluoro-1-oxo-4-((7-(spiro[3.3]heptan-2-ylamino)heptyl)thio)isoindolin-2-yl)piperidine-2,6-dione;
3-(6-fluoro-1-oxo-4-((7-(spiro[3.3]heptan-2-ylamino)heptyl)thio)isoindolin-2-yl)piperidine-2,6-dione;
3-(7-fluoro-1-oxo-4-((7-(spiro[3.3]heptan-2-ylamino)heptyl)thio)isoindolin-2-yl)piperidine-2,6-dione;
3-(4-(8-((adamantan-1-yl)amino)octyl)-5-fluoro-1-oxoisoindolin-2-yl)piperidine-2,6-dione;
3-(4-(8-((adamantan-1-yl)amino)octyl)-6-fluoro-1-oxoisoindolin-2-yl)piperidine-2,6-dione;
3-(4-(8-((adamantan-1-yl)amino)octyl)-7-fluoro-1-oxoisoindolin-2-yl)piperidine-2,6-dione;
3-(4-((7-((yl)amino)heptyl)oxy)-5-fluoro-1-oxoisoindolin-2-yl)piperidine-2,6-dione;
3-(4-((7-((adamantan-1-yl)amino)heptyl)oxy)-6-fluoro-1-oxoisoindolin-2-yl)piperidine-2,6-dione;
3-(4-((7-((adamantan-1-yl)amino)heptyl)oxy)-7-fluoro-1-oxoisoindolin-2-yl)piperidine-2,6-dione;
3-(4-((7-((adamantan-1-yl)amino)heptyl)amino)-5-fluoro-1-oxoisoindolin-2-yl)piperidine-2,6-dione;
3-(4-((7-((adamantan-1-yl)amino)heptyl)amino)-6-fluoro-1-oxoisoindolin-2-yl)piperidine-2,6-dione;
3-(4-((7-((adamantan-1-yl)amino)heptyl)amino)-7-fluoro-1-oxoisoindolin-2-yl)piperidine-2,6-dione;
3-(5-fluoro-4-((3-fluoro-4-(piperidin-1-ylmethyl)benzyl)thio)-1-oxoisoindolin-2-yl)piperidine-2,6-dione;
3-(6-fluoro-4-((3-fluoro-4-(piperidin-1-ylmethyl)benzyl)thio)-1-oxoisoindolin-2-yl)piperidine-2,6-dione;
3-(7-fluoro-4-((3-fluoro-4-(piperidin-1-ylmethyl)benzyl)thio)-1-oxoisoindolin-2-yl)piperidine-2,6-dione;
3-(4-((4-((4,4-difluoropiperidin-1-yl)methyl)-3-fluorobenzyl)thio)-1-oxoisoindolin-2-yl)piperidine-2,6-dione;
3-(4-((4-((4,4-difluoropiperidin-1-yl)methyl)-3-fluorobenzyl)thio)-5-fluoro-1-oxoisoindolin-2-yl)piperidine-2,6-dione;
3-(4-((4-((4,4-difluoropiperidin-1-yl)methyl)-3-fluorobenzyl)thio)-6-fluoro-1-oxoisoindolin-2-yl)piperidine-2,6-dione;
3-(4-((4-((4,4-difluoropiperidin-1-yl)methyl)-3-fluorobenzyl)thio)-7-fluoro-1-oxoisoindolin-2-yl)piperidine-2,6-dione;
3-(4-((4-(2-((adamantan-1-yl)amino)ethyl)benzyl)thio)-5-fluoro-1-oxoisoindolin-2-yl)piperidine-2,6-dione;
3-(4-((4-(2-((adamantan-1-yl)amino)ethyl)benzyl)thio)-6-fluoro-1-oxoisoindolin-2-yl)piperidine-2,6-dione;
3-(4-((4-(2-((adamantan-1-yl)amino)ethyl)benzyl)thio)-7-fluoro-1-oxoisoindolin-2-yl)piperidine-2,6-dione;
3-(4-(((4-(((adamantan-1-yl)amino)methyl)thiazol-2-yl)methyl)thio)-5-fluoro-1-oxoisoindolin-2-yl)piperidine-2,6-dione;
3-(4-(((4-(((adamantan-1-yl)amino)methyl)thiazol-2-yl)methyl)thio)-6-fluoro-1-oxoisoindolin-2-yl)piperidine-2,6-dione;
3-(4-(((4-(((adamantan-1-yl)amino)methyl)thiazol-2-yl)methyl)thio)-7-fluoro-1-oxoisoindolin-2-yl)piperidine-2,6-dione;
2-(2,6-dioxopiperidin-3-yl)-5-fluoro-4-((8-morpholinooctyl)thio)isoindoline-1,3-dione;
2-(2,6-dioxopiperidin-3-yl)-6-fluoro-4-((8-morpholinooctyl)thio)isoindoline-1,3-dione;
2-(2,6-dioxopiperidin-3-yl)-4-fluoro-7-((8-morpholinooctyl)thio)isoindoline-1,3-dione;
4-((8-(diethylamino)octyl)thio)-2-(2,6-dioxopiperidin-3-yl)-5-fluoroisoindoline-1,3-dione;
4-((8-(diethylamino)octyl)thio)-2-(2,6-dioxopiperidin-3-yl)-6-fluoroisoindoline-1,3-dione;
4-((8-(diethylamino)octyl)thio)-2-(2,6-dioxopiperidin-3-yl)-7-fluoroisoindoline-1,3-dione;
2-(2,6-dioxopiperidin-3-yl)-5-fluoro-4-((7-(piperidin-1-yl)heptyl)thio)isoindoline-1,3-dione;
2-(2,6-dioxopiperidin-3-yl)-6-fluoro-4-((7-(piperidin-1-yl)heptyl)thio)isoindoline-1,3-dione;
2-(2,6-dioxopiperidin-3-yl)-4-fluoro-7-((7-(piperidin-1-yl)heptyl)thio)isoindoline-1,3-dione;
4-((7-(4,4-difluoropiperidin-1-yl)heptyl)thio)-2-(2,6-dioxopiperidin-3-yl)isoindoline-1,3-dione;
4-((7-(4,4-difluoropiperidin-1-yl)heptyl)thio)-2-(2,6-dioxopiperidin-3-yl)-5-fluoroisoindoline-1,3-dione;
4-((7-(4,4-difluoropiperidin-1-yl)heptyl)thio)-2-(2,6-dioxopiperidin-3-yl)-6-fluoroisoindoline-1,3-dione;
4-((7-(4,4-difluoropiperidin-1-yl)heptyl)thio)-2-(2,6-dioxopiperidin-3-yl)-7-fluoroisoindoline-1,3-dione;
2-(2,6-dioxopiperidin-3-yl)-5-fluoro-4-((7-oxo-7-(piperidin-1-yl)heptyl)thio)isoindoline-1,3-dione;
2-(2,6-dioxopiperidin-3-yl)-6-fluoro-4-((7-oxo-7-(piperidin-1-yl)heptyl)thio)isoindoline-1,3-dione;
2-(2,6-dioxopiperidin-3-yl)-4-fluoro-7-((7-oxo-7-(piperidin-1-yl)heptyl)thio)isoindoline-1,3-dione;
4-((7-(4,4-difluoropiperidin-1-yl)-7-oxoheptyl)thio)-2-(2,6-dioxopiperidin-3-yl)-5-fluoroisoindoline-1,3-dione;
4-((7-(4,4-difluoropiperidin-1-yl)-7-oxoheptyl)thio)-2-(2,6-dioxopiperidin-3-yl)-6-fluoroisoindoline-1,3-dione;
4-((7-(4,4-difluoropiperidin-1-yl)-7-oxoheptyl)thio)-2-(2,6-dioxopiperidin-3-yl)-7-fluoroisoindoline-1,3-dione;
7-((2-(2,6-dioxopiperidin-3-yl)-5-fluoro-1,3-dioxoisoindolin-4-yl)thio)-N,N-diisopropylheptanamide;
7-((2-(2,6-dioxopiperidin-3-yl)-6-fluoro-1,3-dioxoisoindolin-4-yl)thio)-N,N-diisopropylheptanamide;
7-((2-(2,6-dioxopiperidin-3-yl)-7-fluoro-1,3-dioxoisoindolin-4-yl)thio)-N,N-diisopropylheptanamide;
4-((7-((adamantan-1-yl)amino)heptyl)thio)-2-(2,6-dioxopiperidin-3-yl)-5-fluoroisoindoline-1,3-dione;
4-((7-((adamantan-1-yl)amino)heptyl)thio)-2-(2,6-dioxopiperidin-3-yl)-6-fluoroisoindoline-1,3-dione;
4-((7-((adamantan-1-yl)amino)heptyl)thio)-2-(2,6-dioxopiperidin-3-yl)-7-fluoroisoindoline-1,3-dione;
2-(2,6-dioxopiperidin-3-yl)-5-fluoro-4-((4-(piperidin-1-ylmethyl)benzyl)thio)isoindoline-1,3-dione;
2-(2,6-dioxopiperidin-3-yl)-6-fluoro-4-((4-(piperidin-1-ylmethyl)benzyl)thio)isoindoline-1,3-dione;
2-(2,6-dioxopiperidin-3-yl)-4-fluoro-7-((4-(piperidin-1-ylmethyl)benzyl)thio)isoindoline-1,3-dione;
4-((4-((4,4-difluoropiperidin-1-yl)methyl)benzyl)thio)-2-(2,6-dioxopiperidin-3-yl)isoindoline-1,3-dione;
4-((4-((4,4-difluoropiperidin-1-yl)methyl)benzyl)thio)-2-(2,6-dioxopiperidin-3-yl)-5-fluoroisoindoline-1,3-dione;
4-((4-((4,4-difluoropiperidin-1-yl)methyl)benzyl)thio)-2-(2,6-dioxopiperidin-3-yl)-6-fluoroisoindoline-1,3-dione;
4-((4-((4,4-difluoropiperidin-1-yl)methyl)benzyl)thio)-2-(2,6-dioxopiperidin-3-yl)-7-fluoroisoindoline-1,3-dione;
4-((7-(cyclohexylamino)heptyl)thio)-2-(2,6-dioxopiperidin-3-yl)-5-fluoroisoindoline-1,3-dione;
4-((7-(cyclohexylamino)heptyl)thio)-2-(2,6-dioxopiperidin-3-yl)-6-fluoroisoindoline-1,3-dione;
4-((7-(cyclohexylamino)heptyl)thio)-2-(2,6-dioxopiperidin-3-yl)-7-fluoroisoindoline-1,3-dione;
4-((7-((adamantan-1-yl)(methyl)amino)heptyl)thio)-2-(2,6-dioxopiperidin-3-yl)-5-fluoroisoindoline-1,3-dione;
4-((7-((adamantan-1-yl)(methyl)amino)heptyl)thio)-2-(2,6-dioxopiperidin-3-yl)-6-fluoroisoindoline-1,3-dione;
4-((7-((adamantan-1-yl)(methyl)amino)heptyl)thio)-2-(2,6-dioxopiperidin-3-yl)-7-fluoroisoindoline-1,3-dione;
2-(2,6-dioxopiperidin-3-yl)-5-fluoro-4-((7-(((1R,2S,4R)-1,7,7-trimethylbicyclo[2.2.1]heptan-2-yl)amino)heptyl)thio)isoindoline-1,3-dione;
2-(2,6-dioxopiperidin-3-yl)-6-fluoro-4-((7-(((1R,2S,4R)-1,7,7-trimethylbicyclo[2.2.1]heptan-2-yl)amino)heptyl)thio)isoindoline-1,3-dione;
2-(2,6-dioxopiperidin-3-yl)-4-fluoro-7-((7-(((1R,2S,4R)-1,7,7-trimethylbicyclo[2.2.1]heptan-2-yl)amino)heptyl)thio)isoindoline-1,3-dione;
2-(2,6-dioxopiperidin-3-yl)-5-fluoro-4-((4-((((1R,2S,4R)-1,7,7-trimethylbicyclo[2.2.1]heptan-2-yl)amino)methyl)benzyl)thio)isoindoline-1,3-dione;
2-(2,6-dioxopiperidin-3-yl)-6-fluoro-4-((4-((((1R,2S,4R)-1,7,7-trimethylbicyclo[2.2.1]heptan-2-yl)amino)methyl)benzyl)thio)isoindoline-1,3-dione;
2-(2,6-dioxopiperidin-3-yl)-4-fluoro-7-((4-((((1R,2S,4R)-1,7,7-trimethylbicyclo[2.2.1]heptan-2-yl)amino)methyl)benzyl)thio)isoindoline-1,3-dione;
4-((4-(((adamantan-1-yl)amino)methyl)benzyl)thio)-2-(2,6-dioxopiperidin-3-yl)-5-fluoroisoindoline-1,3-dione;
4-((4-(((adamantan-1-yl)amino)methyl)benzyl)thio)-2-(2,6-dioxopiperidin-3-yl)-6-fluoroisoindoline-1,3-dione;
4-((4-(((adamantan-1-yl)amino)methyl)benzyl)thio)-2-(2,6-dioxopiperidin-3-yl)-7-fluoroisoindoline-1,3-dione;
2-(2,6-dioxopiperidin-3-yl)-5-fluoro-4-((7-(spiro[3.3]heptan-2-ylamino)heptyl)thio)isoindoline-1,3-dione;
2-(2,6-dioxopiperidin-3-yl)-6-fluoro-4-((7-(spiro[3.3]heptan-2-ylamino)heptyl)thio)isoindoline-1,3-dione;
2-(2,6-dioxopiperidin-3-yl)-4-fluoro-7-((7-(spiro[3.3]heptan-2-ylamino)heptyl)thio)isoindoline-1,3-dione;
4-(8-((adamantan-1-yl)amino)octyl)-2-(2,6-dioxopiperidin-3-yl)-5-fluoroisoindoline-1,3-dione;
4-(8-((adamantan-1-yl)amino)octyl)-2-(2,6-dioxopiperidin-3-yl)-6-fluoroisoindoline-1,3-dione;
4-(8-((adamantan-1-yl)amino)octyl)-2-(2,6-dioxopiperidin-3-yl)-7-fluoroisoindoline-1,3-dione;
4-((7-((adamantan-1-yl)amino)heptyl)oxy)-2-(2,6-dioxopiperidin-3-yl)-5-fluoroisoindoline-1,3-dione;
4-((7-((adamantan-1-yl)amino)heptyl)oxy)-2-(2,6-dioxopiperidin-3-yl)-6-fluoroisoindoline-1,3-dione;
4-((7-((adamantan-1-yl)amino)heptyl)oxy)-2-(2,6-dioxopiperidin-3-yl)-7-fluoroisoindoline-1,3-dione;
4-((7-((adamantan-1-yl)amino)heptyl)amino)-2-(2,6-dioxopiperidin-3-yl)-5-fluoroisoindoline-1,3-dione;
4-((7-((adamantan-1-yl)amino)heptyl)amino)-2-(2,6-dioxopiperidin-3-yl)-6-fluoroisoindoline-1,3-dione;
4-((7-((adamantan-1-yl)amino)heptyl)amino)-2-(2,6-dioxopiperidin-3-yl)-7-fluoroisoindoline-1,3-dione;
2-(2,6-dioxopiperidin-3-yl)-5-fluoro-4-((3-fluoro-4-(piperidin-1-ylmethyl)benzyl)thio)isoindoline-1,3-dione;
2-(2,6-dioxopiperidin-3-yl)-6-fluoro-4-((3-fluoro-4-(piperidin-1-ylmethyl)benzyl)thio)isoindoline-1,3-dione;
2-(2,6-dioxopiperidin-3-yl)-4-fluoro-7-((3-fluoro-4-(piperidin-1-ylmethyl)benzyl)thio)isoindoline-1,3-dione;
4-((4-((4,4-difluoropiperidin-1-yl)methyl)-3-fluorobenzyl)thio)-2-(2,6-dioxopiperidin-3-yl)isoindoline-1,3-dione;
4-((4-((4,4-difluoropiperidin-1-yl)methyl)-3-fluorobenzyl)thio)-2-(2,6-dioxopiperidin-3-yl)-5-fluoroisoindoline-1,3-dione;
4-((4-((4,4-difluoropiperidin-1-yl)methyl)-3-fluorobenzyl)thio)-2-(2,6-dioxopiperidin-3-yl)-6-fluoroisoindoline-1,3-dione;
4-((4-((4,4-difluoropiperidin-1-yl)methyl)-3-fluorobenzyl)thio)-2-(2,6-dioxopiperidin-3-yl)-7-fluoroisoindoline-1,3-dione;
4-((4-(2-((adamantan-1-yl)amino)ethyl)benzyl)thio)-2-(2,6-dioxopiperidin-3-yl)-5-fluoroisoindoline-1,3-dione;
4-((4-(2-((adamantan-1-yl)amino)ethyl)benzyl)thio)-2-(2,6-dioxopiperidin-3-yl)-6-fluoroisoindoline-1,3-dione;
4-((4-(2-((adamantan-1-yl)amino)ethyl)benzyl)thio)-2-(2,6-dioxopiperidin-3-yl)-7-fluoroisoindoline-1,3-dione;
4-(((4-(((adamantan-1-yl)amino)methyl)thiazol-2-yl)methyl)thio)-2-(2,6-dioxopiperidin-3-yl)-5-fluoroisoindoline-1,3-dione;
4-(((4-(((adamantan-1-yl)amino)methyl)thiazol-2-yl)methyl)thio)-2-(2,6-dioxopiperidin-3-yl)-6-fluoroisoindoline-1,3-dione; and
4-(((4-(((adamantan-1-yl)amino)methyl)thiazol-2-yl)methyl)thio)-2-(2,6-dioxopiperidin-3-yl)-7-fluoroisoindoline-1,3-dione.

14. A pharmaceutical composition comprising the compound of Formula (I) of claim 1 or a pharmaceutically acceptable salt thereof, and at least one pharmaceutically acceptable carrier;
optionally comprising at least one additional therapeutic agent.

15. The pharmaceutical composition of claim 14, wherein said at least one additional therapeutic agent is used for treating or preventing cancer or tumor.

16. The compound of Formula (I) of claim 1 or a pharmaceutically acceptable salt thereof, for use as a medicament.

17. A method for treating or preventing a disease or disorder associated with cereblon protein in a subject, comprising administering to the subject a therapeutically effective amount of the compound of Formula (I) of claim 1 or a pharmaceutically acceptable salt thereof, or a pharmaceutical composition comprising the compound of Formula (I) or pharmaceutically acceptable salts thereof.

18. The method of claim 17, wherein the disease or disorder associated with cereblon protein is selected from the group consisting of: tumor, infectious disease, inflammatory disease, autoimmune disease, anemia, hemorrhagic shock, transplant rejection, multiple organ dysfunction syndrome (MODS), sarcoidosis, adult respiratory distress syndrome, cardiovascular disease, Richter syndrome (RS), acute liver failure, or diabetes.

19. The method of claim 17, wherein the disease or disorder associated with cereblon protein is selected from the group consisting of: myeloma, including multiple myeloma, plasma cell myeloma, smoldering myeloma, smoldering multiple myeloma; myelofibrosis; bone marrow disease; myelodysplastic syndrome (MDS); previously treated myelodysplastic syndrome; transplantation-related cancer; neutropenia; leukemia, including acute myeloid leukemia, chronic myelogenous leukemia, B-cell chronic lymphocytic leukemia, leukemia-associated anemia, acute myeloid leukemia (AML); lymphoma, including diffuse large B-cell lymphoma, non-Hodgkin's lymphoma, Hodgkin's lymphoma, anaplastic lymphoma, anaplastic large cell lymphoma, CD20 positive lymphoma, mantle cell lymphoma, primary lymphoma, B-cell lymphoma, recurrent B-cell non-Hodgkin's lymphoma, recurrent diffuse large B-cell lymphoma, recurrent mediastinal (thymic) large B-cell lymphoma, primary mediastinal (thymic) large B-cell lymphoma, recurrent transformed non-Hodgkin's lymphoma, refractory B-cell non-Hodgkin's lymphoma, refractory diffuse large B-cell lymphoma, refractory primary mediastinal (thymic) large B-cell lymphoma, refractory transformed non-Hodgkin's lymphoma; Burkitt's lymphoma; thyroid cancer; melanoma; lung cancer, including lung adenocarcinoma, lung squamous cell carcinoma; inflammatory myofibroblastoma; colorectal cancer; intestinal cancer; brain glioma; astroblastoma; ovarian cancer; bronchial cancer; prostate cancer; breast cancer, including triple negative breast cancer, sporadic breast cancer and patients with Cowden syndrome; pancreatic cancer; central nervous system tumor; neuroblastoma; neuroglioma; Peripheral neuroepithelioma; extramedullary plasmacytoma; plasmacytoma; gastric cancer; gastrointestinal stromal tumors; esophageal cancer; colorectal adenocarcinoma; esophageal squamous cell carcinoma; liver cancer; renal cell carcinoma; bladder cancer; endometrial cancer; metrocarcinoma; head and neck cancer; brain cancer; oral cancer; sarcoma, including rhabdomyosarcoma, various lipogenic tumors, Ewing's sarcoma/primitive neuroectodermal tumors (Ewing/PNETs), and leiomyosarcoma; urothelial carcinoma; basal cell carcinoma; oral squamous cell carcinoma; cholangiocarcinoma; bone cancer; cervical cancer; skin cancer; Richter syndrome (RS); sepsis syndrome; autoimmune diseases, including rheumatoid arthritis, autoimmune encephalomyelitis, ankylosing spondylitis, psoriasis, systemic lupus erythematosus, multiple sclerosis, recurrent oral ulcers, Kawasaki disease, polymyositis/dermatomyositis, Sjogren's syndrome, and atopic dermatitis; keratoconjunctivitis; inflammatory diseases, including Crohn's disease and ulcerative colitis, pneumonia, osteoarthritis, synovitis, systemic inflammatory response syndrome, airway inflammation, bronchitis; cerebral malaria; infectious diseases, including viral pneumonia, Acquired immunodeficiency syndrome (AIDS), COVID-19 novel coronavirus infection, gram-negative bacteria infection, gram-positive bacteria infection, tuberculosis, etc.; septic shock; tuberculosis; bacterial meningitis; chronic obstructive pulmonary disease; asthma; hemorrhagic shock; organ (including kidney, heart, lung) or tissue transplantation rejection; diabetes; sarcoidosis; adult respiratory distress syndrome; anemia; pediatric aplastic anemia; cardiovascular diseases (e.g., coronary heart disease, congestive heart failure, myocardial infarction, atherosclerosis); multiple organ dysfunction caused by cachexia and septic shock; or acute liver failure.
